(19) United States
(12) Patent Application Publication
(10) Pub. No.: US 2022/0133808 A1
(43) Pub. Date: May 5, 2022

(54) COMPOSITIONS AND METHODS FOR TREATING NEUROCOGNITIVE DISORDERS

(71) Applicant: AvroBio, Inc., Cambridge, MA (US)

(72) Inventors: Chris MASON, Cambridge, MA (US); Oliver COOPER, Jamaica Plain, MA (US); Mark DEANDRADE, Boston, MA (US); Robert PLASSCHAERT, Cambridge, MA (US); Nico Peter (Niek) VAN TIL, Leiden (NL)

(21) Appl. No.: 17/427,252

(22) PCT Filed: Jan. 31, 2020
(86) PCT No.: PCT/US2020/016206

§ 371 Date: Jul. 30, 2021

Related U.S. Application Data
(60) Provisional application No. 62/800,177, filed on Feb. 1, 2019.

(51) Int. Cl. A61K 35/545; A61K 38/17; A61K 35/15; C12N 15/86; A61P 25/16; A61P 25/28; A61K 35/28

(52) U.S. Cl. CPC A61K 35/545; A61K 38/17; A61K 35/15; A61K 35/28; A61P 25/16; A61P 25/28; C12N 15/86

(57) ABSTRACT

Described herein are compositions and methods for treating a patient having or at risk of developing a neurocognitive disorder, such as Alzheimer's disease, Parkinson's disease, and/or a frontotemporal lobar dementia. Using the compositions and methods of the disclosure, a patient, such as an adult human patient, may be provided one or more agents that elevate the expression and/or activity levels of a protein or series of proteins whose deficiency is associated with the corresponding disease. Exemplary agents that may be used in conjunction with the compositions and methods of the disclosure for this purpose include cells, such as cells, that contain nucleic acids encoding the protein or proteins of interest, as well as vectors, such as viral vectors, encoding the protein or proteins of interest. Additional examples of such agents include the protein or proteins themselves, as well as interfering RNA molecules that stimulate their endogenous expression.

### SEQUENCE LISTING

The instant application contains a Sequence Listing which has been submitted electronically in ASCII format and is hereby incorporated by reference in its entirety. Said ASCII copy, created on Jan. 29, 2020 is named “51182-020WO2_Sequence_Listing_1.29.20_ST25” and is 605,736 bytes in size.

### FIELD OF THE INVENTION

The disclosure relates to compositions and methods for treating various neurocognitive disorders, such as Alzheimer's disease, Parkinson's disease, and frontotemporal lobar dementia.

### BACKGROUND

To date, the treatment of neurocognitive disorders has posed a challenge to the medical community. Examples of these disorders include Alzheimer's disease, Parkinson's disease, and frontotemporal lobar dementia. Alzheimer's disease is a late-onset neurodegenerative disorder responsible for the majority of dementia cases in the elderly. Alzheimer's disease patients suffer from a progressive cognitive decline characterized by symptoms including an insidious loss of short- and long-term memory, attention deficits, language-specific problems, disorientation, impulse control, social withdrawal, anhedonia, and other symptoms. Current treatments for this indication strive to ameliorate disease symptomology, but therapies targeting the underlying neurodegeneration are lacking. Similarly, treatments for Parkinson's disease, a progressive disorder of the nervous system that affects movement and produces symptoms such as resting tremor, rigidity, and bradykinesia, primarily focus on increasing dopamine levels, underscoring the need for therapies that target the underlying biochemical etiology. Additionally, treatments for frontotemporal lobar degeneration, a neurodegenerative disorder characterized by a complex clinical presentation that may include deficits in speech comprehension and production, poor motor planning and coordination, and/or loss of executive function characterized by lack of impulse control and a preference for perseverative behaviors, strive to ameliorate disease symptomology. There remains a need for improved therapeutic modalities that target the underlying causes of these classes of diseases at the genomic and proteomic level.

### SUMMARY OF THE INVENTION

The present disclosure relates to compositions and methods for the treatment of a neurocognitive disorder (NCD), such as Alzheimer's disease, Parkinson disease, and frontotemporal lobar degeneration, in a patient, such as a human patient. Using the compositions and methods of the disclosure, a patient, such as an adult human patient suffering from an NCD described herein, may be provided an agent or a plurality of agents that, together, elevate the expression and/or activity of one or more proteins in the patient. The patient may be suffering, for example, from an NCD such as Alzheimer's disease, Parkinson's disease, or frontotemporal lobar degeneration (FTLD). The provision of such agents to the patient may serve to reverse the pathophysiology of the disease. Without being limited by mechanism, modulating a patient's gene expression and/or protein activity patterns using the compositions and methods of the disclosure may restore physiologically normal quantities and functionalities of proteins whose deficiencies are associated with the foregoing disorders, thereby treating underlying disease etiology. The compositions and methods described herein may thus be used not only to ameliorate one or more symptoms associated with an NCD but may also be used as curative therapeutics.

For example, using the compositions and methods described herein, a patient, such as an adult human patient, may be administered one or more agents that together function to elevate the level of expression and/or activity of a protein or a subset of proteins whose deficiencies are found to be associated with the onset of the pathology. Particularly, the compositions and methods of the disclosure may be used to provide a patient having an NCD (e.g., Alzheimer's disease) with one or more agents that together augment the expression and/or activity of one or more proteins selected from APP, PSEN1, PSEN2, APOE, TOMM40, GAB2, APOC1, TREM2, ABI3, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1 L, STK24, DISCI , MPZL1, SLC4A1AP, TRIP4, MSRA, HS3ST1, ZNF224, and AP2A2, such as one or more agents that together augment the expression and/or activity of one or more proteins selected from PSEN1, GAB2, APOC1, TREM2, ABI3, BIN1, HLA-DRB5, HLA-DRB1, CD2AP, PTK2B, INPP5D, MEF2C, CD33, MS4A4A, RIN3, PICALM, CASS4, SORL1, PLCG2, SCIMP, FRMD4A, SPPL2A, MTHFD1L, DISC1, TRIP4, and HS3ST1. The one or more agents may, for example, serve to elevate the expression and/or activity level of a subset of the foregoing proteins, such as a subset of two, three, four, five, six, seven, eight, nine, ten, or more, of these proteins.

Similarly, the compositions and methods of the disclosure may be used to provide a patient having an NCD (e.g., Parkinson's disease) with one or more agents that together augment the expression and/or activity of one or more proteins selected from FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1 L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1B, NUCKS1, and ACMSD, such as one or more agents that together augment the expression and/or activity of one or more proteins selected from FCGR2A, SCAF11, DNAJC13, GCH1, LRRK2, GBA, GAK, FGF20, HLA-DQB1, and NOD2. The one or more agents may, for example, serve to elevate the expression and/or activity level of a subset of the foregoing proteins, such as a subset of two, three, four, five, six, seven, eight, nine, ten, or more, of these proteins.

As another example, the compositions and methods of the disclosure may be used to provide a patient having an NCD (e.g., FTLD) with one or more agents that together augment the expression and/or activity of one or more proteins selected from HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT, such as one or more agents that together augment the expression and/or activity of one or more proteins selected from HLA-DRA, HLA-DRB5, C9ORF72, SQSTM1, TBK1, PSEN1, GRN, and CTSF. The one or more agents may, for example, serve to elevate the expression and/or activity level of a subset of the foregoing proteins, such as a subset of two, three, four, five, six, seven, eight, nine, ten, or more, of these proteins.

As yet another example, the compositions and methods of the disclosure may be used to provide a patient having an NCD (e.g., AD, PD, or FTLD) with one or more agents that together augment the expression and/or activity of one or more proteins selected from APP, PSEN1, PSEN2, APOE, TOMM40, GAB2, APOC1, TREM2, ABI3, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1L, STK24, DISCI , MPZL1, SLC4A1AP, TRIP4, MSRA, HS3ST1, ZNF224, AP2A2, FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1B, NUCKS1, ACMSD, HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT. The one or more agents may, for example, serve to elevate the expression and/or activity level of a subset of the foregoing proteins, such as a subset of two, three, four, five, six, seven, eight, nine, ten, or more, of these proteins.

Agents that elevate the expression and/or activity level of one or more proteins of interest and that may be used in conjunction with the compositions and methods of the disclosure include nucleic acids that encode the protein or plurality of proteins (e.g., such as, e.g., nucleic acids capable of expression in a macrophage or a microglial cell). Such nucleic acid molecules may be provided to a patient (e.g., a patient diagnosed with an NCD such as, e.g., Alzheimer's disease, Parkinson's disease, or FTLD) in the form, for example, of a population of cells, such as a population of cells, such as pluripotent cells (e.g., embryonic stem cells (ESCs) or induced pluripotent stem cells (ISPCs)), multipotent cells (e.g., CD34+ cells such as, e.g., hematopoietic stem cells (HSCs) or myeloid precursor cells (MPCs)), blood lineage progenitor cells (BLPCS; e.g., monocytes), macrophages, microglial progenitor cells, or microglia that contain the nucleic acid molecules. Such cells may contain the nucleic acid molecules of interest, for example, in episomal form or as an integrated component of the cellular genome. Additionally or alternatively, nucleic acid molecules encoding one or more of the proteins of interest may be provided to the patient in the form of one or more viral vectors that collectively encode the one or more proteins.

Exemplary viral vectors that may be used in conjunction with the compositions and methods of the disclosure include Retroviridae family viral vectors, such as a lentivirus, alpharetrovirus, or gammaretrovirus, among others described herein. In some embodiments, the nucleic acid molecule(s) are administered directly to the patient. Additional agents that may be provided to a patient for the purpose of augmenting the level of one or more proteins of interest include interfering RNA molecules, such as short interfering RNA (siRNA), short hairpin RNA (shRNA), and micro RNA (miRNA) molecules, as well as small molecule agents that modulate gene expression, in addition to the one or more proteins themselves.

The compositions and methods of the disclosure are based, in part, on the discovery that modulating the expression levels of particular genes and/or the activities of the corresponding protein product in a patient having an NCD can effectively treat the disease and alleviate accompanying symptomology. Additionally, the present disclosure stems, in part, from the surprising discovery that altering the expression patterns and/or activity levels of various groupings of genes and their protein products, respectively, can also be used to treat the foregoing disorders. This latter concept is particularly innovative. To date, many gene therapy technologies have focused on the delivery to a patient of a single gene for the treatment of a single congenital disorder. The instant disclosure is unique, for example, in that it provides compositions and methods for the manipulation of a plurality of gene expression levels and/or corresponding protein activity levels in order to treat a given NCD.

The compositions and methods of the disclosure provide a series of important clinical benefits. For example, using the compositions and methods described herein, a patient suffering from an NCD can be treated in a manner that both targets underlying genetic etiologies of the disease and that ameliorates associated symptoms. Further, compositions and methods that involve manipulation of two or more genes or protein products provide the added benefit of facilitating the treatment of larger patient populations as compared to patient groups that are amenable to gene or protein monotherapy approaches. This is due, in part, to the present discovery that compositions that augment the expression and/or activity levels of multiple proteins can be safely administered to a patient that is deficient only in one of these proteins. This unexpected discovery renders possible the use of a single therapeutic product, such as a single population of cells, viral vectors, or other agents promoting the expression and/or activity of a plurality of proteins, for the treatment of larger patient populations comprised of patients harboring deleterious mutations across different genes. Using traditional monotherapy approaches, each patient in such a patient population would require a unique gene or protein delivery vehicle based on the particular protein deficiency exhibited by that patient. The compositions and methods of the disclosure provide the advantageous effect of being able to treat a diverse patient population using a single therapeutic product that modulates the expression and/or activity of multiple proteins, despite any redundancy that may exist between the proteins upregulated by the therapeutic product and those already expressed endogenously by a patient.

In a first aspect, the disclosure provides a method of treating an NCD (e.g., Alzheimer's disease) in a patient (e.g., a mammalian patient, such as a human patient (e.g., an adult human patient)) in need thereof by providing to the patient one or more agents that collectively increase expression and/or activity of one or more proteins selected from APP, PSEN1, PSEN2, APOE, TOMM40, GAB2, APOC1, TREM2, ABI3, BIN1, CR1, ABCA7, FERMT2, HLA-DRB5,, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1 L, STK24, DISC1, MPZL1, SLC4A1AP, TRIP4, MSRA, HS3ST1, ZNF224, and AP2A2, such as one or more proteins selected from PSEN1, GAB2, APOC1, TREM2, ABI3, BIN1, HLA-DRB5, HLA-DRB1, CD2AP, PTK2B, INPP5D, MEF2C, CD33, MS4A4A, RIN3, PICALM, CASS4, SORL1, PLCG2, SCIMP, FRMD4A, SPPL2A, MTHFD1 L, DISC1, TRIP4, and HS3ST1.

In some embodiments of the foregoing aspect, the one or more agents collectively increase expression and/or activity of two or more of the proteins selected from APP, PSEN1, PSEN2, APOE, TOMM40, GAB2, APOC1, TREM2, ABI3, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1L, STK24, DISCI , MPZL1, SLC4A1AP, TRIP4, MSRA, HS3ST1, ZNF224, and AP2A2, such as two or more proteins selected from PSEN1, GAB2, APOC1, TREM2, ABI3, BIN1, HLA-DRB5, HLA-DRB1, CD2AP, PTK2B, INPP5D, MEF2C, CD33, MS4A4A, RIN3, PICALM, CASS4, SORL1, PLCG2, SCIMP, FRMD4A, SPPL2A, MTHFD1 L, DISCI , TRIP4, and HS3ST1. For example, the one or more agents may collectively increase expression and/or activity of three, four, five, six, seven, eight, nine, ten, 11, 12, 13, 14, 15, 17, 18, 19, 20, or more, of APP, PSEN1, PSEN2, APOE, TOMM40, GAB2, APOC1, TREM2, ABI3, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1 L, STK24, DISC1, MPZL1, SLC4A1AP, TRIP4, MSRA, HS3ST1, ZNF224, and AP2A2, such as three, four, five, six, seven, eight, nine, ten, 11, 12, 13, 14, 15, 17, 18, 19, 20, or more, of PSEN1, GAB2, APOC1, TREM2, ABI3, BIN1, HLA-DRB5, HLA-DRB1, CD2AP, PTK2B, INPP5D, MEF2C, CD33, MS4A4A, RIN3, PICALM, CASS4, SORL1, PLCG2, SCIMP, FRMD4A, SPPL2A, MTHFD1 L, DISC1, TRIP4, and HS3ST1.

In some embodiments of the foregoing aspect, the one or more agents collectively increase expression and/or activity of from two to 20 of the proteins, such as from two to 19, two to 18, two to 17, two to 16, two to 15, two to 14, two to 13, two to 12, two to 11, two to ten, two to nine, two to eight, two to seven, two to six, two to five, two to four, three to 20, three to 19, three to 18, three to 17, three to 16, three to 15, three to 14, three to 13, three to 12, three to 11, three to ten, three to nine, three to eight, three to seven, three to six, three to five, four to 20, four to 19, four to 18, four to 17, four to 16, four to 15, four to 14, four to 13, four to 12, four to 11, four to ten, four to nine, four to eight, four to seven, four to six, five to 20, five to 19, five to 18, five to 17, five to 16, five to 15, five to 14, five to 13, five to 12, five to 11, five to ten, five to nine, five to eight, five to seven, six to 20, six to 19, six to 18, six to 17, six to 16, six to 15, six to 14, six to 13, six to 12, six to 11, six to ten, six to nine, six to eight, seven to 20, seven to 19, seven to 18, seven to 17, seven to 16, seven to 15, seven to 14, seven to 13, seven to 12, seven to 11, seven to ten, seven to nine, eight to 20, eight to 19, eight to 18, eight to 17, eight to 16, eight to 15, eight to 14, eight to 13, eight to 12, eight to 11, eight to ten, nine to 20, nine to 19, nine to 18, nine to 17, nine to 16, nine to 15, nine to 14, nine to 13, nine to 12, nine to 11, ten to 20, ten to 19, ten to 18, ten to 17, ten to 16, ten to 15, ten to 14, ten to 13, ten to 12, 11 to 20, 11 to 19, 11 to 18, 11 to 17, 11 to 16, 11 to 15, 11 to 14, 11 to 13, 12 to 20, 12 to 19, 12 to 18, 12 to 17, 12 to 16, 12 to 15, 12 to 14, 13 to 20, 13 to 19, 13 to 18, 13 to 17, 13 to 16, 13 to 15, 14 to 20, 14 to 19, 14 to 18, 14 to 17, 14 to 16, 15 to 20, 15 to 19, 15 to 18, 15 to 17, 16 to 20, 16 to 19, 16 to 18, 17 to 20, 17 to 19, or 18 to 20 of proteins APP, PSEN1, PSEN2, APOE, TOMM40, GAB2, APOC1, TREM2, ABI3, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1L, STK24, DISCI , MPZL1, SLC4A1AP, TRIP4, MSRA, HS3ST1, ZNF224, and AP2A2 (e.g., from two to 19, two to 18, two to 17, two to 16, two to 15, two to 14, two to 13, two to 12, two to 11, two to ten, two to nine, two to eight, two to seven, two to six, two to five, two to four, three to 20, three to 19, three to 18, three to 17, three to 16, three to 15, three to 14, three to 13, three to 12, three to 11, three to ten, three to nine, three to eight, three to seven, three to six, three to five, four to 20, four to 19, four to 18, four to 17, four to 16, four to 15, four to 14, four to 13, four to 12, four to 11, four to ten, four to nine, four to eight, four to seven, four to six, five to 20, five to 19, five to 18, five to 17, five to 16, five to 15, five to 14, five to 13, five to 12, five to 11, five to ten, five to nine, five to eight, five to seven, six to 20, six to 19, six to 18, six to 17, six to 16, six to 15, six to 14, six to 13, six to 12, six to 11, six to ten, six to nine, six to eight, seven to 20, seven to 19, seven to 18, seven to 17, seven to 16, seven to 15, s even to 14, seven to 13, seven to 12, seven to 11, seven to ten, seven to nine, eight to 20, eight to 19, eight to 18, eight to 17, eight to 16, eight to 15, eight to 14, eight to 13, eight to 12, eight to 11, eight to ten, nine to 20, nine to 19, nine to 18, nine to 17, nine to 16, nine to 15, nine to 14, nine to 13, nine to 12, nine to 11, ten to 20, ten to 19, ten to 18, ten to 17, ten to 16, ten to 15, ten to 14, ten to 13, ten to 12, 11 to 20, 11 to 19, 11 to 18, 11 to 17, 11 to 16, 11 to 15, 11 to 14, 11 to 13, 12 to 20, 12 to 19, 12 to 18, 12 to 17, 12 to 16, 12 to 15, 12 to 14, 13 to 20, 13 to 19, 13 to 18, 13 to 17, 13 to 16, 13 to 15, 14 to 20, 14 to 19, 14 to 18, 14 to 17, 14 to 16, 15 to 20, 15 to 19, 15 to 18, 15 to 17, 16 to 20, 16 to 19, 16 to 18, 17 to 20, 17 to 19, or 18 to 20 of proteins PSEN1, GAB2, APOC1, TREM2, ABI3, BIN1, HLA-DRB5, HLA-DRB1, CD2AP, PTK2B, INPP5D, MEF2C, CD33, MS4A4A, RIN3, PICALM, CASS4, SORL1, PLCG2, SCIMP, FRMD4A, SPPL2A, MTHFD1 L, DISC1, TRIP4, and HS3ST1).

In some embodiments, the proteins include a panel set forth in Table 1, below. Each row within Table 1 denotes a pairwise “panel” of proteins.

TABLE 1
Exemplary panels of proteins useful for
the treatment of Alzheimer's disease
Panel

| | |
|---|---|
| PSEN1 | GAB2 |
| PSEN1 | APOC1 |
| PSEN1 | TREM2 |
| PSEN1 | ABI3 |
| PSEN1 | BIN1 |
| PSEN1 | HLA-DRB5 |
| PSEN1 | HLA-DRB1 |
| PSEN1 | CD2AP |
| PSEN1 | PTK2B |
| PSEN1 | INPP5D |
| PSEN1 | MEF2C |
| PSEN1 | CD33 |
| PSEN1 | MS4A4A |
| PSEN1 | RIN3 |
| PSEN1 | PICALM |
| PSEN1 | CASS4 |
| PSEN1 | SORL1 |
| PSEN1 | PLCG2 |
| PSEN1 | SCIMP |
| PSEN1 | FRMD4A |
| PSEN1 | SPPL2A |
| PSEN1 | MTHFD1L |
| PSEN1 | DISC1 |
| PSEN1 | TRIP4 |
| PSEN1 | HS3ST1 |
| GAB2 | APOC1 |
| GAB2 | TREM2 |
| GAB2 | ABI3 |
| GAB2 | BIN1 |
| GAB2 | HLA-DRB5 |
| GAB2 | HLA-DRB1 |
| GAB2 | CD2AP |
| GAB2 | PTK2B |
| GAB2 | INPP5D |
| GAB2 | MEF2C |
| GAB2 | CD33 |
| GAB2 | MS4A4A |
| GAB2 | RIN3 |
| GAB2 | PICALM |
| GAB2 | CASS4 |
| GAB2 | SORL1 |
| GAB2 | PLCG2 |
| GAB2 | SCIMP |
| GAB2 | FRMD4A |
| GAB2 | SPPL2A |
| GAB2 | MTHFD1L |
| GAB2 | DISC1 |
| GAB2 | TRIP4 |
| GAB2 | HS3ST1 |
| APOC1 | TREM2 |
| APOC1 | ABI3 |
| APOC1 | BIN1 |
| APOC1 | HLA-DRB5 |
| APOC1 | HLA-DRB1 |
| APOC1 | CD2AP |
| APOC1 | PTK2B |
| APOC1 | INPP5D |
| APOC1 | MEF2C |
| APOC1 | CD33 |
| APOC1 | MS4A4A |
| APOC1 | RIN3 |
| APOC1 | PICALM |
| APOC1 | CASS4 |
| APOC1 | SORL1 |
| APOC1 | PLCG2 |
| APOC1 | SCIMP |
| APOC1 | FRMD4A |
| APOC1 | SPPL2A |
| APOC1 | MTHFD1L |
| APOC1 | DISC1 |
| APOC1 | TRIP4 |
| APOC1 | HS3ST1 |
| TREM2 | ABI3 |
| TREM2 | BIN1 |
| TREM2 | HLA-DRB5 |
| TREM2 | HLA-DRB1 |
| TREM2 | CD2AP |
| TREM2 | PTK2B |
| TREM2 | INPP5D |
| TREM2 | MEF2C |
| TREM2 | CD33 |
| TREM2 | MS4A4A |
| TREM2 | RIN3 |
| TREM2 | PICALM |
| TREM2 | CASS4 |
| TREM2 | SORL1 |
| TREM2 | PLCG2 |
| TREM2 | SCIMP |
| TREM2 | FRMD4A |
| TREM2 | SPPL2A |
| TREM2 | MTHFD1L |
| TREM2 | DISC1 |
| TREM2 | TRIP4 |
| TREM2 | HS3ST1 |
| ABI3 | BIN1 |
| ABI3 | HLA-DRB5 |
| ABI3 | HLA-DRB1 |
| ABI3 | CD2AP |
| ABI3 | PTK2B |
| ABI3 | INPP5D |
| ABI3 | MEF2C |
| ABI3 | CD33 |
| ABI3 | MS4A4A |
| ABI3 | RIN3 |
| ABI3 | PICALM |
| ABI3 | CASS4 |
| ABI3 | SORL1 |
| ABI3 | PLCG2 |
| ABI3 | SCIMP |
| ABI3 | FRMD4A |
| ABI3 | SPPL2A |
| ABI3 | MTHFD1L |
| ABI3 | DISC1 |
| ABI3 | TRIP4 |
| ABI3 | HS3ST1 |
| BIN1 | HLA-DRB5 |
| BIN1 | HLA-DRB1 |
| BIN1 | CD2AP |
| BIN1 | PTK2B |
| BIN1 | INPP5D |
| BIN1 | MEF2C |
| BIN1 | CD33 |
| BIN1 | MS4A4A |
| BIN1 | RIN3 |
| BIN1 | PICALM |
| BIN1 | CASS4 |
| BIN1 | SORL1 |
| BIN1 | PLCG2 |
| BIN1 | SCIMP |
| BIN1 | FRMD4A |
| BIN1 | SPPL2A |
| BIN1 | MTHFD1L |
| BIN1 | DISC1 |
| BIN1 | TRIP4 |
| BIN1 | HS3ST1 |
| HLA-DRB5 | HLA-DRB1 |
| HLA-DRB5 | CD2AP |
| HLA-DRB5 | PTK2B |
| HLA-DRB5 | INPP5D |
| HLA-DRB5 | MEF2C |
| HLA-DRB5 | CD33 |
| HLA-DRB5 | MS4A4A |
| HLA-DRB5 | RIN3 |
| HLA-DRB5 | PICALM |
| HLA-DRB5 | CASS4 |
| HLA-DRB5 | SORL1 |
| HLA-DRB5 | PLCG2 |
| HLA-DRB5 | SCIMP |
| HLA-DRB5 | FRMD4A |
| HLA-DRB5 | SPPL2A |
| HLA-DRB5 | MTHFD1L |
| HLA-DRB5 | DISC1 |
| HLA-DRB5 | TRIP4 |
| HLA-DRB5 | HS3ST1 |
| HLA-DRB1 | CD2AP |
| HLA-DRB1 | PTK2B |
| HLA-DRB1 | INPP5D |
| HLA-DRB1 | MEF2C |
| HLA-DRB1 | CD33 |
| HLA-DRB1 | MS4A4A |
| HLA-DRB1 | RIN3 |
| HLA-DRB1 | PICALM |
| HLA-DRB1 | CASS4 |
| HLA-DRB1 | SORL1 |
| HLA-DRB1 | PLCG2 |
| HLA-DRB1 | SCIMP |
| HLA-DRB1 | FRMD4A |
| HLA-DRB1 | SPPL2A |
| HLA-DRB1 | MTHFD1L |
| HLA-DRB1 | DISC1 |
| HLA-DRB1 | TRIP4 |
| HLA-DRB1 | HS3ST1 |
| CD2AP | PTK2B |
| CD2AP | INPP5D |
| CD2AP | MEF2C |
| CD2AP | CD33 |
| CD2AP | MS4A4A |
| CD2AP | RIN3 |
| CD2AP | PICALM |
| CD2AP | CASS4 |
| CD2AP | SORL1 |
| CD2AP | PLCG2 |
| CD2AP | SCIMP |
| CD2AP | FRMD4A |
| CD2AP | SPPL2A |
| CD2AP | MTHFD1L |
| CD2AP | DISC1 |
| CD2AP | TRIP4 |
| CD2AP | HS3ST1 |
| PTK2B | INPP5D |
| PTK2B | MEF2C |
| PTK2B | CD33 |
| PTK2B | MS4A4A |
| PTK2B | RIN3 |
| PTK2B | PICALM |
| PTK2B | CASS4 |
| PTK2B | SORL1 |
| PTK2B | PLCG2 |
| PTK2B | SCIMP |
| PTK2B | FRMD4A |
| PTK2B | SPPL2A |
| PTK2B | MTHFD1L |
| PTK2B | DISC1 |
| PTK2B | TRIP4 |
| PTK2B | HS3ST1 |
| INPP5D | MEF2C |
| INPP5D | CD33 |
| INPP5D | MS4A4A |
| INPP5D | RIN3 |
| INPP5D | PICALM |
| INPP5D | CASS4 |
| INPP5D | SORL1 |
| INPP5D | PLCG2 |
| INPP5D | SCIMP |
| INPP5D | FRMD4A |
| INPP5D | SPPL2A |
| INPP5D | MTHFD1L |
| INPP5D | DISC1 |
| INPP5D | TRIP4 |
| INPP5D | HS3ST1 |
| MEF2C | CD33 |
| MEF2C | MS4A4A |
| MEF2C | RIN3 |
| MEF2C | PICALM |
| MEF2C | CASS4 |
| MEF2C | SORL1 |
| MEF2C | PLCG2 |
| MEF2C | SCIMP |
| MEF2C | FRMD4A |
| MEF2C | SPPL2A |
| MEF2C | MTHFD1L |
| MEF2C | DISC1 |
| MEF2C | TRIP4 |
| MEF2C | HS3ST1 |
| CD33 | MS4A4A |
| CD33 | RIN3 |
| CD33 | PICALM |
| CD33 | CASS4 |
| CD33 | SORL1 |
| CD33 | PLCG2 |
| CD33 | SCIMP |
| CD33 | FRMD4A |
| CD33 | SPPL2A |
| CD33 | MTHFD1L |
| CD33 | DISC1 |
| CD33 | TRIP4 |
| CD33 | HS3ST1 |
| MS4A4A | RIN3 |
| MS4A4A | PICALM |
| MS4A4A | CASS4 |
| MS4A4A | SORL1 |
| MS4A4A | PLCG2 |
| MS4A4A | SCIMP |
| MS4A4A | FRMD4A |
| MS4A4A | SPPL2A |
| MS4A4A | MTHFD1L |
| MS4A4A | DISC1 |
| MS4A4A | TRIP4 |
| MS4A4A | HS3ST1 |
| RIN3 | PICALM |
| RIN3 | CASS4 |
| RIN3 | SORL1 |
| RIN3 | PLCG2 |
| RIN3 | SCIMP |
| RIN3 | FRMD4A |
| RIN3 | SPPL2A |
| RIN3 | MTHFD1L |
| RIN3 | DISC1 |
| RIN3 | TRIP4 |
| RIN3 | HS3ST1 |
| PICALM | CASS4 |
| PICALM | SORL1 |
| PICALM | PLCG2 |
| PICALM | SCIMP |
| PICALM | FRMD4A |
| PICALM | SPPL2A |
| PICALM | MTHFD1L |
| PICALM | DISC1 |
| PICALM | TRIP4 |
| PICALM | HS3ST1 |
| CASS4 | SORL1 |
| CASS4 | PLCG2 |
| CASS4 | SCIMP |
| CASS4 | FRMD4A |
| CASS4 | SPPL2A |
| CASS4 | MTHFD1L |
| CASS4 | DISC1 |
| CASS4 | TRIP4 |
| CASS4 | HS3ST1 |
| SORL1 | PLCG2 |
| SORL1 | SCIMP |
| SORL1 | FRMD4A |
| SORL1 | SPPL2A |
| SORL1 | MTHFD1L |
| SORL1 | DISC1 |
| SORL1 | TRIP4 |
| SORL1 | HS3ST1 |
| PLCG2 | SCIMP |
| PLCG2 | FRMD4A |
| PLCG2 | SPPL2A |
| PLCG2 | MTHFD1L |
| PLCG2 | DISC1 |
| PLCG2 | TRIP4 |
| PLCG2 | HS3ST1 |
| SCIMP | FRMD4A |
| SCIMP | SPPL2A |
| SCIMP | MTHFD1L |
| SCIMP | DISC1 |
| SCIMP | TRIP4 |
| SCIMP | HS3ST1 |
| FRMD4A | SPPL2A |
| FRMD4A | MTHFD1L |
| FRMD4A | DISC1 |
| FRMD4A | TRIP4 |
| FRMD4A | HS3ST1 |
| SPPL2A | MTHFD1L |
| SPPL2A | DISC1 |
| SPPL2A | TRIP4 |
| SPPL2A | HS3ST1 |
| MTHFD1L | DISC1 |
| MTHFD1L | TRIP4 |
| MTHFD1L | HS3ST1 |
| DISC1 | TRIP4 |
| DISC1 | HS3ST1 |
| TRIP4 | HS3ST1 |

In some embodiments of the foregoing aspect, the patient is diagnosed with an NCD. In some embodiments, the NCD is a major NCD. In some embodiments, the major NCD interferes with the patient's independence and/or normal daily functioning (e.g., social, occupational, or academic functioning, personal hygiene, grooming, dressing, toilet hygiene, functional mobility (e.g., ability to walk, get in and out of bed), and self-feeding. In some embodiments, the major NCD is associated with a score obtained by the patient on a cognitive test that is at least two standard deviations away from the mean score of a reference population. In some embodiments, the NCD is a mild NCD. In some embodiments, the mild NCD does not interfere with the patient's independence and/or normal daily functioning. In some embodiments, the mild NCD is associated with a score obtained by the patient on a cognitive test that is between one to two standard deviations away from the mean score of a reference population. In some embodiments, the cognitive test is selected from the group consisting of ADB, AWV, GPCOG, HRA, MIS, MMSE, MoCA, SLUMS, and Short IQCODE. In some embodiments, the NCD is associated with impairment in one or more of complex attention, executive function, learning and memory, language, perceptual-motor function, and social cognition. In some embodiments, the NCD is not due to delirium or other mental disorder (e.g., schizophrenia, bipolar disorder, or major depression). In some embodiments, the reference population is a general population. In some embodiments, the reference population is selected on the basis of the patient's age, medical history, education, socioeconomic status, and lifestyle. In some embodiments, the NCD is Alzheimer's disease.

In a second aspect, the disclosure provides a method of treating an NCD (e.g., Parkinson's disease) in a patient (e.g., a mammalian patient, such as a human patient (e.g., an adult human patient)) in need thereof by providing to the patient one or more agents that collectively increase expression and/or activity of one or more proteins selected from FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1B, NUCKS1, and ACMSD, such as one or more proteins selected from FCGR2A, SCAF11, DNAJC13, GCH1, LRRK2, GBA, GAK, FGF20, HLA-DQB1, and NOD2.

In some embodiments of the foregoing aspect, the one or more agents collectively increase expression and/or activity of two or more of the proteins selected from FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1 L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1B, NUCKS1, and ACMSD, such as two or more proteins selected from FCGR2A, SCAF11, DNAJC13, GCH1, LRRK2, GBA, GAK, FGF20, HLA-DQB1, and NOD2. For example, the one or more agents may collectively increase expression and/or activity of three, four, five, six, seven, eight, nine, ten, 11, 12, 13, 14, 15, 17, 18, 19, 20, or more, of FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1 B, NUCKS1, and ACMSD, such as three, four, five, six, seven, eight, nine, or more, of FCGR2A, SCAF11, DNAJC13, GCH1, LRRK2, GBA, GAK, FGF20, HLA-DQB1, and NOD2.

In some embodiments of the foregoing aspect, the one or more agents collectively increase expression and/or activity of from two to 20 of the proteins, such as from two to 19, two to 18, two to 17, two to 16, two to 15, two to 14, two to 13, two to 12, two to 11, two to ten, two to nine, two to eight, two to seven, two to six, two to five, two to four, three to 20, three to 19, three to 18, three to 17, three to 16, three to 15, three to 14, three to 13, three to 12, three to 11, three to ten, three to nine, three to eight, three to seven, three to six, three to five, four to 20, four to 19, four to 18, four to 17, four to 16, four to 15, four to 14, four to 13, four to 12, four to 11, four to ten, four to nine, four to eight, four to seven, four to six, five to 20, five to 19, five to 18, five to 17, five to 16, five to 15, five to 14, five to 13, five to 12, five to 11, five to ten, five to nine, five to eight, five to seven, six to 20, six to 19, six to 18, six to 17, six to 16, six to 15, six to 14, six to 13, six to 12, six to 11, six to ten, six to nine, six to eight, seven to 20, seven to 19, seven to 18, seven to 17, seven to 16, seven to 15, seven to 14, seven to 13, seven to 12, seven to 11, seven to ten, seven to nine, eight to 20, eight to 19, eight to 18, eight to 17, eight to 16, eight to 15, eight to 14, eight to 13, eight to 12, eight to 11, eight to ten, nine to 20, nine to 19, nine to 18, nine to 17, nine to 16, nine to 15, nine to 14, nine to 13, nine to 12, nine to 11, ten to 20, ten to 19, ten to 18, ten to 17, ten to 16, ten to 15, ten to 14, ten to 13, ten to 12, 11 to 20, 11 to 19, 11 to 18, 11 to 17, 11 to 16, 11 to 15, 11 to 14, 11 to 13, 12 to 20, 12 to 19, 12 to 18, 12 to 17, 12 to 16, 12 to 15, 12 to 14, 13 to 20, 13 to 19, 13 to 18, 13 to 17, 13 to 16, 13 to 15, 14 to 20, 14 to 19, 14 to 18, 14 to 17, 14 to 16, 15 to 20, 15 to 19, 15 to 18, 15 to 17, 16 to 20, 16 to 19, 16 to 18, 17 to 20, 17 to 19, or 18 to 20 of proteins FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1 L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1 B, NUCKS1, and ACMSD (e.g., from two to ten, two to nine, two to eight, two to seven, two to six, two to five, two to four, three to ten, three to nine, three to eight, three to seven, three to six, three to five, four to ten, four to nine, four to eight, four to seven, four to six, five to ten, five to nine, five to eight, five to seven, six to ten, six to nine, six to eight, seven to ten, seven to nine, or eight to ten of proteins FCGR2A, SCAF11, DNAJC13, GCH1, LRRK2, GBA, GAK, FGF20, HLA-DQB1, and NOD2).

In some embodiments, the proteins include a panel set forth in Table 2, below. Each row within Table 2 denotes a pairwise “panel” of proteins.

TABLE 2
Exemplary panels of proteins useful for
the treatment of Parkinson's disease
Panel

| | |
|---|---|
| FCGR2A | SCAF11 |
| FCGR2A | DNAJC13 |
| FCGR2A | GCH1 |
| FCGR2A | LRRK2 |
| FCGR2A | GBA |
| FCGR2A | GAK |
| FCGR2A | FGF20 |
| FCGR2A | HLA-DQB1 |
| FCGR2A | NOD2 |
| SCAF11 | DNAJC13 |
| SCAF11 | GCH1 |
| SCAF11 | LRRK2 |
| SCAF11 | GBA |
| SCAF11 | GAK |
| SCAF11 | FGF20 |
| SCAF11 | HLA-DQB1 |
| SCAF11 | NOD2 |
| DNAJC13 | GCH1 |
| DNAJC13 | LRRK2 |
| DNAJC13 | GBA |
| DNAJC13 | GAK |
| DNAJC13 | FGF20 |
| DNAJC13 | HLA-DQB1 |
| DNAJC13 | NOD2 |
| GCH1 | LRRK2 |
| GCH1 | GBA |
| GCH1 | GAK |
| GCH1 | FGF20 |
| GCH1 | HLA-DQB1 |
| GCH1 | NOD2 |
| LRRK2 | GBA |
| LRRK2 | GAK |
| LRRK2 | FGF20 |
| LRRK2 | HLA-DQB1 |
| LRRK2 | NOD2 |
| GBA | GAK |
| GBA | FGF20 |
| GBA | HLA-DQB1 |
| GBA | NOD2 |
| GAK | FGF20 |
| GAK | HLA-DQB1 |
| GAK | NOD2 |
| FGF20 | HLA-DQB1 |
| FGF20 | NOD2 |
| HLA-DQB1 | NOD2 |
| SCARB2 | GBA |

In some embodiments of the second aspect, the patient is diagnosed with an NCD. In some embodiments, the NCD is a major NCD. In some embodiments, the major NCD interferes with the patient's independence and/or normal daily functioning (e.g., social, occupational, or academic functioning, personal hygiene, grooming, dressing, toilet hygiene, functional mobility (e.g., ability to walk, get in and out of bed), and self-feeding. In some embodiments, the major NCD is associated with a score obtained by the patient on a cognitive test that is at least two standard deviations away from the mean score of a reference population. In some embodiments, the NCD is a mild NCD. In some embodiments, the mild NCD does not interfere with the patient's independence and/or normal daily functioning. In some embodiments, the mild NCD is associated with a score obtained by the patient on a cognitive test that is between one to two standard deviations away from the mean score of a reference population. In some embodiments, the cognitive test is selected from the group consisting of ADB, AWV, GPCOG, HRA, MIS, MMSE, MoCA, SLUMS, and Short IQCODE. In some embodiments, the NCD is associated with impairment in one or more of complex attention, executive function, learning and memory, language, perceptual-motor function, and social cognition. In some embodiments, the NCD is not due to delirium or other mental disorder (e.g., schizophrenia, bipolar disorder, or major depression). In some embodiments, the reference population is a general population. In some embodiments, the reference population is selected on the basis of the patient's age, medical history, education, socioeconomic status, and lifestyle. In some embodiments, the NCD is a movement disorder. In some embodiments, the movement disorder is Parkinson disease.

In a third aspect, the disclosure provides a method of treating an NCD (e.g., FTLD, such as behavioral-variant frontotemporal dementia, semantic dementia, or progressive nonfluent aphasia) in a patient (e.g., a mammalian patient, such as a human patient (e.g., an adult human patient)) in need thereof by providing to the patient one or more agents that collectively increase expression and/or activity of one or more proteins selected from HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT, such as one or more proteins selected from HLA-DRA, HLA-DRB5, C9ORF72, SQSTM1, TBK1, PSEN1, GRN, and CTSF.

In some embodiments of the foregoing aspect, the one or more agents collectively increase expression and/or activity of two or more of the proteins selected from HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT, such as two or more proteins selected from HLA-DRA, HLA-DRB5, C9ORF72, SQSTM1, TBK1, PSEN1, GRN, and CTSF. For example, the one or more agents may collectively increase expression and/or activity of three, four, five, six, seven, eight, nine, ten, 11, 12, 13, 14, 15, or more, of HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT, such as three, four, five, six, or more, of HLA-DRA, HLA-DRB5, C9ORF72, SQSTM1, TBK1, PSEN1, GRN, and CTSF.

In some embodiments of the foregoing aspect, the one or more agents collectively increase expression and/or activity of from two to 15 of the proteins, such as from two to 14, two to 13, two to 12, two to 11, two to ten, two to nine, two to eight, two to seven, two to six, two to five, two to four, three to 15, three to 14, three to 13, three to 12, three to 11, three to ten, three to nine, three to eight, three to seven, three to six, three to five, four to 15, four to 14, four to 13, four to 12, four to 11, four to ten, four to nine, four to eight, four to seven, four to six, five to 15, five to 14, five to 13, five to 12, five to 11, five to ten, five to nine, five to eight, five to seven, six to 15, six to 14, six to 13, six to 12, six to 11, six to ten, six to nine, six to eight, seven to 15, seven to 14, seven to 13, seven to 12, seven to 11, seven to ten, seven to nine, eight to 15, eight to 14, eight to 13, eight to 12, eight to 11, eight to ten, nine to 15, nine to 14, nine to 13, nine to 12, nine to 11, ten to 15, ten to 14, ten to 13, ten to 12, 11 to 15, 11 to 14, 11 to 13, 12 to 15, or 12 to 14 of proteins HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT, such as two or more proteins selected from HLA-DRA, HLA-DRB5, C9ORF72, SQSTM1, TBK1, PSEN1, GRN, and CTSF (e.g., from two to six, two to five, two to four, three to six, three to five, four to ten, or four to six, of proteins HLA-DRA, HLA-DRB5, C9ORF72, SQSTM1, TBK1, PSEN1, GRN, and CTSF).

In some embodiments, the proteins include a panel set forth in Table 3, below. Each row within Table 3 denotes a pairwise “panel” of proteins.

TABLE 3
Exemplary panels of proteins useful for the
treatment of a frontotemporal lobar degeneration
Panel

| | |
|---|---|
| HLA-DRA | HLA-DRB5 |
| HLA-DRA | C9ORF72 |
| HLA-DRA | SQSTM1 |
| HLA-DRA | TBK1 |
| HLA-DRA | PSEN1 |
| HLA-DRA | GRN |
| HLA-DRA | CTSF |
| HLA-DRB5 | C9ORF72 |
| HLA-DRB5 | SQSTM1 |
| HLA-DRB5 | TBK1 |
| HLA-DRB5 | PSEN1 |
| HLA-DRB5 | GRN |
| HLA-DRB5 | CTSF |
| C9ORF72 | SQSTM1 |
| C9ORF72 | TBK1 |
| C9ORF72 | PSEN1 |
| C9ORF72 | GRN |
| C9ORF72 | CTSF |
| SQSTM1 | TBK1 |
| SQSTM1 | PSEN1 |
| SQSTM1 | GRN |
| SQSTM1 | CTSF |
| TBK1 | PSEN1 |
| TBK1 | GRN |
| TBK1 | CTSF |
| PSEN1 | GRN |
| PSEN1 | CTSF |
| GRN | CTSF |

In some embodiments of the third aspect, the patient is diagnosed with an NCD. In some embodiments, the NCD is a major NCD. In some embodiments, the major NCD interferes with the patient's independence and/or normal daily functioning (e.g., social, occupational, or academic functioning, personal hygiene, grooming, dressing, toilet hygiene, functional mobility (e.g., ability to walk, get in and out of bed), and self-feeding. In some embodiments, the major NCD is associated with a score obtained by the patient on a cognitive test that is at least two standard deviations away from the mean score of a reference population. In some embodiments, the NCD is a mild NCD. In some embodiments, the mild NCD does not interfere with the patient's independence and/or normal daily functioning. In some embodiments, the mild NCD is associated with a score obtained by the patient on a cognitive test that is between one to two standard deviations away from the mean score of a reference population. In some embodiments, the cognitive test is selected from the group consisting of ADB, AWV, GPCOG, HRA, MIS, MMSE, MoCA, SLUMS, and Short IQCODE. In some embodiments, the NCD is associated with impairment in one or more of complex attention, executive function, learning and memory, language, perceptual-motor function, and social cognition. In some embodiments, the NCD is not due to delirium or other mental disorder (e.g., schizophrenia, bipolar disorder, or major depression). In some embodiments, the reference population is a general population. In some embodiments, the reference population is selected on the basis of the patient's age, medical history, education, socioeconomic status, and lifestyle. In some embodiments, the NCD is a frontotemporal NCD. In some embodiments, the frontotemporal NCD is FTLD. In some embodiments, the FTLD is behavioral-variant frontotemporal dementia. In some embodiments, the FTLD is semantic dementia. In some embodiments, the FTLD is progressive nonfluent aphasia.

In a fourth aspect, the disclosure provides a method of treating an NCD (e.g., Alzheimer's disease, Parkinson disease, or frontotemporal lobar degeneration) in a patient in need thereof by providing to the patient one or more agents that collectively increase expression and/or activity of one or more proteins selected from APP, PSEN1, PSEN2, APOE, TOMM40, GAB2, APOC1, TREM2, ABI3, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1L, STK24, DISCI , MPZL1, SLC4A1AP, TRIP4, MSRA, HS3ST1, ZNF224, AP2A2, FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1 B, NUCKS1, ACMSD, HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT. For example, the one or more agents may collectively increase expression and/or activity of two, three, four, five, six, seven, eight, nine, ten, 11, 12, 13, 14, 15, 17, 18, 19, 20, or more of APP, PSEN1, PSEN2, APOE, TOMM40, GAB2, APOC1, TREM2, ABI3, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1L, STK24, DISCI , MPZL1, SLC4A1AP, TRIP4, MSRA, HS3ST1, ZNF224, AP2A2, FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1 L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1B, NUCKS1, ACMSD, HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT.

In some embodiments of the foregoing aspect, the one or more agents collectively increase expression and/or activity of from two to 20 of the proteins, such as from two to 19, two to 18, two to 17, two to 16, two to 15, two to 14, two to 13, two to 12, two to 11, two to ten, two to nine, two to eight, two to seven, two to six, two to five, two to four, three to 20, three to 19, three to 18, three to 17, three to 16, three to 15, three to 14, three to 13, three to 12, three to 11, three to ten, three to nine, three to eight, three to seven, three to six, three to five, four to 20, four to 19, four to 18, four to 17, four to 16, four to 15, four to 14, four to 13, four to 12, four to 11, four to ten, four to nine, four to eight, four to seven, four to six, five to 20, five to 19, five to 18, five to 17, five to 16, five to 15, five to 14, five to 13, five to 12, five to 11, five to ten, five to nine, five to eight, five to seven, six to 20, six to 19, six to 18, six to 17, six to 16, six to 15, six to 14, six to 13, six to 12, six to 11, six to ten, six to nine, six to eight, seven to 20, seven to 19, seven to 18, seven to 17, seven to 16, seven to 15, seven to 14, seven to 13, seven to 12, seven to 11, seven to ten, seven to nine, eight to 20, eight to 19, eight to 18, eight to 17, eight to 16, eight to 15, eight to 14, eight to 13, eight to 12, eight to 11, eight to ten, nine to 20, nine to 19, nine to 18, nine to 17, nine to 16, nine to 15, nine to 14, nine to 13, nine to 12, nine to 11, ten to 20, ten to 19, ten to 18, ten to 17, ten to 16, ten to 15, ten to 14, ten to 13, ten to 12, 11 to 20, 11 to 19, 11 to 18, 11 to 17, 11 to 16, 11 to 15, 11 to 14, 11 to 13, 12 to 20, 12 to 19, 12 to 18, 12 to 17, 12 to 16, 12 to 15, 12 to 14, 13 to 20, 13 to 19, 13 to 18, 13 to 17, 13 to 16, 13 to 15, 14 to 20, 14 to 19, 14 to 18, 14 to 17, 14 to 16, 15 to 20, 15 to 19, 15 to 18, 15 to 17, 16 to 20, 16 to 19, 16 to 18, 17 to 20, 17 to 19, or 18 to 20 of proteins APP, PSEN1, PSEN2, APOE, TOMM40, GAB2, APOC1, TREM2, ABI3, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1L, STK24, DISCI , MPZL1, SLC4A1AP, TRIP4, MSRA, HS3ST1, ZNF224, AP2A2, FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1B, NUCKS1, ACMSD, HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT (e.g., from two to 19, two to 18, two to 17, two to 16, two to 15, two to 14, two to 13, two to 12, two to 11, two to ten, two to nine, two to eight, two to seven, two to six, two to five, two to four, three to 20, three to 19, three to 18, three to 17, three to 16, three to 15, three to 14, three to 13, three to 12, three to 11, three to ten, three to nine, three to eight, three to seven, three to six, three to five, four to 20, four to 19, four to 18, four to 17, four to 16, four to 15, four to 14, four to 13, four to 12, four to 11, four to ten, four to nine, four to eight, four to seven, four to six, five to 20, five to 19, five to 18, five to 17, five to 16, five to 15, five to 14, five to 13, five to 12, five to 11, five to ten, five to nine, five to eight, five to seven, six to 20, six to 19, six to 18, six to 17, six to 16, six to 15, six to 14, six to 13, six to 12, six to 11, six to ten, six to nine, six to eight, seven to 20, seven to 19, seven to 18, seven to 17, seven to 16, seven to 15, seven to 14, seven to 13, seven to 12, seven to 11, seven to ten, seven to nine, eight to 20, eight to 19, eight to 18, eight to 17, eight to 16, eight to 15, eight to 14, eight to 13, eight to 12, eight to 11, eight to ten, nine to 20, nine to 19, nine to 18, nine to 17, nine to 16, nine to 15, nine to 14, nine to 13, nine to 12, nine to 11, ten to 20, ten to 19, ten to 18, ten to 17, ten to 16, ten to 15, ten to 14, ten to 13, ten to 12, 11 to 20, 11 to 19, 11 to 18, 11 to 17, 11 to 16, 11 to 15, 11 to 14, 11 to 13, 12 to 20, 12 to 19, 12 to 18, 12 to 17, 12 to 16, 12 to 15, 12 to 14, 13 to 20, 13 to 19, 13 to 18, 13 to 17, 13 to 16, 13 to 15, 14 to 20, 14 to 19, 14 to 18, 14 to 17, 14 to 16, 15 to 20, 15 to 19, 15 to 18, 15 to 17, 16 to 20, 16 to 19, 16 to 18, 17 to 20, 17 to 19, or 18 to 20 of proteins APP, PSEN1, PSEN2, APOE, TOMM40, GAB2, APOC1, TREM2, ABI3, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1 L, STK24, DISCI , MPZL1, SLC4A1AP, TRIP4, MSRA, HS3ST1, ZNF224, AP2A2, FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1 L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1B, NUCKS1, ACMSD, HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT).

In some embodiments, the proteins include a panel set forth in Table 4, below. Each row within Table 4 denotes a pairwise “panel” of proteins.

TABLE 4
Exemplary panels of proteins useful for the treatment of Alzheimer's
disease, Parkinson disease, or a frontotemporal lobar degeneration
Panel

| | |
|---|---|
| APR | PSEN1 |
| APP | PSEN2 |
| APP | APOE |
| APP | TOMM40 |
| APP | GAB2 |
| APP | APOC1 |
| APP | TREM2 |
| APP | ABI3 |
| APP | BIN1 |
| APP | CR1 |
| APP | ABCA7 |
| APP | FERMT2 |
| APP | HLA |
| APP | DRB5 |
| APP | HLA |
| APP | DRB1 |
| APP | CD2AP |
| APP | PTK2B |
| APP | CELF1 |
| APP | INPP5D |
| APP | MEF2C |
| APP | ZCWPW1 |
| APP | CD33 |
| APP | MS4A4A |
| APP | RIN3 |
| APP | EPHA1 |
| APP | PICALM |
| APP | CASS4 |
| APP | CLU |
| APP | SORL1 |
| APP | PLCG2 |
| APP | SCIMP |
| APP | FRMD4A |
| APP | SPPL2A |
| APP | MTHFD1L |
| APP | STK24 |
| APR | DISC1 |
| APP | MPZL1 |
| APP | SLC4A1AP |
| APP | TRIP4 |
| APP | MSRA |
| APP | HS3ST1 |
| APP | ZNF224 |
| APP | AP2A2 |
| APP | FCGR2A |
| APP | SCAF11 |
| APP | HLA |
| APP | DQB1 |
| APP | NOD2 |
| PSEN1 | PSEN2 |
| PSEN1 | APOE |
| PSEN1 | TOMM40 |
| PSEN1 | GAB2 |
| PSEN1 | APOC1 |
| PSEN1 | TREM2 |
| PSEN1 | ABI3 |
| PSEN1 | BIN1 |
| PSEN1 | CR1 |
| PSEN1 | ABCA7 |
| PSEN1 | FERMT2 |
| PSEN1 | HLA |
| PSEN1 | DRB5 |
| PSEN1 | HLA |
| PSEN1 | DRB1 |
| PSEN1 | CD2AP |
| PSEN1 | PTK2B |
| PSEN1 | CELF1 |
| PSEN1 | INPP5D |
| PSEN1 | MEF2C |
| PSEN1 | ZCWPW1 |
| PSEN1 | CD33 |
| PSEN1 | MS4A4A |
| PSEN1 | RIN3 |
| PSEN1 | EPHA1 |
| PSEN1 | PICALM |
| PSEN1 | CASS4 |
| PSEN1 | CLU |
| PSEN1 | SORL1 |
| PSEN1 | PLCG2 |
| PSEN1 | SCIMP |
| PSEN1 | FRMD4A |
| PSEN1 | SPPL2A |
| PSEN1 | MTHFD1L |
| PSEN1 | STK24 |
| PSEN1 | DISC1 |
| PSEN1 | MPZL1 |
| PSEN1 | SLC4A1AP |
| PSEN1 | TRIP4 |
| PSEN1 | MSRA |
| PSEN1 | HS3ST1 |
| PSEN1 | ZNF224 |
| PSEN1 | AP2A2 |
| PSEN1 | FCGR2A |
| PSEN1 | SCAF11 |
| PSEN1 | HLA |
| PSEN1 | DQB1 |
| PSEN1 | NOD2 |
| PSEN2 | APOE |
| PSEN2 | TOMM40 |
| PSEN2 | GAB2 |
| PSEN2 | APOC1 |
| PSEN2 | TREM2 |
| PSEN2 | ABI3 |
| PSEN2 | BIN1 |
| PSEN2 | CR1 |
| PSEN2 | ABCA7 |
| PSEN2 | FERMT2 |
| PSEN2 | HLA |
| PSEN2 | DRB5 |
| PSEN2 | HLA |
| PSEN2 | DRB1 |
| PSEN2 | CD2AP |
| PSEN2 | PTK2B |
| PSEN2 | CELF1 |
| PSEN2 | INPP5D |
| PSEN2 | MEF2C |
| PSEN2 | ZCWPW1 |
| PSEN2 | CD33 |
| PSEN2 | MS4A4A |
| PSEN2 | RIN3 |
| PSEN2 | EPHA1 |
| PSEN2 | PICALM |
| PSEN2 | CASS4 |
| PSEN2 | CLU |
| PSEN2 | SORL1 |
| PSEN2 | PLCG2 |
| PSEN2 | SCIMP |
| PSEN2 | FRMD4A |
| PSEN2 | SPPL2A |
| PSEN2 | MTHFD1L |
| PSEN2 | STK24 |
| PSEN2 | DISC1 |
| PSEN2 | MPZL1 |
| PSEN2 | SLC4A1AP |
| PSEN2 | TRIP4 |
| PSEN2 | MSRA |
| PSEN2 | HS3ST1 |
| PSEN2 | ZNF224 |
| PSEN2 | AP2A2 |
| PSEN2 | FCGR2A |
| PSEN2 | SCAF11 |
| PSEN2 | HLA |
| PSEN2 | DQB1 |
| PSEN2 | NOD2 |
| APOE | TOMM40 |
| APOE | GAB2 |
| APOE | APOC1 |
| APOE | TREM2 |
| APOE | ABI3 |
| APOE | BIN1 |
| APOE | CR1 |
| APOE | ABCA7 |
| APOE | FERMT2 |
| APOE | HLA |
| APOE | DRB5 |
| APOE | HLA |
| APOE | DRB1 |
| APOE | CD2AP |
| APOE | PTK2B |
| APOE | CELF1 |
| APOE | INPP5D |
| APOE | MEF2C |
| APOE | ZCWPW1 |
| APOE | CD33 |
| APOE | MS4A4A |
| APOE | RIN3 |
| APOE | EPHA1 |
| APOE | PICALM |
| APOE | CASS4 |
| APOE | CLU |
| APOE | SORL1 |
| APOE | PLCG2 |
| APOE | SCIMP |
| APOE | FRMD4A |
| APOE | SPPL2A |
| APOE | MTHFD1L |
| APOE | STK24 |
| APOE | DISC1 |
| APOE | MPZL1 |
| APOE | SLC4A1AP |
| APOE | TRIP4 |
| APOE | MSRA |
| APOE | HS3ST1 |
| APOE | ZNF224 |
| APOE | AP2A2 |
| APOE | FCGR2A |
| APOE | SCAF11 |
| APOE | HLA |
| APOE | DQB1 |
| APOE | NOD2 |
| TOMM40 | GAB2 |
| TOMM40 | APOC1 |
| TOMM40 | TREM2 |
| TOMM40 | ABI3 |
| TOMM40 | BIN1 |
| TOMM40 | CR1 |
| TOMM40 | ABCA7 |
| TOMM40 | FERMT2 |
| TOMM40 | HLA |
| TOMM40 | DRB5 |
| TOMM40 | HLA |
| TOMM40 | DRB1 |
| TOMM40 | CD2AP |
| TOMM40 | PTK2B |
| TOMM40 | CELF1 |
| TOMM40 | INPP5D |
| TOMM40 | MEF2C |
| TOMM40 | ZCWPW1 |
| TOMM40 | CD33 |
| TOMM40 | MS4A4A |
| TOMM40 | RIN3 |
| TOMM40 | EPHA1 |
| TOMM40 | PICALM |
| TOMM40 | CASS4 |
| TOMM40 | CLU |
| TOMM40 | SORL1 |
| TOMM40 | PLCG2 |
| TOMM40 | SCIMP |
| TOMM40 | FRMD4A |
| TOMM40 | SPPL2A |
| TOMM40 | MTHFD1L |
| TOMM40 | STK24 |
| TOMM40 | DISC1 |
| TOMM40 | MPZL1 |
| TOMM40 | SLC4A1AP |
| TOMM40 | TRIP4 |
| TOMM40 | MSRA |
| TOMM40 | HS3ST1 |
| TOMM40 | ZNF224 |
| TOMM40 | AP2A2 |
| TOMM40 | FCGR2A |
| TOMM40 | SCAF11 |
| TOMM40 | HLA |
| TOMM40 | DQB1 |
| TOMM40 | NOD2 |
| GAB2 | APOC1 |
| GAB2 | TREM2 |
| GAB2 | ABI3 |
| GAB2 | BIN1 |
| GAB2 | CR1 |
| GAB2 | ABCA7 |
| GAB2 | FERMT2 |
| GAB2 | HLA |
| GAB2 | DRB5 |
| GAB2 | HLA |
| GAB2 | DRB1 |
| GAB2 | CD2AP |
| GAB2 | PTK2B |
| GAB2 | CELF1 |
| GAB2 | INPP5D |
| GAB2 | MEF2C |
| GAB2 | ZCWPW1 |
| GAB2 | CD33 |
| GAB2 | MS4A4A |
| GAB2 | RIN3 |
| GAB2 | EPHA1 |
| GAB2 | PICALM |
| GAB2 | CASS4 |
| GAB2 | CLU |
| GAB2 | SORL1 |
| GAB2 | PLCG2 |
| GAB2 | SCIMP |
| GAB2 | FRMD4A |
| GAB2 | SPPL2A |
| GAB2 | MTHFD1L |
| GAB2 | STK24 |
| GAB2 | DISC1 |
| GAB2 | MPZL1 |
| GAB2 | SLC4A1AP |
| GAB2 | TRIP4 |
| GAB2 | MSRA |
| GAB2 | HS3ST1 |
| GAB2 | ZNF224 |
| GAB2 | AP2A2 |
| GAB2 | FCGR2A |
| GAB2 | SCAF11 |
| GAB2 | HLA |
| GAB2 | DQB1 |
| GAB2 | NOD2 |
| APOC1 | TREM2 |
| APOC1 | ABI3 |
| APOC1 | BIN1 |
| APOC1 | CR1 |
| APOC1 | ABCA7 |
| APOC1 | FERMT2 |
| APOC1 | HLA |
| APOC1 | DRB5 |
| APOC1 | HLA |
| APOC1 | DRB1 |
| APOC1 | CD2AP |
| APOC1 | PTK2B |
| APOC1 | CELF1 |
| APOC1 | INPP5D |
| APOC1 | MEF2C |
| APOC1 | ZCWPW1 |
| APOC1 | CD33 |
| APOC1 | MS4A4A |
| APOC1 | RIN3 |
| APOC1 | EPHA1 |
| APOC1 | PICALM |
| APOC1 | CASS4 |
| APOC1 | CLU |
| APOC1 | SORL1 |
| APOC1 | PLCG2 |
| APOC1 | SCIMP |
| APOC1 | FRMD4A |
| APOC1 | SPPL2A |
| APOC1 | MTHFD1L |
| APOC1 | STK24 |
| APOC1 | DISC1 |
| APOC1 | MPZL1 |
| APOC1 | SLC4A1AP |
| APOC1 | TRIP4 |
| APOC1 | MSRA |
| APOC1 | HS3ST1 |
| APOC1 | ZNF224 |
| APOC1 | AP2A2 |
| APOC1 | FCGR2A |
| APOC1 | SCAF11 |
| APOC1 | HLA |
| APOC1 | DQB1 |

In some embodiments of the foregoing aspect, the patient is diagnosed with an NCD. In some embodiments, the NCD is a major NCD. In some embodiments, the major NCD interferes with the patient's independence and/or normal daily functioning (e.g., social, occupational, or academic functioning, personal hygiene, grooming, dressing, toilet hygiene, functional mobility (e.g., ability to walk, get in and out of bed), and self-feeding. In some embodiments, the major NCD is associated with a score obtained by the patient on a cognitive test that is at least two standard deviations away from the mean score of a reference population. In some embodiments, the NCD is a mild NCD. In some embodiments, the mild NCD does not interfere with the patient's independence and/or normal daily functioning. In some embodiments, the mild NCD is associated with a score obtained by the patient on a cognitive test that is between one to two standard deviations away from the mean score of a reference population. In some embodiments, the cognitive test is selected from the group consisting of ADB, AWV, GPCOG, HRA, MIS, MMSE, MoCA, SLUMS, and Short IQCODE. In some embodiments, the NCD is associated with impairment in one or more of complex attention, executive function, learning and memory, language, perceptual-motor function, and social cognition. In some embodiments, the NCD is not due to delirium or other mental disorder (e.g., schizophrenia, bipolar disorder, or major depression). In some embodiments, the reference population is a general population. In some embodiments, the reference population is selected on the basis of the patient's age, medical history, education, socioeconomic status, and lifestyle. In some embodiments, the NCD is Alzheimer's disease. In some embodiments, the NCD is a movement disorder. In some embodiments, the movement disorder is Parkinson disease. In some embodiments, the NCD is a frontotemporal NCD. In some embodiments, the frontotemporal NCD is a FTLD. In some embodiments, the FTLD is a behavioral-variant frontotemporal dementia. In some embodiments, the FTLD is a semantic dementia. In some embodiments, the FTLD is a progressive nonfluent aphasia.

In some embodiments of any of the foregoing aspects of the disclosure, the one or more agents contain (i) one or more nucleic acid molecules that collectively encode the protein or proteins (such as, e.g., nucleic acids capable of expression in macrophages or microglia), (ii) one or more interfering RNA molecules that collectively increase expression and/or activity of the protein or proteins, (iii) one or more nucleic acid molecules encoding the one or more interfering RNA molecules (e.g., short interfering RNA (siRNA), short hairpin RNA (shRNA), and/or micro RNA (miRNA)), (iv) one or more of the proteins themselves, and/or (v) one or more small molecules that collectively increase expression and/or activity of the protein or proteins.

In some embodiments, the one or more agents contain one or more nucleic acid molecules that collectively encode the protein or proteins. For example, in cases of treating Alzheimer's disease, the patient may be provided one or more nucleic acid molecules that collectively encode one or more proteins selected from APP, PSEN1, PSEN2, APOE, TOMM40, GAB2, APOC1, TREM2, ABI3, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1 L, STK24, DISC1, MPZL1, SLC4A1 AP, TRIP4, MSRA, HS3ST1, ZNF224, and AP2A2, such as one or more proteins selected from PSEN1, GAB2, APOC1, TREM2, ABI3, BIN1, HLA-DRB5, HLA-DRB1, CD2AP, PTK2B, INPP5D, MEF2C, CD33, MS4A4A, RIN3, PICALM, CASS4, SORL1, PLCG2, SCIMP, FRMD4A, SPPL2A, MTHFD1 L, DISC1, TRIP4, and HS3ST1.

For example, in cases of treating Alzheimer's disease, the patient may be provided one or more nucleic acid molecules that collectively encode of two or more of the proteins selected from APP, PSEN1, PSEN2, APOE, TOMM40, GAB2, APOC1, TREM2, ABI3, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1L, STK24, DISCI , MPZL1, SLC4A1AP, TRIP4, MSRA, HS3ST1, ZNF224, and AP2A2, such as two or more proteins selected from PSEN1, GAB2, APOC1, TREM2, ABI3, BIN1, HLA-DRB5, HLA-DRB1, CD2AP, PTK2B, INPP5D, MEF2C, CD33, MS4A4A, RIN3, PICALM, CASS4, SORL1, PLCG2, SCIMP, FRMD4A, SPPL2A, MTHFD1 L, DISCI , TRIP4, and HS3ST1. For example, the one or more nucleic acid molecules may collectively encode three, four, five, six, seven, eight, nine, ten, 11, 12, 13, 14, 15, 17, 18, 19, 20, or more, of APP, PSEN1, PSEN2, APOE, TOMM40, GAB2, APOC1, TREM2, ABI3, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1 L, STK24, DISC1, MPZL1, SLC4A1AP, TRIP4, MSRA, HS3ST1, ZNF224, and AP2A2, such as three, four, five, six, seven, eight, nine, ten, 11, 12, 13, 14, 15, 17, 18, 19, 20, or more, of PSEN1, GAB2, APOC1, TREM2, ABI3, BIN1, HLA-DRB5, HLA-DRB1, CD2AP, PTK2B, INPP5D, MEF2C, CD33, MS4A4A, RIN3, PICALM, CASS4, SORL1, PLCG2, SCIMP, FRMD4A, SPPL2A, MTHFD1 L, DISCI , TRIP4, and HS3ST1.

In some embodiments, the one or more nucleic acid molecules collectively encode from two to 20 of the proteins, such as from two to 19, two to 18, two to 17, two to 16, two to 15, two to 14, two to 13, two to 12, two to 11, two to ten, two to nine, two to eight, two to seven, two to six, two to five, two to four, three to 20, three to 19, three to 18, three to 17, three to 16, three to 15, three to 14, three to 13, three to 12, three to 11, three to ten, three to nine, three to eight, three to seven, three to six, three to five, four to 20, four to 19, four to 18, four to 17, four to 16, four to 15, four to 14, four to 13, four to 12, four to 11, four to ten, four to nine, four to eight, four to seven, four to six, five to 20, five to 19, five to 18, five to 17, five to 16, five to 15, five to 14, five to 13, five to 12, five to 11, five to ten, five to nine, five to eight, five to seven, six to 20, six to 19, six to 18, six to 17, six to 16, six to 15, six to 14, six to 13, six to 12, six to 11, six to ten, six to nine, six to eight, seven to 20, seven to 19, seven to 18, seven to 17, seven to 16, seven to 15, seven to 14, seven to 13, seven to 12, seven to 11, seven to ten, seven to nine, eight to 20, eight to 19, eight to 18, eight to 17, eight to 16, eight to 15, eight to 14, eight to 13, eight to 12, eight to 11, eight to ten, nine to 20, nine to 19, nine to 18, nine to 17, nine to 16, nine to 15, nine to 14, nine to 13, nine to 12, nine to 11, ten to 20, ten to 19, ten to 18, ten to 17, ten to 16, ten to 15, ten to 14, ten to 13, ten to 12, 11 to 20, 11 to 19, 11 to 18, 11 to 17, 11 to 16, 11 to 15, 11 to 14, 11 to 13, 12 to 20, 12 to 19, 12 to 18, 12 to 17, 12 to 16, 12 to 15, 12 to 14, 13 to 20, 13 to 19, 13 to 18, 13 to 17, 13 to 16, 13 to 15, 14 to 20, 14 to 19, 14 to 18, 14 to 17, 14 to 16, 15 to 20, 15 to 19, 15 to 18, 15 to 17, 16 to 20, 16 to 19, 16 to 18, 17 to 20, 17 to 19, or 18 to 20 of proteins APP, PSEN1, PSEN2, APOE, TOMM40, GAB2, APOC1, TREM2, ABI3, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1 L, STK24, DISC1, MPZL1, SLC4A1 AP, TRIP4, MSRA, HS3ST1, ZNF224, and AP2A2 (e.g., from two to 19, two to 18, two to 17, two to 16, two to 15, two to 14, two to 13, two to 12, two to 11, two to ten, two to nine, two to eight, two to seven, two to six, two to five, two to four, three to 20, three to 19, three to 18, three to 17, three to 16, three to 15, three to 14, three to 13, three to 12, three to 11, three to ten, three to nine, three to eight, three to seven, three to six, three to five, four to 20, four to 19, four to 18, four to 17, four to 16, four to 15, four to 14, four to 13, four to 12, four to 11, four to ten, four to nine, four to eight, four to seven, four to six, five to 20, five to 19, five to 18, five to 17, five to 16, five to 15, five to 14, five to 13, five to 12, five to 11, five to ten, five to nine, five to eight, five to seven, six to 20, six to 19, six to 18, six to 17, six to 16, six to 15, six to 14, six to 13, six to 12, six to 11, six to ten, six to nine, six to eight, seven to 20, seven to 19, seven to 18, seven to 17, seven to 16, seven to 15, seven to 14, seven to 13, seven to 12, seven to 11, seven to ten, seven to nine, eight to 20, eight to 19, eight to 18, eight to 17, eight to 16, eight to 15, eight to 14, eight to 13, eight to 12, eight to 11, eight to ten, nine to 20, nine to 19, nine to 18, nine to 17, nine to 16, nine to 15, nine to 14, nine to 13, nine to 12, nine to 11, ten to 20, ten to 19, ten to 18, ten to 17, ten to 16, ten to 15, ten to 14, ten to 13, ten to 12, 11 to 20, 11 to 19, 11 to 18,11 to 17,11 to 16,11 to 15,11 to 14,11 to 13, 12 to 20, 12 to 19, 12 to 18, 12 to 17, 12 to 16, 12 to 15, 12 to 14, 13 to 20, 13 to 19, 13 to 18, 13 to 17, 13 to 16, 13 to 15, 14 to 20, 14 to 19, 14 to 18, 14 to 17, 14 to 16, 15 to 20, 15 to 19, 15 to 18, 15 to 17, 16 to 20, 16 to 19, 16 to 18, 17 to 20, 17 to 19, or 18 to 20 of proteins PSEN1, GAB2, APOC1, TREM2, ABI3, BIN1, HLA-DRB5, HLA-DRB1, CD2AP, PTK2B, INPP5D, MEF2C, CD33, MS4A4A, RIN3, PICALM, CASS4, SORL1, PLCG2, SCIMP, FRMD4A, SPPL2A, MTHFD1 L, DISCI , TRIP4, and HS3ST1). In some embodiments, the one or more nucleic acid molecules collectively encode a panel of proteins set forth in Table 1, herein.

Similarly, in cases of treating Parkinson's disease, the patient may be provided one or more nucleic acid molecules that collectively encode one or more proteins selected from FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1 L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1 B, NUCKS1, and ACMSD, such as one or more proteins selected from FCGR2A, SCAF11, DNAJC13, GCH1, LRRK2, GBA, GAK, FGF20, HLA-DQB1, and NOD2.

For example, in cases of treating Parkinson's disease, the patient may be provided one or more nucleic acid molecules that collectively encode of two or more of the proteins selected from FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1 L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1 B, NUCKS1, and ACMSD, such as two or more proteins selected from FCGR2A, SCAF11, DNAJC13, GCH1, LRRK2, GBA, GAK, FGF20, HLA-DQB1, and NOD2. For example, the one or more nucleic acid molecules may collectively encode three, four, five, six, seven, eight, nine, ten, 11, 12, 13, 14, 15, 17, 18, 19, 20, or more, of FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1B, NUCKS1, and ACMSD, such as three, four, five, six, seven, eight, nine, or more, of FCGR2A, SCAF11, DNAJC13, GCH1, LRRK2, GBA, GAK, FGF20, HLA-DQB1, and NOD2.

In some embodiments, the one or more nucleic acid molecules collectively encode from two to 20 of the proteins, such as from two to 19, two to 18, two to 17, two to 16, two to 15, two to 14, two to 13, two to 12, two to 11, two to ten, two to nine, two to eight, two to seven, two to six, two to five, two to four, three to 20, three to 19, three to 18, three to 17, three to 16, three to 15, three to 14, three to 13, three to 12, three to 11, three to ten, three to nine, three to eight, three to seven, three to six, three to five, four to 20, four to 19, four to 18, four to 17, four to 16, four to 15, four to 14, four to 13, four to 12, four to 11, four to ten, four to nine, four to eight, four to seven, four to six, five to 20, five to 19, five to 18, five to 17, five to 16, five to 15, five to 14, five to 13, five to 12, five to 11, five to ten, five to nine, five to eight, five to seven, six to 20, six to 19, six to 18, six to 17, six to 16, six to 15, six to 14, six to 13, six to 12, six to 11, six to ten, six to nine, six to eight, seven to 20, seven to 19, seven to 18, seven to 17, seven to 16, seven to 15, seven to 14, seven to 13, seven to 12, seven to 11, seven to ten, seven to nine, eight to 20, eight to 19, eight to 18, eight to 17, eight to 16, eight to 15, eight to 14, eight to 13, eight to 12, eight to 11, eight to ten, nine to 20, nine to 19, nine to 18, nine to 17, nine to 16, nine to 15, nine to 14, nine to 13, nine to 12, nine to 11, ten to 20, ten to 19, ten to 18, ten to 17, ten to 16, ten to 15, ten to 14, ten to 13, ten to 12, 11 to 20, 11 to 19, 11 to 18, 11 to 17, 11 to 16, 11 to 15, 11 to 14, 11 to 13, 12 to 20, 12 to 19, 12 to 18, 12 to 17, 12 to 16, 12 to 15, 12 to 14, 13 to 20, 13 to 19, 13 to 18, 13 to 17, 13 to 16, 13 to 15, 14 to 20, 14 to 19, 14 to 18, 14 to 17, 14 to 16, 15 to 20, 15 to 19, 15 to 18, 15 to 17, 16 to 20, 16 to 19, 16 to 18, 17 to 20, 17 to 19, or 18 to 20 of proteins FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1B, NUCKS1, and ACMSD (e.g., from two to ten, two to nine, two to eight, two to seven, two to six, two to five, two to four, three to ten, three to nine, three to eight, three to seven, three to six, three to five, four to ten, four to nine, four to eight, four to seven, four to six, five to ten, five to nine, five to eight, five to seven, six to ten, six to nine, six to eight, seven to ten, seven to nine, or eight to ten of proteins FCGR2A, SCAF11, DNAJC13, GCH1, LRRK2, GBA, GAK, FGF20, HLA-DQB1, and NOD2). In some embodiments, the one or more nucleic acid molecules collectively encode a panel of proteins set forth in Table 2, herein.

Similarly, in cases of treating a FTLD, such as behavioral-variant frontotemporal dementia, semantic dementia, or progressive nonfluent aphasia, the patient may be provided one or more nucleic acid molecules that collectively encode one or more proteins selected from HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT, such as one or more proteins selected from HLA-DRA, HLA-DRB5, C9ORF72, SQSTM1, TBK1, PSEN1, GRN, and CTSF.

For example, in cases of treating a FTLD, the patient may be provided one or more nucleic acid molecules that collectively encode of two or more of the proteins selected from HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT, such as two or more proteins selected from HLA-DRA, HLA-DRB5, C9ORF72, SQSTM1, TBK1, PSEN1, GRN, and CTSF. For example, the one or more nucleic acid molecules may collectively encode three, four, five, six, seven, eight, nine, ten, 11, 12, 13, 14, 15, or more, of HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT, such as three, four, five, six, or more, of HLA-DRA, HLA-DRB5, C9ORF72, SQSTM1, TBK1, PSEN1, GRN, and CTSF.

In some embodiments, the one or more nucleic acid molecules collectively encode from two to 15 of the proteins, such as from two to 14, two to 13, two to 12, two to 11, two to ten, two to nine, two to eight, two to seven, two to six, two to five, two to four, three to 15, three to 14, three to 13, three to 12, three to 11, three to ten, three to nine, three to eight, three to seven, three to six, three to five, four to 15, four to 14, four to 13, four to 12, four to 11, four to ten, four to nine, four to eight, four to seven, four to six, five to 15, five to 14, five to 13, five to 12, five to 11, five to ten, five to nine, five to eight, five to seven, six to 15, six to 14, six to 13, six to 12, six to 11, six to ten, six to nine, six to eight, seven to 15, seven to 14, seven to 13, seven to 12, seven to 11, seven to ten, seven to nine, eight to 15, eight to 14, eight to 13, eight to 12, eight to 11, eight to ten, nine to 15, nine to 14, nine to 13, nine to 12, nine to 11, ten to 15, ten to 14, ten to 13, ten to 12, 11 to 15, 11 to 14, 11 to 13, 12 to 15, or 12 to 14 of proteins HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT, such as two or more proteins selected from HLA-DRA, HLA-DRB5, C9ORF72, SQSTM1, TBK1, PSEN1, GRN, and CTSF (e.g., from two to six, two to five, two to four, three to six, three to five, four to ten, or four to six, of proteins HLA-DRA, HLA-DRB5, C9ORF72, SQSTM1, TBK1, PSEN1, GRN, and CTSF). In some embodiments, the one or more nucleic acid molecules collectively encode a panel of proteins set forth in Table 3, herein.

Similarly, in cases of treating a patient diagnosed with Alzheimer's disease, Parkinson disease, or a FTLD, such as behavioral-variant frontotemporal dementia, semantic dementia, or progressive nonfluent aphasia, the patient may be provided one or more nucleic acid molecules that collectively encode one or more proteins selected from APP, PSEN1, PSEN2, APOE, TOMM40, GAB2, APOC1, TREM2, ABI3, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1L, STK24, DISCI , MPZL1, SLC4A1AP, TRIP4, MSRA, HS3ST1, ZNF224, AP2A2, FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1B, NUCKS1, ACMSD, HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT.

For example, in cases of treating Alzheimer's disease, Parkinson disease, or a FTLD, the patient may be provided one or more nucleic acid molecules that collectively encode of two or more of the proteins selected from APP, PSEN1, PSEN2, APOE, TOMM40, GAB2, APOC1, TREM2, ABI3, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1L, STK24, DISCI , MPZL1, SLC4A1AP, TRIP4, MSRA, HS3ST1, ZNF224, AP2A2, FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1 L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1B, NUCKS1, ACMSD, HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT. For example, the one or more nucleic acid molecules may collectively encode two, three, four, five, six, seven, eight, nine, ten, 11, 12, 13, 14, 15, or more, of APP, PSEN1, PSEN2, APOE, TOMM40, GAB2, APOC1, TREM2, ABI3, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1L, STK24, DISCI , MPZL1, SLC4A1AP, TRIP4, MSRA, HS3ST1, ZNF224, AP2A2, FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1B, NUCKS1, ACMSD, HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT.

In some embodiments, the one or more nucleic acid molecules collectively encode from two to 15 of the proteins, such as from two to 14, two to 13, two to 12, two to 11, two to ten, two to nine, two to eight, two to seven, two to six, two to five, two to four, three to 15, three to 14, three to 13, three to 12, three to 11, three to ten, three to nine, three to eight, three to seven, three to six, three to five, four to 15, four to 14, four to 13, four to 12, four to 11, four to ten, four to nine, four to eight, four to seven, four to six, five to 15, five to 14, five to 13, five to 12, five to 11, five to ten, five to nine, five to eight, five to seven, six to 15, six to 14, six to 13, six to 12, six to 11, six to ten, six to nine, six to eight, seven to 15, seven to 14, seven to 13, seven to 12, seven to 11, seven to ten, seven to nine, eight to 15, eight to 14, eight to 13, eight to 12, eight to 11, eight to ten, nine to 15, nine to 14, nine to 13, nine to 12, nine to 11, ten to 15, ten to 14, ten to 13, ten to 12, 11 to 15, 11 to 14, 11 to 13, 12 to 15, or 12 to 14 of proteins APP, PSEN1, PSEN2, APOE, TOMM40, GAB2, APOC1, TREM2, ABI3, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1 L, STK24, DISCI , MPZL1, SLC4A1AP, TRIP4, MSRA, HS3ST1, ZNF224, AP2A2, FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1 L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1B, NUCKS1, ACMSD, HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT. In some embodiments, the one or more nucleic acid molecules collectively encode a panel of proteins set forth in Table 4, herein.

In some embodiments of any of the foregoing aspects of the disclosure, the one or more nucleic acid molecules are provided to the patient by administering to the patient a composition containing a population of cells that together contain one or more transgenes encoding the one or more proteins. The cells may be cells such as, e.g., pluripotent cells, ESCs, iPSCs, multipotent cells, CD34+ cells, HSCs, MPCs, BLPCs, monocytes, macrophages, microglial progenitor cells, or microglia. The population may be a uniform population of cells that contain nucleic acids encoding one or more proteins. The uniform population may be, for example, a population of cells in which at least 50%, 55%, 60%, 65%, 70%, 75%, 80%, 85%, 90%, 95%, 96%, 97%, 98%, 99%, 99.9%, 99.99%, or more (e.g., 100%) of the cells contain a nucleic acid encoding the one or more proteins. In some embodiments, the population is a heterogeneous population of cells that together contain a nucleic acid encoding the one or more proteins.

In some embodiments of any of the foregoing aspects of the disclosure, the composition is administered systemically to the patient. For example, the composition may be administered to the patient by way of intravenous injection. In some embodiments, the composition is administered directly to the central nervous system of the patient, such as directly to the cerebrospinal fluid (CSF) of the patient. In some embodiments, the composition if administered to the patient by way of intracerebroventricular (ICV) injection, intrathecal injection, stereotactic injection, intraparenchymal injection, or a combination thereof.

In some embodiments, the patient is diagnosed with an NCD. In some embodiments, the NCD is a major NCD. In some embodiments, the major NCD interferes with the patient's independence and/or normal daily functioning (e.g., social, occupational, or academic functioning, personal hygiene, grooming, dressing, toilet hygiene, functional mobility (e.g., ability to walk, get in and out of bed), and self-feeding. In some embodiments, the major NCD is associated with a score obtained by the patient on a cognitive test that is at least two standard deviations away from the mean score of a reference population. In some embodiments, the NCD is a mild NCD. In some embodiments, the mild NCD does not interfere with the patient's independence and/or normal daily functioning. In some embodiments, the mild NCD is associated with a score obtained by the patient on a cognitive test that is between one to two standard deviations away from the mean score of a reference population. In some embodiments, the cognitive test is selected from the group consisting of ADB, AWV, GPCOG, HRA, MIS, MMSE, MoCA, SLUMS, and Short IQCODE. In some embodiments, the NCD is associated with impairment in one or more of complex attention, executive function, learning and memory, language, perceptual-motor function, and social cognition. In some embodiments, the NCD is not due to delirium or other mental disorder (e.g., schizophrenia, bipolar disorder, or major depression). In some embodiments, the reference population is a general population. In some embodiments, the reference population is selected on the basis of the patient's age, medical history, education, socioeconomic status, and lifestyle. In some embodiments, the NCD is Alzheimer's disease. In some embodiments, the NCD is a movement disorder. In some embodiments, the movement disorder is Parkinson disease. In some embodiments, the NCD is a frontotemporal NCD. In some embodiments, the frontotemporal NCD is a FTLD. In some embodiments, the FTLD is a behavioral-variant frontotemporal dementia. In some embodiments, the FTLD is a semantic dementia. In some embodiments, the FTLD is a progressive nonfluent aphasia.

In some embodiments, the composition is administered to the patient both systemically and directly to the central nervous system. For example, the composition may be administered to the patient by way of intravenous injection and directly to the CSF of the patient. In some embodiments, the composition is administered to the patient by way of intravenous injection and by way of ICV injection, intrathecal injection, stereotactic injection, intraparenchymal injection, or a combination thereof.

In some embodiments, the cells are autologous cells. In some embodiments, the cells are allogeneic cells.

In some embodiments, the cells are transduced ex vivo to express the one or more proteins. For example, the cells may be transduced with a viral vector selected from the group consisting of an adeno-associated virus (AAV), an adenovirus, a parvovirus, a coronavirus, a rhabdovirus, a paramyxovirus, a picornavirus, an alphavirus, a herpes virus, a poxvirus, and a Retroviridae family virus. In some embodiments, the viral vector is a Retroviridae family viral vector, such as a lentiviral vector, alpharetroviral vector, or gammaretroviral vector. In some embodiments, the Retroviridae family viral vector contains a central polypurine tract, a woodchuck hepatitis virus post-transcriptional regulatory element, a 5′-LTR, HIV signal sequence, HIV Psi signal 5′-splice site, delta-GAG element, 3′-splice site, and a 3′-self inactivating LTR. In some embodiments, the viral vector is an AAV selected from the group consisting of AAV1, AAV2, AAV3, AAV4, AAVS, AAV6, AAV7, AAV8, AAV9, AAV10, and AAVrh74. In some embodiments, the viral vector is a pseudotyped viral vector, such as a pseudotyped viral vector selected from the group consisting of a pseudotyped AAV, a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus.

In some embodiments, the cells are transfected ex vivo to express the one or more proteins. For example, the cells may be transfected using an agent selected from the group consisting of a cationic polymer, diethylaminoethyldextran, polyethylenimine, a cationic lipid, a liposome, calcium phosphate, an activated dendrimer, and a magnetic bead. In some embodiments, the cells are transfected using a technique selected from the group consisting of electroporation, Nucleofection, squeeze-poration, sonoporation, optical transfection, Magnetofection, and impalefection.

In some embodiments, the one or more nucleic acid molecules are provided to the patient by administering to the patient one or more viral vectors that together contain the one or more nucleic acid molecules. In some embodiments, the patient is administered a plurality of viral vectors that together contain the one or more nucleic acid molecules. In some embodiments, the patient is administered a plurality of viral vectors that each individually contain the one or more nucleic acid molecules. In some embodiments, the patient is administered a single viral vector that contains the one or more nucleic acid molecules.

In some embodiments, the one or more viral vectors are administered systemically to the patient. For example, the one or more viral vectors may be administered to the patient by way of intravenous injection. In some embodiments, the one or more viral vectors are administered directly to the central nervous system of the patient, such as directly to the cerebrospinal fluid (CSF) of the patient. In some embodiments, the one or more viral vectors are administered to the patient by way of intracerebroventricular (ICV) injection, intrathecal injection, stereotactic injection, intraparenchymal injection, or a combination thereof.

In some embodiments, the one or more viral vectors are administered to the patient both systemically and directly to the central nervous system. For example, the one or more viral vectors may be administered to the patient by way of intravenous injection and directly to the CSF of the patient. In some embodiments, the one or more viral vectors are administered to the patient by way of intravenous injection and by way of ICV injection, intrathecal injection, stereotactic injection, intraparenchymal injection, or a combination thereof.

In some embodiments, the one or more viral vectors contain an AAV, an adenovirus, a parvovirus, a coronavirus, a rhabdovirus, a paramyxovirus, a picornavirus, an alphavirus, a herpes virus, a poxvirus, or a Retroviridae family virus. In some embodiments, the viral vector is a Retroviridae family viral vector, such as a lentiviral vector, alpharetroviral vector, or gammaretroviral vector. In some embodiments, the Retroviridae family viral vector contains a central polypurine tract, a woodchuck hepatitis virus post-transcriptional regulatory element, a 5′-LTR, HIV signal sequence, HIV Psi signal 5′-splice site, delta-GAG element, 3′-splice site, and a 3′-self inactivating LTR. In some embodiments, the viral vector is an AAV selected from the group consisting of AAV1, AAV2, AAV3, AAV4, AAV5, AAV6, AAV7, AAV8, AAV9, AAV10, and AAVrh74. In some embodiments, the viral vector is a pseudotyped viral vector, such as a pseudotyped viral vector selected from the group consisting of a pseudotyped AAV, a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus.

In some embodiments of any of the foregoing aspects of the disclosure, the one or more nucleic acid molecules contain a transgene encoding one or more of the proteins operably linked to a ubiquitous promoter. The ubiquitous promoter may be, for example, an elongation factor 1-alpha promoter or a phosphoglycerate kinase 1 promoter. In some embodiments, the one or more nucleic acid molecules contain a transgene encoding one or more of the proteins operably linked to a cell lineage-specific promoter. The cell lineage-specific promoter may be, for example, a PGRN promoter, a CD11 b promoter, CD68 promoter, a C—X3-C motif chemokine receptor 1 promoter, an allograft inflammatory factor 1 promoter, a purinergic receptor P2Y12 promoter, a transmembrane protein 119 promoter, or a colony stimulating factor 1 receptor promoter. In some embodiments, the one or more nucleic acid molecules contain a transgene encoding one or more of the proteins operably linked to a synthetic promoter.

In some embodiments, one or more of the proteins further contains a receptor-binding (Rb) domain of apolipoprotein E (ApoE). The Rb domain may contain a portion of ApoE, such as a portion having the amino acid sequence of residues 25-185, 50-180, 75-175, 100-170, 125-160, or 130-150 of SEQ ID NO: 105. In some embodiments, the Rb domain contains a region having at least 70% sequence identity (e.g., a region having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, or 100% sequence identity) to the amino acid sequence of residues 159-167 of SEQ ID NO: 105.

In some embodiments, the one or more nucleic acid molecules contain a micro RNA (miRNA) targeting sequence in the 3′-UTR. In some embodiments, the miRNA targeting sequence is a miR-126 targeting sequence.

In some embodiments, upon providing the one or more nucleic acid molecules to the patient, the one or more proteins penetrate the blood-brain barrier in the patient.

In some embodiments, a population of endogenous microglia in the patient has been ablated prior to providing the patient with the composition (e.g., the one or more nucleic acid molecules). In some embodiments, the method includes ablating a population of endogenous microglia in the patient prior to providing the patient with the composition (e.g., the one or more nucleic acid molecules). The microglia may be ablated, for example, using an agent selected from busulfan, PLX3397, PLX647, PLX5622, treosulfan, and clodronate liposomes; by radiation therapy; or a combination thereof.

In some embodiments, prior to providing the patient with the composition (e.g., the one or more nucleic acid molecules), endogenous expression of one or more of the proteins is disrupted in the cells administered to the patient. Endogenous expression of the one or more proteins may be disrupted in the cells administered to the patient, for example, by contacting the cells with a nuclease that catalyzes cleavage of an endogenous gene encoding one of the proteins. The nuclease may be a clustered regularly interspaced short palindromic repeats (CRISPR)-associated protein, such as CRISPR-associated protein 9 (Cas9) or CRISPR-associated protein is CRISPR-associated protein 12a (Cas12a), among others. In some embodiments, the nuclease is a transcription activator-like effector nuclease, a meganuclease, or a zinc finger nuclease.

Additionally or alternatively, endogenous expression of the one or more proteins may be disrupted in the cells administered to the patient by contacting the cells with an inhibitory RNA molecule, such as a siRNA, a shRNA, or a miRNA that is specific for (e.g., that anneals to), and suppresses the expression of, a gene encoding one of the proteins.

In some embodiments, prior to providing the patient with the composition (e.g., the one or more nucleic acid molecules), endogenous expression of one or more of the proteins is disrupted in the patient. For example, in some embodiments, prior to providing the patient with the composition (e.g., the one or more nucleic acid molecules), endogenous expression of one or more of the proteins is disrupted in a population of neurons in the patient. Endogenous expression of one or more of the proteins may be disrupted by contacting the cells with an inhibitory RNA molecule, such as a siRNA, a shRNA, or a miRNA that is specific for (e.g., that anneals to), and suppresses the expression of, a gene encoding one of the proteins.

In a fifth aspect, the disclosure provides a pharmaceutical composition containing a population of cells that together contain one or more nucleic acids encoding one or more proteins selected from APP, PSEN1, PSEN2, APOE, TOMM40, GAB2, APOC1, TREM2, AB13, BIN1, CR1, ABCA7, FERMT2, HLA- DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1 L, STK24, DISCI , MPZL1, SLC4A1AP, TRIP4, MSRA, HS3ST1, ZNF224, and AP2A2, such as one or more proteins selected from PSEN1, GAB2, APOC1, TREM2, AB13, BIN1, HLA-DRB5, HLA-DRB1, CD2AP, PTK2B, INPP5D, MEF2C, CD33, MS4A4A, RIN3, PICALM, CASS4, SORL1, PLCG2, SCIMP, FRMD4A, SPPL2A, MTHFD1 L, DISC1, TRIP4, and HS3ST1.

In some embodiments of the foregoing aspect, the cells together contain one or more nucleic acids encoding two or more of the proteins selected from APP, PSEN1, PSEN2, APOE, TOMM40, GAB2, APOC1, TREM2, AB13, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1L, STK24, DISCI , MPZL1, SLC4A1AP, TRIP4, MSRA, HS3ST1, ZNF224, and AP2A2, such as two or more proteins selected from PSEN1, GAB2, APOC1, TREM2, AB13, BIN1, HLA-DRB5, HLA-DRB1, CD2AP, PTK2B, INPP5D, MEF2C, CD33, MS4A4A, RIN3, PICALM, CASS4, SORL1, PLCG2, SCIMP, FRMD4A, SPPL2A, MTHFD1 L, DISC1, TRIP4, and HS3ST1. For example, the cells may together contain one or more nucleic acids encoding three, four, five, six, seven, eight, nine, ten, 11, 12, 13, 14, 15, 17, 18, 19, 20, or more, of APP, PSEN1, PSEN2, APOE, TOMM40, GAB2, APOC1, TREM2, ABI3, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1L, STK24, DISCI , MPZL1, SLC4A1AP, TRIP4, MSRA, HS3ST1, ZNF224, and AP2A2, such as three, four, five, six, seven, eight, nine, ten, 11, 12, 13, 14, 15, 17, 18, 19, 20, or more, of PSEN1, GAB2, APOC1, TREM2, ABI3, BIN1, HLA-DRB5, HLA-DRB1, CD2AP, PTK2B, INPP5D, MEF2C, CD33, MS4A4A, RIN3, PICALM, CASS4, SORL1, PLCG2, SCIMP, FRMD4A, SPPL2A, MTHFD1 L, DISC1, TRIP4, and HS3ST1.

In some embodiments of the foregoing aspect, the cells together contain one or more nucleic acids encoding from two to 20 of the proteins, such as from two to 19, two to 18, two to 17, two to 16, two to 15, two to 14, two to 13, two to 12, two to 11, two to ten, two to nine, two to eight, two to seven, two to six, two to five, two to four, three to 20, three to 19, three to 18, three to 17, three to 16, three to 15, three to 14, three to 13, three to 12, three to 11, three to ten, three to nine, three to eight, three to seven, three to six, three to five, four to 20, four to 19, four to 18, four to 17, four to 16, four to 15, four to 14, four to 13, four to 12, four to 11, four to ten, four to nine, four to eight, four to seven, four to six, five to 20, five to 19, five to 18, five to 17, five to 16, five to 15, five to 14, five to 13, five to 12, five to 11, five to ten, five to nine, five to eight, five to seven, six to 20, six to 19, six to 18, six to 17, six to 16, six to 15, six to 14, six to 13, six to 12, six to 11, six to ten, six to nine, six to eight, seven to 20, seven to 19, seven to 18, seven to 17, seven to 16, seven to 15, seven to 14, seven to 13, seven to 12, seven to 11, seven to ten, seven to nine, eight to 20, eight to 19, eight to 18, eight to 17, eight to 16, eight to 15, eight to 14, eight to 13, eight to 12, eight to 11, eight to ten, nine to 20, nine to 19, nine to 18, nine to 17, nine to 16, nine to 15, nine to 14, nine to 13, nine to 12, nine to 11, ten to 20, ten to 19, ten to 18, ten to 17, ten to 16, ten to 15, ten to 14, ten to 13, ten to 12, 11 to 20, 11 to 19, 11 to 18, 11 to 17, 11 to 16, 11 to 15, 11 to 14, 11 to 13, 12 to 20, 12 to 19, 12 to 18, 12 to 17, 12 to 16, 12 to 15, 12 to 14, 13 to 20, 13 to 19, 13 to 18, 13 to 17, 13 to 16, 13 to 15, 14 to 20, 14 to 19, 14 to 18, 14 to 17, 14 to 16, 15 to 20, 15 to 19, 15 to 18, 15 to 17, 16 to 20, 16 to 19, 16 to 18, 17 to 20, 17 to 19, or 18 to 20 of proteins APP, PSEN1, PSEN2, APOE, TOMM40, GAB2, APOC1, TREM2, ABI3, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1L, STK24, DISCI , MPZL1, SLC4A1AP, TRIP4, MSRA, HS3ST1, ZNF224, and AP2A2 (e.g., from two to 19, two to 18, two to 17, two to 16, two to 15, two to 14, two to 13, two to 12, two to 11, two to ten, two to nine, two to eight, two to seven, two to six, two to five, two to four, three to 20, three to 19, three to 18, three to 17, three to 16, three to 15, three to 14, three to 13, three to 12, three to 11, three to ten, three to nine, three to eight, three to seven, three to six, three to five, four to 20, four to 19, four to 18, four to 17, four to 16, four to 15, four to 14, four to 13, four to 12, four to 11, four to ten, four to nine, four to eight, four to seven, four to six, five to 20, five to 19, five to 18, five to 17, five to 16, five to 15, five to 14, five to 13, five to 12, five to 11, five to ten, five to nine, five to eight, five to seven, six to 20, six to 19, six to 18, six to 17, six to 16, six to 15, six to 14, six to 13, six to 12, six to 11, six to ten, six to nine, six to eight, seven to 20, seven to 19, seven to 18, seven to 17, seven to 16, seven to 15, seven to 14, seven to 13, seven to 12, seven to 11, seven to ten, seven to nine, eight to 20, eight to 19, eight to 18, eight to 17, eight to 16, eight to 15, eight to 14, eight to 13, eight to 12, eight to 11, eight to ten, nine to 20, nine to 19, nine to 18, nine to 17, nine to 16, nine to 15, nine to 14, nine to 13, nine to 12, nine to 11, ten to 20, ten to 19, ten to 18, ten to 17, ten to 16, ten to 15, ten to 14, ten to 13, ten to 12, 11 to 20, 11 to 19, 11 to 18, 11 to 17, 11 to 16, 11 to 15, 11 to 14, 11 to 13, 12 to 20, 12 to 19, 12 to 18, 12 to 17, 12 to 16, 12 to 15, 12 to 14, 13 to 20, 13 to 19, 13 to 18, 13 to 17, 13 to 16, 13 to 15, 14 to 20, 14 to 19, 14 to 18, 14 to 17, 14 to 16, 15 to 20, 15 to 19, 15 to 18, 15 to 17, 16 to 20, 16 to 19, 16 to 18, 17 to 20, 17 to 19, or 18 to 20 of proteins PSEN1, GAB2, APOC1, TREM2, ABI3, BIN1, HLA-DRB5, HLA-DRB1, CD2AP, PTK2B, INPP5D, MEF2C, CD33, MS4A4A, RIN3, PICALM, CASS4, SORL1, PLCG2, SCIMP, FRMD4A, SPPL2A, MTHFD1 L, DISC1, TRIP4, and HS3ST1). In some embodiments, the proteins include a panel set forth in Table 1, herein.

In a sixth aspect, the disclosure provides a population of cells that together contain one or more nucleic acids encoding one or more proteins selected from FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1 L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1B, NUCKS1, and ACMSD, such as one or more proteins selected from FCGR2A, SCAF11, DNAJC13, GCH1, LRRK2, GBA, GAK, FGF20, HLA-DQB1, and NOD2.

In some embodiments of the foregoing aspect, the cells together contain one or more nucleic acids encoding two or more of the proteins selected from FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1 L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1B, NUCKS1, and ACMSD, such as two or more proteins selected from FCGR2A, SCAF11, DNAJC13, GCH1, LRRK2, GBA, GAK, FGF20, HLA-DQB1, and NOD2. For example, the cells may together contain one or more nucleic acids encoding three, four, five, six, seven, eight, nine, ten, 11, 12, 13, 14, 15, 17, 18, 19, 20, or more, of FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1 L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1 B, NUCKS1, and ACMSD, such as three, four, five, six, seven, eight, nine, or more, of FCGR2A, SCAF11, DNAJC13, GCH1, LRRK2, GBA, GAK, FGF20, HLA-DQB1, and NOD2.

In some embodiments of the foregoing aspect, the cells together contain one or more nucleic acids encoding from two to 20 of the proteins, such as from two to 19, two to 18, two to 17, two to 16, two to 15, two to 14, two to 13, two to 12, two to 11, two to ten, two to nine, two to eight, two to seven, two to six, two to five, two to four, three to 20, three to 19, three to 18, three to 17, three to 16, three to 15, three to 14, three to 13, three to 12, three to 11, three to ten, three to nine, three to eight, three to seven, three to six, three to five, four to 20, four to 19, four to 18, four to 17, four to 16, four to 15, four to 14, four to 13, four to 12, four to 11, four to ten, four to nine, four to eight, four to seven, four to six, five to 20, five to 19, five to 18, five to 17, five to 16, five to 15, five to 14, five to 13, five to 12, five to 11, five to ten, five to nine, five to eight, five to seven, six to 20, six to 19, six to 18, six to 17, six to 16, six to 15, six to 14, six to 13, six to 12, six to 11, six to ten, six to nine, six to eight, seven to 20, seven to 19, seven to 18, seven to 17, seven to 16, seven to 15, seven to 14, seven to 13, seven to 12, seven to 11, seven to ten, seven to nine, eight to 20, eight to 19, eight to 18, eight to 17, eight to 16, eight to 15, eight to 14, eight to 13, eight to 12, eight to 11, eight to ten, nine to 20, nine to 19, nine to 18, nine to 17, nine to 16, nine to 15, nine to 14, nine to 13, nine to 12, nine to 11, ten to 20, ten to 19, ten to 18, ten to 17, ten to 16, ten to 15, ten to 14, ten to 13, ten to 12, 11 to 20, 11 to 19, 11 to 18, 11 to 17, 11 to 16, 11 to 15, 11 to 14, 11 to 13, 12 to 20, 12 to 19, 12 to 18, 12 to 17, 12 to 16, 12 to 15, 12 to 14, 13 to 20, 13 to 19, 13 to 18, 13 to 17, 13 to 16, 13 to 15, 14 to 20, 14 to 19, 14 to 18, 14 to 17, 14 to 16, 15 to 20, 15 to 19, 15 to 18, 15 to 17, 16 to 20, 16 to 19, 16 to 18, 17 to 20, 17 to 19, or 18 to 20 of proteins FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1 L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1B, NUCKS1, and ACMSD (e.g., from two to ten, two to nine, two to eight, two to seven, two to six, two to five, two to four, three to ten, three to nine, three to eight, three to seven, three to six, three to five, four to ten, four to nine, four to eight, four to seven, four to six, five to ten, five to nine, five to eight, five to seven, six to ten, six to nine, six to eight, seven to ten, seven to nine, or eight to ten of proteins FCGR2A, SCAF11, DNAJC13, GCH1, LRRK2, GBA, GAK, FGF20, HLA-DQB1, and NOD2). In some embodiments, the proteins include a panel set forth in Table 2, herein.

In a seventh aspect, the disclosure provides a population of cells that together contain one or more nucleic acids encoding one or more proteins selected from HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT, such as one or more proteins selected from HLA-DRA, HLA-DRB5, C9ORF72, SQSTM1, TBK1, PSEN1, GRN, and CTSF.

In some embodiments of the foregoing aspect, the cells together contain one or more nucleic acids encoding two or more of the proteins selected from HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT, such as two or more proteins selected from HLA-DRA, HLA-DRB5, C9ORF72, SQSTM1, TBK1, PSEN1, GRN, and CTSF. For example, the cells may together contain one or more nucleic acids encoding three, four, five, six, seven, eight, nine, ten, 11, 12, 13, 14, 15, or more, of HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT, such as three, four, five, six, or more, of HLA-DRA, HLA-DRB5, C9ORF72, SQSTM1, TBK1, PSEN1, GRN, and CTSF.

In some embodiments of the foregoing aspect, the cells together contain one or more nucleic acids encoding from two to 15 of the proteins, such as from two to 14, two to 13, two to 12, two to 11, two to ten, two to nine, two to eight, two to seven, two to six, two to five, two to four, three to 15, three to 14, three to 13, three to 12, three to 11, three to ten, three to nine, three to eight, three to seven, three to six, three to five, four to 15, four to 14, four to 13, four to 12, four to 11, four to ten, four to nine, four to eight, four to seven, four to six, five to 15, five to 14, five to 13, five to 12, five to 11, five to ten, five to nine, five to eight, five to seven, six to 15, six to 14, six to 13, six to 12, six to 11, six to ten, six to nine, six to eight, seven to 15, seven to 14, seven to 13, seven to 12, seven to 11, seven to ten, seven to nine, eight to 15, eight to 14, eight to 13, eight to 12, eight to 11, eight to ten, nine to 15, nine to 14, nine to 13, nine to 12, nine to 11, ten to 15, ten to 14, ten to 13, ten to 12, 11 to 15, 11 to 14, 11 to 13, 12 to 15, or 12 to 14 of proteins HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT, such as two or more proteins selected from HLA-DRA, HLA-DRB5, C9ORF72, SQSTM1, TBK1, PSEN1, GRN, and CTSF (e.g., from two to six, two to five, two to four, three to six, three to five, four to ten, or four to six, of proteins HLA-DRA, HLA-DRB5, C9ORF72, SQSTM1, TBK1, PSEN1, GRN, and CTSF). In some embodiments, the proteins include a panel set forth in Table 3, herein. In an eigth aspect, the disclosure provides a population of cells that together contain one or more nucleic acids encoding one or more proteins selected from APP, PSEN1, PSEN2, APOE, TOMM40, GAB2, APOC1, TREM2, ABI3, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1L, STK24, DISCI , MPZL1, SLC4A1AP, TRIP4, MSRA, HS3ST1, ZNF224, AP2A2, FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1B, NUCKS1, ACMSD, HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT.

In some embodiments of the foregoing aspect, the cells together contain one or more nucleic acids encoding two or more of the proteins selected from APP, PSEN1, PSEN2, APOE, TOMM40, GAB2, APOC1, TREM2, ABI3, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1L, STK24, DISCI , MPZL1, SLC4A1AP, TRIP4, MSRA, HS3ST1, ZNF224, AP2A2, FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1B, NUCKS1, ACMSD, HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT. For example, the cells may together contain one or more nucleic acids encoding three, four, five, six, seven, eight, nine, ten, 11, 12, 13, 14, 15, or more, of APP, PSEN1, PSEN2, APOE, TOMM40, GAB2, APOC1, TREM2, ABI3, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1 L, STK24, DISCI , MPZL1, SLC4A1AP, TRIP4, MSRA, HS3ST1, ZNF224, AP2A2, FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1 L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1B, NUCKS1, ACMSD, HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT.

In some embodiments of the foregoing aspect, the cells together contain one or more nucleic acids encoding from two to 15 of the proteins, such as from two to 14, two to 13, two to 12, two to 11, two to ten, two to nine, two to eight, two to seven, two to six, two to five, two to four, three to 15, three to 14, three to 13, three to 12, three to 11, three to ten, three to nine, three to eight, three to seven, three to six, three to five, four to 15, four to 14, four to 13, four to 12, four to 11, four to ten, four to nine, four to eight, four to seven, four to six, five to 15, five to 14, five to 13, five to 12, five to 11, five to ten, five to nine, five to eight, five to seven, six to 15, six to 14, six to 13, six to 12, six to 11, six to ten, six to nine, six to eight, seven to 15, seven to 14, seven to 13, seven to 12, seven to 11, seven to ten, seven to nine, eight to 15, eight to 14, eight to 13, eight to 12, eight to 11, eight to ten, nine to 15, nine to 14, nine to 13, nine to 12, nine to 11, ten to 15, ten to 14, ten to 13, ten to 12, 11 to 15, 11 to 14, 11 to 13, 12 to 15, or 12 to 14 of proteins APP, PSEN1, PSEN2, APOE, TOMM40, GAB2, APOC1, TREM2, AB13, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1L, STK24, DISCI , MPZL1, SLC4A1AP, TRIP4, MSRA, HS3ST1, ZNF224, AP2A2, FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1 L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1B, NUCKS1, ACMSD, HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT). In some embodiments, the proteins include a panel set forth in Table 4, herein. In some embodiments of any of the foregoing aspects, the population of cells is a uniform population of cells. In some embodiments, the population of cells is a heterogeneous population of cells. In some embodiments, the cells are embryonic stem cells or induced cells. In some embodiments, the cells are pluripotent cells. In some embodiments, the pluripotent cells are ESCs. In some embodiments, the pluripotent cells are iPSCs. In some embodiments, the cells are CD34+ cells. In some embodiments, the cells are multipotent cells. In some embodiments, the multipotent cells are CD34+ cells. In some embodiments, the CD34+ cells are hematopoietic stem cells. In some embodiments, the CD34+ cells are myeloid progenitor cells. In some embodiments, the cells are blood line progenitor cells (BLPCs). In some embodiments, the BLPCs are monocytes. In some embodiments the cells are macrophages. In some embodiments, the cells are microglial progenitor cells. In some embodiments, the cells are microglia.

In some embodiments of any of the foregoing aspects, the composition is formulated for systemic administration to a patient. In some embodiments, the composition is formulated for intravenous injection to the patient. In some embodiments, the composition is formulated for direct administration to the central nervous system of a patient (e.g., a mammalian patient, such as a human patient. In some embodiments, the composition is formulated for direct administration to the CSF of the patient. In some embodiments, the composition is formulated for ICV injection, intrathecal injection, stereotactic injection, intraparenchymal injection, or a combination thereof, to the patient.

In some embodiments of any of the foregoing aspects, the composition is formulated for systemic administration and direct administration to the central nervous system of a patient (e.g., a mammalian patient, such as a human patient. In some embodiments, the composition is formulated for intravenous injection and for direct administration to the CSF of the patient. In some embodiments, the composition is formulated for intravenous injection and ICV injection, intrathecal injection, stereotactic injection, intraparenchymal injection, or a combination thereof, to the patient.

In some embodiments of any of the foregoing aspects, the cells are autologous cells. In some embodiments, the cells are allogeneic cells.

In some embodiments of any of the foregoing aspects, the cells contain a transgene encoding one or more of the proteins operably linked to a ubiquitous promoter. In some embodiments, the ubiquitous promoter is an elongation factor 1-alpha promoter or a phosphoglycerate kinase 1 promoter. The cells may contain a transgene encoding one or more of the proteins operably linked to a cell lineage-specific promoter, such as a PGRN promoter, a CD11 b promoter, a CD68 promoter, a C—X3-C motif chemokine receptor 1 promoter, an allograft inflammatory factor 1 promoter, a purinergic receptor P2Y12 promoter, a transmembrane protein 119 promoter, or a colony stimulating factor 1 receptor promoter. In some embodiments, the cells contain a transgene encoding one or more of the proteins operably linked to a synthetic promoter.

In some embodiments of any of the foregoing aspects, one or more of the proteins further contains an Rb domain of ApoE. The Rb domain may, for example, contain a portion of ApoE having the amino acid sequence of residues 25-185, 50-180, 75-175, 100-170, 125-160, or 130-150 of SEQ ID NO: 105. In some embodiments, the Rb domain contains a region having at least 70% sequence identity (e.g., a region having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, or 100% sequence identity) to the amino acid sequence of residues 159-167 of SEQ ID NO: 105.

In some embodiments of any of the foregoing aspects, the cells contain a transgene encoding one or more of the proteins and containing a miRNA targeting sequence in the 3′-UTR, such as a miR-126 targeting sequence.

In a ninth aspect, the disclosure provides a pharmaceutical composition containing a population of viral vectors that together encode one or more proteins selected from APP, PSEN1, PSEN2, APOE, TOMM40, GAB2, APOC1, TREM2, ABI3, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1L, STK24, DISCI , MPZL1, SLC4A1AP, TRIP4, MSRA, HS3ST1, ZNF224, and AP2A2, such as one or more proteins selected from PSEN1, GAB2, APOC1, TREM2, ABI3, BIN1, HLA-DRB5, HLA-DRB1, CD2AP, PTK2B, INPP5D, MEF2C, CD33, MS4A4A, RIN3, PICALM, CASS4, SORL1, PLCG2, SCIMP, FRMD4A, SPPL2A, MTHFD1 L, DISCI , TRIP4, and HS3ST1.

In some embodiments of the foregoing aspect, the viral vectors together encode two or more of the proteins selected from APP, PSEN1, PSEN2, APOE, TOMM40, GAB2, APOC1, TREM2, ABI3, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1 L, STK24, DISCI , MPZL1, SLC4A1AP, TRIP4, MSRA, HS3ST1, ZNF224, and AP2A2, such as two or more proteins selected from PSEN1, GAB2, APOC1, TREM2, ABI3, BIN1, HLA-DRB5, HLA-DRB1, CD2AP, PTK2B, INPP5D, MEF2C, CD33, MS4A4A, RIN3, PICALM, CASS4, SORL1, PLCG2, SCIMP, FRMD4A, SPPL2A, MTHFD1 L, DISCI , TRIP4, and HS3ST1. For example, the viral vectors may together encode three, four, five, six, seven, eight, nine, ten, 11, 12, 13, 14, 15, 17, 18, 19, 20, or more, of APP, PSEN1, PSEN2, APOE, TOMM40, GAB2, APOC1, TREM2, ABI3, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1 L, STK24, DISC1, MPZL1, SLC4A1AP, TRIP4, MSRA, HS3ST1, ZNF224, and AP2A2, such as three, four, five, six, seven, eight, nine, ten, 11, 12, 13, 14, 15, 17, 18, 19, 20, or more, of PSEN1, GAB2, APOC1, TREM2, ABI3, BIN1, HLA-DRB5, HLA-DRB1, CD2AP, PTK2B, INPP5D, MEF2C, CD33, MS4A4A, RIN3, PICALM, CASS4, SORL1, PLCG2, SCIMP, FRMD4A, SPPL2A, MTHFD1 L, DISC1, TRIP4, and HS3ST1.

In some embodiments of the foregoing aspect, the viral vectors together encode from two to 20 of the proteins, such as from two to 19, two to 18, two to 17, two to 16, two to 15, two to 14, two to 13, two to 12, two to 11, two to ten, two to nine, two to eight, two to seven, two to six, two to five, two to four, three to 20, three to 19, three to 18, three to 17, three to 16, three to 15, three to 14, three to 13, three to 12, three to 11, three to ten, three to nine, three to eight, three to seven, three to six, three to five, four to 20, four to 19, four to 18, four to 17, four to 16, four to 15, four to 14, four to 13, four to 12, four to 11, four to ten, four to nine, four to eight, four to seven, four to six, five to 20, five to 19, five to 18, five to 17, five to 16, five to 15, five to 14, five to 13, five to 12, five to 11, five to ten, five to nine, five to eight, five to seven, six to 20, six to 19, six to 18, six to 17, six to 16, six to 15, six to 14, six to 13, six to 12, six to 11, six to ten, six to nine, six to eight, seven to 20, seven to 19, seven to 18, seven to 17, seven to 16, seven to 15, seven to 14, seven to 13, seven to 12, seven to 11, seven to ten, seven to nine, eight to 20, eight to 19, eight to 18, eight to 17, eight to 16, eight to 15, eight to 14, eight to 13, eight to 12, eight to 11, eight to ten, nine to 20, nine to 19, nine to 18, nine to 17, nine to 16, nine to 15, nine to 14, nine to 13, nine to 12, nine to 11, ten to 20, ten to 19, ten to 18, ten to 17, ten to 16, ten to 15, ten to 14, ten to 13, ten to 12, 11 to 20, 11 to 19, 11 to 18, 11 to 17, 11 to 16, 11 to 15, 11 to 14, 11 to 13, 12 to 20, 12 to 19, 12 to 18, 12 to 17, 12 to 16, 12 to 15, 12 to 14, 13 to 20, 13 to 19, 13 to 18, 13 to 17, 13 to 16, 13 to 15, 14 to 20, 14 to 19, 14 to 18, 14 to 17, 14 to 16, 15 to 20, 15 to 19, 15 to 18, 15 to 17, 16 to 20, 16 to 19, 16 to 18, 17 to 20, 17 to 19, or 18 to 20 of proteins APP, PSEN1, PSEN2, APOE, TOMM40, GAB2, APOC1, TREM2, ABI3, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1 L, STK24, DISC1, MPZL1, SLC4A1 AP, TRIP4, MSRA, HS3ST1, ZNF224, and AP2A2 (e.g., from two to 19, two to 18, two to 17, two to 16, two to 15, two to 14, two to 13, two to 12, two to 11, two to ten, two to nine, two to eight, two to seven, two to six, two to five, two to four, three to 20, three to 19, three to 18, three to 17, three to 16, three to 15, three to 14, three to 13, three to 12, three to 11, three to ten, three to nine, three to eight, three to seven, three to six, three to five, four to 20, four to 19, four to 18, four to 17, four to 16, four to 15, four to 14, four to 13, four to 12, four to 11, four to ten, four to nine, four to eight, four to seven, four to six, five to 20, five to 19, five to 18, five to 17, five to 16, five to 15, five to 14, five to 13, five to 12, five to 11, five to ten, five to nine, five to eight, five to seven, six to 20, six to 19, six to 18, six to 17, six to 16, six to 15, six to 14, six to 13, six to 12, six to 11, six to ten, six to nine, six to eight, seven to 20, seven to 19, seven to 18, seven to 17, seven to 16, seven to 15, seven to 14, seven to 13, seven to 12, seven to 11, seven to ten, seven to nine, eight to 20, eight to 19, eight to 18, eight to 17, eight to 16, eight to 15, eight to 14, eight to 13, eight to 12, eight to 11, eight to ten, nine to 20, nine to 19, nine to 18, nine to 17, nine to 16, nine to 15, nine to 14, nine to 13, nine to 12, nine to 11, ten to 20, ten to 19, ten to 18, ten to 17, ten to 16, ten to 15, ten to 14, ten to 13, ten to 12, 11 to 20, 11 to 19, 11 to 18,11 to 17,11 to 16,11 to 15,11 to 14,11 to 13, 12 to 20, 12 to 19, 12 to 18, 12 to 17, 12 to 16, 12 to 15, 12 to 14, 13 to 20, 13 to 19, 13 to 18, 13 to 17, 13 to 16, 13 to 15, 14 to 20, 14 to 19, 14 to 18, 14 to 17, 14 to 16, 15 to 20, 15 to 19, 15 to 18, 15 to 17, 16 to 20, 16 to 19, 16 to 18, 17 to 20, 17 to 19, or 18 to 20 of proteins PSEN1, GAB2, APOC1, TREM2, ABI3, BIN1, HLA-DRB5, HLA-DRB1, CD2AP, PTK2B, INPP5D, MEF2C, CD33, MS4A4A, RIN3, PICALM, CASS4, SORL1, PLCG2, SCIMP, FRMD4A, SPPL2A, MTHFD1 L, DISCI , TRIP4, and HS3ST1). In some embodiments, the proteins include a panel set forth in Table 1, herein.

In a tenth aspect, the disclosure provides a population of viral vectors that together encode one or more proteins selected from FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1 B, NUCKS1, and ACMSD, such as one or more proteins selected from FCGR2A, SCAF11, DNAJC13, GCH1, LRRK2, GBA, GAK, FGF20, HLA-DQB1, and NOD2.

In some embodiments of the foregoing aspect, the viral vectors together encode two or more of the proteins selected from FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1 B, NUCKS1, and ACMSD, such as two or more proteins selected from FCGR2A, SCAF11, DNAJC13, GCH1, LRRK2, GBA, GAK, FGF20, HLA-DQB1, and NOD2. For example, the viral vectors may together encode three, four, five, six, seven, eight, nine, ten, 11, 12, 13, 14, 15, 17, 18, 19, 20, or more, of FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1 L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1B, NUCKS1, and ACMSD, such as three, four, five, six, seven, eight, nine, or more, of FCGR2A, SCAF11, DNAJC13, GCH1, LRRK2, GBA, GAK, FGF20, HLA-DQB1, and NOD2.

In some embodiments of the foregoing aspect, the viral vectors together encode from two to 20 of the proteins, such as from two to 19, two to 18, two to 17, two to 16, two to 15, two to 14, two to 13, two to 12, two to 11, two to ten, two to nine, two to eight, two to seven, two to six, two to five, two to four, three to 20, three to 19, three to 18, three to 17, three to 16, three to 15, three to 14, three to 13, three to 12, three to 11, three to ten, three to nine, three to eight, three to seven, three to six, three to five, four to 20, four to 19, four to 18, four to 17, four to 16, four to 15, four to 14, four to 13, four to 12, four to 11, four to ten, four to nine, four to eight, four to seven, four to six, five to 20, five to 19, five to 18, five to 17, five to 16, five to 15, five to 14, five to 13, five to 12, five to 11, five to ten, five to nine, five to eight, five to seven, six to 20, six to 19, six to 18, six to 17, six to 16, six to 15, six to 14, six to 13, six to 12, six to 11, six to ten, six to nine, six to eight, seven to 20, seven to 19, seven to 18, seven to 17, seven to 16, seven to 15, seven to 14, seven to 13, seven to 12, seven to 11, seven to ten, seven to nine, eight to 20, eight to 19, eight to 18, eight to 17, eight to 16, eight to 15, eight to 14, eight to 13, eight to 12, eight to 11, eight to ten, nine to 20, nine to 19, nine to 18, nine to 17, nine to 16, nine to 15, nine to 14, nine to 13, nine to 12, nine to 11, ten to 20, ten to 19, ten to 18, ten to 17, ten to 16, ten to 15, ten to 14, ten to 13, ten to 12, 11 to 20, 11 to 19, 11 to 18, 11 to 17, 11 to 16, 11 to 15, 11 to 14, 11 to 13, 12 to 20, 12 to 19, 12 to 18, 12 to 17, 12 to 16, 12 to 15, 12 to 14, 13 to 20, 13 to 19, 13 to 18, 13 to 17, 13 to 16, 13 to 15, 14 to 20, 14 to 19, 14 to 18, 14 to 17, 14 to 16, 15 to 20, 15 to 19, 15 to 18, 15 to 17, 16 to 20, 16 to 19, 16 to 18, 17 to 20, 17 to 19, or 18 to 20 of proteins FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1B, NUCKS1, and ACMSD (e.g., from two to ten, two to nine, two to eight, two to seven, two to six, two to five, two to four, three to ten, three to nine, three to eight, three to seven, three to six, three to five, four to ten, four to nine, four to eight, four to seven, four to six, five to ten, five to nine, five to eight, five to seven, six to ten, six to nine, six to eight, seven to ten, seven to nine, or eight to ten of proteins FCGR2A, SCAF11, DNAJC13, GCH1, LRRK2, GBA, GAK, FGF20, HLA-DQB1, and NOD2). In some embodiments, the proteins include a panel set forth in Table 2, herein.

In an eleventh aspect, the disclosure provides a population of viral vectors that together encode one or more proteins selected from HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT, such as one or more proteins selected from HLA-DRA, HLA-DRB5, C9ORF72, SQSTM1, TBK1, PSEN1, GRN, and CTSF.

In some embodiments of the foregoing aspect, the viral vectors together encode two or more of the proteins selected from HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT, such as two or more proteins selected from HLA-DRA, HLA-DRB5, C9ORF72, SQSTM1, TBK1, PSEN1, GRN, and CTSF. For example, the viral vectors may together encode three, four, five, six, seven, eight, nine, ten, 11, 12, 13, 14, 15, or more, of HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT, such as three, four, five, six, or more, of HLA-DRA, HLA-DRB5, C9ORF72, SQSTM1, TBK1, PSEN1, GRN, and CTSF.

In some embodiments of the foregoing aspect, the viral vectors together encode from two to 15 of the proteins, such as from two to 14, two to 13, two to 12, two to 11, two to ten, two to nine, two to eight, two to seven, two to six, two to five, two to four, three to 15, three to 14, three to 13, three to 12, three to 11, three to ten, three to nine, three to eight, three to seven, three to six, three to five, four to 15, four to 14, four to 13, four to 12, four to 11, four to ten, four to nine, four to eight, four to seven, four to six, five to 15, five to 14, five to 13, five to 12, five to 11, five to ten, five to nine, five to eight, five to seven, six to 15, six to 14, six to 13, six to 12, six to 11, six to ten, six to nine, six to eight, seven to 15, seven to 14, seven to 13, seven to 12, seven to 11, seven to ten, seven to nine, eight to 15, eight to 14, eight to 13, eight to 12, eight to 11, eight to ten, nine to 15, nine to 14, nine to 13, nine to 12, nine to 11, ten to 15, ten to 14, ten to 13, ten to 12, 11 to 15, 11 to 14, 11 to 13, 12 to 15, or 12 to 14 of proteins HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT, such as two or more proteins selected from HLA-DRA, HLA-DRB5, C9ORF72, SQSTM1, TBK1, PSEN1, GRN, and CTSF (e.g., from two to six, two to five, two to four, three to six, three to five, four to ten, or four to six, of proteins HLA-DRA, HLA-DRB5, C9ORF72, SQSTM1, TBK1, PSEN1, GRN, and CTSF). In some embodiments, the proteins include a panel set forth in Table 3, herein.

In a twelfth aspect, the disclosure provides a population of viral vectors that together encode one or more proteins selected from APP, PSEN1, PSEN2, APOE, TOMM40, GAB2, APOC1, TREM2, ABI3, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1L, STK24, DISCI , MPZL1, SLC4A1AP, TRIP4, MSRA, HS3ST1, ZNF224, AP2A2, FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1 B, NUCKS1, ACMSD, HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT.

In some embodiments of the foregoing aspect, the viral vectors together encode two or more of the proteins selected from APP, PSEN1, PSEN2, APOE, TOMM40, GAB2, APOC1, TREM2, ABI3, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1L, STK24, DISCI , MPZL1, SLC4A1AP, TRIP4, MSRA, HS3ST1, ZNF224, AP2A2, FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1 L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1B, NUCKS1, ACMSD, HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT. For example, the viral vectors may together encode three, four, five, six, seven, eight, nine, ten, 11, 12, 13, 14, 15, or more, of APP, PSEN1, PSEN2, APOE, TOMM40, GAB2, APOC1, TREM2, ABI3, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1L, STK24, DISCI , MPZL1, SLC4A1AP, TRIP4, MSRA, HS3ST1, ZNF224, AP2A2, FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1 L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1B, NUCKS1, ACMSD, HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT.

In some embodiments of the foregoing aspect, the viral vectors together encode from two to 15 of the proteins, such as from two to 14, two to 13, two to 12, two to 11, two to ten, two to nine, two to eight, two to seven, two to six, two to five, two to four, three to 15, three to 14, three to 13, three to 12, three to 11, three to ten, three to nine, three to eight, three to seven, three to six, three to five, four to 15, four to 14, four to 13, four to 12, four to 11, four to ten, four to nine, four to eight, four to seven, four to six, five to 15, five to 14, five to 13, five to 12, five to 11, five to ten, five to nine, five to eight, five to seven, six to 15, six to 14, six to 13, six to 12, six to 11, six to ten, six to nine, six to eight, seven to 15, seven to 14, seven to 13, seven to 12, seven to 11, seven to ten, seven to nine, eight to 15, eight to 14, eight to 13, eight to 12, eight to 11, eight to ten, nine to 15, nine to 14, nine to 13, nine to 12, nine to 11, ten to 15, ten to 14, ten to 13, ten to 12, 11 to 15, 11 to 14, 11 to 13, 12 to 15, or 12 to 14 of proteins APP, PSEN1, PSEN2, APOE, TOMM40, GAB2, APOC1, TREM2, AB13, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1L, STK24, DISCI , MPZL1, SLC4A1AP, TRIP4, MSRA, HS3ST1, ZNF224, AP2A2, FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2,

FAM47E, CCDC62, TMEM229B, MAPT, SPPL2B, ITGA8, ATP13A2, DGKQ, STX1B, NUCKS1, ACMSD, HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT). In some embodiments, the proteins include a panel set forth in Table 4, herein.

In some embodiments of any of the foregoing aspects, the viral vectors contain an AAV, an adenovirus, a parvovirus, a coronavirus, a rhabdovirus, a paramyxovirus, a picornavirus, an alphavirus, a herpes virus, a poxvirus, and/or a Retroviridae family virus. In some embodiments, the viral vector is a Retroviridae family viral vector, such as a lentiviral vector, alpharetroviral vector, or gammaretroviral vector. In some embodiments, the Retroviridae family viral vector contains a central polypurine tract, a woodchuck hepatitis virus post-transcriptional regulatory element, a 5′-LTR, HIV signal sequence, HIV Psi signal 5′-splice site, delta-GAG element, 3′-splice site, and a 3′-self inactivating LTR. In some embodiments, the viral vector is an AAV selected from the group consisting of AAV1, AAV2, AAV3, AAV4, AAVS, AAV6, AAV7, AAV8, AAV9, AAV10, and AAVrh74. In some embodiments, the viral vector is a pseudotyped viral vector, such as a pseudotyped viral vector selected from the group consisting of a pseudotyped AAV, a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus.

In some embodiments of any of the foregoing aspects, the composition is formulated for systemic administration to a patient. In some embodiments, the composition is formulated for intravenous injection to the patient. In some embodiments, the composition is formulated for direct administration to the central nervous system of a patient. In some embodiments, the composition is formulated for direct administration to the CSF of the patient. In some embodiments, the composition is formulated for ICV injection, intrathecal injection, stereotactic injection, intraparenchymal injection, or a combination thereof, to the patient. In some embodiments of any of the foregoing aspects, the composition is formulated for systemic administration and direct administration to the central nervous system of a patient (e.g., a mammalian patient, such as a human patient. In some embodiments, the composition is formulated for intravenous injection and for direct administration to the CSF of the patient. In some embodiments, the composition is formulated for intravenous injection and ICV injection, intrathecal injection, stereotactic injection, intraparenchymal injection, or a combination thereof, to the patient.

In some embodiments, the patient is diagnosed with an NCD. In some embodiments, the NCD is a major NCD. In some embodiments, the major NCD interferes with the patient's independence and/or normal daily functioning (e.g., social, occupational, or academic functioning, personal hygiene, grooming, dressing, toilet hygiene, functional mobility (e.g., ability to walk, get in and out of bed), and self-feeding. In some embodiments, the major NCD is associated with a score obtained by the patient on a cognitive test that is at least two standard deviations away from the mean score of a reference population. In some embodiments, the NCD is a mild NCD. In some embodiments, the mild NCD does not interfere with the patient's independence and/or normal daily functioning. In some embodiments, the mild NCD is associated with a score obtained by the patient on a cognitive test that is between one to two standard deviations away from the mean score of a reference population. In some embodiments, the cognitive test is selected from the group consisting of ADB, AWV, GPCOG, HRA, MIS, MMSE, MoCA, SLUMS, and Short IQCODE. In some embodiments, the NCD is associated with impairment in one or more of complex attention, executive function, learning and memory, language, perceptual-motor function, and social cognition. In some embodiments, the NCD is not due to delirium or other mental disorder (e.g., schizophrenia, bipolar disorder, or major depression). In some embodiments, the reference population is a general population. In some embodiments, the reference population is selected on the basis of the patient's age, medical history, education, socioeconomic status, and lifestyle. In some embodiments, the NCD is Alzheimer's disease. In some embodiments, the NCD is a movement disorder. In some embodiments, the movement disorder is Parkinson disease. In some embodiments, the NCD is a frontotemporal NCD. In some embodiments, the frontotemporal NCD is FTLD. In some embodiments, the FTLD is behavioral-variant frontotemporal dementia. In some embodiments, the FTLD is semantic dementia. In some embodiments, the FTLD is progressive nonfluent aphasia.

In some embodiments, one or more of the viral vectors contains a transgene encoding one or more of the proteins operably linked to a ubiquitous promoter. The ubiquitous promoter may be, for example, an elongation factor 1-alpha promoter or a phosphoglycerate kinase 1 promoter. In some embodiments, one or more of the viral vectors contains a transgene encoding one or more of the proteins operably linked to a cell lineage-specific promoter, such as a PGRN promoter, a CD11 b promoter, a CD68 promoter, a C—X3-C motif chemokine receptor 1 promoter, an allograft inflammatory factor 1 promoter, a purinergic receptor P2Y12 promoter, a transmembrane protein 119 promoter, or a colony stimulating factor 1 receptor promoter. In some embodiments, one or more of the viral vectors contains a transgene encoding one or more of the proteins operably linked to a synthetic promoter.

In some embodiments of any of the foregoing aspects, one or more of the proteins further contains an Rb domain of ApoE. The Rb domain may contain a portion of ApoE, such as a portion having the amino acid sequence of residues 25-185, 50-180, 75-175, 100-170, 125-160, or 130-150 of SEQ ID NO: 105. In some embodiments, the Rb domain contains a region having at least 70% sequence identity (e.g., a region having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, or 100% sequence identity) to the amino acid sequence of residues 159-167 of SEQ ID NO: 105.

In some embodiments, one or more of the viral vectors contains a transgene encoding one or more of the proteins, and the transgene may, for example, further contain a miRNA targeting sequence in the 3′-UTR. In some embodiments, the miRNA targeting sequence is a miR-126 targeting sequence.

In an additional aspect, the disclosure features a kit containing the pharmaceutical composition of the fifth or ninth aspects above. The kit may further contain a package insert instructing a user of the kit to administer the pharmaceutical composition to a patient (e.g., a mammalian patient, such as a human patient (e.g., an adult human patient)) having an NCD. In some embodiments, the patient (e.g., a human) is diagnosed with an NCD. In some embodiments, the NCD is a major NCD. In some embodiments, the major NCD interferes with the patient's independence and/or normal daily functioning (e.g., social, occupational, or academic functioning, personal hygiene, grooming, dressing, toilet hygiene, functional mobility (e.g., ability to walk, get in and out of bed), and self-feeding. In some embodiments, the major NCD is associated with a score obtained by the patient on a cognitive test that is at least two standard deviations away from the mean score of a reference population. In some embodiments, the NCD is a mild NCD. In some embodiments, the mild NCD does not interfere with the patient's independence and/or normal daily functioning. In some embodiments, the mild NCD is associated with a score obtained by the patient on a cognitive test that is between one to two standard deviations away from the mean score of a reference population. In some embodiments, the cognitive test is selected from the group consisting of AD8, AWV, GPCOG, HRA, MIS, MMSE, MoCA, SLUMS, and Short IQCODE. In some embodiments, the NCD is associated with impairment in one or more of complex attention, executive function, learning and memory, language, perceptual-motor function, and social cognition. In some embodiments, the NCD is not due to delirium or other mental disorder (e.g., schizophrenia, bipolar disorder, or major depression). In some embodiments, the reference population is a general population. In some embodiments, the reference population is selected on the basis of the patient's age, medical history, education, socioeconomic status, and lifestyle. In some embodiments, the NCD is Alzheimer's disease.

In another aspect, the disclosure features a kit containing the pharmaceutical composition of the sixth or tenth aspects above. The kit may further contain a package insert instructing a user of the kit to administer the pharmaceutical composition to a patient (e.g., a mammalian patient, such as a human patient (e.g., an adult human patient)) having an NCD. In some embodiments, the patient (e.g., a human) is diagnosed with an NCD. In some embodiments, the NCD is a major NCD. In some embodiments, the major NCD interferes with the patient's independence and/or normal daily functioning (e.g., social, occupational, or academic functioning, personal hygiene, grooming, dressing, toilet hygiene, functional mobility (e.g., ability to walk, get in and out of bed), and self-feeding. In some embodiments, the major NCD is associated with a score obtained by the patient on a cognitive test that is at least two standard deviations away from the mean score of a reference population. In some embodiments, the NCD is a mild NCD. In some embodiments, the mild NCD does not interfere with the patient's independence and/or normal daily functioning. In some embodiments, the mild NCD is associated with a score obtained by the patient on a cognitive test that is between one to two standard deviations away from the mean score of a reference population. In some embodiments, the cognitive test is selected from the group consisting of AD8, AWV, GPCOG, HRA, MIS, MMSE, MoCA, SLUMS, and Short IQCODE. In some embodiments, the NCD is associated with impairment in one or more of complex attention, executive function, learning and memory, language, perceptual-motor function, and social cognition. In some embodiments, the NCD is not due to delirium or other mental disorder (e.g., schizophrenia, bipolar disorder, or major depression). In some embodiments, the reference population is a general population. In some embodiments, the reference population is selected on the basis of the patient's age, medical history, education, socioeconomic status, and lifestyle. In some embodiments, the NCD is a movement disorder. In some embodiments, the movement disorder is Parkinson disease.

In a further aspect, the disclosure features a kit containing the pharmaceutical composition of the seventh or eleventh aspects above. The kit may further contain a package insert instructing a user of the kit to administer the pharmaceutical composition to a patient (e.g., a mammalian patient, such as a human patient (e.g., an adult human patient)) having an NCD. In some embodiments, the patient (e.g., a human) is diagnosed with an NCD. In some embodiments, the NCD is a major NCD. In some embodiments, the major NCD interferes with the patient's independence and/or normal daily functioning (e.g., social, occupational, or academic functioning, personal hygiene, grooming, dressing, toilet hygiene, functional mobility (e.g., ability to walk, get in and out of bed), and self-feeding. In some embodiments, the major NCD is associated with a score obtained by the patient on a cognitive test that is at least two standard deviations away from the mean score of a reference population. In some embodiments, the NCD is a mild NCD. In some embodiments, the mild NCD does not interfere with the patient's independence and/or normal daily functioning. In some embodiments, the mild NCD is associated with a score obtained by the patient on a cognitive test that is between one to two standard deviations away from the mean score of a reference population. In some embodiments, the cognitive test is selected from the group consisting of AD8, AWV, GPCOG, HRA, MIS, MMSE, MoCA, SLUMS, and Short IQCODE. In some embodiments, the NCD is associated with impairment in one or more of complex attention, executive function, learning and memory, language, perceptual-motor function, and social cognition. In some embodiments, the NCD is not due to delirium or other mental disorder (e.g., schizophrenia, bipolar disorder, or major depression). In some embodiments, the reference population is a general population. In some embodiments, the reference population is selected on the basis of the patient's age, medical history, education, socioeconomic status, and lifestyle. In some embodiments, the NCD is a frontotemporal NCD. In some embodiments, the frontotemporal NCD is FTLD. In some embodiments, the FTLD is behavioral-variant frontotemporal dementia. In some embodiments, the FTLD is semantic dementia. In some embodiments, the FTLD is progressive nonfluent aphasia.

In a further aspect, the disclosure features a kit containing the pharmaceutical composition of the eighth or twelfth aspects above. The kit may further contain a package insert instructing a user of the kit to administer the pharmaceutical composition to a patient (e.g., a mammalian patient, such as a human patient (e.g., an adult human patient)) having an NCD. In some embodiments, the patient (e.g., a human) is diagnosed with an NCD. In some embodiments, the NCD is a major NCD. In some embodiments, the major NCD interferes with the patient's independence and/or normal daily functioning (e.g., social, occupational, or academic functioning, personal hygiene, grooming, dressing, toilet hygiene, functional mobility (e.g., ability to walk, get in and out of bed), and self-feeding. In some embodiments, the major NCD is associated with a score obtained by the patient on a cognitive test that is at least two standard deviations away from the mean score of a reference population. In some embodiments, the NCD is a mild NCD. In some embodiments, the mild NCD does not interfere with the patient's independence and/or normal daily functioning. In some embodiments, the mild NCD is associated with a score obtained by the patient on a cognitive test that is between one to two standard deviations away from the mean score of a reference population. In some embodiments, the cognitive test is selected from the group consisting of AD8, AWV, GPCOG, HRA, MIS, MMSE, MoCA, SLUMS, and Short IQCODE. In some embodiments, the NCD is associated with impairment in one or more of complex attention, executive function, learning and memory, language, perceptual-motor function, and social cognition. In some embodiments, the NCD is not due to delirium or other mental disorder (e.g., schizophrenia, bipolar disorder, or major depression). In some embodiments, the reference population is a general population. In some embodiments, the reference population is selected on the basis of the patient's age, medical history, education, socioeconomic status, and lifestyle. In some embodiments, the NCD is Alzheimer's disease. In some embodiments, the NCD is a movement disorder. In some embodiments, the movement disorder is Parkinson disease. In some embodiments, the NCD is a frontotemporal NCD. In some embodiments, the frontotemporal NCD is FTLD. In some embodiments, the FTLD is behavioral-variant frontotemporal dementia. In some embodiments, the FTLD is semantic dementia. In some embodiments, the FTLD is progressive nonfluent aphasia.

Additional embodiments of the present invention are provided in the enumerated paragraphs below.
E1. A method of treating a patient diagnosed as having a neurocognitive disorder (NCD), the method comprising providing to the patient one or more agents that collectively increase expression and/or activity of two or more proteins selected from APP, PSEN1, PSEN2, APOE, TOMM40, GAB2, APOC1, TREM2, AB13, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1L, STK24, DISCI , MPZL1, SLC4A1AP, TRIP4, MSRA, HS3ST1, ZNF224, and AP2A2.
E2. The method of E1, wherein the proteins are selected from PSEN1, GAB2, APOC1, TREM2, AB13, BIN1, HLA-DRB5, HLA-DRB1, CD2AP, PTK2B, INPP5D, MEF2C, CD33, MS4A4A, RIN3, PICALM, CASS4, SORL1, PLCG2, SCIMP, FRMD4A, SPPL2A, MTHFD1 L, DISCI , TRIP4, and HS3ST1, optionally wherein the proteins comprise a panel set forth in Table 1.
E3. The method of E1 or E2, wherein the NCD is a major NCD.
E4. The method of E3, wherein the major NCD interferes with the patient's independence and/or normal daily functioning.
E5. The method of E3 or E4, wherein the major NCD is associated with a score obtained by the patient on a cognitive test that is at least two standard deviations away from the mean score of a reference population.
E6. The method of E1 or E2, wherein the NCD is a mild NCD.
E7. The method of E6, wherein the mild NCD does not interfere with the patient's independence and/or normal daily functioning.
E8. The method of E6 or E7, wherein the mild NCD is associated with a score obtained by the patient on a cognitive test that is between one to two standard deviations away from the mean score of a reference population.
E9. The method of E5 or E8, wherein the reference population is a general population.
E10. The method of E5, E8, or E9, wherein the cognitive test is selected from the group consisting of Eight-item Informant Interview to Differentiate Aging and Dementia (AD8), Annual Wellness Visit (AWV), General Practitioner Assessment of Cognition (GPCOG), Health Risk Assessment (HRA), Memory Impairment Screen (MIS), Mini Mental Status Exam (MMSE), Montreal Cognitive Assessment (MoCA), St. Louis University Mental Status Exam (SLUMS), and Short Informant Questionnaire on Cognitive Decline in the Elderly (Short IQCODE).

E11. The method of any one of E1 -E10, wherein the NCD is associated with impairment in one or more of complex attention, executive function, learning and memory, language, perceptual-motor function, and social cognition.
E12. The method of any one of E1-E11, wherein the NCD is not due to delirium or other mental disorder.
E13. The method of any one of E1-E12, wherein the NCD is Alzheimer's disease.
E14. A method of treating a patient diagnosed as having an NCD, the method comprising providing to the patient one or more agents that collectively increase expression and/or activity of two or more proteins selected from FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1 B, NUCKS1, and ACMSD.
E15. The method of E14, wherein the proteins are selected from FCGR2A, SCAF11, DNAJC13, GCH1, LRRK2, GBA, GAK, FGF20, HLA-DQB1, and NOD2, optionally wherein the proteins comprise a panel set forth in Table 2.
E16. The method of E14 or E15, wherein the NCD is a major NCD.
E17. The method of E16, wherein the major NCD interferes with the patient's independence and/or normal daily functioning.
E18. The method of E16 or E17, wherein the major NCD is associated with a score obtained by the patient on a cognitive test that is at least two standard deviations away from the mean score of a reference population.
E19. The method of E14 or E15, wherein the NCD is a mild NCD.
E20. The method of E19, wherein the mild NCD does not interfere with the patient's independence and/or normal daily functioning.
E21. The method of E19 or E20, wherein the mild NCD is associated with a score obtained by the patient on a cognitive test that is between one to two standard deviations away from the mean score of a reference population.
E22. The method of E18 or E21, wherein the reference population is a general population.
E23. The method of E18, E21, or E22, wherein the cognitive test is selected from the group consisting of ADB, AWV, GPCOG, HRA, MIS, MMSE, MoCA, SLUMS, and Short IQCODE.
E24. The method of any one of E14-E23, wherein the NCD is associated with impairment in one or more of complex attention, executive function, learning and memory, language, perceptual-motor function, and social cognition.
E25. The method of any one of E14-E24, wherein the NCD is not due to delirium or other mental disorder.
E26. The method of any one of E14-E25, wherein the NCD is a movement disorder.
E27. The method of E26, wherein the movement disorder is Parkinson disease.
E28. A method of treating a patient diagnosed as having an NCD, the method comprising providing to the patient one or more agents that collectively increase expression and/or activity of two or more proteins selected from HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT.
E29. The method of E28, wherein the proteins are selected from HLA-DRA, HLA-DRB5, C9ORF72, SQSTM1, TBK1, PSEN1, GRN, and CTSF, optionally wherein the proteins comprise a panel set forth in Table 3.
E30. The method of E28 or E29, wherein the NCD is a major NCD.
E31. The method of E30, wherein the major NCD interferes with the patient's independence and/or normal daily functioning.
E32. The method of E30 or E31, wherein the major NCD is associated with a score obtained by the patient on a cognitive test that is at least two standard deviations away from the mean score of a reference population.
E33. The method of E28 or E29, wherein the NCD is a mild NCD.
E34. The method of E33, wherein the mild NCD does not interfere with the patient's independence and/or normal daily functioning.
E35. The method of E33 or E34, wherein the mild NCD is associated with a score obtained by the patient on a cognitive test that is between one to two standard deviations away from the mean score of a reference population.
E36. The method of E32 or E35, wherein the reference population is a general population.
E37. The method of E32, E35, or E36, wherein the cognitive test is selected from the group consisting of ADB, AWV, GPCOG, HRA, MIS, MMSE, MoCA, SLUMS, and Short IQCODE.
E38. The method of any one of E28-E37, wherein the NCD is associated with impairment in one or more of complex attention, executive function, learning and memory, language, perceptual-motor function, and social cognition.
E39. The method of any one of E28-E38, wherein the NCD is not due to delirium or other mental disorder.
E40. The method of any one of E28-E39, wherein the NCD is a frontotemporal NCD.
E41. The method of E40, wherein the frontotemporal NCD is frontotemporal lobar degeneration (FTLD).
E42. The method of E40, wherein the FTLD is behavioral-variant frontotemporal dementia.
E43. The method of E40, wherein the FTLD is semantic dementia.
E44. The method of E40, wherein the FTLD is progressive nonfluent aphasia.
E45. The method of any one of E1 -E44, wherein the one or more agents collectively increase expression and/or activity of three or more of the proteins.
E46. The method of E45, wherein the one or more agents collectively increase expression and/or activity of four or more of the proteins.
E47. The method of E46, wherein the one or more agents collectively increase expression and/or activity of five or more of the proteins.
E48. The method of any one of E1 -El 3, wherein the one or more agents collectively increase expression and/or activity of from five to 20 of the proteins.
E49. The method of E48, wherein the one or more agents collectively increase expression and/or activity of from eight to 18 of the proteins
E50. The method of E49, wherein the one or more agents collectively increase expression and/or activity of from 10 to 15 of the proteins.
E51. The method of any one of E14-E27, wherein the one or more agents collectively increase expression and/or activity of from three to 10 of the proteins.
E52. The method of E51, wherein the one or more agents collectively increase expression and/or activity of from four to eight of the proteins.
E53. The method of E52, wherein the one or more agents collectively increase expression and/or activity of from five to seven of the proteins.
E54. The method of any one of E28-E44, wherein the one or more agents collectively increase expression and/or activity of from two to seven of the proteins.
E55. The method of E54, wherein the one or more agents collectively increase expression and/or activity of from three to six of the proteins.
E56. The method of E55, wherein the one or more agents collectively increase expression and/or activity of four or five of the proteins.
E57. The method of any one of E1 -E56, wherein the one or more agents comprise (i) one or more nucleic acid molecules that collectively encode the two or more proteins, (ii) one or more interfering RNA molecules that collectively increase expression and/or activity of the two or more proteins, (iii) one or more nucleic acid molecules encoding the one or more interfering RNA molecules, (iv) two or more of the proteins, and/or (v) one or more small molecules that collectively increase expression and/or activity of the two or more proteins.
E58. The method of E57, wherein the one or more interfering RNA molecules comprise short interfering RNA (siRNA), short hairpin RNA (shRNA), and/or micro RNA (miRNA).
E59. The method of E57, wherein the one or more agents comprise one or more nucleic acid molecules that collectively encode the two or more proteins.
E60. The method of E59, wherein the one or more nucleic acid molecules collectively encode three or more of the proteins.
E61. The method of E60, wherein the one or more nucleic acid molecules collectively encode four or more of the proteins.
E62. The method of E61, wherein the one or more nucleic acid molecules collectively encode five or more of the proteins.
E63. The method of any one of E1 -E13, wherein the one or more agents comprise one or more nucleic acid molecules that collectively encode from five to 20 of the proteins.
E64. The method of E63, wherein the one or more nucleic acid molecules collectively encode from eight to 18 of the proteins.
E65. The method of E64, wherein the one or more nucleic acid molecules collectively encode from 10 to 15 of the proteins.
E66. The method of any one of E14-E27, wherein the one or more agents comprise one or more nucleic acid molecules that collectively encode from three to 10 of the proteins.
E67. The method of E66, wherein the one or more nucleic acid molecules collectively encode from four to eight of the proteins.
E68. The method of E67, wherein the one or more nucleic acid molecules collectively encode from five to seven of the proteins.
E69. The method of any one of E28-E44, wherein the one or more agents comprise one or more nucleic acid molecules that collectively encode from two to seven of the proteins.
E70. The method of E69, wherein the one or more nucleic acid molecules collectively encode from three to six of the proteins.
E71. The method of E70, wherein the one or more nucleic acid molecules collectively encode four or five of the proteins.
E72. The method of any one of E59-E71, wherein the one or more nucleic acid molecules are provided to the patient by administering to the patient a composition comprising a population of cells that together contain nucleic acids encoding the proteins.
E73. The method of E72, wherein the population is a uniform population of cells that contain nucleic acids encoding the proteins.
E74. The method of E72, wherein the population is a heterogeneous population of cells that together contain nucleic acids encoding the proteins.
E75. The method of any one of E72-E74, wherein the cells are ESCs.
E76. The method of any one of E72-E74, wherein the cells are iPSCs.
E77. The method of any one of E72-E74, wherein the cells are CD34+ cells.
E78. The method of E77, wherein the CD34+ cells are HSCs.
E79. The method of E77, wherein the CD34+ cells are MPCs.
E80. The method of any one of E72-E79, wherein the composition is administered systemically to the patient.
E81. The method of E80, wherein the composition is administered to the patient by way of intravenous injection.
E82. The method of any one of E72-E79, wherein the composition is administered directly to the central nervous system of the patient.
E83. The method of E72, wherein the composition is administered directly to the cerebrospinal fluid (CSF) of the patient.
E84. The method of E72 or 83, wherein the composition is administered to the patient by way of intracerebroventricular (ICV) injection, intrathecal injection, stereotactic injection, intraparenchymal injection, or a combination thereof.
E85. The method of any one of E72-E79, wherein the composition is administered to the patient systemically and directly to the central nervous system of the patient.
E86. The method of E85, wherein the composition is administered to the patient by way of intravenous injection and directly to the CSF of the patient.
E87. The method of E85, wherein the composition is administered to the patient by way of intravenous injection and by way of ICV injection, intrathecal injection, stereotactic injection, intraparenchymal injection, or a combination thereof.
E88. The method of any one of E72-E85, wherein the cells are autologous cells.
E89. The method of any one of E72-E85, wherein the cells are allogeneic cells.
E90. The method of any one of E72-E89, wherein the cells are transduced ex vivo to express the proteins.
E91. The method of E90, wherein the cells are transduced with a viral vector selected from the group consisting of an adeno-associated virus (AAV), an adenovirus, a parvovirus, a coronavirus, a rhabdovirus, a paramyxovirus, a picornavirus, an alphavirus, a herpes virus, a poxvirus, and a Retroviridae family virus.
E92. The method of E91, wherein the viral vector is a Retroviridae family viral vector.
E93. The method of E92, wherein the Retroviridae family viral vector is a lentiviral vector.
E94. The method of E92, wherein the Retroviridae family viral vector is an alpharetroviral vector.
E95. The method of E94, wherein the Retroviridae family viral vector is a gammaretroviral vector.
E96. The method of any one of E92-E95, wherein the Retroviridae family viral vector comprises a central polypurine tract, a woodchuck hepatitis virus post-transcriptional regulatory element, a 5′-LTR, HIV signal sequence, HIV Psi signal 5′-splice site, delta-GAG element, 3′-splice site, and a 3′-self inactivating LTR.
E97. The method of E91, wherein the viral vector is an AAV selected from the group consisting of AAV1, AAV2, AAV3, AAV4, AAV5, AAV6, AAV7, AAV8, AAV9, AAV10, and AAVrh74.
E98. The method of any one of E91-E97, wherein the viral vector is a pseudotyped viral vector.
E99. The method of E98, wherein the pseudotyped viral vector selected from the group consisting of a pseudotyped AAV, a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus.
E100. The method of any one of E72-E99, wherein the cells are transfected ex vivo to express the proteins.
E101. The method of E100, wherein the cells are transfected using: a) an agent selected from the group consisting of a cationic polymer, diethylaminoethyldextran, polyethylenimine, a cationic lipid, a liposome, calcium phosphate, an activated dendrimer, and a magnetic bead; or b) a technique selected from the group consisting of electroporation, Nucleofection, squeeze-poration, sonoporation, optical transfection, Magnetofection, and impalefection.
E102. The method of any one of E59-E71, wherein the one or more nucleic acid molecules are provided to the patient by administering to the patient one or more viral vectors that together comprise the one or more nucleic acid molecules.
E103. The method of E102, wherein the patient is administered a plurality of viral vectors that together comprise the one or more nucleic acid molecules.
E104. The method of E102, wherein the patient is administered a plurality of viral vectors that each individually comprise the one or more nucleic acid molecules.
E105. The method of any one of E102-E104, wherein the one or more viral vectors are administered systemically to the patient.
E106. The method of E105, wherein the one or more viral vectors are administered to the patient by way of intravenous injection.
E107. The method of any one of E102-E104, wherein the one or more viral vectors are administered directly to the central nervous system of the patient.
E108. The method of E107, wherein the one or more viral vectors are administered directly to the CSF of the patient.
E109. The method of E107 or 108, wherein the one or more viral vectors are administered to the patient by way of ICV injection, intrathecal injection, stereotactic injection, intraparenchymal injection, or a combination thereof.
E110. The method of any one of E102-E104, wherein the one or more viral vectors are administered to the patient systemically and directly to the central nervous system of the patient.
E111. The method of E110, wherein the one or more viral vectors are is administered to the patient by way of intravenous injection and directly to the CSF of the patient.
E112. The method of E111, wherein the one or more viral vectors are is administered to the patient by way of intravenous injection and by way of ICV injection, intrathecal injection, stereotactic injection, intraparenchymal injection, or a combination thereof.
E113. The method of any one of E102-E112, wherein the one or more viral vectors comprise an AAV, an adenovirus, a parvovirus, a coronavirus, a rhabdovirus, a paramyxovirus, a picornavirus, an alphavirus, a herpes virus, a poxvirus, or a Retroviridae family virus.
E114. The method of E113, wherein the viral vector is a Retroviridae family viral vector.
E11 5. The method of E113, wherein the Retroviridae family viral vector is a lentiviral vector.
E11 6. The method of E113, wherein the Retroviridae family viral vector is an alpharetroviral vector.
E11 7. The method of E113, wherein the Retroviridae family viral vector is a gammaretroviral vector.
E11 8. The method of any one of E113-E1 17, wherein the Retroviridae family viral vector comprises a central polypurine tract, a woodchuck hepatitis virus post-transcriptional regulatory element, a 5′-LTR, HIV signal sequence, HIV Psi signal 5′-splice site, delta-GAG element, 3′-splice site, and a 3′-self inactivating LTR.
E11 9. The method of E113, wherein the viral vector is an AAV selected from the group consisting of AAV1, AAV2, AAV3, AAV4, AAVS, AAV6, AAV7, AAV8, AAV9, AAV10, and AAVrh74.
E120. The method of any one of E113-119, wherein the viral vector is a pseudotyped viral vector.
E121. The method of E120, wherein the pseudotyped viral vector selected from the group consisting of a pseudotyped AAV, a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus.
E122. The method of any one of E59-E121, wherein one or more of the nucleic acid molecules comprises a transgene encoding one or more of the proteins operably linked to a ubiquitous promoter.
E123. The method of E122, wherein the ubiquitous promoter is selected from the group consisting of an elongation factor 1-alpha promoter and a phosphoglycerate kinase 1 promoter.
E124. The method of any one of E59-E123, wherein one or more of the nucleic acid molecules comprises a transgene encoding one or more of the proteins operably linked to a cell lineage-specific promoter.
E125. The method of E124, wherein the cell lineage-specific promoter is selected from the group consisting of a PGRN promoter, CD11 b promoter, CD68 promoter, a C—X3-C motif chemokine receptor 1 promoter, an allograft inflammatory factor 1 promoter, a purinergic receptor P2Y12 promoter, a transmembrane protein 119 promoter, and a colony stimulating factor 1 receptor promoter.
E126. The method of any one of E59-E125, wherein one or more of the nucleic acid molecules comprises a transgene encoding one or more of the proteins operably linked to a synthetic promoter.
E127. The method of any one of E59-E126, wherein one or more of the proteins further comprises a receptor-binding (Rb) domain of apolipoprotein E (ApoE).
E128. The method of E127, wherein the Rb domain comprises a portion of ApoE having the amino acid sequence of residues 25-185, 50-180, 75-175, 100-170, 125-160, or 130-150 of SEQ ID NO: 105.
E129. The method of E127 or E128, wherein the Rb domain comprises a region having at least 70% sequence identity to the amino acid sequence of residues 159-167 of SEQ ID NO: 105.
E130. The method of any one of E59-E129, wherein the one or more nucleic acid molecules comprise a micro RNA (miRNA) targeting sequence in the 3′-UTR.
E131. The method of E130, wherein the miRNA targeting sequence is a miR-126 targeting sequence.
E132. The method of any one of E59-E131, wherein upon providing the one or more nucleic acid molecules to the patient, the proteins penetrate the blood-brain barrier in the patient.
E133. The method of any one of E59-E132, wherein a population of endogenous microglia in the patient has been ablated prior to providing the patient with the one or more nucleic acid molecules.
E134. The method of any one of E59-E132, the method comprising ablating a population of endogenous microglia in the patient prior to providing the patient with the one or more nucleic acid molecules.
E135. The method of E133 or E134, wherein the microglia are ablated using an agent selected from the group consisting of busulfan, PLX3397, PLX647, PLX5622, treosulfan, and clodronate liposomes, by radiation therapy, or a combination thereof.
E136. The method of any one of E72-E101 or E122-E135, wherein, prior to providing the patient with the one or more nucleic acid molecules, endogenous expression of one or more of the proteins is disrupted in the cells.
E137. The method of any one of E59-E136, wherein, prior to providing the patient with the one or more nucleic acid molecules, endogenous expression of one or more of the proteins is disrupted in the patient.
E138. The method of E137, wherein, prior to providing the patient with the one or more nucleic acid molecules, endogenous expression of one or more of the proteins is disrupted in a population of neurons in the patient.
E139. The method of E136, wherein the endogenous expression is disrupted by contacting the cells with a nuclease that catalyzes cleavage of an endogenous gene encoding one of the proteins.
E140. The method of E139, wherein the nuclease is a clustered regularly interspaced short palindromic repeats (CRISPR)-associated protein.
E141. The method of E140, wherein the CRISPR-associated protein is CRISPR-associated protein 9 (Cas9).
E142. The method of E140, wherein the CRISPR-associated protein is CRISPR-associated protein 12a (Cas12a)
E143. The method of E139, wherein the nuclease is a transcription activator-like effector nuclease, a meganuclease, or a zinc finger nuclease.
E144. The method of any one of E136-E140, wherein endogenous expression of one or more of the proteins is disrupted by administering an inhibitory RNA molecule to the cells, the patient, or the population of neurons.
E145. The method of E144, wherein the inhibitory RNA molecule is a siRNA, a shRNA, or a miRNA.
E146. The method of any one of E1-E145, wherein the patient is a human.
E147. A pharmaceutical composition comprising a population of cells that together contain nucleic acids encoding two or more proteins selected from APP, PSEN1, PSEN2, APOE, TOMM40, GAB2, APOC1, TREM2, ABI3, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1L, STK24, DISCI , MPZL1, SLC4A1AP, TRIP4, MSRA, HS3ST1, ZNF224, and AP2A2.
E148. The pharmaceutical composition of E147, wherein the proteins are selected from PSEN1, GAB2, APOC1, TREM2, ABI3, BIN1, HLA-DRB5, HLA-DRB1, CD2AP, PTK2B, INPP5D, MEF2C, CD33, MS4A4A, RIN3, PICALM, CASS4, SORL1, PLCG2, SCIMP, FRMD4A, SPPL2A, MTHFD1 L, DISCI , TRIP4, and HS3ST1.
E149. A pharmaceutical composition comprising a population of cells that together contain nucleic acids encoding two or more proteins selected from FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1B, NUCKS1, and ACMSD.
E150. The pharmaceutical composition of E149, wherein the proteins are selected from FCGR2A, SCAF11, DNAJC13, GCH1, LRRK2, GBA, GAK, FGF20, HLA-DQB1, and NOD2.
E151. A pharmaceutical composition comprising a population of cells that together contain nucleic acids encoding two or more proteins selected from HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT.
E152. The pharmaceutical composition of E151, wherein the proteins are selected from HLA-DRA, HLA-DRB5, C9ORF72, SQSTM1, TBK1, PSEN1, GRN, and CTSF.
E153. The pharmaceutical composition of any one of E147-E152, wherein the cells together contain nucleic acids encoding three or more of the proteins.
E154. The pharmaceutical composition of E153, wherein the cells together contain nucleic acids encoding four or more of the proteins.
E155. The pharmaceutical composition of E154, wherein the cells together contain nucleic acids encoding five or more of the proteins.
E156. The pharmaceutical composition of E147 or E148, wherein the cells together contain nucleic acids encoding from five to 20 of the proteins.
E157. The pharmaceutical composition of E156, wherein the cells together contain nucleic acids encoding from eight to 18 of the proteins.
E158. The pharmaceutical composition of E157, wherein the cells together contain nucleic acids encoding from 10 to 15 of the proteins.
E159. The pharmaceutical composition of E149 or E150, wherein the cells together contain nucleic acids encoding from three to 10 of the proteins.
E160. The pharmaceutical composition of E159, wherein the cells together contain nucleic acids encoding from four to eight of the proteins.
E161. The pharmaceutical composition of E160, wherein the cells together contain nucleic acids encoding from five to seven of the proteins.
E162. The pharmaceutical composition of E151 or E152, wherein the cells together contain nucleic acids encoding from two to seven of the proteins.
E163. The pharmaceutical composition of E162, wherein the cells together contain nucleic acids encoding from three to six of the proteins.
E164. The pharmaceutical composition of E163, wherein the cells together contain nucleic acids encoding four or five of the proteins.
E165. The pharmaceutical composition of any one of E147-E164, wherein the population is a uniform population of cells that contain nucleic acids encoding the proteins.
E166. The pharmaceutical composition of any one of E147-E164, wherein the population is a heterogeneous population of cells that together contain nucleic acids encoding the proteins.
E167. The pharmaceutical composition of any one of E147-E166, wherein the cells are ESCs.
E168. The pharmaceutical composition of any one of E147-E166, wherein the cells are iPSCs.
E169. The pharmaceutical composition of any one of E147-E166, wherein the cells are CD34+ cells.
E170. The pharmaceutical composition of E169, wherein the CD34+ cells are HSCs.
E171. The pharmaceutical composition of E169, wherein the CD34+ cells are MPCs.
E172. The pharmaceutical composition of any one of E147-E171, wherein the composition is formulated for systemic administration to a human patient.
E173. The pharmaceutical composition of E172, wherein the patient is diagnosed with an NCD.
E174. The pharmaceutical composition of E173, wherein the NCD is a major NCD.
E175. The pharmaceutical composition of E174, wherein the major NCD interferes with the patient's independence and/or normal daily functioning.
E176. The pharmaceutical composition of E174 or E175, wherein the major NCD is associated with a score obtained by the patient on a cognitive test that is at least two standard deviations away from the mean score of a reference population.
E177. The pharmaceutical composition of E173, wherein the NCD is a mild NCD.
E178. The pharmaceutical composition of E177, wherein the mild NCD does not interfere with the patient's independence and/or normal daily functioning.
E179. The pharmaceutical composition of E177 or E178, wherein the mild NCD is associated with a score obtained by the patient on a cognitive test that is between one to two standard deviations away from the mean score of a reference population.
E180. The pharmaceutical composition of E177 or E179, wherein the reference population is a general population.
E181. The pharmaceutical composition of E176, E179, or E180, wherein the cognitive test is selected from the group consisting of ADB, AWV, GPCOG, HRA, MIS, MMSE, MoCA, SLUMS, and Short IQCODE.
E182. The pharmaceutical composition of any one of E173-E181, wherein the NCD is Alzheimer's disease.
E183. The pharmaceutical composition of any one of E173-E181, wherein the NCD is a movement disorder.
E184. The pharmaceutical composition of E183, wherein the movement disorder is Parkinson disease.
E185. The pharmaceutical composition of any one of E173-E181, wherein the NCD is a frontotemporal NCD.
E186. The pharmaceutical composition of E185, wherein the frontotemporal NCD is FTLD.
E187. The pharmaceutical composition of E186, wherein the FTLD is behavioral-variant frontotemporal dementia.
E188. The pharmaceutical composition of E186, wherein the FTLD is semantic dementia.
E189. The pharmaceutical composition of E186, wherein the FTLD is progressive nonfluent aphasia.
E190. The pharmaceutical composition of any one of E147-E189, wherein the composition is formulated for intravenous injection to the human patient.
E191. The pharmaceutical composition of any one of E147-E189, wherein the composition is formulated for direct administration to the central nervous system of a human patient.
E192. The pharmaceutical composition of E191, wherein the composition is formulated for direct administration to the CSF of the human patient.
E193. The pharmaceutical composition of E191 or E192, wherein the composition is formulated for ICV injection, intrathecal injection, stereotactic injection, intraparenchymal injection, or a combination thereof, to the human patient.
E194. The pharmaceutical composition of any one of E147-E189, wherein the composition is formulated for systemic administration and direct administration to the central nervous system of a human patient.
E195. The pharmaceutical composition of E194, wherein the composition is formulated for intravenous injection and for direct administration to the CSF of the human patient.
E196. The pharmaceutical composition of E195, wherein the composition is formulated for intravenous injection and ICV injection, intrathecal injection, stereotactic injection, intraparenchymal injection, or a combination thereof, to the human patient.
E197. The pharmaceutical composition of any one of E147-E196, wherein the cells are autologous cells.
E198. The pharmaceutical composition of any one of E147-E196, wherein the cells are allogeneic cells.
E199. The pharmaceutical composition of any one of E147-E198, wherein the cells comprise a transgene encoding one or more of the proteins operably linked to a ubiquitous promoter.
E200. The pharmaceutical composition of E199, wherein the ubiquitous promoter is selected from the group consisting of an elongation factor 1-alpha promoter and a phosphoglycerate kinase 1 promoter.
E201. The pharmaceutical composition of any one of E147-E200, wherein the cells comprise a transgene encoding one or more of the proteins operably linked to a cell lineage-specific promoter.
E202. The pharmaceutical composition of E201, wherein the cell lineage-specific promoter is selected from the group consisting of a PGRN promoter, CD11 b promoter, CD68 promoter, a C—X3-C motif chemokine receptor 1 promoter, an allograft inflammatory factor 1 promoter, a purinergic receptor P2Y12 promoter, a transmembrane protein 119 promoter, and a colony stimulating factor 1 receptor promoter.
E203. The pharmaceutical composition of any one of E147-E202, wherein the cells comprise a transgene encoding one or more of the proteins operably linked to a synthetic promoter.
E204. The pharmaceutical composition of any one of E147-E203, wherein one or more of the proteins further comprises an Rb domain of ApoE.
E205. The pharmaceutical composition of E204, wherein the Rb domain comprises a portion of ApoE having the amino acid sequence of residues 25-185, 50-180, 75-175, 100-170, 125-160, or 130-150 of SEQ ID NO: 105.
E206. The pharmaceutical composition of E204 or E205, wherein the Rb domain comprises a region having at least 70% sequence identity to the amino acid sequence of residues 159-167 of SEQ ID NO: 105.
E207. The pharmaceutical composition of any one of E147-E206, wherein the one or more nucleic acid molecules comprise a miRNA targeting sequence in the 3′-UTR.
E208. The pharmaceutical composition of E207, wherein the miRNA targeting sequence is a miR-126 targeting sequence.
E209. A pharmaceutical composition comprising a population of viral vectors that together encode two or more proteins selected from APP, PSEN1, PSEN2, APOE, TOMM40, GAB2, APOC1, TREM2, ABI3, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1 L, STK24, DISC1, MPZL1, SLC4A1AP, TRIP4, MSRA, HS3ST1, ZNF224, and AP2A2.
E210. The pharmaceutical composition of E209, wherein the proteins are selected from PSEN1, GAB2, APOC1, TREM2, ABI3, BIN1, HLA-DRB5, HLA-DRB1, CD2AP, PTK2B, INPP5D, MEF2C, CD33, MS4A4A, RIN3, PICALM, CASS4, SORL1, PLCG2, SCIMP, FRMD4A, SPPL2A, MTHFD1L, DISCI , TRIP4, and HS3ST1.
E211. A pharmaceutical composition comprising a population of viral vectors that together encode two or more proteins selected from FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1 B, NUCKS1, and ACMSD.
E212. The pharmaceutical composition of E211, wherein the proteins are selected from FCGR2A, SCAF11, DNAJC13, GCH1, LRRK2, GBA, GAK, FGF20, HLA-DQB1, and NOD2.
E213. A pharmaceutical composition comprising a population of viral vectors that together encode two or more proteins selected from HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT.
E214. The pharmaceutical composition of E213, wherein the proteins are selected from HLA-DRA, HLA-DRB5, C9ORF72, SQSTM1, TBK1, PSEN1, GRN, and CTSF.
E215. The pharmaceutical composition of any one of E209-E214, wherein the viral vectors together encode three or more of the proteins.
E216. The pharmaceutical composition of E215, wherein the viral vectors together encode four or more of the proteins.
E217. The pharmaceutical composition of E216, wherein the viral vectors together encode five or more of the proteins.
E218. The pharmaceutical composition of E209 or E210, wherein the viral vectors together encode from five to 20 of the proteins.
E219. The pharmaceutical composition of E218, wherein the viral vectors together encode from eight to 18 of the proteins.
E220. The pharmaceutical composition of E219, wherein the viral vectors together encode from 10 to 15 of the proteins.
E221. The pharmaceutical composition of E211 or E212, wherein the viral vectors together encode from three to 10 of the proteins.
E222. The pharmaceutical composition of E221, wherein the viral vectors together encode from four to eight of the proteins.
E223. The pharmaceutical composition of E222, wherein the viral vectors together encode from five to seven of the proteins.
E224. The pharmaceutical composition of E213 or E214, wherein the viral vectors together encode from two to seven of the proteins.
E225. The pharmaceutical composition of E224, wherein the viral vectors together encode from three to six of the proteins.
E226. The pharmaceutical composition of E225, wherein the viral vectors together encode four or five of the proteins.
E227. The pharmaceutical composition of any one of E209-E226, wherein the viral vectors comprise an AAV, an adenovirus, a parvovirus, a coronavirus, a rhabdovirus, a paramyxovirus, a picornavirus, an alphavirus, a herpes virus, a poxvirus, and/or a Retroviridae family virus.
E228. The pharmaceutical composition of E227, wherein the viral vectors comprise a Retroviridae family viral vector.
E229. The pharmaceutical composition of E228, wherein the Retroviridae family viral vector is a lentiviral vector.
E230. The pharmaceutical composition of E228, wherein the Retroviridae family viral vector is an alpharetroviral vector.
E231. The pharmaceutical composition of E228, wherein the Retroviridae family viral vector is a gammaretroviral vector.
E232. The pharmaceutical composition of any one of E228-E231, wherein the Retroviridae family viral vector comprises a central polypurine tract, a woodchuck hepatitis virus post-transcriptional regulatory element, a 5′-LTR, HIV signal sequence, HIV Psi signal 5′-splice site, delta-GAG element, 3′-splice site, and a 3′-self inactivating LTR.
E233. The pharmaceutical composition of E232, wherein the viral vector is an AAV selected from the group consisting of AAV1, AAV2, AAV3, AAV4, AAVS, AAV6, AAV7, AAV8, AAV9, AAV10, and AAVrh74.
E234. The pharmaceutical composition of any one of E209-E233, wherein the viral vectors comprise a pseudotyped viral vector.
E235. The pharmaceutical composition of E234, wherein the pseudotyped viral vector selected from the group consisting of a pseudotyped AAV, a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus.
E236. The pharmaceutical composition of any one of E209-E235, wherein the composition is formulated for systemic administration to a human patient.
E237. The pharmaceutical composition of E236, wherein the composition is formulated for intravenous injection to the human patient.
E238. The pharmaceutical composition of any one of E209-E235, wherein the composition is formulated for direct administration to the central nervous system of a human patient.
E239. The pharmaceutical composition of E238, wherein the composition is formulated for direct administration to the CSF of the human patient. E240. The pharmaceutical composition of E238 or E239, wherein the composition is formulated for ICV injection, intrathecal injection, stereotactic injection, intraparenchymal injection, or a combination thereof, to the human patient.
E241. The pharmaceutical composition of any one of E209-E235, wherein the composition is formulated for systemic administration and direct administration to the central nervous system of a human patient.
E242. The pharmaceutical composition of E241, wherein the composition is formulated for intravenous injection and for direct administration to the CSF of the human patient.
E243. The pharmaceutical composition of E242, wherein the composition is formulated for intravenous injection and ICV injection, intrathecal injection, stereotactic injection, intraparenchymal injection, or a combination thereof, to the human patient.
E244. The pharmaceutical composition of any one of E209-E243, wherein one or more of the viral vectors comprises a transgene encoding one or more of the proteins operably linked to a ubiquitous promoter.
E245. The pharmaceutical composition of E244, wherein the ubiquitous promoter is selected from the group consisting of an elongation factor 1-alpha promoter and a phosphoglycerate kinase 1 promoter.
E246. The pharmaceutical composition of any one of E209-E243, wherein one or more of the viral vectors comprises a transgene encoding one or more of the proteins operably linked to a cell lineage-specific promoter.
E247. The pharmaceutical composition of E246, wherein the cell lineage-specific promoter is selected from the group consisting of a PGRN promoter, CD11 b promoter, CD68 promoter, a C—X3-C motif chemokine receptor 1 promoter, an allograft inflammatory factor 1 promoter, a purinergic receptor P2Y12 promoter, a transmembrane protein 119 promoter, and a colony stimulating factor 1 receptor promoter.
E248. The pharmaceutical composition of any one of E209-E243, wherein one or more of the viral vectors comprises a transgene encoding one or more of the proteins operably linked to a synthetic promoter.
E249. The pharmaceutical composition of any one of E209-E248, wherein one or more of the proteins further comprises an Rb domain of ApoE.
E250. The pharmaceutical composition of E249, wherein the Rb domain comprises a portion of ApoE having the amino acid sequence of residues 25-185, 50-180, 75-175, 100-170, 125-160, or 130-150 of SEQ ID NO: 105.
E251. The pharmaceutical composition of E249 or E250, wherein the Rb domain comprises a region having at least 70% sequence identity to the amino acid sequence of residues 159-167 of SEQ ID NO: 105.
E252. The pharmaceutical composition of any one of E209-E251, wherein one or more of the viral vectors comprises a transgene encoding one or more of the proteins, and wherein the transgene further encodes a miRNA targeting sequence in the 3′-UTR. E253. The pharmaceutical composition of E252, wherein the miRNA targeting sequence is a miR-126 targeting sequence.
E254. A kit comprising the pharmaceutical composition of any one of E209, E210, E215-E220, and
E227-E253, wherein the kit further comprises a package insert instructing a user of the kit to administer the pharmaceutical composition to a human patient having an NCD.
E255. The kit of E254, wherein the NCD is a major NCD.
E256. The kit of E255, wherein the major NCD interferes with the patient's independence and/or normal daily functioning.
E257. The kit of E255 or E256, wherein the major NCD is associated with a score obtained by the patient on a cognitive test that is at least two standard deviations away from the mean score of a reference population.
E258. The kit of E254, wherein the NCD is a mild NCD.
E259. The kit of E256, wherein the mild NCD does not interfere with the patient's independence and/or normal daily functioning.
E260. The kit of E258 or E259, wherein the mild NCD is associated with a score obtained by the patient on a cognitive test that is between one to two standard deviations away from the mean score of a reference population.
E261. The kit of E257 or E260, wherein the reference population is a general population.
E262. The kit of E257, E260, or E261, wherein the cognitive test is selected from the group consisting of AD8, AWV, GPCOG, HRA, MIS, MMSE, MoCA, SLUMS, and Short IQCODE.
E263. The kit of any one of E254-E262, wherein the NCD is Alzheimer's disease.
E264. A kit comprising the pharmaceutical composition of any one of E211, E212, E221-E226, and
E227-E253, wherein the kit further comprises a package insert instructing a user of the kit to administer the pharmaceutical composition to a human patient having an NCD.
E265. The kit of E264, wherein the NCD is a movement disorder.
E266. The kit of E265, wherein the movement disorder is Parkinson disease.
E267. A kit comprising the pharmaceutical composition of any one of E213, E214, E224-E226, and
E227-E253, wherein the kit further comprises a package insert instructing a user of the kit to administer the pharmaceutical composition to a human patient having an NCD.
E268. The kit of E267, wherein the NCD is a major NCD.
E269. The kit of E268, wherein the major NCD interferes with the patient's independence and/or normal daily functioning.
E270. The kit of E268 or E269, wherein the major NCD is associated with a score obtained by the patient on a cognitive test that is at least two standard deviations away from the mean score of a reference population.
E271. The kit of E268, wherein the NCD is a mild NCD.
E272. The kit of E271, wherein the mild NCD does not interfere with the patient's independence and/or normal daily functioning.
E273. The kit of E271 or E272, wherein the mild NCD is associated with a score obtained by the patient on a cognitive test that is between one to two standard deviations away from the mean score of a reference population.
E274. The kit of E270 or E273, wherein the reference population is a general population.
E275. The kit of E270, E273, or E274, wherein the cognitive test is selected from the group consisting of AD8, AWV, GPCOG, HRA, MIS, MMSE, MoCA, SLUMS, and Short IQCODE.
E276. The kit of any one of E267-E275, wherein the NCD is a frontotemporal NCD.
E277. The kit of E276, wherein the frontotemporal NCD is FTLD.
E278. The kit of E277, wherein the FTLD is behavioral-variant frontotemporal dementia.
E279. The kit of E277, wherein the FTLD is semantic dementia.
E280. The kit of E277, wherein the FTLD is progressive nonfluent aphasia.
E281. A method of treating a patient diagnosed as having an NCD, the method comprising providing to the patient one or more agents that collectively increase expression and/or activity of two or more proteins selected from APP, PSEN1, PSEN2, APOE, TOMM40, GAB2, APOC1, TREM2, ABI3, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1L, STK24, DISCI , MPZL1, SLC4A1AP, TRIP4, MSRA, HS3ST1, ZNF224, AP2A2, FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1 L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1B, NUCKS1, ACMSD, HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT.
E282. The method of E281, wherein the NCD is a major NCD.
E283. The method of E282, wherein the major NCD interferes with the patient's independence and/or normal daily functioning.
E284. The method of E282 or E283, wherein the major NCD is associated with a score obtained by the patient on a cognitive test that is at least two standard deviations away from the mean score of a reference population.
E285. The method of E281, wherein the NCD is a mild NCD.
E286. The method of E285, wherein the mild NCD does not interfere with the patient's independence and/or normal daily functioning.
E287. The method of E285 or E286, wherein the mild NCD is associated with a score obtained by the patient on a cognitive test that is between one to two standard deviations away from the mean score of a reference population.
E288. The method of E284 or E287, wherein the reference population is a general population.
E289. The method of E284, E287, or E288, wherein the cognitive test is selected from the group consisting of ADB, AWV, GPCOG, HRA, MIS, MMSE, MoCA, SLUMS, Short IQCODE.
E290. The method of any one of E281-E289, wherein the NCD is associated with impairment in one or more of complex attention, executive function, learning and memory, language, perceptual-motor function, and social cognition.
E291. The method of any one of E281-E290, wherein the NCD is not due to delirium or other mental disorder.
E292. The method of any one of E281-E291, wherein the NCD is Alzheimer's disease.
E293. The method of any one of E281-E291, wherein the NCD is a movement disorder.
E294. The method of any one of E293, wherein the movement disorder is Parkinson disease.
E295. The method of any one of E281-E291, wherein the NCD is a frontotemporal NCD.
E296. The method of E295, wherein the frontotemporal NCD is FTLD.
E297. The method of any one of E1 -E150, wherein the cells are pluripotent cells (e.g., ESCs, iPSCs), multipotent cells (e.g., CD34+ cells, such as, e.g., HSGs or MPCs), BLPCs, monocytes, macrophages, microglial progenitor cells, or microglia.
E298. The method of any one of E1 -E146, wherein the transgene is capable of expression in a macrophage or a microglial cell.
E299. A pharmaceutical composition comprising a population of cells that together contain nucleic acids encoding two or more proteins selected from APP, PSEN1, PSEN2, APOE, TOMM40, GAB2, APOC1, TREM2, ABI3, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1L, STK24, DISCI , MPZL1, SLC4A1AP, TRIP4, MSRA, HS3ST1, ZNF224, AP2A2, FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1 L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1B, NUCKS1, ACMSD, HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT.
E300. A pharmaceutical composition comprising a population of viral vectors that together encode two or more proteins selected from APP, PSEN1, PSEN2, APOE, TOMM40, GAB2, APOC1, TREM2, ABI3, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1L, STK24, DISCI , MPZL1, SLC4A1AP, TRIP4, MSRA, HS3ST1, ZNF224, AP2A2, FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1 B, NUCKS1, ACMSD, HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT.
E301. A kit comprising the pharmaceutical composition of any one of E227 or E299, wherein the kit further comprises a package insert instructing a user of the kit to administer the pharmaceutical composition to a human patient having an NCD.
E302. The kit of E301, wherein the NCD is a major NCD.
E303. The kit of E302, wherein the major NCD interferes with the patient's independence and/or normal daily functioning.
E304. The kit of E302 or E303, wherein the major NCD is associated with a score obtained by the patient on a cognitive test that is at least two standard deviations away from the mean score of a reference population.
E305. The kit of E301, wherein the NCD is a mild NCD.
E306. The kit of E305, wherein the mild NCD does not interfere with the patient's independence and/or normal daily functioning.
E307. The kit of E305 or E306, wherein the mild NCD is associated with a score obtained by the patient on a cognitive test that is between one to two standard deviations away from the mean score of a reference population.
E308. The kit of E304 or E307, wherein the reference population is a general population.
E309. The kit of E304, E307, or E308, wherein the cognitive test is selected from the group consisting of ADB, AWV, GPCOG, HRA, MIS, MMSE, MoCA, SLUMS, and Short IQCODE.
E310. The kit of any one of E301-E309, wherein the NCD is Alzheimer's disease.
E311. The kit of any one of E301-E309, wherein the NCD is a movement disorder.
E312. The kit of E311, wherein the movement disorder is Parkinson disease.
E313. The kit of any one of E301-E309, wherein the NCD is a frontotemporal NCD.
E314. The kit of E313, wherein the frontotemporal NCD is FTLD.
E315. The kit of E314, wherein the FTLD is behavioral-variant frontotemporal dementia.
E316. The kit of E314, wherein the FTLD is semantic dementia.
E317. The kit of E314, wherein the FTLD is progressive nonfluent aphasia.

### BRIEF DESCRIPTION OF THE DRAWINGS

FIG. 1 is a Western blot showing expression of the human triggering receptor expressed on myeloid cells 2 (TREM2) protein in murine macrophages transduced with a lentiviral vector encoding TREM2. Cell lysates were generated from the RAW murine macrophage cells transduced with an MND.TREM2 viral vector (MND.TREM2), an MND.green fluorescent protein (GFP) viral vector (MND.GFP) at multiplicity of infection (MOI) of 10, 50, 100, or 200, or from non-transduced control (NTC) cells. TREM2 expression was assessed using an antibody raised against human TREM2 (FIG. 1).

FIG. 2 is a Western blot showing expression of the human TREM2 protein in murine microglial cells transduced with a lentiviral vector encoding TREM2. Cell lysates were generated from primary murine microglia non-transduced (NT) or transduced with an MND.TREM2 viral vector (MND-TREM2) or an MND.GFP viral vector (MND-GFP). TREM2 expression was assessed using an antibody raised against human TREM2 (FIG. 2).

FIG. 3 is a Western blot showing expression of the human TREM2 protein in lineage negative (Lin−) cells transduced with a lentiviral vector encoding TREM2. Cell lysates from Lin− murine cells transduced with an MND.TREM2 viral vector (Lenti TREM2) or an MND.GFP viral vector. TREM2 expression was assessed using an antibody raised against human TREM2 (FIG. 3).

FIGS. 4A-4B are a series of plots showing transduction of human cells with a lentiviral vector containing a transgene encoding the human progranulin (PGRN) protein. Cell lysates were generated from human 239T cells transduced with a lentiviral vector encoding PGRN (MND.GRN) or green fluorescent protein (GFP; MND.GFP) at a multiplicity of infection (MOI) of 10, 50, 100, or 200. A separate set of control cells were not transduced (NTC). Densitometry was used to quantify PGRN levels over actin (FIG. 4A). Western blots using an antibody raised against human PGRN indicate stable PGRN expression in 239T cells, with highest expression observed at MOI 200 (FIG. 4B). All groups were showed statistically significant differences, except for the NTC cells and MOI 10 GFP cells. Statistical analysis was performed using ANOVA.

FIG. 5 is a Western blot showing expression of human PGRN in murine lineage negative (Lin-) cells transduced with a lentiviral vector containing a transgene encoding human PGRN (i.e., a MND.GRN vector). Conditioned media generated from Lin− mouse cells non-transduced (−) or transduced with MND.GRN lentiviral vector (+) were analyzed using Western blot with an antibody raised against human PGRN, showing release of human PGRN protein into the growth media by the transduced cells (FIG. 5).

FIG. 6 is a Western blot showing immortalized cell lines transduced with a lentiviral vector containing a transgene encoding human PGRN is N-linked glycosylated. Cell lysates were generated from human 239 T cell lines non-transduced (NT1, NT2, NT3, and NT4) or transduced with a lentiviral vector encoding human PGRN (MND.GRN-1, MND.GRN-2, MND.GRN-3, and MND.GRN-4) were generated in four independent rounds of transduction. Cell lysates were enzymatically digested with either EndoH (E.) or PNGase (P.) enzymes, or heated (H.) and analyzed using Western blot with an antibody raised against human progranulin. Enzymatic digestion by EndoH and PNGase indicate that the human PGRN protein produced by the transduced cells is N-linked glycosylated (FIG. 6).

### Definitions

As used herein, the terms “ablate,” “ablating,” “ablation,” and the like refer to the depletion of one or more cells in a population of cells in vivo or ex vivo. In some embodiments of the present disclosure, it may be desirable to ablate endogenous cells within a patient (e.g., a patient undergoing treatment for a disease described herein, such as a neurocognitive disorder (NCD; e.g., Alzheimer's disease, Parkinson's disease, or a frontotemporal lobar dementia (FTLD))) before administering a therapeutic composition, such as a therapeutic population of cells, to the patient. This can be beneficial, for example, in order to provide newly-administered cells with an environment within which the cells may engraft. Ablation of a population of endogenous cells can be performed in a manner that selectively targets a specific cell type, for example, using antibody-drug conjugates that bind to an antigen expressed on the target cell and subsequently engender the killing of the target cell. Additionally or alternatively, ablation may be performed in a non-specific manner using cytotoxins that do not localize to a particular cell type but are instead capable of exerting their cytotoxic effects on a variety of different cells. Exemplary agents that may be used to ablate a population of endogenous cells in a patient, such as a population of endogenous microglia or microglial precursor cells in a patient undergoing therapy, e.g., for the treatment of an NCD, are busulfan, PLX3397, PLX647, PLX5622, treosulfan, clodronate liposomes, and combinations thereof. Examples of ablation include depletion of at least 5% of cells (e.g., at least 5%, 10%, 15%, 20%, 25%, 30%, 35%, 40%, 45%, 50%, or more) in a population of cells in vivo or in vitro. Quantifying cell counts within a sample of cells can be performed using a variety of cell-counting techniques, such as through the use of a counting chamber, a Coulter counter, flow cytometry, or other cell-counting methods known in the art.

As used herein in the context of a protein of interest, the term “activity” refers to the biological functionality that is associated with a wild-type form of the protein. For example, in the context of an enzyme, the term “activity” refers to the ability of the protein to effectuate substrate turnover in a manner that yields the product of a corresponding chemical reaction. Activity levels of enzymes can be detected and quantitated, for example, using substrate turnover assays known in the art. As another example, in the context of a membrane-bound receptor, the term “activity” may refer to signal transduction initiated by the receptor, e.g., upon binding to its cognate ligand. Activity levels of receptors involved in signal transduction pathways can be detected and quantitated, for example, by observing an increase in the outcome of receptor signaling, such as an increase in the transcription of one or more genes (which may be detected, e.g., using polymerase chain reaction techniques known in the art).

As used herein, the terms “administering,” “administration,” and the like refer to directly giving a patient a therapeutic agent (e.g., a population of cells, such as a population of cells (e.g., pluripotent cells (e.g., embryonic stem cells (ESCs) or induced pluripotent stem cells (ISPCs)), multipotent cells (e.g., CD34+ cells such as, e.g., hematopoietic stem cells (HSCs) or myeloid precursor cells (MPCs)), blood lineage progenitor cells (BLPCS; e.g., monocytes), macrophages, microglial progenitor cells, or microglia), that together contain nucleic acids encoding one or more proteins described herein (e.g., nucleic acids capable of expression in macrophages or microglia) by any effective route. Exemplary routes of administration are described herein and include systemic administration routes, such as intravenous injection, as well as routes of administration directly to the central nervous system of the patient, such as by way of intracerebroventricular injection, intrathecal injection, and stereotactic injection, among others.

As used herein, the term “allogeneic” refers to cells, tissues, nucleic acid molecules, or other substances obtained or derived from a different patient of the same species. For example, in the context of a population of cells expressing one or more proteins described herein, allogeneic cells include those that are (i) obtained from a patient that is not undergoing therapy and are then (ii) transduced or transfected with a vector that directs the expression of one or more desired proteins. The phrase “directs expression” refers to the inclusion of one or more polynucleotides encoding the one or more proteins to be expressed. The polynucleotide may contain additional sequence motifs that enhances expression of the protein of interest.

As used herein, the term “autologous” refers to cells, tissues, nucleic acid molecules, or other substances obtained or derived from an individual's own cells, tissues, nucleic acid molecules, or the like. For example, in the context of a population of cells expressing one or more proteins described herein, autologous cells include those that are obtained from the patient undergoing therapy that are then transduced or transfected with a vector that directs the expression of one or more proteins of interest.

As used herein, the term “ApoE” refers to apolipoprotein E, a member of a class of proteins involved in lipid transport. Apolipoprotein E is a fat-binding protein (apolipoprotein) that is part of the chylomicron and intermediate-density lipoprotein (IDLs). These are essential for the normal processing (catabolism) of triglyceride-rich lipoproteins. ApoE is encoded by the APOE gene. The term “ApoE” also refers to variants of the wild type ApoE protein, such as proteins having at least 70% identity (e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to the amino acid sequence of wild type ApoE, which is set forth in SEQ ID NO: 105.

As used herein, the term “blood lineage progenitor cell” or “BLPC” refers to any cell (e.g., a mammalian cell) capable of differentiating into one or more (e.g., 2, 3, 4, 5 or more) types of hematopoietic (i.e., blood) cells. A BLPC may differentiate into erythrocytes, leukocytes (e.g., such as granulocytes (e.g., basophils, eosinophils, neutrophils, and mast cells) or agranulocytes (e.g., lymphocytes and monocytes)), or thrombocytes. A BLPC may also include a differentiated blood cell (e.g., a monocyte) that can further differentiate into another blood cell type (e.g., a macrophage).

As used herein, the term “cell type” refers to a group of cells sharing a phenotype that is statistically separable based on gene expression data. For example, cells of a common cell type may share similar structural and/or functional characteristics, such as similar gene activation patterns and antigen presentation profiles. Cells of a common cell type may include those that are isolated from a common tissue (e.g., epithelial tissue, neural tissue, connective tissue, or muscle tissue) and/or those that are isolated from a common organ, tissue system, blood vessel, or other structure and/or region in an organism.

As used herein, “codon optimization” refers a process of modifying a nucleic acid sequence in accordance with the principle that the frequency of occurrence of synonymous codons (e.g., codons that code for the same amino acid) in coding DNA is biased in different species. Such codon degeneracy allows an identical polypeptide to be encoded by a variety of nucleotide sequences. Sequences modified in this way are referred to herein as “codon-optimized.” This process may be performed on any of the sequences described in this specification to enhance expression or stability. Codon optimization may be performed in a manner such as that described in, e.g., U.S. Pat. Nos. 7,561,972, 7,561,973, and 7,888,112, each of which is incorporated herein by reference in its entirety. The sequence surrounding the translational start site can be converted to a consensus Kozak sequence according to known methods. See, e.g., Kozak et al, Nucleic Acids Res. 15:8125-48 (1987), incorporated herein by reference in its entirety. Multiple stop codons can be incorporated.

As used herein, the term “cognitive test” refers to a test that can be performed by a skilled practitioner in order to assess the cognitive capabilities of humans and other animals. A cognitive test may be used to assess inductive reasoning skills, intelligence quotient, cognitive development, memory, knowledge organization, metacognition, thought, mental chronometry. A cognitive test may be used to assess the performance of a patient across several cognitive domains, including, but not limited to executive function, learning and memory, language, perceptual-motor function, and social cognition. Examples of cognitive tests include, but are not limited to Eight-item Informant Interview to Differentiate Aging and Dementia (AD8), Annual Wellness Visit (AWV), General Practitioner Assessment of Cognition (GPCOG), Health Risk Assessment (HRA), Memory Impairment Screen (MIS), Mini Mental Status Exam (MMSE), Montreal Cognitive Assessment (MoCA), St. Louis University Mental Status Exam (SLUMS), and Short Informant Questionnaire on Cognitive Decline in the Elderly (Short IQCODE). A skilled practitioner will recognize that other cognitive tests well-known in the art may also be used to assess cognitive function in a patient.

As used herein, the term “complex attention” refers to a cognitive function that describes a patient's (e.g., a human patient's) ability to maintain information in their mind for a short time and to perform an operation on that information (e.g., mental arithmetic). Impairment in complex attention may result in difficulty with focusing on conversations, difficulty filtering out unwanted information, problems with prospective memory (e.g., remembering to remember something later on), and inefficient memory for new information.

As used herein, the terms “condition” and “conditioning” refer to processes by which a patient is prepared for receipt of a transplant containing a population of cells (e.g., a population of cells, such as CD34+ cells, hematopoietic stem cells, or myeloid progenitor cells). Such procedures promote the engraftment of a cell transplant, for example, by selectively depleting endogenous cells (e.g., endogenous CD34+ cells, hematopoietic stem cells, myeloid progenitor cells, or microglial cells, among others) thereby creating a vacancy which is in turn filled by the exogenous cell transplant. According to the methods described herein, a patient may be conditioned for cell transplant procedure by administration to the patient of one or more agents capable of ablating endogenous cells (e.g., CD34+ cells, hematopoietic stem cells, myeloid progenitor cells, or microglial cells, among others), such as busulfan, treosulfan, PLX3397, PLX647, PLX5622, and clodronate liposomes, radiation therapy, or a combination thereof.

Conditioning regimens useful in conjunction with the compositions and methods of the disclosure may be myeloablative or non-myeloablative. Other cell-ablating agents and methods well known in the art (e.g., antibody-drug conjugates) may also be used.

As used herein, the terms “conservative mutation,” “conservative substitution,” “conservative amino acid substitution,” and the like refer to a substitution of one or more amino acids for one or more different amino acids that exhibit similar physicochemical properties, such as polarity, electrostatic charge, and steric volume. These properties are summarized for each of the twenty naturally-occurring amino acids in Table 5 below.

TABLE 5
Representative physicochemical properties
of naturally occurring amino acids

| | | | | Electrostatic | |
|---|---|---|---|---|---|
| | 3 | 1 | Side- | character at | |
| | Letter | Letter | chain | physiological | Steric |
| Amino Acid | Code | Code | Polarity | pH (7.4) | Volume^{†} |
| Alanine | Ala | A | nonpolar | neutral | small |
| Arginine | Arg | R | polar | cationic | large |
| Asparagine | Asn | N | polar | neutral | intermediate |
| Aspartic acid | Asp | D | polar | anionic | intermediate |
| Cysteine | Cys | C | nonpolar | neutral | intermediate |
| Glutamic acid | Glu | E | polar | anionic | intermediate |
| Glutamine | Gln | Q | polar | neutral | intermediate |
| Glycine | Gly | G | nonpolar | neutral | small |
| Histidine | His | H | polar | Both neutral | large |
| | | | | and cationic | |
| | | | | forms in | |
| | | | | equilibrium | |
| | | | | at pH 7.4 | |
| Isoleucine | Ile | I | nonpolar | neutral | large |
| Leucine | Leu | L | nonpolar | neutral | large |
| Lysine | Lys | K | polar | cationic | large |
| Methionine | Met | M | nonpolar | neutral | large |
| Phenylalanine | Phe | F | nonpolar | neutral | large |
| Proline | Pro | P | nonpolar | neutral | intermediate |
| Serine | Ser | S | polar | neutral | small |
| Threonine | Thr | T | polar | neutral | intermediate |
| Tryptophan | Trp | W | nonpolar | neutral | bulky |
| Tyrosine | Tyr | Y | polar | neutral | large |
| Valine | Val | V | nonpolar | neutral | intermediate |

^{†}based on volume in A³: 50-100 is small, 100-150 is intermediate, 150-200 is large, and >200 is bulky

From this table it is appreciated that the conservative amino acid families include (i) G, A, V, L and I; (ii) D and E; (iii) C, S and T; (iv) H, K and R; (v) N and Q; and (vi) F, Y and W. A conservative mutation or substitution is therefore one that substitutes one amino acid for a member of the same amino acid family (e.g., a substitution of Ser for Thr or Lys for Arg).

As used herein, the phrase “delirium or other mental disorder” refers to a condition such as delirium (i.e., a syndrome encompassing impaired attention, consciousness, and cognition that develops over a short period of time (e.g., hours to days)) or another disorder of the mind (e.g., schizophrenia, bipolar disorder, and major depression) that is distinct from a neurocognitive disorder and does not exhibit cognitive impairment as a core symptom. For example, a condition such as delirium or another mental disorder may differ from an NCD in that cognitive impairment may by a symptom that is associated with the disease but is not a central feature of said disease. Delirium or another mental disorder may differ from an NCD with respect to time to onset (e.g., hours to days in delirium versus months to years for an NCD), etiology (e.g., substance-induced delirium), symptom length (e.g., delirium may last hours to days whereas an NCD can last for years), and resolution (e.g., delirium may resolve completely, whereas an NCD does not resolve in most cases).

As used herein in the context of a gene of interest, the term “disrupt” refers to preventing the formation of a functional gene product. A gene product is considered to be functional according to the present disclosure if it fulfills its normal (wild type) function(s). Disruption of the gene prevents expression of a functional factor (e.g., protein) encoded by the gene and may be achieved, for example, by way of an insertion, deletion, or substitution of one or more bases in a sequence encoded by the gene and/or a promoter and/or an operator that is necessary for expression of the gene in a patient. The disrupted gene may be disrupted by, e.g., removal of at least a portion of the gene from a genome of the patient, alteration of the gene to prevent expression of a functional factor (e.g., protein) encoded by the gene, an interfering RNA, or expression of a dominant negative factor by an exogenous gene. Materials and methods for genetically modifying cells so as to disrupt the expression of one or more genes are detailed, for example, in U.S. Pat. No. 8,518,701; U.S. Pat. No. 9,499,808; and US 2012/0222143, the disclosures of each of which are incorporated herein by reference in their entirety (in case of conflict, the instant specification is controlling).

As used herein, the terms “effective amount,” “therapeutically effective amount,” and the like, when used in reference to a therapeutic composition, such as a vector construct, viral vector, or cell described herein, refer to a quantity sufficient to, when administered to the patient, including a mammal, for example a human, effect beneficial or desired results, such as clinical results. For example, in the context of treating an NCD described herein, these terms refer to an amount of the composition sufficient to achieve a treatment response as compared to the response obtained without administration of the composition, vector construct, viral vector or cell. The quantity of a given composition described herein that will correspond to such an amount may vary depending upon various factors, such as the given agent, the pharmaceutical formulation, the route of administration, the type of disease or disorder, the identity of the patient (e.g., age, sex, weight) or host being treated, and the like. An “effective amount,” “therapeutically effective amount,” or the like, of a composition, such as a vector construct, viral vector, or cell of the present disclosure, also include an amount that results in a beneficial or desired result in a patient as compared to a control.

As used herein, the terms “embryonic stem cell” and “ES cell” refer to an embryo-derived totipotent or pluripotent stem cell, derived from the inner cell mass of a blastocyst that can be maintained in an in vitro culture under suitable conditions. ES cells are capable of differentiating into cells of any of the three vertebrate germ layers, e.g., the endoderm, the ectoderm, or the mesoderm. ES cells are also characterized by their ability propagate indefinitely under suitable in vitro culture conditions. ES cells are described, for example, in Thomson et al., Science 282:1145 (1998), the disclosure of which is incorporated herein by reference as it pertains to the structure and functionality of embryonic stem cells.

As used herein, the term “endogenous” describes a molecule (e.g., a polypeptide, nucleic acid, or cofactor) that is found naturally in a particular organism (e.g., a human) or in a particular location within an organism (e.g., an organ, a tissue, or a cell, such as a human cell).

As used herein, the term “engraft” and “engraftment” refer to the process by which hematopoietic stem cells and progenitor cells, whether such cells are produced endogenously within the body or transplanted using any of the administration methods described herein, repopulate a tissue. The term encompasses all events surrounding or leading up to engraftment, such as tissue homing of cells and colonization of cells within the tissue of interest.

As used herein, the term “executive function” refers to a set of cognitive functions that facilitate cognitive control of behavior in a patient (e.g., a human). Executive function encompasses, e.g., selection and monitoring goal-directed behaviors, attentional control, cognitive inhibition, inhibitory control, working memory, and cognitive flexibility. An individual normally acquires or perfects executive functions across the lifespan, although this process may be derailed by the development of an NCD in the patient, which may adversely impact executive function.

As used herein, the term “express” refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein. In the context of a gene that encodes a protein product, the terms “gene expression” and the like are used interchangeably with the terms “protein expression” and the like. Expression of a gene or protein of interest in a patient can manifest, for example, by detecting: an increase in the quantity or concentration of mRNA encoding corresponding protein (as assessed, e.g., using RNA detection procedures described herein or known in the art, such as quantitative polymerase chain reaction (qPCR) and RNA seq techniques), an increase in the quantity or concentration of the corresponding protein (as assessed, e.g., using protein detection methods described herein or known in the art, such as enzyme-linked immunosorbent assays (ELISA), among others), and/or an increase in the activity of the corresponding protein (e.g., in the case of an enzyme, as assessed using an enzymatic activity assay described herein or known in the art) in a sample obtained from the patient. As used herein, a cell is considered to “express” a gene or protein of interest if one or more, or all, of the above events can be detected in the cell or in a medium in which the cell resides. For example, a gene or protein of interest is considered to be “expressed” by a cell or population of cells if one can detect (i) production of a corresponding RNA transcript, such as an mRNA template, by the cell or population of cells (e.g., using RNA detection procedures described herein); (ii) processing of the RNA transcript (e.g., splicing, editing, 5′ cap formation, and/or 3′ end processing, such as using RNA detection procedures described herein); (iii) translation of the RNA template into a protein product (e.g., using protein detection procedures described herein); and/or (iv) post-translational modification of the protein product (e.g., using protein detection procedures described herein).

As used herein, the term “exogenous” describes a molecule (e.g., a polypeptide, nucleic acid, or cofactor) that is not found naturally in a particular organism (e.g., a human) or in a particular location within an organism (e.g., an organ, a tissue, or a cell, such as a human cell). Exogenous materials include those that are provided from an external source to an organism or to cultured matter extracted there from.

As used herein, the term “functional potential” as it pertains to a cell, such as a hematopoietic stem cell, refers to the functional properties of stem cells which include: 1) multi-potency (which refers to the ability to differentiate into multiple different blood lineages including, but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells); 2) self-renewal (which refers to the ability of stem cells to give rise to daughter cells that have equivalent potential as the mother cell, and further that this ability can repeatedly occur throughout the lifetime of an individual without exhaustion); and 3) the ability of stem cells or progeny thereof to be reintroduced into a transplant recipient whereupon they home to the stem cell niche and re-establish productive and sustained cell growth and differentiation.

As used herein, the term “general population” refers to an entire population of individuals having a particular characteristic of interest (e.g., age, medical history, education, socioeconomic status, or lifestyle, among others). Alternatively, the term “general population” may refer to a subset of the entire population of individuals having a particular characteristic of interest, such as, e.g., a random sample having a defined sample size. According to the methods disclosed herein, the general population may serve as a practical referent (e.g., a reference population) to which a measured variable can be compared. For example, a patient diagnosed with an may have their cognition assessed using a cognitive test disclosed herein and the score obtained by the patient on the test may be compared against performance of individuals in the general population (e.g., the entire general population or a random sample of the general population) on the same test. The size of the random sample of the general population may be determined by a skilled practitioner using methods well-known in the art. For example, a skilled practitioner may perform a power analysis prior to collecting data (e.g., prior to conducting a cognitive test on a patient) to determine the smallest sample that is needed to detect a statistically significant effect with a desired level of confidence.

As used herein, the terms “hematopoietic stem cells” and “HSCs” refer to immature blood cells having the capacity to self-renew and to differentiate into mature blood cells of diverse lineages including but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). It is known in the art that such cells may or may not include CD34+ cells. CD34+ cells are immature cells that express the CD34 cell surface marker. In humans, CD34+ cells are believed to include a subpopulation of cells with the stem cell properties defined above, whereas in mice, HSCs are CD34−. In addition, HSCs also refer to long term repopulating HSC (LT-HSC) and short-term repopulating HSC (ST-HSC). LT-HSC and ST-HSC are differentiated, based on functional potential and on cell surface marker expression. For example, human HSC are a CD34+, CD38−, CD45RA-, CD90+, CD49F+, and lin− (negative for mature lineage markers including CO2, CD3, CD4, CD7, CD8, CD10, CD11B, CD19, CD20, CD56, CD235A). In mice, bone marrow LT-HSC are CD34−, SCA-1+, C-kit+, CD135−, Slamf1/CD150+, CD48−, and lin− (negative for mature lineage markers including Ter119, CD11b, Gr1, CD3, CD4, CD8, B220, IL-7ra), whereas ST-HS Care CD34+, SCA-1+, C-kit+, CD135−, Slamf1/CD150+, and lin− (negative for mature lineage markers including Ter119, CD11b, Gr1, CD3, CD4, CD8, B220, IL-7ra). In addition, ST-HSC are less quiescent (i.e., more active) and more proliferative than L T-HSC under homeostatic conditions. However, LT-HSC have greater self-renewal potential (i.e., they survive throughout adulthood, and can be serially transplanted through successive recipients), whereas ST-HSC have limited self-renewal (i.e., they survive for only a limited period of time, and do not possess serial transplantation potential). Any of these HSCs can be used in any of the methods described herein. Optionally, ST-HSCs are useful because they are highly proliferative and thus, can more quickly give rise to differentiated progeny.

As used herein, the term “HLA-matched” refers to a donor-recipient pair in which none of the HLA antigens are mismatched between the donor and recipient, such as a donor providing a hematopoietic stem cell graft to a recipient in need of hematopoietic stem cell transplant therapy. HLA-matched (i.e., where all of the 6 alleles are matched) donor-recipient pairs have a decreased risk of graft rejection, as endogenous T cells and NK cells are less likely to recognize the incoming graft as foreign and are thus less likely to mount an immune response against the transplant.

As used herein, the term “HLA-mismatched” refers to a donor-recipient pair in which at least one HLA antigen, in particular with respect to HLA-A, HLA-B, HLA-C, and HLA-DR, is mismatched between the donor and recipient, such as a donor providing a hematopoietic stem cell graft to a recipient in need of hematopoietic stem cell transplant therapy. In some embodiments, one haplotype is matched and the other is mismatched. HLA-mismatched donor-recipient pairs may have an increased risk of graft rejection relative to HLA-matched donor-recipient pairs, as endogenous T cells and NK cells are more likely to recognize the incoming graft as foreign in the case of an HLA-mismatched donor-recipient pair, and such T cells and NK cells are thus more likely to mount an immune response against the transplant.

As used herein, the phrase “independence or normal daily functioning” refers to the ability of a patient (e.g., a human) to successfully perform everyday activities without assistance from a caretaker or a social worker. Non-limiting examples of activities that enable an individual to independently carry out daily functions include, e.g., social, occupational, or academic functioning, personal hygiene, grooming, dressing, toilet hygiene, functional mobility (e.g., ability to walk, get in and out of bed), and self-feeding. A patient diagnosed with a major NCD, may have difficulty independently performing normal daily functions, whereas a patient diagnosed with mild NCD may not have difficulty independently performing daily tasks.

As used herein, the terms “induced pluripotent stem cell,” “iPS cell,” and “iPSC” refer to a pluripotent stem cell that can be derived directly from a differentiated somatic cell. Human iPS cells can be generated by introducing specific sets of reprogramming factors into a non-cell that can include, for example, Oct3/4, Sox family transcription factors (e.g., Sox1, Sox2, Sox3, Sox15), Myc family transcription factors (e.g., c-Myc, 1-Myc, n-Myc), Kruppel-like family (KLF) transcription factors (e.g., KLF1, KLF2, KLF4, KLF5), and/or related transcription factors, such as NANOG, LIN28, and/or Glis1. Human iPS cells can also be generated, for example, by the use of miRNAs, small molecules that mimic the actions of transcription factors, or lineage specifiers. Human iPS cells are characterized by their ability to differentiate into any cell of the three vertebrate germ layers, e.g., the endoderm, the ectoderm, or the mesoderm. Human iPS cells are also characterized by their ability propagate indefinitely under suitable in vitro culture conditions. Human iPS cells are described, for example, in Takahashi and Yamanaka, Cell 126:663 (2006), the disclosure of which is incorporated herein by reference as it pertains to the structure and functionality of iPS cells.

As used herein, the term “IRES” refers to an internal ribosome entry site. In general, an IRES sequence is a feature that allows eukaryotic ribosomes to bind an mRNA transcript and begin translation without binding to a 5′ capped end. An mRNA containing an IRES sequence produces two translation products, one initiating form the 5′ end of the mRNA and the other from an internal translation mechanism mediated by the IRES.

As used herein, the phrase “learning and memory” refer to a cognitive ability that encompasses the acquisition of skills or knowledge and expression of acquired skills or knowledge (e.g., learning to say a new word and uttering the new word, respectively). “Learning and memory” may refer to two independent processes of 1) acquiring new skills or knowledge (i.e., learning); and 2) processing, storing, and recalling the learned skill or knowledge (i.e., memory), which may differ by timescales (learning is generally slower and more effortful than recalling a memory or performing a learned skill) and neurobiological basis. A patient diagnosed with an NCD may have impaired learning and memory relative to a healthy patient.

As used herein, the term “macrophage” refers to a type of white blood cell that engulfs and digests cellular debris, foreign substances, microbes, cancer cells, and anything else that does not have 15 the types of proteins specific to healthy body cells on its surface in a process called phagocytosis. Macrophages are found in essentially all tissues, where they patrol for potential pathogens by amoeboid movement. They take various forms (with various names) throughout the body (e.g., histiocytes, Kupffer cells, alveolar macrophages, microglia, and others), but all are part of the mononuclear phagocyte system. Besides phagocytosis, they play a critical role in non-specific defense (innate immunity) and also 20 help initiate specific defense mechanisms (adaptive immunity) by recruiting other immune cells such as lymphocytes. For example, they are important as antigen presenters to T cells. Beyond increasing inflammation and stimulating the immune system, macrophages also play an important anti-inflammatory role and can decrease immune reactions through the release of cytokines.

As used herein, the terms “microglia” or “microglial cell” refer to a type of neuroglial cell found in the brain and spinal cord that function as resident macrophage cells and the principal line of immune defense in the central nervous system. Primary functions of microglial cells include immune surveillance, phagocytosis, extracellular signaling (e.g., production and release of cytokines, chemokines, prostaglandins, and reactive oxygen species), antigen presentation, and promotion of tissue repair and regeneration.

As used herein, the term “microglial progenitor cell” refers to a precursor cell that gives rise to microglial cells. Microglial precursor cells originate in the yolk sac during a limited period of embryonic development, infiltrate the brain mesenchyme, and perpetually renew themselves throughout life.

As used herein, the term “miRNA targeting sequence” refers to a nucleotide sequence located in the 3′-UTR of a target mRNA molecule which is complementary to a specific miRNA molecule (e.g. miR-126) such that they may hybridize and promote RNA-induced silencing complex-dependent and Dicer-dependent mRNA destabilization and/or cleavage, thereby preventing the expression of an mRNA transcript.

As used herein, the term “monocyte” refers to a type of white blood cell (i.e., a leukocyte) that is capable of differentiating into macrophages and myeloid lineage dendritic cells. Monocytes constitute an important component of the vertebrate adaptive immune response. Three different types of monocytes are known to exist, including classical monocytes characterized by strong expression of the CD14 cell surface receptor and no CD16 expression (i.e., CD14++ CD16−), non-classical monocytes exhibiting low levels of CD14 expression and co-expression of 016 (CD14+ CD16++), and intermediate monocytes exhibiting high levels of CD14 expression and low levels of CD6 expression (CD14++CD16+). Monocytes perform a variety of functions that serve the immune system, including phagocytosis, antigen presentation, and cytokine secretion.

As used herein, the term “multipotent cell” refers to a cell that possesses the ability to develop into multiple (e.g., 2, 3, 4, 5, or more) but not all differentiated cell types. Non-limiting examples of multipotent cells include cells of the hematopoietic lineage (e.g., granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Examples of multipotent cells are CD34+ cells.

As used herein, the term “movement disorder” refers to a set of clinical disorders or conditions characterized by abnormal voluntary or involuntary muscle movements that are unrelated to muscle weakness, fatigue, or spasticity. Movement disorders may be associated with excessive movement (e.g., a hyperkinetic movement disorder) or a lack of movement (e.g., a hypokinetic movement disorder). Non-limiting examples of symptoms associated with a hyperkinetic movement disorders include dyskinesia. Examples of symptoms associated with hypokinetic movement disorders include akinesia, hypokinesia, bradykinesia, and rigidity. Movement disorders are most frequently associated with disorders of basal ganglia and extrapyramidal motor control circuits of the central nervous system. Non-limiting examples of movement disorders include Parkinsonism (e.g., Parkinson disease, atypical parkinsonism, secondary parkinsonism, and functional parkinsonism), choreiform disorders, dystonic disorders, ataxic disorders, disorders associated with tremor, tic disorders, and myoclonic disorders.

As used herein, the term “mutation” refers to a change in the nucleotide sequence of a gene. Mutations in a gene may occur naturally as a result of, for example, errors in DNA replication, DNA repair, irradiation, and exposure to carcinogens or mutations may be induced as a result of administration of a transgene expressing a mutant gene. Mutations may result in a substitution of a single amino acid within the peptide chain. An exemplary nomenclature used herein for describing mutations resulting amino acid substitutions uses the format “p.AnB,” where “p” designates the variation at the level of the protein, “A” designates the amino acid found in the wild type variant of the protein, “n” designates the number of the amino acid within the peptide chain, and “B” designates the new amino acid that resulted from the substitution. For example, a p.R47H mutation corresponds to a change in a given protein at amino acid 47, where an arginine is substituted for histidine.

As used herein, the term “myeloablative” or “myeloablation” refers to a conditioning regiment that substantially impairs or destroys the hematopoietic system, typically by exposure to a cytotoxic agent (e.g., busulfan) or radiation. Myeloablation encompasses complete myeloablation brought on by high doses of cytotoxic agent or total body irradiation that destroys the hematopoietic system.

As used herein, the term “non-myeloablative” or “myelosuppressive” refers to a conditioning regiment that does not eliminate substantially all hematopoietic cells of host origin.

As used herein, the terms “neurocognitive disorder” or “NCD” refer to a set of clinical disorders or syndromes in which the primary clinical deficit is cognitive function, such as a deficit in, e.g., complex attention, executive function, learning and memory, language, perceptual-motor function, and social cognition. NCD is characterized as an acquired condition, rather than a developmental one. For example, an NCD is a condition in which disrupted cognition was not evident since birth or very early life, therefore requiring that cognitive function in NCD declined from a previously acquired level. NCD is distinguished from other disorders in which patients present with cognitive impairment in that NCD includes only disorders in which the core deficits are cognitive. NCD may be “major NCD” or “mild NCD.” Major NCD is characterized by significant cognitive decline that interferes with personal independence and normal daily functioning and is not due to delirium or other mental disorder. Mild NCD is characterized by moderate cognitive decline that does not interfere with personal independence and normal daily functioning and is not due to delirium or other mental disorder. Major and mild NCD may also be differentiated on the basis of quantitative cognitive testing across any one of the specific cognitive functions described above. For example, major NCD can be characterized by a score obtained on a cognitive test by a patient identified as having or at risk of developing NCD that is more than two standard deviations away from the mean score of a reference population (e.g., the mean score of a general population) or a score that is in the third percentile of the distribution of scores of the reference population. Mild NCD can be characterized by a score obtained on a cognitive test by a patient identified as having or at risk of developing NCD that is between one to two standard deviations away from the mean score of a reference population or a score that is between the 3rd and 16th percentile of the distribution of scores of the reference population. Non-limiting examples of cognitive tests that can be used to categorize an NCD patient as having either major or mild NCD include AWV, GPCOG, HRA, MIS, MMSE, MoCA, SLUMS, and Short IQCODE. Furthermore, NCD (e.g., major or mild NCD) includes syndrome subtypes that designate the particular etiological origin of the NCD, such as, e.g., Alzheimer's disease, Parkinson disease, or frontotemporal lobar degeneration (FTLD). As used herein, the terms “NCD due to Alzheimer's disease,” “NCD due to a movement disorder,” and “frontotemporal NCD” correspond to NCD caused by Alzheimer's disease, a movement disorder (e.g., Parkinson disease), and FTLD, respectively.

As used herein, the term “pluripotent cell” refers to a cell that possesses the ability to develop into more than one differentiated cell type, such as a cell type of the hematopoietic lineage (e.g., granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Examples of pluripotent cells are ESCs and iPSCs.

As used herein, the term “plasmid” refers to a to an extrachromosomal circular double stranded DNA molecule into which additional DNA segments may be ligated. A plasmid is a type of vector, a nucleic acid molecule capable of transporting another nucleic acid to which it has been linked. Certain plasmids are capable of autonomous replication in a host cell into which they are introduced (e.g., bacterial plasmids having a bacterial origin of replication and episomal mammalian plasmids). Other vectors (e.g., non-episomal mammalian vectors) can be integrated into the genome of a host cell upon introduction into the host cell, and thereby are replicated along with the host genome. Certain plasmids are capable of directing the expression of genes to which they are operably linked.

As used herein, the term “promoter” refers to a recognition site on DNA that is bound by an RNA polymerase. The polymerase drives transcription of the transgene. Exemplary promoters suitable for use with the compositions and methods described herein are described, for example, in Sandelin et al., Nature Reviews Genetics 8:424 (2007), the disclosure of which is incorporated herein by reference as it pertains to nucleic acid regulatory elements. Additionally, the term “promoter” may refer to a synthetic promoter, which are regulatory DNA sequences that do not occur naturally in biological systems. Synthetic promoters contain parts of naturally occurring promoters combined with polynucleotide sequences that do not occur in nature and can be optimized to express recombinant DNA using a variety of transgenes, vectors, and target cell types.

As used herein, a therapeutic agent is considered to be “provided” to a patient if the patient is directly administered the therapeutic agent or if the patient is administered a substance that is processed or metabolized in vivo so as to yield the therapeutic agent endogenously. For example, a patient, such as a patient having an NCD described herein, may be provided a protein of the disclosure (e.g., granulin) by direct administration of the protein or by administration of a substance (e.g., a progranulin gene or protein) that is processed or metabolized in vivo so as to yield the desired protein endogenously. Additional examples of “providing” a protein of interest to a patient are instances in which the patient is administered (i) a nucleic acid molecule encoding the protein of interest, (ii) a vector (e.g., a viral vector) containing such a nucleic acid molecule, (iii) a cell or population of cells containing such a vector or nucleic acid molecule, (iv) an interfering RNA molecule, such as a siRNA, shRNA, or miRNA molecule, that stimulates expression of the protein endogenously upon administration to the patient, or (v) a protein precursor that is processed, for example, by way of one or more post-translational modifications, to yield the desired protein endogenously.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

As used herein, the term “pharmaceutically acceptable” refers to those compounds, materials, compositions and/or dosage forms, which are suitable for contact with the tissues of a patient, such as a mammal (e.g., a human) without excessive toxicity, irritation, allergic response and other problem complications commensurate with a reasonable benefit/risk ratio.

As used herein, a “receptor-binding peptide (Rb) derived from ApoE” is a portion of the ApoE protein that has the ability to translocate proteins across the blood-brain barrier (BBB) into the brain when incorporated into a fusion protein. This methodology can therefore function to selectively open the BBB for therapeutic agents (e.g., proteins described herein) when engineered as fusion constructs. Such peptides can be readily attached to diagnostic or therapeutic agents without jeopardizing their biological functions or interfering with the important biological functions of ApoE due to the utilization of the Rb domain of ApoE, rather than the entire ApoE protein. Exemplary Rb domains that may be used in conjunction with the compositions and methods of the disclosure are those found in the N-terminus of ApoE. For example, Rb domains useful in conjunction with the compositions and methods described herein include polypeptides having the amino acid sequence of residues 1 to 191 of SEQ ID NO: 105, residues 25 to 185 of SEQ ID NO: 105, residues 50 to 180 of SEQ ID NO: 105, residues 75 to 175 of SEQ ID NO: 105, residues 100 to 170 of SEQ ID NO: 105, or residues 125 to 165 of SEQ ID NO: 105, as well as variants thereof, such as polypeptides having at least 70% sequence identity (e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or greater, sequence identity) to any of the foregoing sequences. Exemplary Rb domains useful in conjunction with the compositions and methods of the disclosure are the region of ApoE having the amino acid sequence of residues 159 to 167 of SEQ ID NO: 105, as well as domains having at least 70% sequence identity (e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or greater, sequence identity) to this sequence.

As used herein, the term “regulatory sequence” includes promoters, enhancers and other expression control elements (e.g., polyadenylation signals) that control the transcription or translation of the antibody chain genes. Such regulatory sequences are described, for example, in Perdew et al., Regulation of Gene Expression (Humana Press, New York, NY, (2014)); incorporated herein by reference.

As used herein, the term “sample” refers to a specimen (e.g., blood, blood component (e.g., serum or plasma), urine, saliva, amniotic fluid, cerebrospinal fluid, tissue (e.g., placental or dermal), pancreatic fluid, chorionic villus sample, and cells) isolated from a patient.

As used herein, the term “signal peptide” refers to a short (usually between 16-30 amino acids) peptide region that directs translocation of the translated protein from the cytoplasm of the host to the lipid membrane for anchoring. Such signal peptides are generally located at the amino terminus of the newly translated protein. In some embodiments, the signal peptide is linked to the amino terminus. Typically, signal peptides are cleaved during transit through the endoplasmic reticulum.

As used herein, the term “social cognition” refers to a cognitive function that encompasses a set of skills that govern how patients (e.g., humans) process, store, and apply information about other conspecific patients (e.g., other humans) and social situations. Non-limiting examples of social cognition include, e.g., emotional responses to social stimuli, performance on theory of mind tasks, ability to recognize faces, impulse control in social contexts, and joint attention. A patient diagnosed with an NCD may exhibit impaired social cognition relative to a healthy patient.

As used herein, the term “splice variant” refers to a transcribed product (i.e. RNA) of a single gene that can be processed to produce different mRNA molecules as a result of alternative inclusion or exclusion of specific exons (e.g. exon skipping) within the precursor mRNA. Proteins produced from translation of specific splice variants may differ in their structure and biological activity.

As used herein, the terms “stem cell” and “undifferentiated cell” refer to a cell in an undifferentiated or partially differentiated state that has the developmental potential to differentiate into multiple cell types. A stem cell is capable of proliferation and giving rise to more such stem cells while maintaining its functional potential. Stem cells can divide asymmetrically, which is known as obligatory asymmetrical differentiation, with one daughter cell retaining the functional potential of the parent stem cell and the other daughter cell expressing some distinct other specific function, phenotype and/or developmental potential from the parent cell. The daughter cells themselves can be induced to proliferate and produce progeny that subsequently differentiate into one or more mature cell types, while also retaining one or more cells with parental developmental potential. A differentiated cell may derive from a multipotent cell, which itself is derived from a multipotent cell, and so on. Alternatively, some of the stem cells in a population can divide symmetrically into two stem cells. Accordingly, the term “stem cell” refers to any subset of cells that have the developmental potential, under particular circumstances, to differentiate to a more specialized or differentiated phenotype, and which retain the capacity, under certain circumstances, to proliferate without substantially differentiating. In some embodiments, the term stem cell refers generally to a naturally occurring parent cell whose descendants (progeny cells) specialize, often in different directions, by differentiation, e.g., by acquiring completely individual characters, as occurs in progressive diversification of embryonic cells and tissues. Some differentiated cells also have the capacity to give rise to cells of greater developmental potential. Such capacity may be natural or may be induced artificially upon treatment with various factors. Cells that begin as stem cells might proceed toward a differentiated phenotype, but then can be induced to “reverse” and re-express the stem cell phenotype, a term often referred to as “dedifferentiation” or “reprogramming” or “retrodifferentiation” by persons of ordinary skill in the art.

As used herein, the term “transfection” refers to any of a wide variety of techniques commonly used for the introduction of exogenous DNA into a prokaryotic or eukaryotic host cell, e.g., electroporation, lipofection, calcium-phosphate precipitation, DEAE-dextran transfection, Nucleofection, squeeze-poration, sonoporation, optical transfection, Magnetofection, impalefection, and the like.

As used herein, the term “transgene” refers to a recombinant nucleic acid (e.g., DNA or cDNA) encoding a gene product (e.g., a gene product described herein). The gene product may be an RNA, peptide, or protein. In addition to the coding region for the gene product, the transgene may include or be operably linked to one or more elements to facilitate or enhance expression, such as a promoter, enhancer(s), destabilizing domain(s), response element(s), reporter element(s), insulator element(s), polyadenylation signal(s), and/or other functional elements. Embodiments of the disclosure may utilize any known suitable promoter, enhancer(s), destabilizing domain(s), response element(s), reporter element(s), insulator element(s), polyadenylation signal(s), and/or other functional elements.

As used herein, the terms “subject” and “patient” are used interchangeably and refer to an organism (e.g., a mammal, such as a human) that has been diagnosed as having, and/or is undergoing treatment for, a disease, such as an NCD described herein. For example, patients and subjects that may be treated using the compositions and methods of the disclosure include those that have been diagnosed as having an NCD, as well as individuals that are at risk of developing one or more of these conditions. Diagnosis may be performed by any method or technique known in the art. One skilled in the art will understand that a patient to be treated according to the present disclosure may have been subjected to standard tests or may have been identified, without examination, as one at risk due to the presence of one or more risk factors associated with the disease or condition.

As used herein in the context of a plurality of agents that together or collectively perform a particular activity, the terms “together” and “collectively” are used interchangeably and describe instances in which each agent, individually, may or may not achieve the indicated function, but when the agents are combined, the indicated function is achieved. As an example, a plurality of nucleic acid molecules that “together” or “collectively” encode a panel of proteins may include constituent nucleic acid molecules that, individually, encode a single protein within the panel, but when combined, encode the entirety of the proteins within the panel. Similarly, a plurality of agents that “together” or “collectively” increase the expression and/or activity of a panel of proteins may include constituent agents, such as host cells, viral vectors, nucleic acid molecules, or small molecules of the disclosure, that, individually, increase expression and/or activity of a single protein within the panel, but when combined, increase expression and/or activity of the entirety of proteins within the panel.

As used herein, the terms “transduction” and “transduce” refer to a method of introducing a viral vector construct or a part thereof into a cell and subsequent expression of a transgene encoded by the vector construct or part thereof in the cell.

As used herein, “treatment” and “treating” refer to an approach for obtaining beneficial or desired results, e.g., clinical results. Beneficial or desired results can include, but are not limited to, alleviation or amelioration of one or more symptoms or conditions; diminishment of extent of disease or condition; stabilized (i.e., not worsening) state of disease, disorder, or condition; preventing spread of disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to or at risk of developing the condition or disorder, as well as those in which the condition or disorder is to be prevented.

As used herein in the context of cells, such as genetically modified cells (e.g., cells that have been transfected or transduced so as to express a desired gene or protein), the term “uniform population” refers to a collection of cells, or progeny thereof, that have been modified ex vivo to contain nucleic acids encoding one or more proteins, such as a panel of proteins containing one or more, or all, of APP, PSEN1, PSEN2, APOE, TOMM40, GAB2, APOC1, TREM2, ABI3, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1 L, STK24, DISC1, MPZL1, SLC4A1AP, TRIP4, MSRA, HS3ST1, ZNF224, and AP2A2 (e.g., a panel of proteins selected from PSEN1, GAB2, APOC1, TREM2, ABI3, BIN1, HLA-DRB5, HLA-DRB1, CD2AP, PTK2B, INPP5D, MEF2C, CD33, MS4A4A, RIN3, PICALM, CASS4, SORL1, PLCG2, SCIMP, FRMD4A, SPPL2A, MTHFD1 L, DISCI , TRIP4, and HS3ST1), a panel of proteins containing one or more, or all of

FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1 B, NUCKS1, and ACMSD (e.g., a panel of proteins selected from FCGR2A, SCAF11, DNAJC13, GCH1, LRRK2, GBA, GAK, FGF20, HLA-DQB1, and NOD2), a panel of proteins containing one or more, or all of HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT (e.g., a panel of proteins selected from HLA-DRA, HLA-DRB5, C9ORF72, SQSTM1, TBK1, PSEN1, GRN, and CTSF), or a panel of proteins containing one or more or all of APP, PSEN1, PSEN2, APOE, TOMM40, GAB2, APOC1, TREM2, ABI3, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1L, STK24, DISCI , MPZL1, SLC4A1AP, TRIP4, MSRA, HS3ST1, ZNF224, AP2A2, FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1B, NUCKS1, ACMSD, HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT. A population is considered to be a “uniform population” if, for example, at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, 99.9%, 99.99%, or more (e.g., 100%) of the cells contain nucleic acids encoding the full panel of desired proteins. Cells may be transfected to contain nucleic acids encoding the desired proteins using genetic engineering techniques described herein, including by way of viral transduction (e.g., using a Retroviridae family virus, such as a lentivirus), as well as by cell transformation techniques, including electroporation and calcium phosphate-mediated nucleic acid transfer, among other strategies described herein. Methods of determining transgene expression are described herein and known in the art, and include, for example, RNAseq and RT-PCT assays used to quantify transgene expression at the RNA transcript level, as well as enzyme-linked immunosorbent assays (ELISA) used to quantify transgene expression at the protein level.

As used herein in the context of cells, such as genetically modified cells (e.g., cells that have been transfected or transduced so as to express a desired gene or protein), the term “heterogeneous population” refers to a collection of cells, or progeny thereof, that have been modified ex vivo to collectively contain nucleic acids encoding one or more of a panel of proteins, such as a panel of proteins described above. A population is considered to be a “heterogeneous population” if the population is substantially free of cells that individually contain nucleic acids encoding all of the proteins in a desired panel, but the cells combine to contain nucleic acids encoding all of the proteins in the desired panel. Methods of determining transgene expression are described herein and known in the art, and include, for example, RNAseq and RT-PCT assays used to quantify transgene expression at the RNA transcript level, as well as enzyme-linked immunosorbent assays (ELISA) used to quantify transgene expression at the protein level.

As used herein, the term “vector” includes a nucleic acid vector, e.g., a DNA vector, such as a plasmid, a RNA vector, virus, or other suitable replicon (e.g., viral vector). A variety of vectors have been developed for the delivery of polynucleotides encoding exogenous proteins into a prokaryotic or eukaryotic cell. Examples of such expression vectors are disclosed in, e.g., WO 1994/011026; incorporated herein by reference as it pertains to vectors suitable for the expression of a gene of interest. Expression vectors suitable for use with the compositions and methods described herein contain a polynucleotide sequence as well as, e.g., additional sequence elements used for the expression of proteins and/or the integration of these polynucleotide sequences into the genome of a mammalian cell. Vectors that can be used for the expression of a protein or proteins described herein include plasmids that contain regulatory sequences, such as promoter and enhancer regions, which direct gene transcription. Additionally, useful vectors for expression of a protein or proteins described herein may contain polynucleotide sequences that enhance the rate of translation of the corresponding gene or genes or improve the stability or nuclear export of the mRNA that results from gene transcription. Examples of such sequence elements are 5′ and 3′ untranslated regions, an IRES, and a polyadenylation signal site in order to direct efficient transcription of a gene or genes carried on an expression vector. Expression vectors suitable for use with the compositions and methods described herein may also contain a polynucleotide encoding a marker for selection of cells that contain such a vector. Examples of a suitable marker are genes that encode resistance to antibiotics, such as ampicillin, chloramphenicol, kanamycin, nourseothricin, or zeocin, among others.

As used herein, the terms “triggering receptor expressed on myeloid cells two” and “TREM2” refer to the transmembrane glycoprotein belonging to the immunoglobulin variable domain receptor family. The gene is located on human chromosome 6p21.1. The terms “triggering receptor expressed on myeloid cells two” and “TREM2” also refer to variants of wild type TREM2 peptides and nucleic acids encoding the same, including splice variants resulting from alternative splicing of TREM2 primary transcripts, such as variant proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to the amino acid sequence of a wild type TREM2 peptide (e.g., SEQ ID NO: 103) or polynucleotides having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to the nucleic acid sequence of a wild type TREM2 gene (European Nucleotide Archive Reference No. (ENA) AF213457.1), provided that the TREM2 isoform encoded retains the therapeutic function of wild type TREM2. The terms “triggering receptor expressed on myeloid cells two” and “TREM2” may also refer to a TREM2 protein in which the natural signal peptide is present. Furthermore, the terms “triggering receptor expressed on myeloid cells two” and “TREM2” may refer to all products of TREM2 proteolytic cleavage including soluble TREM2 (sTREM2), the TREM2 C-terminal fragment (CTF), the TREM2 intracellular domain (TREM2-ICD), and TREM2-A 3-like peptides (T2β). TREM2 cleavage occurs once the mature polypeptide has been translocated to the membrane following posttranslational processing within the endoplasmic reticulum and is mediated by members of the disintegrin and metalloprotease (ADAM) family. The full-length TREM2 peptide is first cleaved at the ectodomain to produce an extracellular sTREM2 peptide and the transmembrane TREM2-CTF, the latter of which may be further cleaved by the y-secretase complex to produce the cytoplasmic TREM2-ICD and the extracellular TREM-T2β peptides. The terms “triggering receptor expressed on myeloid cells two” and “TREM2” may refer to a TREM2 protein lacking a functional ectodomain cleavage site. The terms “triggering receptor expressed on myeloid cells two” and “TREM2” may also refer to a TREM2 protein lacking a functional intramembrane cleavage site within the TREM2-CTF. Additionally, the terms “triggering receptor expressed on myeloid cells two” and “TREM2” may refer to a “TREM2 fusion protein,” which is a protein in which the TREM2 is operably linked to another polypeptide, half-life-modifying agent, or therapeutic agent, such as an ApoE Rb domain (such as an Rb domain having the amino acid sequence of residues 25-185, 50-180, 75-175, 100-170, 125-160, or 130-150 of SEQ ID NO: 105). As used herein, “TREM2” may refer to the peptide or the gene encoding this protein, depending upon the context, as will be appreciated by one of skill in the art.

As used herein, the term “functional ectodomain cleavage site” as it pertains to the TREM2 ectodomain cleavage site refers to amino acid residues within the full-length TREM2 peptide that undergo proteolytic cleavage by extracellular proteases (e.g., disintegrin and metalloprotease family) ectodomain to produce soluble TREM2 as well as the TREM2 C-terminal fragment. The TREM2 ectodomain cleavage site may be rendered non-functional as a result of, for example, a mutation in the TREM2 gene that alters the amino acid sequence within the ectodomain cleavage site or affects the tertiary protein structure in such a way as to sterically protect the ectodomain cleavage site from proteolytic cleavage.

As used herein, the term “functional intramembrane cleavage site” as it pertains to the TREM2 C-terminal fragment intramembrane cleavage site refers to amino acid residues within the TREM2 C-terminal fragment that undergo proteolytic cleavage by the y-secretase complex to produce the TREM2 intracellular domain and TREM2-A β-like peptide. The TREM2 C-terminal fragment intramembrane cleavage site may be rendered non-functional as a result of, for example, a mutation in the TREM2 gene that alters the amino acid sequence within the intramembrane cleavage site or affects the tertiary protein structure in such a way as to sterically protect the intramembrane cleavage site from proteolytic cleavage.

As used herein, patients suffering from “triggering receptor expressed on myeloid cells two-associated Alzheimer's disease” and “TREM2-associated Alzheimer's disease” are those patients that have been diagnosed as having Alzheimer's disease and that also contain a deleterious mutation in the endogenous TREM2 gene. Over 40 mutations have been reported in the human TREM2 gene, which have variable effects on downstream signaling, trafficking, ligand binding, and cell surface expression. TREM2 mutations are discussed in in Guerreiro et al., The New England Journal of Medicine 368:117-27, (2013), Jonsson et al., The New England Journal of Medicine, 368:107-16 (2013), and Ulrich et al., Neuron Review 94:237-48, (2017), the disclosures of which are incorporated herein by reference as they pertain to human TREM2 mutations in Alzheimer's disease.

As used herein, the terms “glucocerebrosidase” and “GBA” refer to the lysosomal enzyme responsible for the metabolism of glucocerebroside (also known as glucosylceramide) to glucose and ceramide. The gene is located on chromosome 1q21 and is also known as GBA1. The terms “glucocerebrosidase” and “GBA” also refer to variants of wild-type glucocerebrosidase enzymes and nucleic acids encoding the same, such as variant proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to the amino acid sequence of a wild-type GBA enzyme (e.g., SEQ ID NO: 104) or polynucleotides having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to the nucleic acid sequence of a wild-type GBA gene (e.g., ENA M16328.1), provided that the GBA analog encoded retains the therapeutic function of wild-type GBA. “GBA” may also refer to a GBA protein in which the natural signal peptide is present. Alternatively, “GBA” may refer to a GBA protein in which the natural signal peptide has been removed (e.g., the mature protein). GBA may also refer to the catalytic domain of GBA, or a variant having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to such a domain. Additionally, the terms “glucocerebrosidase” and “GBA” may refer to a “GBA fusion protein,” which is a protein in which the GBA is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, GBA may refer to the lysosomal enzyme or the gene encoding this protein, depending upon the context, as will be appreciated by one of skill in the art.

As used herein, patients suffering from “GBA-associated Parkinson's disease” or “GBA-associated PD” are those patients that have been diagnosed as having Parkinson's disease and also contain a deleterious mutation in the GBA gene. Severely pathogenic mutations include c.84GGlns, IVS2+1 G>A, p.V394L, p.D409H, p.L444P and RecTL, which are linked to a 9.92 to 21.29 odds-ratio of developing PD. Mild GBA mutations p.N370S and p.R496H are linked to an odds-ratio of 2.84-4.94 of developing PD. The mutation p.E326K has also been identified as a PD risk factor. GBA mutations are discussed in in Barkhuizen et al., Neurochemistry International 93:6 (2016) and Sidransky and Lopez, Lancet Neurol. 11:986 (2012), the disclosures of which are incorporated herein by reference as they pertain to human GBA mutations.

As used herein, the terms “granulin” and “GRN” refer to the peptide products resulting from cleavage of the precursor protein PGRN. GRN peptides are involved in a variety of biological functions including development, immunity, cell survival and proliferation, and tumorigenesis. Full-length wild-type human PGRN peptide has 7.5 GRN domains (e.g., 7 GRN domains, each approximately 60 amino acids in length), and a 30 amino acid paragranulin (para-GRN) domain, that can be individually cleaved by proteases. The terms “granulin” and “GRN” also refer to variants of wild-type human granulin peptides and nucleic acids encoding the same, such as variant proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type GRN peptide (e.g., SEQ ID NO: 106), provided that the GRN variant encoded retains the therapeutic function of the wild-type GRN. The terms “granulin” and “GRN” may also refer to a GRN protein in which the natural secretory signal peptide is present. Additionally, the terms “granulin” and “GRN” may refer to a “GRN fusion protein,” which is a protein in which the GRN is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “GRN” may refer to the peptide or the gene encoding this protein, depending upon the context, as will be appreciated by one of skill in the art.

As used herein, the terms “progranulin” and “PGRN” refer to the secreted trophic factor and precursor peptide for granulin. The gene is located on chromosome 17q21.31 and is also known as granulin precursor, proepithelin, PEPI, PC cell-derived growth factor, granulin-epithelin, CLN11, PCDFGF, GP88, GEP, granulins, acrogranin. The terms “progranulin” and “PGRN” also refer to variants of wild-type human PGRN peptides and nucleic acids encoding the same, such as variant proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to the amino acid sequence of a wild-type PGRN peptide or polynucleotides having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to the nucleic acid sequence of a wild-type PGRN gene, provided that the PGRN variant encoded retains the therapeutic function of the wild-type PGRN. The terms “progranulin” and “PGRN” may also refer to variants of PGRN having 2 or more (e.g., 2, 3, 4, 5, 6, 7, 8, or more) granulin (GRN) domains. The terms “progranulin” and “PGRN” may also refer to variants of PGRN having from 2 to 16 (e.g., 2, 3, 4, 5, 6, 7, 8, 9, 10, 11, 12, 13, 14, 15, 16) GRN domains. The terms “progranulin” and “PGRN” may also refer to a PGRN protein in which the natural secretory signal peptide is present. Additionally, the terms “progranulin” and “PGRN” may refer to a “PG RN fusion protein,” which is a protein in which the PGRN is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “PGRN” may refer to the peptide or the gene encoding this protein, depending upon the context, as will be appreciated by one of skill in the art.

As used herein, the terms “frontotemporal lobar degeneration” and “FTLD” refer to a complex clinical syndrome characterized by degeneration of brain tissue within the frontal and temporal lobes of the cerebral cortex. The terms “frontotemporal lobar degeneration” and “FTLD” may refer to any one of three clinically distinct variants of FTLD including: 1) behavioral-variant frontotemporal dementia (BVFTD), characterized by changes in behavior and personality, apathy, social withdrawal, perseverative behaviors, attentional deficits, disinhibition, and a pronounced degeneration of the frontal lobe. Additionally, BVFTD has a strong association with amyotrophic lateral sclerosis; 2) semantic dementia (SD) is characterized by fluent, anomic aphasia, progressive loss of semantic knowledge of words, objects, and concepts and a pronounced degeneration of the anterior temporal lobes. Furthermore, SD variant of FTLD exhibit a flat affect, social deficits, perseverative behaviors, and disinhibition; 3) progressive nonfluent aphasia (PNA) is characterized by motor deficits in speech production, reduced language expression, and pronounced degeneration of the perisylvian cortex. Histopathological profiles of FTLD patients generally fall into one of three broad phenotypes including those that exhibit aggregation and deposition of (i) microtubule-associated tau protein inclusions; (ii) tau-negative, ubiquitin and TAR DNA-binding protein 43 (TDP-43)-positive protein inclusions, or (iii) ubiquitin and fused in sarcoma (FUS)-positive protein inclusions. A comprehensive description of the clinical presentation and histopathology of FTLD is set forth in Rabinovici and Miller, CNS Drugs 24:375-398 (2010), the disclosure of which is incorporated herein by reference in its entirety.

As used herein, patients suffering from “progranulin-associated FTLD” and “PGRN-associated FTLD” are those patients that have been diagnosed as having FTLD and also contain a deleterious mutation in the PGRN gene. Over 70 pathogenic mutations have been reported in the PGRN gene, the majority of which result in a premature stop codon and nonsense-mediated decay of truncated PGRN mRNA. PGRN mutations are described in Gijselinck et al., Human Mutation 29:1373-86 (2012) and Pottier et al., Journal of Neurochemistry 138:32-53 (2016), the disclosures of each of which are incorporated herein by reference as they pertain to human PGRN mutations.

As used herein, the term “APP” refers to the gene encoding Amyloid-beta A4 protein, or the corresponding protein product. The terms “APP” and “Amyloid-beta A4 protein” include wild-type forms of the APP gene or protein, as well as variants (e.g., splice variants, truncations, concatemers, and fusion constructs, among others) of wild-type APP proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type APP protein (e.g., SEQ ID NO: 1), provided that the APP variant retains the therapeutic function of a wild-type APP. Additionally, the terms “APP” and “Amyloid-beta A4 protein” may refer to an “APP fusion protein,” which is a protein in which the APP is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “APP” may refer to the protein or the gene encoding this protein, depending upon the context, as will be appreciated by one of skill in the art.

As used herein, the term “PSEN1” refers to the gene encoding presenilin-1, or the corresponding protein product. The terms “PSEN1” and “presenilin-1” include wild-type forms of the PSEN1 gene or protein, as well as variants (e.g., splice variants, truncations, concatemers, and fusion constructs, among others) of wild-type PSEN1 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type PSEN1 protein (e.g., SEQ ID NO: 2), provided that the PSEN1 variant retains the therapeutic function of a wild-type PSEN1. Additionally, the terms “PSEN1” and “presenilin-1” may refer to a “PSEN1 fusion protein,” which is a protein in which the PSEN1 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “PSEN1” may refer to the protein or the gene encoding this protein, depending upon the context, as will be appreciated by one of skill in the art.

As used herein, the term “PSEN2” refers to the gene encoding presenilin-2, or the corresponding protein product. The terms “PSEN2” and “presenilin-2” include wild-type forms of the PSEN2 gene or protein, as well as variants (e.g., splice variants, truncations, concatemers, and fusion constructs, among others) of wild-type PSEN2 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type PSEN2 protein (e.g., SEQ ID NO: 3), provided that the PSEN2 variant retains the therapeutic function of a wild-type PSEN2. Additionally, the terms “PSEN2” and “presenilin-2” may refer to a “PSEN2 fusion protein,” which is a protein in which the PSE21 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “PSEN2” may refer to the protein or the gene encoding this protein, depending upon the context, as will be appreciated by one of skill in the art.

As used herein, the term “TOMM40” refers to the gene encoding mitochondrial import receptor subunit TOM40 homolog, or the corresponding protein product. The terms “TOMM40” and “mitochondrial import receptor subunit TOM40 homolog” include wild-type forms of the TOMM40 gene or protein, as well as variants (e.g., splice variants, truncations, concatemers, and fusion constructs, among others) of wild-type TOMM40 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type TOMM40 protein (e.g., SEQ ID NO: 4), provided that the TOMM40 variant retains the therapeutic function of a wild-type TOMM40. Additionally, the terms “TOMM40” and “mitochondrial import receptor subunit TOM40 homolog” may refer to a “TOMM40 fusion protein,” which is a protein in which the TOMM40 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “TOMM40” may refer to the protein or the gene encoding this protein, depending upon the context, as will be appreciated by one of skill in the art.

As used herein, the term “GAB2” refers to the gene encoding GRB2-associated-binding protein 2, or the corresponding protein product. The terms “GAB2” and “GRB2-associated-binding protein 2” include wild-type forms of the GAB2 gene or protein, as well as variants (e.g., splice variants, truncations, concatemers, and fusion constructs, among others) of wild-type GAB2 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type GAB2 protein (e.g., SEQ ID NO: 5), provided that the GAB2 variant retains the therapeutic function of a wild-type GAB2. Additionally, the terms “GAB2” and “GRB2-associated-binding protein 2” may refer to a “GAB2 fusion protein,” which is a protein in which the GAB2 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “GAB2” may refer to the protein or the gene encoding this protein, depending upon the context, as will be appreciated by one of skill in the art.

As used herein, the term “APOC1” refers to the gene encoding apolipoprotein C-1, or the corresponding protein product. The terms “APOC1” and “apolipoprotein C-1” include wild-type forms of the APOC1 gene or protein, as well as variants (e.g., splice variants, truncations, concatemers, and fusion constructs, among others) of wild-type APOC1 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type APOC1 protein (e.g., SEQ ID NO: 6), provided that the APOC1 variant retains the therapeutic function of a wild-type APOC1. Additionally, the terms “APOC1” and “apolipoprotein C-1” may refer to an “APOC1 fusion protein,” which is a protein in which the APOC1 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “APOC1” may refer to the protein or the gene encoding this protein, depending upon the context, as will be appreciated by one of skill in the art.

As used herein, the term “ABI3” refers to the gene encoding ABI gene family member 3, or the corresponding protein product. The terms “ABI3” and “ABI gene family member 3” include wild-type forms of the ABI3 gene or protein, as well as variants (e.g., splice variants, truncations, concatemers, and fusion constructs, among others) of wild-type ABI3 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type ABI3 protein (e.g., SEQ ID NO: 7), provided that the ABI3 variant retains the therapeutic function of a wild-type ABI3. Additionally, the terms “ABI3” and “ABI gene family member 3” may refer to an “ABI3 fusion protein,” which is a protein in which the ABI3 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “ABI3” may refer to the protein or the gene encoding this protein, depending upon the context, as will be appreciated by one of skill in the art.

As used herein, the term “BIN1” refers to the gene encoding myc box-dependent-interacting protein 1, or the corresponding protein product. The terms “BIN1” and “myc box-dependent-interacting protein 1” include wild-type forms of the BIN1 gene or protein, as well as variants (e.g., splice variants, truncations, concatemers, and fusion constructs, among others) of wild-type BIN1 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type BIN1 protein (e.g., SEQ ID NO: 8), provided that the BIN1 variant retains the therapeutic function of a wild-type BIN1. Additionally, the terms “BIN1” and “myc box-dependent-interacting protein 1” may refer to a “BIN1 fusion protein,” which is a protein in which the BIN1 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “BIN1” may refer to the protein or the gene encoding this protein, depending upon the context, as will be appreciated by one of skill in the art.

As used herein, the term “CR1” refers to the gene encoding complement receptor type 1, or the corresponding protein product. The terms “CR1” and “complement receptor type 1” include wild-type forms of the CR1 gene or protein, as well as variants (e.g., splice variants, truncations, concatemers, and fusion constructs, among others) of wild-type CR1 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type CR1 protein (e.g., SEQ ID NO: 9), provided that the CR1 variant retains the therapeutic function of a wild-type CR1. Additionally, the terms “CR1” and “complement receptor type 1” may refer to a “CR1 fusion protein,” which is a protein in which the CR1 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “CR1” may refer to the protein or the gene encoding this protein, depending upon the context, as will be appreciated by one of skill in the art.

As used herein, the term “ABCA7” refers to the gene encoding ATP-binding cassette sub-family A member 7, or the corresponding protein product. The terms “ABCA7” and “ATP-binding cassette sub-family A member 7” include wild-type forms of the ABCA7 gene or protein, as well as variants (e.g., splice variants, truncations, concatemers, and fusion constructs, among others) of wild-type CR1 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type ABCA7 protein (e.g., SEQ ID NO: 10), provided that the ABCA7 variant retains the therapeutic function of a wild-type ABCA7. Additionally, the terms “ABCA7” and “ATP-binding cassette sub-family A member 7” may refer to an “ABCA7 fusion protein,” which is a protein in which the ABCA7 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “ABCA7” may refer to the protein or the gene encoding this protein, depending upon the context, as will be appreciated by one of skill in the art.

As used herein, the term “FERMT2” refers to the gene encoding fermitin family homolog 2, or the corresponding protein product. The terms “FERMT2” and “Fermitin family homolog 2” include wild-type forms of the FERMT2 gene or protein, as well as variants (e.g., splice variants, truncations, concatemers, and fusion constructs, among others) of wild-type FERMT2 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type FERMT2 protein (e.g., SEQ ID NO: 11), provided that the FERMT2 variant retains the therapeutic function of a wild-type FERMT2. Additionally, the terms “FERMT2” and “fermitin family homolog 2” may refer to a “FERMT2 fusion protein,” which is a protein in which the FERMT2 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “FERMT2” may refer to the protein or the gene encoding this protein, depending upon the context, as will be appreciated by one of skill in the art.

As used herein, the term “HLA-DRB5” refers to the gene encoding HLA class II histocompatibility antigen, DR beta 5 chain, or the corresponding protein product. The terms “HLA-DRB5” and “HLA class II histocompatibility antigen, DR beta 5 chain” include wild-type forms of the HLA-DRB5 gene or protein, as well as variants (e.g., splice variants, truncations, concatemers, and fusion constructs, among others) of wild-type HLA-DRB5 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type HLA-DRB5 protein (e.g., SEQ ID NO: 12), provided that the HLA-DRB5 variant retains the therapeutic function of a wild-type HLA-DRB5. Additionally, the terms “HLA-DRB5” and “HLA class II histocompatibility antigen, DR beta 5 chain” may refer to a “HLA-DRB5 fusion protein,” which is a protein in which the HLA-DRB5 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “HLA-DRB5” may refer to the protein or the gene encoding this protein, depending upon the context, as will be appreciated by one of skill in the art.

As used herein, the term “HLA-DRB1” refers to the gene encoding HLA class II histocompatibility antigen, DR beta 1 chain, or the corresponding protein product. The terms “HLA-DRB1” and “HLA class II histocompatibility antigen, DR beta 1 chain” include wild-type forms of the HLA-DRB1 gene or protein, as well as variants (e.g., splice variants, truncations, concatemers, and fusion constructs, among others) of wild-type HLA-DRB1 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type HLA-DRB1 protein (e.g., SEQ ID NO: 13), provided that the HLA-DRB1 variant retains the therapeutic function of a wild-type HLA-DRB1. Additionally, the terms “HLA-DRB1” and “HLA class II histocompatibility antigen, DR beta 1 chain” may refer to a “HLA-DRB1 fusion protein,” which is a protein in which the HLA-DRB1 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “HLA-DRB1” may refer to the protein or the gene encoding this protein, depending upon the context, as will be appreciated by one of skill in the art.

As used herein, the term “CD2AP” refers to the gene encoding CD2-associated protein, or the corresponding protein product. The terms “CD2AP” and “CD2-associated protein” include wild-type forms of the CD2AP gene or protein, as well as variants (e.g., splice variants, truncations, concatemers, and fusion constructs, among others) of wild-type CD2AP proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type CD2AP protein (e.g., SEQ ID NO: 14), provided that the CD2AP variant retains the therapeutic function of a wild-type CD2AP. Additionally, the terms “CD2AP” and “CD2-associated protein” may refer to a “CD2AP fusion protein,” which is a protein in which the CD2AP is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “CD2AP” may refer to the protein or the gene encoding this protein, depending upon the context, as will be appreciated by one of skill in the art.

As used herein, the term “PTK2B” refers to the gene encoding protein-tyrosine kinase 2-beta, or the corresponding protein product. The terms “PTK2B” and “protein-tyrosine kinase 2-beta” include wild-type forms of the PTK2B gene or protein, as well as variants (e.g., splice variants, truncations, concatemers, and fusion constructs, among others) of wild-type PTK2B proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type PTK2B protein (e.g., SEQ ID NO: 15), provided that the PTK2B variant retains the therapeutic function of a wild-type PTK2B. Additionally, the terms “PTK2B” and “protein-tyrosine kinase 2-beta” may refer to a “PTK2B fusion protein,” which is a protein in which the PTK2B is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “PTK2B” may refer to the protein or the gene encoding this protein, depending upon the context, as will be appreciated by one of skill in the art.

As used herein, the term “CELF1” refers to the gene encoding CUGBP Elav-like family member 1, or the corresponding protein product. The terms “CELF1” and “CUGBP Elav-like family member 1” include wild-type forms of the CELF1 gene or protein, as well as variants (e.g., splice variants, truncations, concatemers, and fusion constructs, among others) of wild-type CELF1 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type CELF1 protein (e.g., SEQ ID NO: 16), provided that the CELF1 variant retains the therapeutic function of a wild-type CELF1. Additionally, the terms “CELF1” and “CUGBP Elav-like family member 1” may refer to a “CELF1 fusion protein,” which is a protein in which the CELF1 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “CELF1” may refer to the protein or the gene encoding this protein, depending upon the context, as will be appreciated by one of skill in the art.

As used herein, the term “INPP5D” refers to the gene encoding phosphatidylinositol 3,4,5-trisphosphate 5-phosphatase 1, or the corresponding protein product. The terms “INPP5D” and “phosphatidylinositol 3,4,5-trisphosphate 5-phosphatase 1” include wild-type forms of the INPP5D gene or protein, as well as variants (e.g., splice variants, truncations, concatemers, and fusion constructs, among others) of wild-type INPP5D proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type INPP5D protein (e.g., SEQ ID NO: 17), provided that the INPP5D variant retains the therapeutic function of a wild-type INPP5D. Additionally, the terms “INPP5D” and “phosphatidylinositol 3,4,5-trisphosphate 5-phosphatase 1” may refer to a “INPP5D fusion protein,” which is a protein in which the INPP5D is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “INPP5D” may refer to the protein or the gene encoding this protein, depending upon the context, as will be appreciated by one of skill in the art.

As used herein, the term “MEF2C” refers to the gene encoding myocyte-specific enhancer factor 2C, or the corresponding protein product. The terms “MEF2C” and “myocyte-specific enhancer factor 2C” include wild-type forms of the MEF2C gene or protein, as well as variants (e.g., splice variants, truncations, concatemers, and fusion constructs, among others) of wild-type MEF2C proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type MEF2C protein (e.g., SEQ ID NO: 18), provided that the MEF2C variant retains the therapeutic function of a wild-type MEF2C. Additionally, the terms “MEF2C” and “myocyte-specific enhancer factor 2C” may refer to a “MEF2C fusion protein,” which is a protein in which the MEF2C is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “MEF2C” may refer to the protein or the gene encoding this protein, depending upon the context, as will be appreciated by one of skill in the art.

As used herein, the term “ZCWPW1” refers to the gene encoding Zinc finger CW-type PWWP domain protein 1, or the corresponding protein product. The terms “ZCWPW1” and “Zinc finger CW-type PWWP domain protein 1” include wild-type forms of the ZCWPW1 gene or protein, as well as variants (e.g., splice variants, truncations, concatemers, and fusion constructs, among others) of wild-type ZCWPW1 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type ZCWPW1 protein (e.g., SEQ ID NO: 19), provided that the ZCWPW1 variant retains the therapeutic function of a wild-type ZCWPW1. Additionally, the terms “ZCWPW1” and “Zinc finger CW-type PWWP domain protein 1” may refer to a “ZCWPW1 fusion protein,” which is a protein in which the ZCWPW1 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “ZCWPW1” may refer to the protein or the gene encoding this protein, depending upon the context, as will be appreciated by one of skill in the art.

As used herein, the term “CD33” refers to the gene encoding Myeloid cell surface antigen CD33, or the corresponding protein product. The terms “CD33” and “Myeloid cell surface antigen CD33” include wild-type forms of the CD33 gene or protein, as well as variants (e.g., splice variants, truncations, concatemers, and fusion constructs, among others) of wild-type CD33 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type CD33 protein (e.g., SEQ ID NO: 20), provided that the CD33 variant retains the therapeutic function of a wild-type CD33. Additionally, the terms “CD33” and “Myeloid cell surface antigen CD33” may refer to a “CD33 fusion protein,” which is a protein in which the CD33 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “CD33” may refer to the protein or the gene encoding this protein, depending upon the context, as will be appreciated by one of skill in the art.

As used herein, the term “MS4A4A” refers to the gene encoding Membrane-spanning 4-domains subfamily A member 4A, or the corresponding protein product. The terms “MS4A4A” and “Membrane-spanning 4-domains subfamily A member 4A” include wild-type forms of the MS4A4A gene or protein, as well as variants (e.g., splice variants, truncations, concatemers, and fusion constructs, among others) of wild-type MS4A4A proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type MS4A4A protein (e.g., SEQ ID NO: 21), provided that the MS4A4A variant retains the therapeutic function of a wild-type MS4A4A. Additionally, the terms “MS4A4A” and “Membrane-spanning 4-domains subfamily A member 4A” may refer to a “MS4A4A fusion protein,” which is a protein in which the MS4A4A is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “MS4A4A” may refer to the protein or the gene encoding this protein, depending upon the context, as will be appreciated by one of skill in the art.

As used herein, the term “RIN3” refers to the gene encoding Ras and Rab interactor 3, or the corresponding protein product. The terms “RIN3” and “Ras and Rab interactor 3” include wild-type forms of the RIN3 gene or protein, as well as variants (e.g., splice variants, truncations, concatemers, and fusion constructs, among others) of wild-type RIN3 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type RIN3 protein (e.g., SEQ ID NO: 22), provided that the RIN3 variant retains the therapeutic function of a wild-type RIN3. Additionally, the terms “RIN3” and “Ras and Rab interactor 3” may refer to a “RIN3 fusion protein,” which is a protein in which the RIN3 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “RIN3” may refer to the protein or the gene encoding this protein, depending upon the context, as will be appreciated by one of skill in the art.

As used herein, the term “EPHA1” refers to the gene encoding Ephrin type-A receptor 1, or the corresponding protein product. The terms “EPHA1” and “Ephrin type-A receptor 1” include wild-type forms of the EPHA1 gene or protein, as well as variants (e.g., splice variants, truncations, concatemers, and fusion constructs, among others) of wild-type EPHA1 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type EPHA1 protein (e.g., SEQ ID NO: 23), provided that the EPHA1 variant retains the therapeutic function of a wild-type EPHA1. Additionally, the terms “EPHA1” and “Ephrin type-A receptor 1” may refer to a “EPHA1 fusion protein,” which is a protein in which the EPHA1 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “EPHA1” may refer to the protein or the gene encoding this protein, depending upon the context, as will be appreciated by one of skill in the art.

As used herein, the term “PICALM” refers to the gene encoding Phosphatidylinositol-binding clathrin assembly protein, or the corresponding protein product. The terms “PICALM” and “Phosphatidylinositol-binding clathrin assembly protein” include wild-type forms of the PICALM gene or protein, as well as variants (e.g., splice variants, truncations, concatemers, and fusion constructs, among others) of wild-type PICALM proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type PICALM protein (e.g., SEQ ID NO: 24), provided that the PICALM variant retains the therapeutic function of a wild-type PICALM. Additionally, the terms “PICALM” and “Phosphatidylinositol-binding clathrin assembly protein” may refer to a “PICALM fusion protein,” which is a protein in which the PICALM is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “PICALM” may refer to the protein or the gene encoding this protein, depending upon the context, as will be appreciated by one of skill in the art.

As used herein, the term “CASS4” refers to the gene encoding Cas scaffolding protein family member 4, or the corresponding protein product. The terms “CASS4” and “Cas scaffolding protein family member 4” include wild-type forms of the CASS4 gene or protein, as well as variants (e.g., splice variants, truncations, concatemers, and fusion constructs, among others) of wild-type CASS4 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type CASS4 protein (e.g., SEQ ID NO: 25), provided that the CASS4 variant retains the therapeutic function of a wild-type CASS4. Additionally, the terms “CASS4” and “Cas scaffolding protein family member 4” may refer to a “CASS4 fusion protein,” which is a protein in which the CASS4 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “CASS4” may refer to the protein or the gene encoding this protein, depending upon the context, as will be appreciated by one of skill in the art.

As used herein, the term “CLU” refers to the gene encoding Clusterin, or the corresponding protein product. The terms “CLU” and “Clusterin” include wild-type forms of the CLU gene or protein, as well as variants (e.g., splice variants, truncations, concatemers, and fusion constructs, among others) of wild-type CLU proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type CLU protein (e.g., SEQ ID NO: 26), provided that the CLU variant retains the therapeutic function of a wild-type CLU. Additionally, the terms “CLU” and “Clusterin” may refer to a “CLU fusion protein,” which is a protein in which the CLU is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “CLU” may refer to the protein or the gene encoding this protein, depending upon the context, as will be appreciated by one of skill in the art.

As used herein, the term “SORL1” refers to the gene encoding Sortilin-related receptor, or the corresponding protein product. The terms “SORL1” and “Sortilin-related receptor” include wild-type forms of the SORL1 gene or protein, as well as variants (e.g., splice variants, truncations, concatemers, and fusion constructs, among others) of wild-type SORL1 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type SORL1 protein (e.g., SEQ ID NO: 27), provided that the SORL1 variant retains the therapeutic function of a wild-type SORL1. Additionally, the terms “SORL1” and “Sortilin-related receptor” may refer to a “SORL1 fusion protein,” which is a protein in which the SORL1 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “SORL1” may refer to the protein or the gene encoding this protein, depending upon the context, as will be appreciated by one of skill in the art.

As used herein, the term “PLCG2” refers to the gene encoding 1-phosphatidylinositol 4,5-bisphosphate phosphodiesterase gamma-2, or the corresponding protein product. The terms “PLCG2” and “1-phosphatidylinositol 4,5-bisphosphate phosphodiesterase gamma-2” include wild-type forms of the PLCG2 gene or protein, as well as variants (e.g., splice variants, truncations, concatemers, and fusion constructs, among others) of wild-type PLCG2 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type PLCG2 protein (e.g., SEQ ID NO: 28), provided that the PLCG2 variant retains the therapeutic function of a wild-type PLCG2. Additionally, the terms “PLCG2” and “1-phosphatidylinositol 4,5-bisphosphate phosphodiesterase gamma-2” may refer to a “PLCG2 fusion protein,” which is a protein in which the PLCG2 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “PLCG2” may refer to the protein or the gene encoding this protein, depending upon the context, as will be appreciated by one of skill in the art.

As used herein, the term “SCIMP” refers to the gene encoding SLP adapter and CSK-interacting membrane protein, or the corresponding protein product. The terms “SCIMP” and “SLP adapter and CSK-interacting membrane protein” include wild-type forms of the SCIMP gene or protein, as well as variants (e.g., splice variants, truncations, concatemers, and fusion constructs, among others) of wild-type SCIMP proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type SCIMP protein (e.g., SEQ ID NO: 29), provided that the SCIMP variant retains the therapeutic function of a wild-type SCIMP. Additionally, the terms “SCIMP” and “SLP adapter and CSK-interacting membrane protein” may refer to a “SCIMP fusion protein,” which is a protein in which the SCIMP is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “SCIMP” may refer to the protein or the gene encoding this protein, depending upon the context, as will be appreciated by one of skill in the art.

As used herein, the term “FRMD4A” refers to the gene encoding FERM domain-containing protein 4A, or the corresponding protein product. The terms “FRMD4A” and “FERM domain-containing protein 4A” include wild-type forms of the FRMD4A gene or protein, as well as variants (e.g., splice variants, truncations, concatemers, and fusion constructs, among others) of wild-type FRMD4A proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type FRMD4A protein (e.g., SEQ ID NO: 30), provided that the FRMD4A variant retains the therapeutic function of a wild-type FRMD4A. Additionally, the terms “FRMD4A” and “FERM domain-containing protein 4A” may refer to a “FRMD4A fusion protein,” which is a protein in which the FRMD4A is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “FRMD4A” may refer to the protein or the gene encoding this protein, depending upon the context, as will be appreciated by one of skill in the art.

As used herein, the term “SPPL2A” refers to the gene encoding Signal peptide peptidase-like 2A, or the corresponding protein product. The terms “SPPL2A” and “Signal peptide peptidase-like 2A” include wild-type forms of the SPPL2A gene or protein, as well as variants (e.g., splice variants, truncations, concatemers, and fusion constructs, among others) of wild-type SPPL2A proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type SPPL2A protein (e.g., SEQ ID NO: 31), provided that the SPPL2A variant retains the therapeutic function of a wild-type SPPL2A. Additionally, the terms “SPPL2A” and “Signal peptide peptidase-like 2A” may refer to a “SPPL2A fusion protein,” which is a protein in which the SPPL2A is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “SPPL2A” may refer to the protein or the gene encoding this protein, depending upon the context, as will be appreciated by one of skill in the art.

As used herein, the term “MTHFD1 L” refers to the gene encoding Mitochondrial monofunctional C1-tetrahydrofolate synthase, or the corresponding protein product. The terms “MTHFD1 L” and “Mitochondrial monofunctional C1-tetrahydrofolate synthase” include wild-type forms of the MTHFD1 L gene or protein, as well as variants (e.g., splice variants, truncations, concatemers, and fusion constructs, among others) of wild-type MTHFD1 L proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type MTHFD1 L protein (e.g., SEQ ID NO: 32), provided that the MTHFD1 L variant retains the therapeutic function of a wild-type MTHFD1 L. Additionally, the terms “MTHFD1 L” and “Mitochondrial monofunctional C1-tetrahydrofolate synthase” may refer to a “MTHFD1 L fusion protein,” which is a protein in which the MTHFD1 L is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “MTHFD1L” may refer to the protein or the gene encoding this protein, depending upon the context, as will be appreciated by one of skill in the art.

As used herein, the term “STK24” refers to the gene encoding Serine/threonine-protein kinase 24, or the corresponding protein product. The terms “STK24” and “Serine/threonine-protein kinase 24” include wild-type forms of the STK24 gene or protein, as well as variants (e.g., splice variants, truncations, concatemers, and fusion constructs, among others) of wild-type STK24 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type STK24 protein (e.g., SEQ ID NO: 33), provided that the STK24 variant retains the therapeutic function of a wild-type STK24. Additionally, the terms “STK24” and “Serine/threonine-protein kinase 24” may refer to a “STK24 fusion protein,” which is a protein in which the STK24 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “STK24” may refer to the protein or the gene encoding this protein, depending upon the context, as will be appreciated by one of skill in the art.

As used herein, the term “DISCI” refers to the gene encoding Disrupted in schizophrenia 1 protein, or the corresponding protein product. The terms “DISC1” and “Disrupted in schizophrenia 1” protein include wild-type forms of the DISCI gene or protein, as well as variants (e.g., splice variants, truncations, concatemers, and fusion constructs, among others) of wild-type DISCI proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type DISCI protein (e.g., SEQ ID NO: 34), provided that the DISCI variant retains the therapeutic function of a wild-type DISCI . Additionally, the terms “DISCI” and “Disrupted in schizophrenia 1 protein” may refer to a “DISCI fusion protein,” which is a protein in which the DISC1 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “DISCI” may refer to the protein or the gene encoding this protein, depending upon the context, as will be appreciated by one of skill in the art.

As used herein, the term “MPZL1” refers to the gene encoding Myelin protein zero-like protein 1, or the corresponding protein product. The terms “MPZL1” and “Myelin protein zero-like protein 1” include wild-type forms of the MPZL1 gene or protein, as well as variants (e.g., splice variants, truncations, concatemers, and fusion constructs, among others) of wild-type MPZL1 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type MPZL1 protein (e.g., SEQ ID NO: 35), provided that the MPZL1 variant retains the therapeutic function of a wild-type MPZL1. Additionally, the terms “MPZL1” and “Myelin protein zero-like protein 1” may refer to a “MPZL1 fusion protein,” which is a protein in which the MPZL1 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “MPZL1” may refer to the protein or the gene encoding this protein, depending upon the context, as will be appreciated by one of skill in the art.

As used herein, the term “SLC4A1AP” refers to the gene encoding Kanadaptin, or the corresponding protein product. The terms “SLC4A1AP” and “Kanadaptin” include wild-type forms of the SLC4A1AP gene or protein, as well as variants (e.g., splice variants, truncations, concatemers, and fusion constructs, among others) of wild-type SLC4A1AP proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type SLC4A1AP protein (e.g., SEQ ID NO: 36), provided that the SLC4A1AP variant retains the therapeutic function of a wild-type SLC4A1AP. Additionally, the terms “SLC4A1AP” and “Kanadaptin” may refer to a “SLC4A1AP fusion protein,” which is a protein in which the SLC4A1AP is operably linked to another polypeptide, half-life-modifying agent, or therapeutic agent, such as an ApoE

Rb domain (such as a Rb domain having the amino acid sequence of residues 25-185, 50-180, 75-175, 100-170, 125-160, or 130-150 of SEQ ID NO: 105). As used herein, the term “SLC4A1AP” may refer to the protein or the gene encoding this protein, depending upon the context, as will be appreciated by one of skill in the art.

As used herein, the term “TRIP4” refers to the gene encoding Activating signal cointegrator 1, or the corresponding protein product. The terms “TRIP4” and “Activating signal cointegrator 1” include wild-type forms of the TRIP4 gene or protein, as well as variants (e.g., splice variants, truncations, concatemers, and fusion constructs, among others) of wild-type TRIP4 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type TRIP4 protein (e.g., SEQ ID NO: 37), provided that the TRIP4 variant retains the therapeutic function of a wild-type TRIP4. Additionally, the terms “TRIP4” and “Activating signal cointegrator 1” may refer to a “TRIP4 fusion protein,” which is a protein in which the TRIP4 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “TRIP4” may refer to the protein or the gene encoding this protein, depending upon the context, as will be appreciated by one of skill in the art.

As used herein, the term “MSRA” refers to the gene encoding Mitochondrial peptide methionine sulfoxide reductase, or the corresponding protein product. The terms “MSRA” and “Mitochondrial peptide methionine sulfoxide reductase” include wild-type forms of the MSRA gene or protein, as well as variants (e.g., splice variants, truncations, concatemers, and fusion constructs, among others) of wild-type MSRA proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type MSRA protein (e.g., SEQ ID NO: 38), provided that the MSRA variant retains the therapeutic function of a wild-type MSRA. Additionally, the terms “MSRA” and “Mitochondrial peptide methionine sulfoxide reductase” may refer to a “MSRA fusion protein,” which is a protein in which the MSRA is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “MSRA” may refer to the protein or the gene encoding this protein, depending upon the context, as will be appreciated by one of skill in the art.

As used herein, the term “HS3ST1” refers to the gene encoding Heparan sulfate glucosamine 3-O-sulfotransferase 1, or the corresponding protein product. The terms “HS3ST1” and “Heparan sulfate glucosamine 3-O-sulfotransferase 1” include wild-type forms of the HS3ST1 gene or protein, as well as variants (e.g., splice variants, truncations, concatemers, and fusion constructs, among others) of wild-type HS3ST1 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type HS3ST1 protein (e.g., SEQ ID NO: 39), provided that the HS3ST1 variant retains the therapeutic function of a wild-type HS3ST1. Additionally, the terms “HS3ST1” and “Heparan sulfate glucosamine 3-0-sulfotransferase 1” may refer to a “HS3ST1 fusion protein,” which is a protein in which the HS3ST1 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “HS3ST1” may refer to the protein or the gene encoding this protein, depending upon the context, as will be appreciated by one of skill in the art.

As used herein, the term “ZNF224” refers to the gene encoding Zinc finger protein 224, or the corresponding protein product. The terms “ZNF224” and “Zinc finger protein 224” include wild-type forms of the ZNF224 gene or protein, as well as variants (e.g., splice variants, truncations, concatemers, and fusion constructs, among others) of wild-type ZNF224 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type ZNF224 protein (e.g., SEQ ID NO: 40), provided that the ZNF224 variant retains the therapeutic function of a wild-type ZNF224. Additionally, the terms “ZNF224” and “Zinc finger protein 224” may refer to a “ZNF224 fusion protein,” which is a protein in which the ZNF224 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “ZNF224” may refer to the protein or the gene encoding this protein, depending upon the context, as will be appreciated by one of skill in the art.

As used herein, the term “AP2A2” refers to the gene encoding AP-2 complex subunit alpha-2, or the corresponding protein product. The terms “AP2A2” and “AP-2 complex subunit alpha-2” include wild-type forms of the AP2A2 gene or protein, as well as variants (e.g., splice variants, truncations, concatemers, and fusion constructs, among others) of wild-type AP2A2 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type AP2A2 protein (e.g., SEQ ID NO: 41), provided that the AP2A2 variant retains the therapeutic function of a wild-type AP2A2. Additionally, the terms “AP2A2” and “AP-2 complex subunit alpha-2” may refer to a “AP2A2 fusion protein,” which is a protein in which the AP2A2 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “AP2A2” may refer to the protein or the gene encoding this protein, depending upon the context, as will be appreciated by one of skill in the art.

As used herein, the term “VPS1” refers to the gene encoding Dynamin-1-like protein, or the corresponding protein product. The terms “VPS1” and “Dynamin-1-like protein” include wild-type forms of the VPS1 gene or protein, as well as variants (e.g., splice variants, truncations, concatemers, and fusion constructs, among others) of wild-type VPS1 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type VPS1 protein (e.g., SEQ ID NO: 42), provided that the VPS1 variant retains the therapeutic function of a wild-type VPS1. Additionally, the terms “VPS1” and “Dynamin-1-like protein” may refer to a “VPS1 fusion protein,” which is a protein in which the VPS1 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “VPS1” may refer to the protein or the gene encoding this protein, depending upon the context, as will be appreciated by one of skill in the art.

As used herein, the term “SCARB2” refers to the gene encoding Lysosome membrane protein 2, or the corresponding protein product. The terms “SCARB2” and “Lysosome membrane protein 2” include wild-type forms of the SCARB2 gene or protein, as well as variants (e.g., splice variants, truncations, concatemers, and fusion constructs, among others) of wild-type SCARB2 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type SCARB2 protein (e.g., SEQ ID NO: 43), provided that the SCARB2 variant retains the therapeutic function of a wild-type SCARB2. Additionally, the terms “SCARB2” and “Lysosome membrane protein 2” may refer to a “SCARB2 fusion protein,” which is a protein in which the SCARB2 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “SCARB2” may refer to the protein or the gene encoding this protein, depending upon the context, as will be appreciated by one of skill in the art.

As used herein, the term “GPNMB” refers to the gene encoding Transmembrane glycoprotein NMB, or the corresponding protein product. The terms “GPNMB” and “Transmembrane glycoprotein NMB” include wild-type forms of the GPNMB gene or protein, as well as variants (e.g., splice variants, truncations, concatemers, and fusion constructs, among others) of wild-type GPNMB proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type GPNMB protein (e.g., SEQ ID NO: 44), provided that the GPNMB variant retains the therapeutic function of a wild-type GPNMB. Additionally, the terms “GPNMB” and “Transmembrane glycoprotein NMB” may refer to a “GPNMB fusion protein,” which is a protein in which the GPNMB is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “GPNMB” may refer to the protein or the gene encoding this protein, depending upon the context, as will be appreciated by one of skill in the art.

As used herein, the term “VPS35” refers to the gene encoding Vacuolar protein sorting-associated protein 35, or the corresponding protein product. The terms “VPS35” and “Vacuolar protein sorting-associated protein 35” include wild-type forms of the VPS35 gene or protein, as well as variants (e.g., splice variants, truncations, concatemers, and fusion constructs, among others) of wild-type VPS35 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type VPS35 protein (e.g., SEQ ID NO: 45), provided that the VPS35 variant retains the therapeutic function of a wild-type VPS35. Additionally, the terms “VPS35” and “Vacuolar protein sorting-associated protein 35” may refer to a “VPS35 fusion protein,” which is a protein in which the VPS35 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “VPS35” may refer to the protein or the gene encoding this protein, depending upon the context, as will be appreciated by one of skill in the art.

As used herein, the term “FBXO7” refers to the gene encoding F-box only protein 7, or the corresponding protein product. The terms “FBXO7” and “F-box only protein 7” include wild-type forms of the FBXO7 gene or protein, as well as variants (e.g., splice variants, truncations, concatemers, and fusion constructs, among others) of wild-type FBXO7 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type FBXO7 protein (e.g., SEQ ID NO: 46), provided that the FBXO7 variant retains the therapeutic function of a wild-type FBXO7. Additionally, the terms “FBXO7” and “F-box only protein 7” may refer to a “FBXO7 fusion protein,” which is a protein in which the FBXO7 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “FBXO7” may refer to the protein or the gene encoding this protein, depending upon the context, as will be appreciated by one of skill in the art.

As used herein, the term “PARK7” refers to the gene encoding Protein/nucleic acid deglycase DJ-1, or the corresponding protein product. The terms “PARK7” and “Protein/nucleic acid deglycase DJ-1” include wild-type forms of the PARK7 gene or protein, as well as variants (e.g., splice variants, truncations, concatemers, and fusion constructs, among others) of wild-type PARK7 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type PARK7 protein (e.g., SEQ ID NO: 47), provided that the PARK7 variant retains the therapeutic function of a wild-type PARK7. Additionally, the terms “PARK7” and “Protein/nucleic acid deglycase DJ-1” may refer to a “PARK7 fusion protein,” which is a protein in which the PARK7 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “PARK7” may refer to the protein or the gene encoding this protein, depending upon the context, as will be appreciated by one of skill in the art.

As used herein, the term “INPP5F” refers to the gene encoding Phosphatidylinositide phosphatase SAC2, or the corresponding protein product. The terms “INPP5F” and “Phosphatidylinositide phosphatase SAC2” include wild-type forms of the INPP5F gene or protein, as well as variants (e.g., splice variants, truncations, concatemers, and fusion constructs, among others) of wild-type INPP5F proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type INPP5F protein (e.g., SEQ ID NO: 48), provided that the INPP5F variant retains the therapeutic function of a wild-type INPP5F. Additionally, the terms “INPP5F” and “Phosphatidylinositide phosphatase SAC2” may refer to a “INPP5F fusion protein,” which is a protein in which the INPP5F is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “INPP5F” may refer to the protein or the gene encoding this protein, depending upon the context, as will be appreciated by one of skill in the art.

As used herein, the term “DNAJC13” refers to the gene encoding DNAJ homolog subfamily C member 13, or the corresponding protein product. The terms “DNAJC13” and “DNAJ homolog subfamily C member 13” include wild-type forms of the DNAJC13 gene or protein, as well as variants (e.g., splice variants, truncations, concatemers, and fusion constructs, among others) of wild-type DNAJC13 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type DNAJC13 protein (e.g., SEQ ID NO: 49), provided that the DNAJC13 variant retains the therapeutic function of a wild-type DNAJC13. Additionally, the terms “DNAJC13” and “DNAJ homolog subfamily C member 13” may refer to a “DNAJC13 fusion protein,” which is a protein in which the DNAJC13 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “DNAJC13” may refer to the protein or the gene encoding this protein, depending upon the context, as will be appreciated by one of skill in the art.

As used herein, the term “GCH1” refers to the gene encoding GTP cyclohydrolase 1, or the corresponding protein product. The terms “GCH1” and “GTP cyclohydrolase 1” include wild-type forms of the GCH1 gene or protein, as well as variants (e.g., splice variants, truncations, concatemers, and fusion constructs, among others) of wild-type GCH1 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type GCH1 protein (e.g., SEQ ID NO: 50), provided that the GCH1 variant retains the therapeutic function of a wild-type GCH1. Additionally, the terms “GCH1” and “GTP cyclohydrolase 1” may refer to a “GCH1 fusion protein,” which is a protein in which the GCH1 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “GCH1” may refer to the protein or the gene encoding this protein, depending upon the context, as will be appreciated by one of skill in the art.

As used herein, the term “NMD3” refers to the gene encoding 60S ribosomal export protein NMD3, or the corresponding protein product. The terms “NMD3” and “60S ribosomal export protein NMD3” include wild-type forms of the NMD3 gene or protein, as well as variants (e.g., splice variants, truncations, concatemers, and fusion constructs, among others) of wild-type NMD3 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type NMD3 protein (e.g., SEQ ID NO: 51), provided that the NMD3 variant retains the therapeutic function of a wild-type NMD3. Additionally, the terms “NMD3” and “60S ribosomal export protein NMD3” may refer to a “NMD3 fusion protein,” which is a protein in which the NMD3 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “NMD3” may refer to the protein or the gene encoding this protein, depending upon the context, as will be appreciated by one of skill in the art.

As used herein, the term “USP25” refers to the gene encoding Ubiquitin carboxyl-terminal hydrolase 25, or the corresponding protein product. The terms “USP25” and “Ubiquitin carboxyl-terminal hydrolase 25” include wild-type forms of the USP25 gene or protein, as well as variants (e.g., splice variants, truncations, concatemers, and fusion constructs, among others) of wild-type USP25 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type USP25 protein (e.g., SEQ ID NO: 52), provided that the USP25 variant retains the therapeutic function of a wild-type USP25. Additionally, the terms “USP25” and “Ubiquitin carboxyl-terminal hydrolase 25” may refer to a “USP25 fusion protein,” which is a protein in which the USP25 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “USP25” may refer to the protein or the gene encoding this protein, depending upon the context, as will be appreciated by one of skill in the art.

As used herein, the term “RAB7L1” refers to the gene encoding Ras-related protein Rab-7L1, or the corresponding protein product. The terms “RAB7L1” and “Ras-related protein Rab-7L1” include wild-type forms of the RAB7L1 gene or protein, as well as variants (e.g., splice variants, truncations, concatemers, and fusion constructs, among others) of wild-type RAB7L1 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type RAB7L1 protein (e.g., SEQ ID NO: 53), provided that the RAB7L1 variant retains the therapeutic function of a wild-type RAB7L1. Additionally, the terms “RAB7L1” and “Ras-related protein Rab-7L1” may refer to a “RAB7L1 fusion protein,” which is a protein in which the RAB7L1 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “RAB7L1” may refer to the protein or the gene encoding this protein, depending upon the context, as will be appreciated by one of skill in the art.

As used herein, the term “SIPA1 L2” refers to the gene encoding Signal-induced proliferation-associated 1-like protein 2, or the corresponding protein product. The terms “SIPA1 L2” and “Signal-induced proliferation-associated 1-like protein 2” include wild-type forms of the SIPA1 L2 gene or protein, as well as variants (e.g., splice variants, truncations, concatemers, and fusion constructs, among others) of wild-type SIPA1 L2 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type SIPA1 L2 protein (e.g., SEQ ID NO: 54), provided that the SI PA1L2 variant retains the therapeutic function of a wild-type SIPA1L2. Additionally, the terms “SIPA1L2” and “Signal-induced proliferation-associated 1-like protein 2” may refer to a “SIPA1L2 fusion protein,” which is a protein in which the SIPA1L2 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “SIPA1 L2” may refer to the protein or the gene encoding this protein, depending upon the context, as will be appreciated by one of skill in the art.

As used herein, the term “MCCC1” refers to the gene encoding Mitochondrial methylcrotonoyl-CoA carboxylase subunit alpha, or the corresponding protein product. The terms “MCCC1” and “Mitochondrial methylcrotonoyl-CoA carboxylase subunit alpha” include wild-type forms of the MCCC1 gene or protein, as well as variants (e.g., splice variants, truncations, concatemers, and fusion constructs, among others) of wild-type MCCC1 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type MCCC1 protein (e.g., SEQ ID NO: 55), provided that the MCCC1 variant retains the therapeutic function of a wild-type MCCC1. Additionally, the terms “MCCC1” and “Mitochondrial methylcrotonoyl-CoA carboxylase subunit alpha” may refer to a “MCCC1 fusion protein,” which is a protein in which the MCCC1 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “MCCC1” may refer to the protein or the gene encoding this protein, depending upon the context, as will be appreciated by one of skill in the art.

As used herein, the term “SYNJ1” refers to the gene encoding Synaptojanin-1, or the corresponding protein product. The terms “SYNJ1” and “Synaptojanin-1” include wild-type forms of the SYNJ1 gene or protein, as well as variants (e.g., splice variants, truncations, concatemers, and fusion constructs, among others) of wild-type SYNJ1 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type SYNJ1 protein (e.g., SEQ ID NO: 56), provided that the SYNJ1 variant retains the therapeutic function of a wild-type SYNJ1. Additionally, the terms “SYNJ1” and “Synaptojanin-1” may refer to a “SYNJ1 fusion protein,” which is a protein in which the SYNJ1 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “SYNJ1” may refer to the protein or the gene encoding this protein, depending upon the context, as will be appreciated by one of skill in the art.

As used herein, the term “LRRK2” refers to the gene encoding Leucine-rich repeat serine/threonine-protein kinase 2, or the corresponding protein product. The terms “LRRK2” and “Leucine-rich repeat serine/threonine-protein kinase 2” include wild-type forms of the LRRK2 gene or protein, as well as variants (e.g., splice variants, truncations, concatemers, and fusion constructs, among others) of wild-type LRRK2 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type LRRK2 protein (e.g., SEQ ID NO: 57), provided that the LRRK2 variant retains the therapeutic function of a wild-type LRRK2. Additionally, the terms “LRRK2” and “Leucine-rich repeat serine/threonine-protein kinase 2” may refer to a “LRRK2 fusion protein,” which is a protein in which the LRRK2 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “LRRK2” may refer to the protein or the gene encoding this protein, depending upon the context, as will be appreciated by one of skill in the art.

As used herein, the term “SNCA” refers to the gene encoding Alpha-synuclein, or the corresponding protein product. The terms “SNCA” and “Alpha-synuclein” include wild-type forms of the SNCA gene or protein, as well as variants (e.g., splice variants, truncations, concatemers, and fusion constructs, among others) of wild-type SNCA proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type SNCA protein (e.g., SEQ ID NO: 58), provided that the SNCA variant retains the therapeutic function of a wild-type SNCA. Additionally, the terms “SNCA” and “Alpha-synuclein” may refer to a “SNCA fusion protein,” which is a protein in which the SNCA is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “SNCA” may refer to the protein or the gene encoding this protein, depending upon the context, as will be appreciated by one of skill in the art.

As used herein, the term “PTRHD1” refers to the gene encoding Peptidyl-tRNA hydrolase PTRHD1, or the corresponding protein product. The terms “PTRHD1” and “Peptidyl-tRNA hydrolase PTRHD1” include wild-type forms of the PTRHD1 gene or protein, as well as variants (e.g., splice variants, truncations, concatemers, and fusion constructs, among others) of wild-type PTRHD1 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type PTRHD1 protein (e.g., SEQ ID NO: 59), provided that the PTRHD1 variant retains the therapeutic function of a wild-type PTRHD1. Additionally, the terms “PTRHD1” and “Peptidyl-tRNA hydrolase PTRHD1” may refer to a “PTRHD1 fusion protein,” which is a protein in which the PTRHD1 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “PTRHD1” may refer to the protein or the gene encoding this protein, depending upon the context, as will be appreciated by one of skill in the art.

As used herein, the term “PINK1” refers to the gene encoding Mitochondrial Serine/threonine-protein kinase PINK1, or the corresponding protein product. The terms “PINK1” and “Mitochondrial Serine/threonine-protein kinase PINK1” include wild-type forms of the PINK1 gene or protein, as well as variants (e.g., splice variants, truncations, concatemers, and fusion constructs, among others) of wild-type PINK1 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type PINK1 protein (e.g., SEQ ID NO: 60), provided that the PINK1 variant retains the therapeutic function of a wild-type PINK1. Additionally, the terms “PINK1” and “Mitochondrial Serine/threonine-protein kinase PINK1” may refer to a “PINK1 fusion protein,” which is a protein in which the PINK1 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “PINK1” may refer to the protein or the gene encoding this protein, depending upon the context, as will be appreciated by one of skill in the art.

As used herein, the term “TMEM163” refers to the gene encoding Transmembrane protein 163, or the corresponding protein product. The terms “TMEM163” and “Transmembrane protein 163” include wild-type forms of the TMEM163 gene or protein, as well as variants (e.g., splice variants, truncations, concatemers, and fusion constructs, among others) of wild-type TMEM163 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type TMEM163 protein (e.g., SEQ ID NO: 61), provided that the TMEM163 variant retains the therapeutic function of a wild-type TMEM163. Additionally, the terms “TMEM163” and “Transmembrane protein 163” may refer to a “TMEM163 fusion protein,” which is a protein in which the TMEM163 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “TMEM163” may refer to the protein or the gene encoding this protein, depending upon the context, as will be appreciated by one of skill in the art.

As used herein, the term “GAK” refers to the gene encoding Cyclin-G-associated kinase, or the corresponding protein product. The terms “GAK” and “Cyclin-G-associated kinase” include wild-type forms of the GAK gene or protein, as well as variants (e.g., splice variants, truncations, concatemers, and fusion constructs, among others) of wild-type GAK proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type GAK protein (e.g., SEQ ID NO: 62), provided that the GAK variant retains the therapeutic function of a wild-type GAK. Additionally, the terms “GAK” and “Cyclin-G-associated kinase” may refer to a “GAK fusion protein,” which is a protein in which the GAK is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “GAK” may refer to the protein or the gene encoding this protein, depending upon the context, as will be appreciated by one of skill in the art.

As used herein, the term “FGF20” refers to the gene encoding Fibroblast growth factor 20, or the corresponding protein product. The terms “FGF20” and “Fibroblast growth factor 20” include wild-type forms of the FGF20 gene or protein, as well as variants (e.g., splice variants, truncations, concatemers, and fusion constructs, among others) of wild-type FGF20 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type FGF20 protein (e.g., SEQ ID NO: 63), provided that the FGF20 variant retains the therapeutic function of a wild-type FGF20. Additionally, the terms “FGF20” and “Fibroblast growth factor 20” may refer to a “FGF20 fusion protein,” which is a protein in which the FGF20 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “FGF20” may refer to the protein or the gene encoding this protein, depending upon the context, as will be appreciated by one of skill in the art.

As used herein, the term “DLG2” refers to the gene encoding Disks large homolog 2, or the corresponding protein product. The terms “DLG2” and “Disks large homolog 2” include wild-type forms of the DLG2 gene or protein, as well as variants (e.g., splice variants, truncations, concatemers, and fusion constructs, among others) of wild-type DLG2 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type DLG2 protein (e.g., SEQ ID NO: 64), provided that the DLG2 variant retains the therapeutic function of a wild-type DLG2. Additionally, the terms “DLG2” and “Disks large homolog 2” may refer to a “DLG2 fusion protein,” which is a protein in which the DLG2 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “DLG2” may refer to the protein or the gene encoding this protein, depending upon the context, as will be appreciated by one of skill in the art.

As used herein, the term “DDRGK1” refers to the gene encoding DDRGK domain-containing protein 1, or the corresponding protein product. The terms “DDRGK1” and “DDRGK domain-containing protein 1” include wild-type forms of the DDRGK1 gene or protein, as well as variants (e.g., splice variants, truncations, concatemers, and fusion constructs, among others) of wild-type DDRGK1 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type DDRGK1 protein (e.g., SEQ ID NO: 65), provided that the DDRGK1 variant retains the therapeutic function of a wild-type DDRGK1. Additionally, the terms “DDRGK1” and “DDRGK domain-containing protein 1” may refer to a “DDRGK1 fusion protein,” which is a protein in which the DDRGK1 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “DDRGK1” may refer to the protein or the gene encoding this protein, depending upon the context, as will be appreciated by one of skill in the art.

As used herein, the term “SREBF” refers to the gene encoding Sterol regulatory element-binding protein 1, or the corresponding protein product. The terms “SREBF” and “Sterol regulatory element-binding protein 1” include wild-type forms of the SREBF gene or protein, as well as variants (e.g., splice variants, truncations, concatemers, and fusion constructs, among others) of wild-type SREBF proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type SREBF protein (e.g., SEQ ID NO: 66), provided that the SREBF variant retains the therapeutic function of a wild-type SREBF. Additionally, the terms “SREBF” and “Sterol regulatory element-binding protein 1” may refer to a “SREBF fusion protein,” which is a protein in which the SREBF is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “SREBF” may refer to the protein or the gene encoding this protein, depending upon the context, as will be appreciated by one of skill in the art.

As used herein, the term “BCKDK” refers to the gene encoding Branched-chain alpha-ketoacid dehydrogenase kinase, or the corresponding protein product. The terms “BCKDK” and “Branched-chain alpha-ketoacid dehydrogenase kinase” include wild-type forms of the BCKDK gene or protein, as well as variants (e.g., splice variants, truncations, concatemers, and fusion constructs, among others) of wild-type BCKDK proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type BCKDK protein (e.g., SEQ ID NO: 67), provided that the BCKDK variant retains the therapeutic function of a wild-type BCKDK. Additionally, the terms “BCKDK” and “Branched-chain alpha-ketoacid dehydrogenase kinase” may refer to a “BCKDK fusion protein,” which is a protein in which the BCKDK is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “BCKDK” may refer to the protein or the gene encoding this protein, depending upon the context, as will be appreciated by one of skill in the art.

As used herein, the term “PARK2” refers to the gene encoding E3 ubiquitin-protein ligase parkin, or the corresponding protein product. The terms “PARK2” and “E3 ubiquitin-protein ligase parkin” include wild-type forms of the PARK2 gene or protein, as well as variants (e.g., splice variants, truncations, concatemers, and fusion constructs, among others) of wild-type PARK2 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type PARK2 protein (e.g., SEQ ID NO: 68), provided that the PARK2 variant retains the therapeutic function of a wild-type PARK2. Additionally, the terms “PARK2” and “E3 ubiquitin-protein ligase parkin” may refer to a “PARK2 fusion protein,” which is a protein in which the PARK2 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “PARK2” may refer to the protein or the gene encoding this protein, depending upon the context, as will be appreciated by one of skill in the art.

As used herein, the term “RAB39B” refers to the gene encoding Ras-related protein Rab-39B, or the corresponding protein product. The terms “RAB39B” and “Ras-related protein Rab-39B” include wild-type forms of the RAB39B gene or protein, as well as variants (e.g., splice variants, truncations, concatemers, and fusion constructs, among others) of wild-type RAB39B proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type RAB39B protein (e.g., SEQ ID NO: 69), provided that the RAB39B variant retains the therapeutic function of a wild-type RAB39B. Additionally, the terms “RAB39B” and “Ras-related protein Rab-39B” may refer to a “RAB39B fusion protein,” which is a protein in which the RAB39B is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “RAB39B” may refer to the protein or the gene encoding this protein, depending upon the context, as will be appreciated by one of skill in the art.

As used herein, the term “DNAJC6” refers to the gene encoding Tyrosine-protein phosphatase auxilin, or the corresponding protein product. The terms “DNAJC6” and “Tyrosine-protein phosphatase auxilin” include wild-type forms of the DNAJC6 gene or protein, as well as variants (e.g., splice variants, truncations, concatemers, and fusion constructs, among others) of wild-type DNAJC6 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type DNAJC6 protein (e.g., SEQ ID NO: 70), provided that the DNAJC6 variant retains the therapeutic function of a wild-type DNAJC6. Additionally, the terms “DNAJC6” and “Tyrosine-protein phosphatase auxilin” may refer to a “DNAJC6 fusion protein,” which is a protein in which the DNAJC6 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “DNAJC6” may refer to the protein or the gene encoding this protein, depending upon the context, as will be appreciated by one of skill in the art.

As used herein, the term “SMPD1” refers to the gene encoding Sphingomyelin phosphodiesterase, or the corresponding protein product. The terms “SMPD1” and “Sphingomyelin phosphodiesterase” include wild-type forms of the SMPD1 gene or protein, as well as variants (e.g., splice variants, truncations, concatemers, and fusion constructs, among others) of wild-type SMPD1 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type SMPD1 protein (e.g., SEQ ID NO: 71), provided that the SMPD1 variant retains the therapeutic function of a wild-type SMPD1.

Additionally, the terms “SMPD1” and “Sphingomyelin phosphodiesterase” may refer to a “SMPD1 fusion protein,” which is a protein in which the SMPD1 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “SMPD1” may refer to the protein or the gene encoding this protein, depending upon the context, as will be appreciated by one of skill in the art.

As used herein, the term “TMEM175” refers to the gene encoding Endosomal/lysosomal potassium channel TMEM175, or the corresponding protein product. The terms “TMEM175” and “Endosomal/lysosomal potassium channel TMEM175” include wild-type forms of the TMEM175 gene or protein, as well as variants (e.g., splice variants, truncations, concatemers, and fusion constructs, among others) of wild-type TMEM175 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type TMEM175 protein (e.g., SEQ ID NO: 72), provided that the TMEM175 variant retains the therapeutic function of a wild-type TMEM175. Additionally, the terms “TMEM175” and “Endosomal/lysosomal potassium channel TMEM175” may refer to a “TMEM175 fusion protein,” which is a protein in which the TMEM175 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “TMEM175” may refer to the protein or the gene encoding this protein, depending upon the context, as will be appreciated by one of skill in the art.

As used herein, the term “STK39” refers to the gene encoding STE20/SPS1-related proline-alanine-rich protein kinase, or the corresponding protein product. The terms “STK39” and “STE20/SPS1-related proline-alanine-rich protein kinase” include wild-type forms of the STK39 gene or protein, as well as variants (e.g., splice variants, truncations, concatemers, and fusion constructs, among others) of wild-type STK39 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type STK39 protein (e.g., SEQ ID NO: 73), provided that the STK39 variant retains the therapeutic function of a wild-type STK39. Additionally, the terms “STK39” and “STE20/SPS1-related proline-alanine-rich protein kinase” may refer to a “STK39 fusion protein,” which is a protein in which the STK39 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “STK39” may refer to the protein or the gene encoding this protein, depending upon the context, as will be appreciated by one of skill in the art.

As used herein, the term “BST1” refers to the gene encoding ADP-ribosyl cyclase/cyclic ADP-ribose hydrolase 2, or the corresponding protein product. The terms “BST1” and “ADP-ribosyl cyclase/cyclic ADP-ribose hydrolase 2” include wild-type forms of the BST1 gene or protein, as well as variants (e.g., splice variants, truncations, concatemers, and fusion constructs, among others) of wild-type BST1 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type BST1 protein (e.g., SEQ ID NO: 74), provided that the BST1 variant retains the therapeutic function of a wild-type BST1. Additionally, the terms “BST1” and “ADP-ribosyl cyclase/cyclic ADP-ribose hydrolase 2” may refer to a “BST1 fusion protein,” which is a protein in which the BST1 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “BST1” may refer to the protein or the gene encoding this protein, depending upon the context, as will be appreciated by one of skill in the art.

As used herein, the term “MMP16” refers to the gene encoding Matrix metalloproteinase-16, or the corresponding protein product. The terms “MMP16” and “Matrix metalloproteinase-16” include wild-type forms of the MMP16 gene or protein, as well as variants (e.g., splice variants, truncations, concatemers, and fusion constructs, among others) of wild-type MMP16 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type MMP16 protein (e.g., SEQ ID NO: 75), provided that the MMP16 variant retains the therapeutic function of a wild-type MMP16. Additionally, the terms “MMP16” and “Matrix metalloproteinase-16” may refer to a “MMP16 fusion protein,” which is a protein in which the MMP16 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “MMP16” may refer to the protein or the gene encoding this protein, depending upon the context, as will be appreciated by one of skill in the art.

As used herein, the term “RIT2” refers to the gene encoding GTP-binding protein Rit2, or the corresponding protein product. The terms “RIT2” and “GTP-binding protein Rit2” include wild-type forms of the RIT2 gene or protein, as well as variants (e.g., splice variants, truncations, concatemers, and fusion constructs, among others) of wild-type RIT2 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type RIT2 protein (e.g., SEQ ID NO: 76), provided that the RIT2 variant retains the therapeutic function of a wild-type RIT2. Additionally, the terms “RIT2” and “GTP-binding protein Rit2” may refer to a “RIT2 fusion protein,” which is a protein in which the RIT2 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “RIT2” may refer to the protein or the gene encoding this protein, depending upon the context, as will be appreciated by one of skill in the art.

As used herein, the term “FAM47E” refers to the gene encoding Protein FAM47E, or the corresponding protein product. The terms “FAM47E” and “Protein FAM47E” include wild-type forms of the FAM47E gene or protein, as well as variants (e.g., splice variants, truncations, concatemers, and fusion constructs, among others) of wild-type FAM47E proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type FAM47E protein (e.g., SEQ ID NO: 77), provided that the FAM47E variant retains the therapeutic function of a wild-type FAM47E. Additionally, the terms “FAM47E” and “Protein FAM47E” may refer to a “FAM47E fusion protein,” which is a protein in which the FAM47E is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “FAM47E” may refer to the protein or the gene encoding this protein, depending upon the context, as will be appreciated by one of skill in the art.

As used herein, the term “CCDC62” refers to the gene encoding Coiled-coil domain-containing protein 62, or the corresponding protein product. The terms “CCDC62” and “Coiled-coil domain-containing protein 62” include wild-type forms of the CCDC62 gene or protein, as well as variants (e.g., splice variants, truncations, concatemers, and fusion constructs, among others) of wild-type CCDC62 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type CCDC62 protein (e.g., SEQ ID NO: 78), provided that the CCDC62 variant retains the therapeutic function of a wild-type CCDC62. Additionally, the terms “CCDC62” and “Coiled-coil domain-containing protein 62” may refer to a “CCDC62 fusion protein,” which is a protein in which the CCDC62 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “CCDC62” may refer to the protein or the gene encoding this protein, depending upon the context, as will be appreciated by one of skill in the art.

As used herein, the term “HLA-DQB1” refers to the gene encoding HLA class II histocompatibility antigen, DQ beta 1 chain, or the corresponding protein product. The terms “HLA-DQB1” and “HLA class II histocompatibility antigen, DQ beta 1 chain” include wild-type forms of the HLA-DQB1 gene or protein, as well as variants (e.g., splice variants, truncations, concatemers, and fusion constructs, among others) of wild-type HLA-DQB1 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type HLA-DQB1 protein (e.g., SEQ ID NO: 79), provided that the HLA-DQB1 variant retains the therapeutic function of a wild-type HLA-DQB1. Additionally, the terms “HLA-DQB1” and “HLA class II histocompatibility antigen, DQ beta 1 chain” may refer to a “HLA-DQB1 fusion protein,” which is a protein in which the HLA-DQB1 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “HLA-DQB1” may refer to the protein or the gene encoding this protein, depending upon the context, as will be appreciated by one of skill in the art.

As used herein, the term “TMEM229B” refers to the gene encoding Transmembrane protein 229B, or the corresponding protein product. The terms “TMEM229B” and “Transmembrane protein 229B” include wild-type forms of the TMEM229B gene or protein, as well as variants (e.g., splice variants, truncations, concatemers, and fusion constructs, among others) of wild-type TMEM229B proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type TMEM229B protein (e.g., SEQ ID NO: 80), provided that the TMEM229B variant retains the therapeutic function of a wild-type TMEM229B. Additionally, the terms “TMEM229B” and “Transmembrane protein 229B” may refer to a “TMEM229B fusion protein,” which is a protein in which the TMEM229B is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “TMEM229B” may refer to the protein or the gene encoding this protein, depending upon the context, as will be appreciated by one of skill in the art.

As used herein, the term “MAPT” refers to the gene encoding Microtubule-associated protein tau, or the corresponding protein product. The terms “MAPT” and “Microtubule-associated protein tau” include wild-type forms of the MAPT gene or protein, as well as variants (e.g., splice variants, truncations, concatemers, and fusion constructs, among others) of wild-type MAPT proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type MAPT protein (e.g., SEQ ID NO: 81), provided that the MAPT variant retains the therapeutic function of a wild-type MAPT. Additionally, the terms “MAPT” and “Microtubule-associated protein tau” may refer to a “MAPT fusion protein,” which is a protein in which the MAPT is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “MAPT” may refer to the protein or the gene encoding this protein, depending upon the context, as will be appreciated by one of skill in the art.

As used herein, the term “SPPL2B” refers to the gene encoding Signal peptide peptidase-like 2B, or the corresponding protein product. The terms “SPPL2B” and “Signal peptide peptidase-like 2B” include wild-type forms of the SPPL2B gene or protein, as well as variants (e.g., splice variants, truncations, concatemers, and fusion constructs, among others) of wild-type SPPL2B proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type SPPL2B protein (e.g., SEQ ID NO: 82), provided that the SPPL2B variant retains the therapeutic function of a wild-type SPPL2B. Additionally, the terms “SPPL2B” and “Signal peptide peptidase-like 2B” may refer to a “SPPL2B fusion protein,” which is a protein in which the SPPL2B is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “SPPL2B” may refer to the protein or the gene encoding this protein, depending upon the context, as will be appreciated by one of skill in the art.

As used herein, the term “ITGA8” refers to the gene encoding Integrin alpha-8, or the corresponding protein product. The terms “ITGA8” and “Integrin alpha-8” include wild-type forms of the ITGA8 gene or protein, as well as variants (e.g., splice variants, truncations, concatemers, and fusion constructs, among others) of wild-type ITGA8 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type ITGA8 protein (e.g., SEQ ID NO: 83), provided that the ITGA8 variant retains the therapeutic function of a wild-type ITGA8. Additionally, the terms “ITGA8” and “Integrin alpha-8” may refer to a “ITGA8 fusion protein,” which is a protein in which the ITGA8 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “ITGA8” may refer to the protein or the gene encoding this protein, depending upon the context, as will be appreciated by one of skill in the art.

As used herein, the term “ATP13A2” refers to the gene encoding Cation-transporting ATPase 13A2, or the corresponding protein product. The terms “ATP13A2” and “Cation-transporting ATPase 13A2” include wild-type forms of the ATP13A2 gene or protein, as well as variants (e.g., splice variants, truncations, concatemers, and fusion constructs, among others) of wild-type ATP13A2 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type ATP13A2 protein (e.g., SEQ ID NO: 84), provided that the ATP13A2 variant retains the therapeutic function of a wild-type ATP13A2. Additionally, the terms “ATP13A2” and “Cation-transporting ATPase 13A2” may refer to a “ATP13A2 fusion protein,” which is a protein in which the ATP13A2 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “ATP13A2” may refer to the protein or the gene encoding this protein, depending upon the context, as will be appreciated by one of skill in the art.

As used herein, the term “DGKQ” refers to the gene encoding Diacylglycerol kinase theta, or the corresponding protein product. The terms “DGKQ” and “Diacylglycerol kinase theta” include wild-type forms of the DGKQ gene or protein, as well as variants (e.g., splice variants, truncations, concatemers, and fusion constructs, among others) of wild-type DGKQ proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type DGKQ protein (e.g., SEQ ID NO: 85), provided that the DGKQ variant retains the therapeutic function of a wild-type DGKQ. Additionally, the terms “DGKQ” and “Diacylglycerol kinase theta” may refer to a “DGKQ fusion protein,” which is a protein in which the DGKQ is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “DGKQ” may refer to the protein or the gene encoding this protein, depending upon the context, as will be appreciated by one of skill in the art.

As used herein, the term “STX1 B” refers to the gene encoding Syntaxin-1 B, or the corresponding protein product. The terms “STX1 B” and “Syntaxin-1 B” include wild-type forms of the STX1 B gene or protein, as well as variants (e.g., splice variants, truncations, concatemers, and fusion constructs, among others) of wild-type STX1 B proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type STX1 B protein (e.g., SEQ ID NO: 86), provided that the STX1B variant retains the therapeutic function of a wild-type STX1 B. Additionally, the terms “STX1 B” and “Syntaxin-1 B” may refer to a “STX1 B fusion protein,” which is a protein in which the STX1 B is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “STX1 B” may refer to the protein or the gene encoding this protein, depending upon the context, as will be appreciated by one of skill in the art.

As used herein, the term “NUCKS1” refers to the gene encoding Nuclear ubiquitous casein and cyclin-dependent kinase substrate 1, or the corresponding protein product. The terms “NUCKS1” and “Nuclear ubiquitous casein and cyclin-dependent kinase substrate 1” include wild-type forms of the NUCKS1 gene or protein, as well as variants (e.g., splice variants, truncations, concatemers, and fusion constructs, among others) of wild-type NUCKS1 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type NUCKS1 protein (e.g., SEQ ID NO: 87), provided that the NUCKS1 variant retains the therapeutic function of a wild-type NUCKS1. Additionally, the terms “NUCKS1” and “Nuclear ubiquitous casein and cyclin-dependent kinase substrate 1” may refer to a “NUCKS1 fusion protein,” which is a protein in which the NUCKS1 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “NUCKS1” may refer to the protein or the gene encoding this protein, depending upon the context, as will be appreciated by one of skill in the art.

As used herein, the term “ACMSD” refers to the gene encoding 2-amino-3-carboxymuconate-6-semialdehyde decarboxylase, or the corresponding protein product. The terms “ACMSD” and “2-amino-3-carboxymuconate-6-semialdehyde decarboxylase” include wild-type forms of the ACMSD gene or protein, as well as variants (e.g., splice variants, truncations, concatemers, and fusion constructs, among others) of wild-type ACMSD proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type ACMSD protein (e.g., SEQ ID NO: 88), provided that the ACMSD variant retains the therapeutic function of a wild-type ACMSD. Additionally, the terms “ACMSD” and “2-amino-3-carboxymuconate-6-semialdehyde decarboxylase” may refer to a “ACMSD fusion protein,” which is a protein in which the ACMSD is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “ACMSD” may refer to the protein or the gene encoding this protein, depending upon the context, as will be appreciated by one of skill in the art.

As used herein, the term “HLA-DRA” refers to the gene encoding HLA class II histocompatibility antigen, DR alpha chain, or the corresponding protein product. The terms “HLA-DRA” and “HLA class II histocompatibility antigen, DR alpha chain” include wild-type forms of the HLA-DRA gene or protein, as well as variants (e.g., splice variants, truncations, concatemers, and fusion constructs, among others) of wild-type HLA-DRA proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type HLA-DRA protein (e.g., SEQ ID NO: 89), provided that the HLA-DRA variant retains the therapeutic function of a wild-type HLA-DRA. Additionally, the terms “HLA-DRA” and “HLA class II histocompatibility antigen, DR alpha chain” may refer to a “HLA-DRA fusion protein,” which is a protein in which the HLA-DRA is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “HLA-DRA” may refer to the protein or the gene encoding this protein, depending upon the context, as will be appreciated by one of skill in the art.

As used herein, the term “C9ORF72” refers to the gene encoding Guanine nucleotide exchange C9orf72, or the corresponding protein product. The terms “C9ORF72” and “Guanine nucleotide exchange C9orf72” include wild-type forms of the C9ORF72 gene or protein, as well as variants (e.g., splice variants, truncations, concatemers, and fusion constructs, among others) of wild-type C9ORF72 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type C9ORF72 protein (e.g., SEQ ID NO: 90), provided that the C9ORF72 variant retains the therapeutic function of a wild-type C9ORF72. Additionally, the terms “C9ORF72” and “Guanine nucleotide exchange C9orf72” may refer to a “C9ORF72 fusion protein,” which is a protein in which the C9ORF72 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “C9ORF72” may refer to the protein or the gene encoding this protein, depending upon the context, as will be appreciated by one of skill in the art.

As used herein, the term “SQSTM1” refers to the gene encoding Sequestosome-1, or the corresponding protein product. The terms “SQSTM1” and “Sequestosome-1” include wild-type forms of the SQSTM1 gene or protein, as well as variants (e.g., splice variants, truncations, concatemers, and fusion constructs, among others) of wild-type SQSTM1 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type SQSTM1 protein (e.g., SEQ ID NO: 91), provided that the SQSTM1 variant retains the therapeutic function of a wild-type SQSTM1. Additionally, the terms “SQSTM1” and “Sequestosome-1” may refer to a “SQSTM1 fusion protein,” which is a protein in which the SQSTM1 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “SQSTM1” may refer to the protein or the gene encoding this protein, depending upon the context, as will be appreciated by one of skill in the art.

As used herein, the term “TARDBP” refers to the gene encoding TAR DNA-binding protein 43, or the corresponding protein product. The terms “TARDBP” and “TAR DNA-binding protein 43” include wild-type forms of the TARDBP gene or protein, as well as variants (e.g., splice variants, truncations, concatemers, and fusion constructs, among others) of wild-type TARDBP proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type TARDBP protein (e.g., SEQ ID NO: 92), provided that the TARDBP variant retains the therapeutic function of a wild-type TARDBP. Additionally, the terms “TARDBP” and “TAR DNA-binding protein 43” may refer to a “TARDBP fusion protein,” which is a protein in which the TARDBP is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “TARDBP” may refer to the protein or the gene encoding this protein, depending upon the context, as will be appreciated by one of skill in the art.

As used herein, the term “TBK1” refers to the gene encoding Serine/threonine-protein kinase TBK1, or the corresponding protein product. The terms “TBK1” and “Serine/threonine-protein kinase TBK1” include wild-type forms of the TBK1 gene or protein, as well as variants (e.g., splice variants, truncations, concatemers, and fusion constructs, among others) of wild-type TBK1 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type TBK1 protein (e.g., SEQ ID NO: 93), provided that the TBK1 variant retains the therapeutic function of a wild-type TBK1. Additionally, the terms “TBK1” and “Serine/threonine-protein kinase TBK1” may refer to a “TBK1 fusion protein,” which is a protein in which the TBK1 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “TBK1” may refer to the protein or the gene encoding this protein, depending upon the context, as will be appreciated by one of skill in the art.

As used herein, the term “VCP” refers to the gene encoding Transitional endoplasmic reticulum ATPase, or the corresponding protein product. The terms “VCP” and “Transitional endoplasmic reticulum ATPase” include wild-type forms of the VCP gene or protein, as well as variants (e.g., splice variants, truncations, concatemers, and fusion constructs, among others) of wild-type VCP proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type VCP protein (e.g., SEQ ID NO: 94), provided that the VCP variant retains the therapeutic function of a wild-type VCP. Additionally, the terms “VCP” and “Transitional endoplasmic reticulum ATPase” may refer to a “VCP fusion protein,” which is a protein in which the VCP is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “VCP” may refer to the protein or the gene encoding this protein, depending upon the context, as will be appreciated by one of skill in the art.

As used herein, the term “FUS” refers to the gene encoding RNA-binding protein FUS, or the corresponding protein product. The terms “FUS” and “RNA-binding protein FUS” include wild-type forms of the FUS gene or protein, as well as variants (e.g., splice variants, truncations, concatemers, and fusion constructs, among others) of wild-type FUS proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type FUS protein (e.g., SEQ ID NO: 95), provided that the FUS variant retains the therapeutic function of a wild-type FUS. Additionally, the terms “FUS” and “RNA-binding protein FUS” may refer to a “FUS fusion protein,” which is a protein in which the FUS is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “FUS” may refer to the protein or the gene encoding this protein, depending upon the context, as will be appreciated by one of skill in the art.

As used herein, the term “CHMP2B” refers to the gene encoding Charged multivesicular body protein 2b, or the corresponding protein product. The terms “CHMP2B” and “Charged multivesicular body protein 2b” include wild-type forms of the CHMP2B gene or protein, as well as variants (e.g., splice variants, truncations, concatemers, and fusion constructs, among others) of wild-type CHMP2B proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type CHMP2B protein (e.g., SEQ ID NO: 96), provided that the CHMP2B variant retains the therapeutic function of a wild-type CHMP2B. Additionally, the terms “CHMP2B” and “Charged multivesicular body protein 2b” may refer to a “CHMP2B fusion protein,” which is a protein in which the CHMP2B is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “CHMP2B” may refer to the protein or the gene encoding this protein, depending upon the context, as will be appreciated by one of skill in the art.

As used herein, the term “UBQLN2” refers to the gene encoding Ubiquilin-2, or the corresponding protein product. The terms “UBQLN2” and “Ubiquilin-2” include wild-type forms of the UBQLN2 gene or protein, as well as variants (e.g., splice variants, truncations, concatemers, and fusion constructs, among others) of wild-type UBQLN2 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type

UBQLN2 protein (e.g., SEQ ID NO: 97), provided that the UBQLN2 variant retains the therapeutic function of a wild-type UBQLN2. Additionally, the terms “UBQLN2” and “Ubiquilin-2” may refer to a “UBQLN2 fusion protein,” which is a protein in which the UBQLN2 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “UBQLN2” may refer to the protein or the gene encoding this protein, depending upon the context, as will be appreciated by one of skill in the art.

As used herein, the term “CHCHD10” refers to the gene encoding Mitochondrial coiled-coil-helix-coiled-coil-helix domain-containing protein 10, or the corresponding protein product. The terms “CHCHD10” and “Mitochondrial coiled-coil-helix-coiled-coil-helix domain-containing protein 10” include wild-type forms of the CHCHD10 gene or protein, as well as variants (e.g., splice variants, truncations, concatemers, and fusion constructs, among others) of wild-type CHCHD10 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type CHCHD10 protein (e.g., SEQ ID NO: 98), provided that the CHCHD10 variant retains the therapeutic function of a wild-type CHCHD10. Additionally, the terms “CHCHD10” and “Mitochondrial coiled-coil-helix-coiled-coil-helix domain-containing protein 10” may refer to a “CHCHD10 fusion protein,” which is a protein in which the CHCHD10 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “CHCHD10” may refer to the protein or the gene encoding this protein, depending upon the context, as will be appreciated by one of skill in the art.

As used herein, the term “RAB38” refers to the gene encoding Ras-related protein Rab-38, or the corresponding protein product. The terms “RAB38” and “Ras-related protein Rab-38” include wild-type forms of the RAB38 gene or protein, as well as variants (e.g., splice variants, truncations, concatemers, and fusion constructs, among others) of wild-type RAB38 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type RAB38 protein (e.g., SEQ ID NO: 99), provided that the RAB38 variant retains the therapeutic function of a wild-type RAB38. Additionally, the terms “RAB38” and “Ras-related protein Rab-38” may refer to a “RAB38 fusion protein,” which is a protein in which the RAB38 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “RAB38” may refer to the protein or the gene encoding this protein, depending upon the context, as will be appreciated by one of skill in the art.

As used herein, the term “CTSF” refers to the gene encoding Cathepsin F, or the corresponding protein product. The terms “CTSF” and “Cathepsin F” include wild-type forms of the CTSF gene or protein, as well as variants (e.g., splice variants, truncations, concatemers, and fusion constructs, among others) of wild-type CTSF proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type CTSF protein (e.g., SEQ ID NO: 100), provided that the CTSF variant retains the therapeutic function of a wild-type CTSF. Additionally, the terms “CTSF” and “Cathepsin F” may refer to a “CTSF fusion protein,” which is a protein in which the CTSF is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “CTSF” may refer to the protein or the gene encoding this protein, depending upon the context, as will be appreciated by one of skill in the art.

As used herein, the term “CYP27A1” refers to the gene encoding Mitochondrial Sterol 26-hydroxylase, or the corresponding protein product. The terms “CYP27A1” and “Mitochondrial Sterol 26-hydroxylase” include wild-type forms of the CYP27A1 gene or protein, as well as variants (e.g., splice variants, truncations, concatemers, and fusion constructs, among others) of wild-type CYP27A1 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type CYP27A1 protein (e.g., SEQ ID NO: 101), provided that the CYP27A1 variant retains the therapeutic function of a wild-type CYP27A1. Additionally, the terms “CYP27A1” and “Mitochondrial Sterol 26-hydroxylase” may refer to a “CYP27A1 fusion protein,” which is a protein in which the CYP27A1 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “CYP27A1” may refer to the protein or the gene encoding this protein, depending upon the context, as will be appreciated by one of skill in the art.

As used herein, the term “BTNL2” refers to the gene encoding Butyrophilin-like protein 2, or the corresponding protein product. The terms “BTNL2” and “Butyrophilin-like protein 2” include wild-type forms of the BTNL2 gene or protein, as well as variants (e.g., splice variants, truncations, concatemers, and fusion constructs, among others) of wild-type BTNL2 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type BTNL2 protein (e.g., SEQ ID NO: 102), provided that the BTNL2 variant retains the therapeutic function of a wild-type BTNL2. Additionally, the terms “BTNL2” and “Butyrophilin-like protein 2” may refer to a “BTNL2 fusion protein,” which is a protein in which the BTNL2 is operably linked to another polypeptide, half-life-modifying agent, or therapeutic agent, such as an ApoE Rb domain (such as a Rb domain having the amino acid sequence of residues 25-185, 50-180, 75-175, 100-170, 125-160, or 130-150 of SEQ ID NO: 105). As used herein, the term “BTNL2” may refer to the protein or the gene encoding this protein, depending upon the context, as will be appreciated by one of skill in the art.

### DETAILED DESCRIPTION

The present disclosure provides compositions and methods for treating an array of neurocognitive disorders (NCDs). The compositions and methods described herein may be used, for example, to treat a patient, such as an adult human patient suffering from or at risk of developing an NCD (e.g., Alzheimer's disease, Parkinson's disease, or a frontotemporal lobar degeneration (FTLD)). Such patients may be treated, for example, by providing to the patients one or more agents that together elevate the expression and/or activity levels of a protein or series of proteins whose deficiency is found to be associated with the corresponding disease. Without being limited by mechanism, the provision of such agents to a patient having an NCD described herein may restore physiologically normal quantities and activity levels of a protein or proteins that the patient under-expresses, and in this way, may treat n underlying biochemical etiology of the disease and reverse its pathophysiology. Thus, using the compositions and methods described herein, a patient may not only be treated in a manner that alleviates one or more symptoms associated with an NCD, but also in a curative fashion.

For examples, the compositions and methods of the disclosure may be used to provide a patient, such as a human patient, having an NCD (e.g., Alzheimer's disease) with one or more agents that together augment the expression and/or activity of one or more proteins selected from APP, PSEN1, PSEN2, APOE, TOMM40, GAB2, APOC1, TREM2, ABI3, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1 L, STK24, DISCI , MPZL1, SLC4A1AP, TRIP4, MSRA, HS3ST1, ZNF224, and AP2A2, such as one or more agents that together augment the expression and/or activity of one or more proteins selected from PSEN1, GAB2, APOC1, TREM2, ABI3, BIN1, HLA-DRB5, HLA-DRB1, CD2AP, PTK2B, INPP5D, MEF2C, CD33, MS4A4A, RIN3, PICALM, CASS4, SORL1, PLCG2, SCIMP, FRMD4A, SPPL2A, MTHFD1 L, DISCI , TRIP4, and HS3ST1. The one or more agents may, for example, elevate the expression and/or activity level of a subset of these proteins, such as a subset of two, three, four, five, six, seven, eight, nine, ten, or more, of these proteins.

Additionally or alternatively, the compositions and methods of the disclosure may be used to provide a patient, such as a human patient, having an NCD (e.g., Parkinson's disease) with one or more agents that together augment the expression and/or activity of one or more proteins selected from FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1 B, NUCKS1, and ACMSD, such as one or more agents that together augment the expression and/or activity of one or more proteins selected from FCGR2A, SCAF11, DNAJC13, GCH1, LRRK2, GBA, GAK, FGF20, HLA-DQB1, and NOD2. The one or more agents may, for example, elevate the expression and/or activity level of a subset of these proteins, such as a subset of two, three, four, five, six, seven, eight, nine, ten, or more, of these proteins.

The compositions and methods of the disclosure may also be used to provide a patient having an NCD (e.g., FTLD, such as behavioral-variant frontotemporal dementia, semantic dementia, or progressive nonfluent aphasia) with one or more agents that together augment the expression and/or activity of one or more proteins selected from HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT, such as one or more agents that together augment the expression and/or activity of one or more proteins selected from HLA-DRA, HLA-DRB5, C9ORF72, SQSTM1, TBK1, PSEN1, GRN, and CTSF. The one or more agents may, for example, elevate the expression and/or activity level of a subset of the foregoing proteins, such as a subset of two, three, four, five, six, seven, eight, nine, ten, or more, of these proteins.

Additionally, The compositions and methods of the disclosure may also be used to provide a patient having an NCD (e.g., Alzheimer's disease, Parkinson disease, or FTLD, such as behavioral-variant frontotemporal dementia, semantic dementia, or progressive nonfluent aphasia) with one or more agents that together augment the expression and/or activity of one or more proteins selected from APP, PSEN1, PSEN2, APOE, TOMM40, GAB2, APOC1, TREM2, ABI3, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1 L, STK24, DISCI , MPZL1, SLC4A1AP, TRIP4, MSRA, HS3ST1, ZNF224, AP2A2, FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1 L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1B, NUCKS1, ACMSD, HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT. The one or more agents may, for example, elevate the expression and/or activity level of a subset of the foregoing proteins, such as a subset of two, three, four, five, six, seven, eight, nine, ten, or more, of these proteins.

The present disclosure is based, in part, on the observation that elevating expression levels of particular genes and/or the activity levels of their protein product(s) in a patient having an NCD can halt and/or reverse disease progression and suppress associated symptoms. The compositions and methods described herein are also based, at least in part, on the discovery that increasing the expression and/or activity levels of certain groups of genes and their protein products can also be used to treat the above disorders. This discovery provides various clinical benefits. Particularly, using compositions and methods of the disclosure that augment expression and or activity levels of two or more proteins facilitate the treatment of larger patient populations relative to patient groups that can be treated using gene or protein monotherapy approaches. This stems from the finding that compositions that promote the expression and/or activity levels of multiple proteins can be safely administered to a patient (e.g., an adult human patient) even if the patient is deficient in only one of these proteins and already expresses the other(s). In view of this surprising observation, a single therapeutic product, such as a single population of cells, viral vectors, or other agents promoting the expression and/or activity of a plurality of proteins, may be used to treat large patient groups made up of individuals that each contain a unique protein deficiency. Using traditional monotherapy methods, each patient in such a population would require a customized agent that delivers only the gene or protein for which the patient is deficient. The present compositions and methods provide the unexpected technical advantage of being able to treat a diverse patient population using a single product that augments the expression and/or activity of multiple proteins, even if the patient is deficient in only one of the corresponding proteins.

Exemplary agents that may be used to elevate protein expression and/or activity levels in accordance with the compositions and methods of the disclosure include, without limitation, populations of cells (e.g., cells, such as CD34+ cells, hematopoietic stem cells, or myeloid progenitor cells) that contain nucleic acids encoding one or more desired proteins (e.g., nucleic acids capable of expression in macrophages or microglia), viral vectors that encode one or more of the desired proteins, and nucleic acid molecules, such as interfering RNA molecules, that stimulate the endogenous expression of one or more of the desired proteins. Additional examples of agents that may be used for this purpose include pharmaceutical compositions containing the one or more proteins themselves. The sections that follow provide a detailed description of such agents and the ways in which they may be provided to a patient, as well as the indications that these agents may be used to treat.

Neurocognitive Disorders

Neurocognitive disorders (NCDs) are defined as a collection of disorders that feature cognitive impairment as a core symptom and that show cognitive decline relative to a previously higher level of cognition (e.g., acquired impairment), rather than a developmental impairment. NCDs are broadly divided into major or mild syndromes (e.g., major NCD and mild NCD) based on the degree of impairment diagnosed in the patient. Furthermore, NCDs can be categorized on the basis of their etiological origin. For example, non-limiting examples of NCD may include NCD due to AD, NCD due to a movement disorder (e.g., Parkinson disease), frontotemporal NCD (e.g., FTLD), vascular NCD, NCD with Lewy bodies, NCD due to Parkinson disease, NCD due to traumatic brain injury, NCD due to HIV infection, substance/medication-induced NCD, NCD due to Huntington's disease, NCD due to prion disease, NCD due to another medical condition, NCD due to multiple etiologies, and unspecified NCD. The compositions and methods disclosed herein are useful for the treatment of NCDs.

### Alzheimer's Disease

Alzheimer's disease is a neurodegenerative disorder characterized by progressive neuronal loss in the frontal, temporal, and parietal lobes of the cerebral cortex as well as subcortical structures like the basal forebrain cholinergic system and the locus coeruleus within the brainstem. The clinical presentation of Alzheimer's disease is a progressive decline in a number of cognitive functions including short and long-term memory, spatial navigation, language fluency, impulse control, anhedonia, and social withdrawal. Neuronal atrophy in brains of Alzheimer's disease patients is linked to accumulation of extracellular and intracellular protein inclusions. Aggregates of insoluble amyloid-β (Aβ) protein are often found in the extracellular space, while neurofibrillary tangles (NFTs) of hyperphosphorylated tau proteins are usually found in intracellular compartments of affected neurons. These neuropathologies are considered to be important in the etiology of Alzheimer's disease.

Clinical management of Alzheimer's disease has employed pharmacological and behavioral interventions to mitigate the symptoms of the disorder. For example, acetylcholinesterase inhibitors have been used to elevate acetylcholine levels in the brain as a means to ameliorate cognitive deficits of Alzheimer's disease as this neurotransmitter is found to be depleted in Alzheimer's disease patients. Additionally, atypical antipsychotics are commonly prescribed to Alzheimer's disease patients for behavioral management. This strategy, however, is targeted at ameliorating the symptoms of the disease without addressing its development and progression. Unlike these treatments, the compositions and methods described herein provide the benefit of treating a different biochemical phenomenon that can underlie the development of Alzheimer's disease. As such, the compositions and methods described herein target the physiological cause of the disease, representing a potential curative therapy.

### Therapeutic Agents

Using the compositions and methods of the disclosure, a patient having Alzheimer's disease may be administered one or more agents that together augment the expression and/or activity of one or more proteins selected from APP, PSEN1, PSEN2, APOE, TOMM40, GAB2, APOC1, TREM2, ABI3, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1 L, STK24, DISC1, MPZL1, SLC4A1AP, TRIP4, MSRA, HS3ST1, ZNF224, and AP2A2, such as one or more agents that together augment the expression and/or activity of one or more proteins selected from PSEN1, GAB2, APOC1, TREM2, ABI3, BIN1, HLA-DRB5, HLA-DRB1, CD2AP, PTK2B, INPP5D, MEF2C, CD33, MS4A4A, RIN3, PICALM, CASS4, SORL1, PLCG2, SCIMP, FRMD4A, SPPL2A, MTHFD1 L, DISCI , TRIP4, and HS3ST1. Exemplary amino acid sequences of these proteins are set forth in Table 6, below. Also included in Table 6 are exemplary nucleic acid sequences of genes encoding each corresponding protein. Nucleic acid sequences are listed using European Nucleotide Archive (ENA) reference identification numbers.

TABLE 6
Exemplary amino acid and nucleic acid sequences of proteins that may be modulated for
the treatment of Alzheimer's disease

| | | | Reference for |
|---|---|---|---|
| | | | Exemplary |
| | | | Nucleic Acid |
| SEQ | | | Sequence |
| ID | | | Encoding |
| NO. | Protein | Exemplary Amino Acid Sequence of Protein Product | Protein Product |
| 1 | APP | MLPGLALLLLAAWTARALEVPTDGNAGLLAEPQIAMFCGRLNMEMNVQNGKWDSDPSGTK | ENA AK312326.1 |
| | | TCIDTKEGILQYCQEVYPELQITNVVEANQPVTIQNWCKRGRKQCKTHPHFVIPYRCLVG | |
| | | EFVSDALLVPDKCKFLHQERMDVCETHLHWHTVAKETCSEKSTNLHDYGMLLPCGIDKFR | |
| | | GVEFVCCPLAEESDNVDSADAEEDDSDVWWGGADTDYADGSEDKVVEVAEEEEVAEVEEE | |
| | | EADDDEDDEDGDEVEEEAEEPYEEATERTTSIATTTTTTTESVEEVVREVCSEQAETGPC | |
| | | RANISRWYFDVTEGKCAPFFYGGCGGNRNNFDTEEYCMAVCGSAMSQSLLKTTQEPLARD | |
| | | PVKLPTTAASTPDAVDKYLETPGDENEHAHFQKAKERLEAKHRERMSQVMREWEEAERQA | |
| | | KNLPKADKKAVIQHFQEKVESLEQEAANERQQLVETHMARVEAMLNDRRRLALENYITAL | |
| | | QAVPPRPRHVFNMLKKYVRAEQKDRQHTLKHFEHVRMVDPKKAAQIRSQVMTHLRVIYER | |
| | | MNQSLSLLYNVPAVAEEIQDEVDELLQKEQNYSDDVLANMISEPRISYGNDALMPSLTET | |
| | | KTTVELLPVNGEFSLDDLQPWHSFGADSVPANTENEVEPVDARPAADRGLTTRPGSGLTN | |
| | | IKTEEISEVKMDAEFRHDSGYEVHHQKLVFFAEDVGSNKGAIIGLMVGGVVIATVIVITL | |
| | | VMLKKKQYTSIHHGVVEVDAAVTPEERHLSKMQQNGYENPTYKFFEQMQN | |
| 2 | PSEN1 | MTELPAPLSYFQNAQMSEDNHLSNTVRSQNDNRERQEHNDRRSLGHPEPLSNGRPQGNSR | ENA U40379.1 |
| | | QVVEQDEEEDEELTLKYGAKHVIMLFVPVTLCMVVVVATIKSVSFYTRKDGQLIYTPFTE | |
| | | DTETVGQRALHSILNAAIMISVIVVMTILLVVLYKYRCYKVIHAWLIISSLLLLFFFSFI | |
| | | YLGEVEKTYNVAVDYITVALLIWNFGVVGMISIHWKGPLRLQQAYLIMISALMALVFIKY | |
| | | LPEWTAWLILAVISVYDLVAVLCPKGPLRMLVETAQERNETLFPALIYSSTMVWLVNMAE | |
| | | GDPEAQRRVSKNSKYNAESTERESQDTVAENDDGGFSEEWEAQRDSHLGPHRSTPESRAA | |
| | | VQELSSSILAGEDPEERGVKLGLGDFIFYSVLVGKASATASGDWNTTIACFVAILIGLCL | |
| | | TLLLLAIFKKALPALPISITFGLVFYFATDYLVQPFMDQLAFHQFYI | |
| 3 | PSEN2 | MLTFMASDSEEEVCDERTSLMSAESPTPRSCQEGRQGPEDGENTAQWRSQENEEDGEEDP | ENA L43964.1 |
| | | DRYVCSGVPGRPPGLEEELTLKYGAKHVIMLFVPVTLCMIVVVATIKSVRFYTEKNGQLI | |
| | | YTPFTEDTPSVGQRLLNSVLNTLIMISVIVVMTIFLVVLYKYRCYKFIHGWLIMSSLMLL | |
| | | FLFTYIYLGEVLKTYNVAMDYPTLLLTVWNFGAVGMVCIHWKGPLVLQQAYLIMISALMA | |
| | | LVFIKYLPEWSAWVILGAISVYDLVAVLCPKGPLRMLVETAQERNEPIFPALIYSSAMVW | |
| | | TVGMAKLDPSSQGALQLPYDPEMEEDSYDSFGEPSYPEVFEPPLTGYPGEELEEEEERGV | |
| | | KLGLGDFIFYSVLVGKAAATGSGDWNTTLACFVAILIGLCLTLLLLAVFKKALPALPISI | |
| | | TFGLIFYFSTDNLVRPFMDTLASHQLYI | |
| 105 | APOE | MKVLWAALLVTFLAGCQAKVEQAVETEPEPELRQQTEWQSGQRWELALGRFWDYLRWVQT | ENA AK314898.1 |
| | | LSEQVQEELLSSQVTQELRALMDETMKELKAYKSELEEQLTPVAEETRARLSKELQAAQA | |
| | | RLGADMEDVCGRLVQYRGEVQAMLGQSTEELRVRLASHLRKLRKRLLRDADDLQKRLAVY | |
| | | QAGAREGAERGLSAIRERLGPLVEQGRVRAATVGSLAGQPLQERAQAWGERLRARMEEMG | |
| | | SRTRDRLDEVKEQVAEVRAKLEEQAQQIRLQAEAFQARLKSWEEPLVEDMQRQWAGLVEK | |
| | | VQAAVGTSAAPVPSDNH | |
| 4 | TOMM40 | MGNVLAASSPPAGPPPPPAPALVGLPPPPPSPPGFTLPPLGGSLGAGTSTSRSSERTPGA | ENA AF043250.1 |
| | | ATASASGAAEDGACGCLPNPGTFEECHRKCKELFPIQMEGVKLTVNKGLSNHFQVNHTVA | |
| | | LSTIGESNYHFGVTYVGTKQLSPTEAFPVLVGDMDNSGSLNAQVIHQLGPGLRSKMAIQT | |
| | | QQSKFVNWQVDGEYRGSDFTAAVTLGNPDVLVGSGILVAHYLQSITPCLALGGELVYHRR | |
| | | PGEEGTVMSLAGKYTLNNWLATVTLGQAGMHATYYHKASDQLQVGVEFEASTRMQDTSVS | |
| | | FGYQLDLPKANLLFKGSVDSNWIVGATLEKKLPPLPLTLALGAFLNHRKNKFQCGFGLII | |
| | | G | |
| 5 | GAB2 | MSGGGDVVCTGWLRKSPPEKKLRRYAWKKRWFILRSGRMSGDPDVLEYYKNDHSKKPLRI | ENA B0131711.1 |
| | | INLNFCEQVDAGLTFNKKELQDSFVFDIKTSERTFYLVAETEEDMNKWVQSICQICGFNQ | |
| | | AEESTDSLRNVSSAGHGPRSSPAELSSSSQHLLRERKSSAPSHSSQPTLFTFEPPVSNHM | |
| | | QPTLSTSAPQEYLYLHQCISRRAENARSASFSQGTRASFLMRSDTAVQKLAQGNGHCVNG | |
| | | ISGQVHGFYSLPKPSRHNTEFRDSTYDLPRSLASHGHTKGSLTGSETDNEDVYTFKTPSN | |
| | | TLCREFGDLLVDNMDVPATPLSAYQIPRTFTLDKNHNAMTVATPGDSAIAPPPRPPKPSQ | |
| | | AETPRWGSPQQRPPISENSRSVAATIPRRNTLPAMDNSRLHRASSCETYEYPQRGGESAG | |
| | | RSAESMSDGVGSFLPGKMIVGRSDSTNSEDNYVPMNPGSSTLLAMERAGDNSQSVYIPMS | |
| | | PGAHHFDSLGYPSTTLPVHRGPSRGSEIQPPPVNRNLKPDRKAKPTPLDLRNNTVIDELP | |
| | | FKSPITKSWSRANHTFNSSSSQYCRPISTQSITSTDSGDSEENYVPMQNPVSASPVPSGT | |
| | | NSPAPKKSTGSVDYLALDFQPSSPSPHRKPSTSSVTSDEKVDYVQVDKEKTQALQNTMQE | |
| | | WTDVRQSSEPSKGAKL | |
| 6 | APOC1 | MRLFLSLPVLVVVLSIVLEGPAPAQGTPDVSSALDKLKEFGNTLEDKARELISRIKQSEL | ENA BT007142.1 |
| | | SAKMREWFSETFQKVKEKLKIDS | |
| 103 | TREM2 | MEPLRLLILLEVTELSGAHNTIVFQGVAGQSLQVSCPYDSMKHWGRRKAWCRQLGEKGPC | ENA AF213457.1 |
| | | QRVVSTHNLWLLSFLRRWNGSTAITDDTLGGTLTITLRNLQPHDAGLYQCQSLHGSEADT | |
| | | LRKVLVEVLADPLDHRDAGDLWFPGESESFEDAHVEHSISRSLLEGEIPFPPTSILLLLA | |
| | | CIFLIKILAASALWAAAWHGQKPGTHPPSELDCGHDPGYQLQTLPGLRDT | |
| 7 | ABI3 | mAELQQLQEFEIPTGREALRGNHSALLRVADYCEDNYVQATDKRKALEETMAFTTQALAS | ENA AB037886.1 |
| | | VAYQVGNLAGHTLRMLDLQGAALRQVEARVSTLGQMVNMHMEKVARREIGTLATVQRLPP | |
| | | GQKVIAPENLPPLTPYCRRPLNFGCLDDIGHGIKDLSTQLSRTGTLSRKSIKAPATPASA | |
| | | TLGRPPRIPEPVHLPVVPDGRLSAASSAFSLASAGSAEGVGGAPTPKGQAAPPAPPLPSS | |
| | | LDPPPPPAAVEVFQRPPTLEELSPPPPDEELPLPLDLPPPPPLDGDELGLPPPPPGFGPD | |
| | | EPSWVPASYLEKVVTLYPYTSQKDNELSFSEGTVICVTRRYSDGWCEGVSSEGTGFFPGN | |
| | | YVEPSC | |
| 8 | BIN1 | MAEMGSKGVTAGKIASNVQKKLTRAQEKVLQKLGKADETKDEQFEQCVQNFNKQLTEGTR | ENA AF004015.1 |
| | | LQKDLRTYLASVKAMHEASKKLNECLQEVYEPDWPGRDEANKIAENNDLLWMDYHQKLVD | |
| | | QALLTMDTYLGQFPDIKSRIAKRGRKLVDYDSARHHYESLQTAKKKDEAKIAKPVSLLEK | |
| | | AAPQWCQGKLQAHLVAQTNLLRNQAEEELIKAQKVFEEMNVDLQEELPSLWNSRVGFYVN | |
| | | TFQSIAGLEENFHKEMSKLNQNLNDVLVGLEKQHGSNTFTVKAQPSDNAPAKGNKSPSPP | |
| | | DGSPAATPEIRVNHEPEPAGGATPGATLPKSPSQLRKGPPVPPPPKHTPSKEVKQEQILS | |
| | | LFEDTFVPEISVTTPSQFEAPGPFSEQASLLDLDFDPLPPVTSPVKAPTPSGQSIPWDLW | |
| | | EPTESPAGSLPSGEPSAAEGTFAVSWPSQTAEPGPAQPAEASEVAGGTQPAAGAQEPGET | |
| | | AASEAASSSLPAVVVETFPATVNGTVEGGSGAGRLDLPPGFMFKVQAQHDYTATDTDELQ | |
| | | LKAGDVVLVIPFQNPEEQDEGWLMGVKESDWNQHKELEKCRGVFPENFTERVP | |
| 9 | CR1 | MGASSPRSPEPVGPPAPGLPFCCGGSLLAVVVLLALPVAWGQCNAPEWLPFARPTNLTDE | ENA Y00816.1 |
| | | FEFPIGTYLNYECRPGYSGRPFSIICLKNSVWTGAKDRCRRKSCRNPPDPVNGMVHVIKG | |
| | | IQFGSQIKYSCTKGYRLIGSSSATCIISGDTVIWDNETPICDRIPCGLPPTITNGDFIST | |
| | | NRENFHYGSVVTYRCNPGSGGRKVFELVGEPSIYCTSNDDQVGIWSGPAPQCIIPNKCTP | |
| | | PNVENGILVSDNRSLFSLNEVVEFRCQPGFVMKGPRRVKCQALNKWEPELPSCSRVCQPP | |
| | | PDVLHAERTQRDKDNESPGQEVEYSCEPGYDLRGAASMRCTPQGDWSPAAPTCEVKSCDD | |
| | | FMGQLLNGRVLFPVNLQLGAKVDFVCDEGFQLKGSSASYCVLAGMESLWNSSVPVCEQIF | |
| | | CPSPPVIPNGRHTGKPLEVFPFGKTVNYTCDPHPDRGTSFDLIGESTIRCTSDPQGNGVW | |
| | | SSPAPRCGILGHCQAPDHELFAKLKTQTNASDFPIGTSLKYECRPEYYGRPFSITCLDNL | |
| | | VWSSPKDVCKRKSCKTPPDPVNGMVHVITDIQVGSRINYSCTTGHRLIGHSSAECILSGN | |
| | | AAHWSTKPPICQRIPCGLPPTIANGDFISTNRENFHYGSVVTYRCNPGSGGRKVFELVGE | |
| | | PSIYCTSNDDQVGIWSGPAPQCIIPNKCTPPNVENGILVSDNRSLFSLNEVVEFRCQPGF | |
| | | VMKGPRRVKCQALNKWEPELPSCSRVCQPPPDVLHAERTQRDKDNFSPGQEVFYSCEPGY | |
| | | DLRGAASMRCTPQGDWSPAAPTCEVKSCDDFMGQLLNGRVLFPVNLQLGAKVDEVCDEGF | |
| | | QLKGSSASYCVLAGMESLWNSSVPVCEQIFCPSPPVIPNGRHTGKPLEVFPFGKAVNYTC | |
| | | DPHPDRGTSFDLIGESTIRCTSDPQGNGVWSSPAPRCGILGHCQAPDHFLFAKLKTQTNA | |
| | | SDFPIGTSLKYECRPEYYGRPFSITCLDNLVWSSPKDVCKRKSCKTPPDPVNGMVHVITD | |
| | | IQVGSRINYSCTTGHRLIGHSSAECILSGNTAHWSTKPPICQRIPCGLPPTIANGDFIST | |
| | | NRENFHYGSVVTYRCNLGSRGRKVFELVGEPSIYCTSNDDQVGIWSGPAPQCIIPNKCTP | |
| | | PNVENGILVSDNRSLFSLNEVVEFRCQPGFVMKGPRRVKCQALNKWEPELPSCSRVCQPP | |
| | | PEILHGEHTPSHQDNFSPGQEVFYSCEPGYDLRGAASLHCTPQGDWSPEAPRCAVKSCDD | |
| | | FLGQLPHGRVLFPLNLQLGAKVSFVCDEGFRLKGSSVSHCVLVGMRSLWNNSVPVCEHIF | |
| | | CPNPPAILNGRHTGTPSGDIPYGKEISYTCDPHPDRGMTFNLIGESTIRCTSDPHGNGVW | |
| | | SSPAPRCELSVRAGHCKTPEQFPFASPTIPINDFEFPVGTSLNYECRPGYFGKMFSISCL | |
| | | ENLVWSSVEDNCRRKSCGPPPEPFNGMVHINTDTQFGSTVNYSCNEGFRLIGSPSTTCLV | |
| | | SGNNVTWDKKAPICIISCEPPPTISNGDFYSNNRTSFHNGTVVTYQCHTGPDGEQLFEL | |
| | | VGERSIYCTSKDDQVGVWSSPPPRCISTNKCTAPEVENAIRVPGNRSFFSLTEIIRFRCQ | |
| | | PGFVMVGSHTVQCQTNGRWGPKLPHCSRVCQPPPEILHGEHTLSHQDNFSPGQEVFYSCE | |
| | | PSYDLRGAASLHCTPQGDWSPEAPRCTVKSCDDFLGQLPHGRVLLPLNLQLGAKVSFVCD | |
| | | EGFRLKGRSASHCVLAGMKALWNSSVPVCEQIFCPNPPAILNGRHTGTPFGDIPYGKEIS | |
| | | YACDTHPDRGMTFNLIGESSIRCTSDPQGNGVWSSPAPRCELSVPAACPHPPKIQNGHYI | |
| | | GGHVSLYLPGMTISYICDPGYLLVGKGFIFCTDQGIWSQLDHYCKEVNCSFPLFMNGISK | |
| | | ELEMKKVYHYGDYVTLKCEDGYTLEGSPWSQCQADDRWDPPLAKCTSRTHDALIVGTLSG | |
| | | TIFFILLIIFLSWIILKHRKGNNAHENPKEVAIHLHSQGGSSVHPRTLQTNEENSRVLP | |
| 10 | ABCA7 | MAFWTQLMLLLWKNFMYRRRQPVQLLVELLWPLFLFFILVAVRHSHPPLEHHECHFPNKP | ENA AF250238.1 |
| | | LPSAGTVPWLQGLICNVNNTCFPQLTPGEEPGRLSNFNDSLVSRLLADARTVLGGASAHR | |
| | | TLAGLGKLIATLRAARSTAQPQPTKQSPLEPPMLDVAELLTSLLRTESLGLALGQAQEPL | |
| | | HSLLEAAEDLAQELLALRSLVELRALLQRPRGTSGPLELLSEALCSVRGPSSTVGPSLNW | |
| | | YEASDLMELVGQLPESALPDSSLSPACSELIGALDSHPLSRLLWRRLKPLILGKLLFAPD | |
| | | TPFTRKLMAQVNRTFEELTLLRDVREVWEMLGPRIFTFMNDSSNVAMLQRLLQMQDEGRR | |
| | | QPRPGGRDHMEALRSFLDPGSGGYSWQDAHADVGHLVGTLGRVTECLSLDKLEAAPSEAA | |
| | | LVSRALQLLAEHRFWAGVVFLGPEDSSDPTHPTPDLGPGHVRIKIRMDIDVVTRTNKIR | |
| | | DRFWDPGPAADPLTDLRYVWGGFVYLQDLVERAAVRVLSGANPRAGLYLQQMPYPCYVDD | |
| | | VFLRVLSRSLPLFLTLAWIYSVTLTVKAVVREKETRLRDTMRAMGLSRAVLWLGWFLSCL | |
| | | GPFLLSAALLVLVLKLGDILPYSHPGVVFLFLAAFAVATVTQSFLLSAFFSRANLAAACG | |
| | | GLAYFSLYLPYVLCVAWRDRLPAGGRVAASLLSPVAFGFGCESLALLEEQGEGAQWHNVG | |
| | | TRPTROVESLAQVSGLLLLDAALYGLATWYLEAVCPGQYGIPEPWNFPFRRSYWCGPRPP | |
| | | KSPAPCPTPLDPKVLVEEAPPGLSPGVSVRSLEKRFPGSPQPALRGLSLDFYQGHITAFL | |
| | | GHNGAGKTTTLSILSGLFPPSGGSAFILGHDVRSSMAAIRPHLGVCPQYNVLFDMLTVDE | |
| | | HVWFYGRLKGLSAAVVGPEQDRLLQDVGLVSKQSVQTRHLSGGMQRKLSVAIAFVGGSQV | |
| | | VILDEPTAGVDPASRRGIWELLLKYREGRTLILSTHHLDEAELLGDRVAVVAGGRLCCCG | |
| | | SPLFLRRHLGSGYYLTLVKARLPLTTNEKADTDMEGSVDTRQEKKNGSQGSRVGTPQLLA | |
| | | LVQHWVPGARLVEELPHELVLVLPYTGAHDGSFATLFRELDTRLAELRLTGYGISDTSLE | |
| | | EIFLKVVEECAADTDMEDGSCGQHLCTGIAGLDVTLRLKMPPQETALENGEPAGSAPETD | |
| | | QGSGPDAVGRVQGWALTRQQLQALLLKRELLARRSRRGLFAQIVLPALEVGLALVFSLIV | |
| | | PPFGHYPALRLSPTMYGAQVSFFSEDAPGDPGRARLLEALLQEAGLEEPPVQHSSHRFSA | |
| | | PEVPAEVAKVLASGNWTPESPSPACQCSRPGARRLLPDCPAAAGGPPPPQAVTGSGEVVQ | |
| | | NLTGRNLSDFLVKTYPRLVRQGLKTKKWVNEVRYGGFSLGGRDPGLPSGQELGRSVEELW | |
| | | ALLSPLPGGALDRVLKNLTAWAHSLDAQDSLKIWFNNKGWHSMVAFVNRASNAILRAHLP | |
| | | PGPARHAHSITTLNHPLNLTKEQLSEGALMASSVDVLVSICVVFAMSFVPASFTLVLIEE | |
| | | RVTRAKHLQLMGGLSPTLYWLGNFLWDMCNYLVPACIVVLIFLAFQQRAYVAPANLPALL | |
| | | LLLLLYGWSITPLMYPASFFFSVPSTAYVVLTCINLFIGINGSMATFVLELFSDQKLQEV | |
| | | SRILKQVFLIFPHFCLGRGLIDMVRNQAMADAFERLGDRQFQSPLRWEVVGKNLLAMVIQ | |
| | | GPLFLLFTLLLQHRSQLLPQPRVRSLPLLGEEDEDVARERERVVQGATQGDVLVLRNLTK | |
| | | VYRGQRMPAVDRLCLGIPPGECFGLLGVNGAGKTSTFRMVTGDTLASRGEAVLAGHSVAR | |
| | | EPSAAHLSMGYCPQSDAIFELLTGREHLELLARLRGVPEAQVAQTAGSGLARLGLSWYAD | |
| | | RPAGTYSGGNKRKLATALALVGDPAVVFLDEPTTGMDPSARRFLWNSLLAVVREGRSVML | |
| | | TSHSMEECEALCSRLAIMVNGRFRCLGSPQHLKGRFAAGHTLTLRVPAARSQPAAAFVAA | |
| | | EFPGAELREAHGGRLRFQLPPGGRCALARVFGELAVHGAEHGVEDFSVSQTMLEEVFLYF | |
| | | SKDQGKDEDTEEQKEAGVGVDPAPGLQHPKRVSQFLDDPSTAETVL | |
| 11 | FERMT2 | MALDGIRMPDGCYADGTWELSVHVTDLNRDVTLRVTGEVHIGGVMLKLVEKLDVKKDWSD | ENA AF443279.1 |
| | | HALWWEKKRTWLLKTHWTLDKYGIQADAKLOFTPQHKLLRLQLPNMKYVKVKVNFSDRVF | |
| | | KAVSDICKTFNIRHPEELSLLKKPRDPTKKKKKKLDDQSEDEALELEGPLITPGSGSIYS | |
| | | SPGLYSKTMTPTYDAHDGSPLSPTSAWFGDSALSEGNPGILAVSQPITSPEILAKMFKPQ | |
| | | ALLDKAKINQGWLDSSRSLMEQDVKENEALLLRFKYYSFFDLNPKYDAIRINQLYEQAKW | |
| | | AILLEEIECTEEEMMMFAALQYHINKLSIMTSENHLNNSDKEVDEVDAALSDLEITLEGG | |
| | | KTSTILGDITSIPELADYIKVFKPKKLTLKGYKQYWCTFKDTSISCYKSKEESSGTPAHQ | |
| | | MNLRGCEVTPDVNISGQKFNIKLLIPVAEGMNEIWLRCDNEKQYAHWMAACRLASKGKTM | |
| | | ADSSYNLEVQNILSFLKMQHLNPDPQLIPEQITTDITPECLVSPRYLKKYKNKQITARIL | |
| | | EAHQNVAQMSLIEAKMRFIQAWQSLPEFGITHFIARFQGGKKEELIGIAYNRLIRMDAST | |
| | | GDAIKTWRFSNMKQWNVNWEIKMVTVEFADEVRLSFICTEVDCKVVHEFIGGYIFLSTRA | |
| | | KDQNESLDEEMFYKLTSGWV | |
| 12 | HLA- | MVCLKLPGGSYMAKLTVTLMVLSSPLALAGDTRPRFLQQDKYECHFFNGTERVRFLHRDI | ENA M20429.1 |
| | DRB5 | YNQEEDLRFDSDVGEYRAVTELGRPDAEYWNSQKDFLEDRRAAVDTYCRHNYGVGESFTV | |
| | | QRRVEPKVTVYPARTQTLQHHNLLVCSVNGFYPGSIEVRWFRNSQEEKAGVVSTGLIQNG | |
| | | DWTFQTLVMLETVPRSGEVYTCQVEHPSVTSPLTVEWRAQSESAQSKMLSGVGGFVLGLL | |
| | | FLGAGLFIYFKNQKGHSGLHPTGLVS | |
| 13 | HLA- | MVCLKLPGGSCMTALTVTLMVLSSPLALAGDTRPRFLWQLKFECHFFNGTERVRLLERCI | ENA X03069.1 |
| | DRB1 | YNQEESVRFDSDVGEYRAVTELGRPDAEYWNSQKDLLEQRRAAVDTYCRHNYGVGESFTV | |
| | | QRRVEPKVTVYPSKTQPLQHHNLLVCSVSGFYPGSIEVRWFRNGQEEKAGVVSTGLIQNG | |
| | | DWTFQTLVMLETVPRSGEVYTCQVEHPSVTSPLTVEWRARSESAQSKMLSGVGGFVLGLL | |
| | | FLGAGLFIYFRNQKGHSGLQPTGFLS | |
| 14 | CD2AP | MVDYIVEYDYDAVHDDELTIRVGEIIRNVKKLQEEGWLEGELNGRRGMFPDNFVKEIKRE | ENA AF146277.1 |
| | | TEFKDDSLPIKRERHGNVASLVQRISTYGLPAGGIQPHPQTKNIKKKTKKRQCKVLFEYI | |
| | | PQNEDELELKVGDIIDINEEVEEGWWSGTLNNKLGLFPSNFVKELEVTDDGETHEAQDDS | |
| | | ETVLAGPTSPIPSLGNVSETASGSVTQPKKIRGIGFGDIFKEGSVKLRTRTSSSETEEKK | |
| | | PEKPLILQSLGPKTQSVEITKTDTEGKIKAKEYCRTLFAYEGTNEDELTFKEGEIIHLIS | |
| | | KETGEAGWWRGELNGKEGVFPDNFAVQINELDKDFPKPKKPPPPAKAPAPKPELIAAEKK | |
| | | YFSLKPEEKDEKSTLEQKPSKPAAPQVPPKKPTPPTKASNLLRSSGTVYPKRPEKPVPPP | |
| | | PPIAKINGEVSSISSKFETEPVSKLKLDSEQLPLRPKSVDFDSLTVRTSKETDVVNFDDI | |
| | | ASSENLLHLTANRPKMPGRRLPGRFNGGHSPTHSPEKILKLPKEEDSANLKPSELKKDTC | |
| | | YSPKPSVYLSTPSSASKANTTAFLTPLEIKAKVETDDVKKNSLDELRAQIIELLCIVEAL | |
| | | KKDHGKELEKLRKDLEEEKTMRSNLEMEIEKLKKAVLSS | |
| 15 | PTK2B | MSGVSEPLSRVKLGTLRRPEGPAEPMVVVPVDVEKEDVRILKVCFYSNSFNPGKNFKLVK | ENA U33284.1 |
| | | CTVQTEIREIITSILLSGRIGPNIRLAECYGLRLKHMKSDEIHWLHPQMTVGEVQDKYEC | |
| | | LHVEAEWRYDLQIRYLPEDFMESLKEDRTTLLYFYQQLRNDYMQRYASKVSEGMALQLGC | |
| | | LELRRFFKDMPHNALDKKSNFELLEKEVGLDLFFPKQMQENLKPKQFRKMIQQTFQQYAS | |
| | | LREEECVMKFFNTLAGFANIDQETYRCELIQGWNITVDLVIGPKGIRQLTSQDAKPTCLA | |
| | | EFKQIRSIRCLPLEEGQAVLQLGIEGAPQALSIKTSSLAEAENMADLIDGYCRLQGEHQG | |
| | | SLIIHPRKDGEKRNSLPQIPMLNLEARRSHLSESCSIESDIYAEIPDETLRRPGGPQYGI | |
| | | AREDVVLNRILGEGFFGEVYEGVYTNHKGEKINVAVKTCKKDCTLDNKEKFMSEAVIMKN | |
| | | LDHPHIVKLIGIIEEEPTWIIMELYPYGELGHYLERNKNSLKVLTLVLYSLQICKAMAYL | |
| | | ESINCVHRDIAVRNILVASPECVKLGDFGLSRYIEDEDYYKASVTRLPIKWMSPESINFR | |
| | | RFTTASDVWMFAVCMWEILSFGKQPFFWLENKDVIGVLEKGDRLPKPDLCPPVLYTLMTR | |
| | | CWDYDPSDRPRFTELVCSLSDVYQMEKDIAMEQERNARYRTPKILEPTAFQEPPPKPSRP | |
| | | KYRPPPQTNLLAPKLQFQVPEGLCASSPTLTSPMEYPSPVNSLHTPPLHRHNVFKRHSMR | |
| | | EEDFIQPSSREEAQQLWEAEKVKMRQILDKQQKQMVEDYQWLRQEEKSLDPMVYMNDKSP | |
| | | LTPEKEVGYLEFTGPPQKPPRLGAQSIQPTANLDRTDDLVYLNVMELVRAVLELKNELCQ | |
| | | LPPEGYVVVVKNVGLILRKLIGSVDDLLPSLPSSSRTEIEGTQKLLNKDLAELINKMRLA | |
| | | QQNAVTSLSEECKRQMLTASHTLAVDAKNLLDAVDQAKVLANLAHPPAE | |
| 16 | CELF1 | MNGTLDHPDQPDLDAIKMFVGQVPRTWSEKDLRELFEQYGAVYEINVLRDRSQNPPQSKG | ENA AJ007988.1 |
| | | CCFVTFYTRKAALEAQNALHNMKVLPGMHHPIQMKPADSEKNNAVEDRKLFIGMISKKCT | |
| | | ENDIRVMFSSFGQIEECRILRGPDGLSRGCAFVTFTTRAMAQTAIKAMHQAQTMEGCSSP | |
| | | MVVKFADTQKDKEQKRMAQQLQQQMQQISAASVWGNLAGLNTLGPQYLALYLQLLQQTAS | |
| | | SGNLNTLSSLHPMGGLNAMQLQNLAALAAAASAAQNTPSGTNALTTSSSPLSVLTSSGSS | |
| | | PSSSSSNSVNPIASLGALQTLAGATAGLNVGSLAGMAALNGGLGSSGLSNGTGSTMEALT | |
| | | QAYSGIQQYAAAALPTLYNQNLLTQQSIGAAGSQKEGPEGANLFIYHLPQEFGDQDLLQM | |
| | | FMPFGNVVSAKVFIDKQTNLSKCFGFVSYDNPVSAQAAIQSMNGFQIGMKRLKVQLKRSK | |
| | | NDSKPY | |
| 17 | INPP5D | MVPCWNHGNITRSKAEELLSRTGKDGSFLVRASESISRAYALCVLYRNCVYTYRILPNED | ENA X98429.1 |
| | | DKFTVQASEGVSMRFFTKLDQLIEFYKKENMGLVTHLQYPVPLEEEDTGDDPEEDTVESV | |
| | | VSPPELPPRNIPLTASSCEAKEVPFSNENPRATETSRPSLSETLFQRLQSMDTSGLPEEH | |
| | | LKAIQDYLSTQLAQDSEFVKTGSSSLPHLKKLTTLLCKELYGEVIRTLPSLESLQRLFDQ | |
| | | QLSPGLRPRPQVPGEANPINMVSKLSQLTSLLSSIEDKVKALLHEGPESPHRPSLIPPVT | |
| | | FEVKAESLGIPQKMQLKVDVESGKLIIKKSKDGSEDKFYSHKKILQLIKSQKFLNKLVIL | |
| | | VETEKEKILRKEYVFADSKKREGFCQLLQQMKNKHSEQPEPDMITIFIGTWNMGNAPPPK | |
| | | KITSWFLSKGQGKTRDDSADYIPHDIYVIGTQEDPLSEKEWLEILKHSLQEITSVTEKTV | |
| | | AIHTLWNIRIVVLAKPEHENRISHICIDNVKTGIANTLGNKGAVGVSFMENGTSLGFVNS | |
| | | HLTSGSEKKLRRNQNYMNILRFLALGDKKLSPFNITHRFTHLFWFGDLNYRVDLPTWEAE | |
| | | TIIQKIKQQQYADLLSHDQLLTERREQKVFLHFEEEEITFAPTYRFERLTRDKYAYTKQK | |
| | | ATGMKYNLPSWCDRVLWKSYPLVHVVCQSYGSTSDIMTSDHSPVFATFEAGVTSQFVSKN | |
| | | GPGTVDSQGQIEFLRCYATLKTKSQTKFYLEFHSSCLESFVKSQEGENEEGSEGELVVKF | |
| | | GETLPKLKPIISDPEYLLDQHILISIKSSDSDESYGEGCIALRLEATETQLPIYTPLTHH | |
| | | GELTGHFQGEIKLQTSQGKTREKLYDFVKTERDESSGPKTLKSLTSHDPMKQWEVTSRAP | |
| | | PCSGSSITEIINPNYMGVGPFGPPMPLHVKQTLSPDQQPTAWSYDQPPKDSPLGPCRGES | |
| | | PPTPPGQPPISPKKFLPSTANRGLPPRTQESRPSDLGKNAGDTLPQEDLPLTKPEMFENP | |
| | | LYGSLSSFPKPAPRKDQESPKMPRKEPPPCPEPGILSPSIVLIKAQEADRGEGPGKQVPA | |
| | | PRLRSFTCSSSAEGRAAGGDKSQGKPKTPVSSQAPVPAKRPIKPSRSEINQQTPPTPTPR | |
| | | PPLPVKSPAVLHLQHSKGRDYRDNTELPHHGKHRPEEGPPGPLGRTAMQ | |
| 18 | MEF2C | MGRKKIQITRIMDERNRQVTFTKRKFGLMKKAYELSVLCDCEIALITENSTNKLFQYAST | ENA L08895.1 |
| | | DMDKVLLKYTEYNEPHESRTNSDIVETLRKKGLNGCDSPDPDADDSVGHSPESEDKYRKI | |
| | | NEDIDLMISRQRLCAVPPPNFEMPVSIPVSSHNSLVYSNPVSSLGNPNLLPLAHPSLQRN | |
| | | SMSPGVTHRPPSAGNTGGLMGGDLTSGAGTSAGNGYGNPRNSPGLLVSPGNLNKNMQAKS | |
| | | PPPMNLGMNNRKPDLRVLIPPGSKNTMPSVSEDVDLLLNQRINNSQSAQSLATPVVSVAT | |
| | | PTLPGQGMGGYPSAISTTYGTEYSLSSADLSSLSGENTASALHLGSVTGWQQQHLHNMPP | |
| | | SALSQLGACTSTHLSQSSNLSLPSTQSLNIKSEPVSPPRDRTTTPSRYPQHTRHEAGRSP | |
| | | VDSLSSCSSSYDGSDREDHRNEFHSPIGLTRPSPDERESPSVKRMRLSEGWAT | |
| 19 | ZCWPW1 | MMTTLQNKEECGKGPKRIFAPPAQKSYSLLPCSPNSPKEETPGISSPETEARISLPKASL | ENA AL136735.1 |
| | | KKKEEKATMKNVPSREQEKKRKAQINKQAEKKEKEKSSLTNAEFEEIVQIVLQKSLQECL | |
| | | GMGSGLDFAETSCAQPVVSTQSDKEPGITASATDTDNANGEEVPHTQEISVSWEGEAAPE | |
| | | IRTSKLGQPDPAPSKKKSNRLTLSKRKKEAHEKVEKTQGGHEHRQEDRLKKTVQDHSQIR | |
| | | DQQKGEISGFGQCLVWVQCSFPNCGKWRRLCGNIDPSVLPDNWSCDQNTDVQYNRCDIPE | |
| | | ETWTGLESDVAYASYIPGSIIWAKQYGYPWWPGMIESDPDLGEYELFTSHLDSLPSKYHV | |
| | | IFEGETVSRAWIPVNMLKNFQELSLELSVMKKRRNDCSQKLGVALMMAQEAEQISIQERV | |
| | | NLFGEWSRFNGSNSNGERKDLQLSGLNSPGSCLEKKEKEEELEKEEGEKTDPILPIRKRV | |
| | | KIQTQKTKPRGLGGDAGTADGRGRTLQRKIMKRSLGRKSTAPPAPRMGRKEGQGNSDSDQ | |
| | | PGPKKKFKAPQSKALAASFSEGKEVRTVPKNLGLSACKGACPSSAKEEPRHREPLTQEAG | |
| | | SVPLEDEASSDLDLEQLMEDVGRELGQSGELQHSNSDGEDFPVALFGK | |
| 20 | CD33 | MPLLLLLPLLWAGALAMDPNFWLQVQESVTVQEGLCVLVPCTFFHPIPYYDKNSPVHGYW | ENA M23197.1 |
| | | FREGAIISRDSPVATNKLDQEVQEETQGRFRLLGDPSRNNCSLSIVDARRRDNGSYFFRM | |
| | | ERGSTKYSYKSPQLSVHVTDLTHRPKILIPGTLEPGHSKNLTCSVSWACEQGTPPIFSWL | |
| | | SAAPTSLGPRTTHSSVLIITPRPQDHGTNLTCQVKFAGAGVTTERTIQLNVTYVPQNPTT | |
| | | GIFPGDGSGKQETRAGVVHGAIGGAGVTALLALCLCLIFFIVKTHRRKAARTAVGRNDTH | |
| | | PTTGSASPKHQKKSKLHGPTETSSCSGAAPTVEMDEELHYASLNFHGMNPSKDTSTEYSE | |
| | | VRTQ | |
| 21 | MS4A4A | MHQTYSRHCRPEESTFSAAMTTMQGMEQAMPGAGPGVPQLGNMAVIHSHLWKGLQEKFLK | ENA AB022821.1 |
| | | GEPKVLGVVQILTALMSLSMGITMMCMASNTYGSNPISVYIGYTIWGSVMFIISGSLSIA | |
| | | AGIRTTKGLVRGSLGMNITSSVLAASGILINTFSLAFYSFHHPYCNYYGNSNNCHGTMSI | |
| | | LMGLDGMVLLLSVLEFCIAVSLSAFGCKVLCCTPGGVVLILPSHSHMAETASPTPLNEV | |
| 22 | RIN3 | MIRHAGAPARGDPTGPVPVVGKGEEEEEEDGMRLCLPANPKNCLPHRRGISILEKLIKTC | ENA AB081753.1 |
| | | PVWLQLSLGQAEVARILHRVVAGMFLVRRDSSSKQLVLCVHFPSLNESSAEVLEYTIKEE | |
| | | KSILYLEGSALVFEDIERLIAFYCVSRDLLPFTLRLPQATLEASSFTDLETIANLGLGEW | |
| | | DSSLNPPQERGKPAEPPRDRAPGFPLVSSLRPTAHDANCACEIELSVGNDRLWFVNPIFI | |
| | | EDCSSALPTDQPPLGNCPARPLPPTSDATSPTSRWAPRRPPPPPPVLPLQPCSPAQPPVL | |
| | | PALAPAPACPLPTSPPVPAPHVTPHAPGPPDHPNQPPMMTCERLPCPTAGLGPLREEAMK | |
| | | PGAASSPLQQVPAPPLPAKKNLPTAPPRRRVSERVSLEDQSPGMAAEGDQLSLPPQGTSD | |
| | | GPEDTPRESTEQGQDTEVKASDPHSMPELPRTAKQPPVPPPRKKRISRQLASTLPAPLEN | |
| | | AELCTQAMALETPTPGPPREGQSPASQAGTQHPPAQATAHSQSSPEFKGSLASLSDSLGV | |
| | | SVMATDQDSYSTSSTEEELEQFSSPSVKKKPSMILGKARHRLSFASFSSMFHAFLSNNRK | |
| | | LYKKVVELAQDKGSYFGSLVQDYKVYSLEMMARQTSSTEMLQEIRTMMTQLKSYLLQSTE | |
| | | LKALVDPALHSEEELEAIVESALYKCVLKPLKEAINSCLHQIHSKDGSLQQLKENQLVIL | |
| | | ATTTTDLGVTTSVPEVPMMEKILQKFTSMHKAYSPEKKISILLKTCKLIYDSMALGNPGK | |
| | | PYGADDFLPVLMYVLARSNLTEMLLNVEYMMELMDPALQLGEGSYYLTTTYGALEHIKSY | |
| | | DKITVTRQLSVEVQDSIHRWERRRTLNKARASRSSVQDFICVSYLEPEQQARTLASRADT | |
| | | QAQALCAQCAEKFAVERPQAHRLFVLVDGRCFQLADDALPHCIKGYLLRSEPKRDFHFVY | |
| | | RPLDGGGGGGGGSPPCLVVREPNFL | |
| 23 | EPHA1 | MERRWPLGLGLVLLLCAPLPPGARAKEVTLMDTSKAQGELGWLLDPPKDGWSEQQQILNG | ENA M18391.1 |
| | | TPLYMYQDCPMQGRRDTDHWLRSNWIYRGEEASRVHVELQFTVRDCKSFPGGAGPLGCKE | |
| | | TFNLLYMESDQDVGIQLRRPLFQKVTTVAADQSFTIRDLVSGSVKLNVERCSLGRLTRRG | |
| | | LYLAFHNPGACVALVSVRVFYQRCPETLNGLAQFPDTLPGPAGLVEVAGTCLPHARASPR | |
| | | PSGAPRMHCSPDGEWLVPVGRCHCEPGYEEGGSGEACVACPSGSYRMDMDTPHCLTCPQQ | |
| | | STAESEGATICTCESGHYRAPGEGPQVACTGPPSAPRNLSFSASGTQLSLRWEPPADTGG | |
| | | RQDVRYSVRCSQCQGTAQDGGPCQPCGVGVHFSPGARGLTTPAVHVNGLEPYANYTFNVE | |
| | | AQNGVSGLGSSGHASTSVSISMGHAESLSGLSLRLVKKEPRQLELTWAGSRPRSPGANLT | |
| | | YELHVLNQDEERYQMVLEPRVLLTELQPDTTYIVRVRMLTPLGPGPFSPDHEFRTSPPVS | |
| | | RGLTGGEIVAVIFGLLLGAALLLGILVFRSRRAQRQRQQRQRDRATDVDREDKLWLKPYV | |
| | | DLQAYEDPAQGALDFTRELDPAWLMVDTVIGEGEFGEVYRGTLRLPSQDCKTVAIKTLKD | |
| | | TSPGGQWWNFLREATIMGQFSHPHILHLEGVVTKRKPIMIITEFMENGALDAFLREREDQ | |
| | | LVPGQLVAMLQGIASGMNYLSNHNYVHRDLAARNILVNQNLCCKVSDFGLTRLLDDFDGT | |
| | | YETQGGKIPIRWTAPEATAHRIFTTASDVWSFGIVMWEVLSFGDKPYGEMSNQEVMKSIE | |
| | | DGYRLPPPVDCPAPLYELMKNCWAYDRARRPHFQKLQAHLEQLLANPHSLRTIANFDPRM | |
| | | TLRLPSLSGSDGIPYRTVSEWLESIRMKRYILHFHSAGLDTMECVLELTAEDLTQMGITL | |
| | | PGHQKRILCSIQGFKD | |
| 24 | PICALM | MSGQSLTDRITAAQHSVTGSAVSKTVCKATTHEIMGPKKKHLDYLIQCTNEMNVNIPQLA | ENA U45976.1 |
| | | DSLFERTTNSSWVVVFKSLITTHHLMVYGNERFIQYLASRNTLFNLSNFLDKSGLQGYDM | |
| | | STFIRRYSRYLNEKAVSYRQVAFDFTKVKRGADGVMRTMNTEKLLKTVPIIQNQMDALLD | |
| | | FNVNSNELTNGVINAAFMLLFKDAIRLFAAYNEGIINLLEKYFDMKKNQCKEGLDIYKKF | |
| | | LTRMTRISEFLKVAEQVGIDRGDIPDLSQAPSSLLDALEQHLASLEGKKIKDSTAASRAT | |
| | | TLSNAVSSLASTGLSLTKVDEREKQAALEEEQARLKALKEQRLKELAKKPHTSLTTAASP | |
| | | VSTSAGGIMTAPAIDIFSTPSSSNSTSKLPNDLLDLQQPTFHPSVHPMSTASQVASTWGD | |
| | | PFSATVDAVDDAIPSLNPFLTKSSGDVHLSISSDVSTFTTRTPTHEMFVGFTPSPVAQPH | |
| | | PSAGLNVDFESVFGNKSTNVIVDSGGFDELGGLLKPTVASQNQNLPVAKLPPSKLVSDDL | |
| | | DSSLANLVGNLGIGNGTTKNDVNWSQPGEKKLTGGSNWQPKVAPTTAWNAATMAPPVMAY | |
| | | PATTPTGMIGYGIPPQMGSVPVMTQPTLIYSQPVMRPPNPFGPVSGAQIQFM | |
| 25 | CASS4 | MKGTGIMDCAPKALLARALYDNCPDCSDELAFSRGDILTILEQHVPESEGWWKCELHGRQ | ENA AJ276678.1 |
| | | GLAPANRLQILTEVAADRPCPPFLRGLEEAPASSEETYQVPTLPRPPTPGPVYEQMRSWA | |
| | | EGPQPPTAQVYEFPDPPTSARIICEKTLSFPKQAILTLPRPVRASLPTLPSQVYDVPTQH | |
| | | RGPVVLKEPEKQQLYDIPASPKKAGLHPPDSQASGQGVPLISVTTLRRGGYSTLPNPQKS | |
| | | EWIYDTPVSPGKASVRNTPLTSFAEESRPHALPSSSSTFYNPPSGRSRSLTPQLNNNVPM | |
| | | QKKLSLPEIPSYGFLVPRGTFPLDEDVSYKVPSSFLIPRVEQQNTKPNIYDIPKATSSVS | |
| | | QAGKELEKAKEVSENSAGHNSSWFSRRTTSPSPEPDRLSGSSSDSRASIVSSCSTTSTDD | |
| | | SSSSSSEESAKELSLDLDVAKETVMALQHKVVSSVAGLMLEVSRKWRFRDYLEANIDATE | |
| | | RSTDHIEESVREFLDFARGVHGTACNLTDSNLQNRIRDQMQTISNSYRILLETKESLDNR | |
| | | NWPLEVLVTDSVQNSPDDLERFVMVARMLPEDIKRFASIVIANGRLLFKRNCEKEETVQL | |
| | | TPNAEFKCEKYIQPPQRETESHQKSTPSTKQREDEHSSELLKKNRANICGQNPGPLIPQP | |
| | | SSQQTPERKPRLSEHCRLYFGALFKAISAFHGSLSSSQPAEIITQSKLVIMVGQKLVDTL | |
| | | CMETQERDVRNEILRGSSHLCSLLKDVALATKNAVLTYPSPAALGHLQAEAEKLEQHTRQ | |
| | | FRGTLG | |
| 26 | CLU | MMKTLLLFVGLLLTWESGQVLGDQTVSDNELQEMSNQGSKYVNKEIQNAVNGVKQIKTLI | ENA M25915.1 |
| | | EKTNEERKTLLSNLEEAKKKKEDALNETRESETKLKELPGVCNETMMALWEECKPCLKQT | |
| | | CMKFYARVCRSGSGLVGRQLEEFLNQSSPFYFWMNGDRIDSLLENDRQQTHMLDVMQDHF | |
| | | SRASSIIDELFQDRFFTREPQDTYHYLPFSLPHRRPHEFFPKSRIVRSLMPFSPYEPLNF | |
| | | HAMFQPFLEMIHEAQQAMDIHFHSPAFQHPPTEFIREGDDDRTVCREIRHNSTGCLRMKD | |
| | | QCDKCREILSVDCSTNNPSQAKLRRELDESLQVAERLTRKYNELLKSYQWKMLNTSSLLE | |
| | | QLNEQFNWVSRLANLTQGEDQYYLRVTTVASHTSDSDVPSGVTEVVVKLFDSDPITVTVP | |
| | | VEVSRKNPKFMETVAEKALQEYRKKHREE | |
| 27 | SORL1 | MATRSSRRESRLPFLFTLVALLPPGALCEVWTQRLHGGSAPLPQDRGFLVVQGDPRELRL | ENA Y08110.1 |
| | | WARGDARGASRADEKPLARKRSAALQPEPIKVYGQVSLNDSHNQMVVHWAGEKSNVIVAL | |
| | | ARDSLALARPKSSDVYVSYDYGKSFKKISDKLNFGLGNRSEAVIAQFYHSPADNKRYIFA | |
| | | DAYAQYLWITFDFCNTLQGFSIPFRAADLLLHSKASNLLLGFDRSHPNKQLWKSDDFGQT | |
| | | WIMIQEHVKSFSWGIDPYDKPNTIYIERHEPSGYSTVFRSTDFFQSRENQEVILEEVRDF | |
| | | QLRDKYMFATKVVHLLGSEQQSSVQLWVSFGRKPMRAAQFVTRHPINEYYIADASEDQVF | |
| | | VCVSHSNNRTNLYISEAEGLKFSLSLENVLYYSPGGAGSDTLVRYFANEPFADFHRVEGL | |
| | | QGVYIATLINGSMNEENMRSVITFDKGGTWEFLQAPAFTGYGEKINCELSQGCSLHLAQR | |
| | | LSQLLNLQLRRMPILSKESAPGLIIATGSVGKNLASKTNVYISSSAGARWREALPGPHYY | |
| | | TWGDHGGIITAIAQGMETNELKYSTNEGETWKTFIFSEKPVFVYGLLTEPGEKSTVFTIF | |
| | | GSNKENVHSWLILQVNATDALGVPCTENDYKLWSPSDERGNECLLGHKTVFKRRTPHATC | |
| | | FNGEDFDRPVVVSNCSCTREDYECDFGFKMSEDLSLEVCVPDPEFSGKSYSPPVPCPVGS | |
| | | TYRRTRGYRKISGDTCSGGDVEARLEGELVPCPLAEENEFILYAVRKSIYRYDLASGATE | |
| | | QLPLTGLRAAVALDFDYEHNCLYWSDLALDVIQRLCLNGSTGQEVIINSGLETVEALAFE | |
| | | PLSQLLYWVDAGFKKIEVANPDGDFRLTIVNSSVLDRPRALVLVPQEGVMFWTDWGDLKP | |
| | | GIYRSNMDGSAAYHLVSEDVKWPNGISVDDQWIYWTDAYLECTERITFSGQQRSVILDNL | |
| | | PHPYAIAVEKNEIYWDDWSQLSIFRASKYSGSQMEILANQLTGLMDMKIFYKGKNTGSNA | |
| | | CVPRPCSLLCLPKANNSRSCRCPEDVSSSVLPSGDLMCDCPQGYQLKNNTCVKQENTCLR | |
| | | NQYRCSNGNCINSIWWCDFDNDCGDMSDERNCPTTICDLDTQFRCQESGTCIPLSYKCDL | |
| | | EDDCGDNSDESHCEMHQCRSDEYNCSSGMCIRSSWVCDGDNDCRDWSDEANCTAIYHTCE | |
| | | ASNFQCRNGHCIPQRWACDGDTDCQDGSDEDPVNCEKKCNGFRCPNGTCIPSSKHCDGLR | |
| | | DCSDGSDEQHCEPLCIHFMDFVCKNRQQCLFESMVCDGIIQCRDGSDEDAAFAGCSQDPE | |
| | | FHKVCDEFGFQCQNGVCISLIWKCDGMDDCGDYSDEANCENPTEAPNCSRYFQFRCENGH | |
| | | CIPNRWKCDRENDCGDWSDEKDCGDSHILPFSTPGPSTCLPNYYRCSSGTCVMDTWVCDG | |
| | | YRDCADGSDEEACPLLANVTAASTPTQLGRCDRFEFECHQPKTCIPNWKRCDGHQDCQDG | |
| | | RDEANCPTHSTLTCMSREFQCEDGEACIVLSERCDGFLDCSDESDEKACSDELTVYKVQN | |
| | | LQWTADFSGDVTLTWMRPKKMPSASCVYNVYYRVVGESIWKTLETHSNKTNTVLKVLKPD | |
| | | TTYQVKVQVQCLSKAHNTNDEVTLRIPEGLPDAPRNLQLSLPREAEGVIVGHWAPPIHTH | |
| | | GLIREYIVEYSRSGSKMWASQRAASNFTEIKNLLVNTLYTVRVAAVTSRGIGNWSDSKSI | |
| | | TTIKGKVIPPPDIHIDSYGENYLSFTLTMESDIKVNGYVVNLFWAFDTHKQERRTLNFRG | |
| | | SILSHKVGNLTAHTSYEISAWAKTDLGDSPLAFEHVMTRGVRPPAPSLKAKAINQTAVEC | |
| | | TWTGPRNVVYGIFYATSFLDLYRNPKSLTTSLHNKTVIVSKDEQYLFLVRVVVPYQGPSS | |
| | | DYVVVKMIPDSRLPPRHLHVVHTGKTSVVIKWESPYDSPDQDLLYAVAVKDLIRKTDRSY | |
| | | KVKSRNSTVEYTLNKLEPGGKYHIIVQLGNMSKDSSIKITTVSLSAPDALKIITENDHVL | |
| | | LFWKSLALKEKHFNESRGYEIHMFDSAMNITAYLGNTTDNFFKISNLKMGHNYTFTVQAR | |
| | | CLFGNQICGEPAILLYDELGSGADASATQAARSTDVAAVVVPILFLILLSLGVGFAILYT | |
| | | KHRRLQSSFTAFANSHYSSRLGSAIFSSGDDLGEDDEDAPMITGFSDDVPMVIA | |
| 28 | PLCG2 | MSTTVNVDSLAEYEKSQIKRALELGTVMTVFSFRKSTPERRTVQVIMETRQVAWSKTADK | ENA M37238.1 |
| | | IEGFLDIMEIKEIRPGKNSKDFERAKAVRQKEDCCFTILYGTQFVLSTLSLAADSKEDAV | |
| | | NWLSGLKILHQEAMNASTPTIIESWLRKQIYSVDQTRRNSISLRELKTILPLINFKVSSA | |
| | | KFLKDKFVEIGAHKDELSFEQFHLFYKKLMFEQQKSILDEFKKDSSVFILGNTDRPDASA | |
| | | VYLHDFQRFLIHEQQEHWAQDLNKVRERMTKFIDDTMRETAEPFLFVDEFLTYLFSRENS | |
| | | IWDEKYDAVDMQDMNNPLSHYWISSSHNTYLTGDQLRSESSPEAYIRCLRMGCRCIELDC | |
| | | WDGPDGKPVIYHGWTRTTKIKFDDVVQAIKDHAFVTSSFPVILSIEEHCSVEQQRHMAKA | |
| | | FKEVFGDLLLTKPTEASADQLPSPSQLREKIIIKHKKLGPRGDVDVNMEDKKDEHKQQGE | |
| | | LYMWDSIDQKWTRHYCAIADAKLSFSDDIEQTMEEEVPQDIPPTELHFGEKWFHKKVEKR | |
| | | TSAEKLLQEYCMETGGKDGTFLVRESETFPNDYTLSFWRSGRVQHCRIRSTMEGGTLKYY | |
| | | LTDNLTFSSIYALIQHYRETHLRCAEFELRLTDPVPNPNPHESKPWYYDSLSRGEAEDML | |
| | | MRIPRDGAFLIRKREGSDSYAITFRARGKVKHCRINRDGRHFVLGTSAYFESLVELVSYY | |
| | | EKHSLYRKMRLRYPVTPELLERYNMERDINSLYDVSRMYVDPSEINPSMPQRTVKALYDY | |
| | | KAKRSDELSFCRGALIHNVSKEPGGWWKGDYGTRIQQYFPSNYVEDISTADFEELEKQII | |
| | | EDNPLGSLCRGILDLNTYNVVKAPQGKNQKSFVFILEPKQQGDPPVEFATDRVEELFEWF | |
| | | QSIREITWKIDTKENNMKYWEKNQSIAIELSDLVVYCKPTSKTKDNLENPDEREIRSFVE | |
| | | TKADSIIRQKPVDLLKYNQKGLTRVYPKGQRVDSSNYDPFRLWLCGSQMVALNFQTADKY | |
| | | MQMNHALFSLNGRTGYVLQPESMRTEKYDPMPPESQRKILMTLTVKVLGARHLPKLGRSI | |
| | | ACPFVEVEICGAEYDNNKFKTTVVNDNGLSPIWAPTQEKVTFEIYDPNLAFLRFVVYEED | |
| | | MFSDPNFLAHATYPIKAVKSGFRSVPLKNGYSEDIELASLLVFCEMRPVLESEEELYSSC | |
| | | RQLRRRQEELNNQLFLYDTHQNLRNANRDALVKEFSVNENQLQLYQEKCNKRLREKRVSN | |
| | | SKFYS | |
| 29 | SCIMP | MDTFTVQDSTAMSWWRNNFWIILAVAIIVVSVGLGLILYCVCKWQLRRGKKWEIAKPLKH | ENA AY358809.1 |
| | | KQVDEEKMYENVLNESPVQLPPLPPRNWPSLEDSSPQEAPSQPPATYSLVNKVKNKKTVS | |
| | | IPSYIEPEDDYDDVEIPANTEKASF | |
| 30 | FRMD4A | MAVQLVPDSALGLLMMTEGRRCQVHLLDDRKLELLVQPKLLAKELLDLVASHFNLKEKEY | ENA AB037715.1 |
| | | EGIAFTDETGHLNWLQLDRRVLEHDFPKKSGPVVLYFCVRFYIESISYLKDNATIELFFL | |
| | | NAKSCIYKELIDVDSEVVFELASYILQEAKGDFSSNEVVRSDLKKLPALPTQALKEHPSL | |
| | | AYCEDRVIEHYKKLNGQTRGQAIVNYMSIVESLPTYGVHYYAVKDKQGIPWWLGLSYKGI | |
| | | FQYDYHDKVKPRKIFQWRQLENLYFREKKFSVEVHDPRRASVIRRTFGHSGIAVHTWYAC | |
| | | PALIKSIWAMAISQHQFYLDRKQSKSKIHAARSLSEIAIDLTETGTLKTSKLANMGSKGK | |
| | | IISGSSGSLLSSGSQESDSSQSAKKDMLAALKSRQEALEETLRQRLEELKKLCLREAELT | |
| | | GKLPVEYPLDPGEEPPIVRRRIGTAFKLDEQKILPKGEEAELERLEREFAIQSQITEAAR | |
| | | RLASDPNVSKKLKKQRKTSYLNALKKLQEIENAINENRIKSGKKPTQRASLIIDDGNIAS | |
| | | EDSSLSDALVLEDEDSQVTSTISPLHSPHKGLPPRPPSHNRPPPPQSLEGLRQMHYHRND | |
| | | YDKSPIKPKMWSESSLDEPYEKVKKRSSHSHSSSHKRFPSTGSCALAGGGSNSLQNSPIR | |
| | | GLPHWNSQSSMPSTPDLRVRSPHYVHSTRSVDISPTRLHSLALHFRHRSSSLESQGKLLG | |
| | | SENDTGSPDFYTPRTRSSNGSDPMDDCSSCTSHSSSEHYYPAQMNANYSTLAEDSPSKAR | |
| | | QRQRQRQRAAGALGSASSGSMPNLAARGGAGGAGGAGGGVYLHSQSQPSSQYRIKEYPLY | |
| | | IEGGATPVVVRSLESDQEGHYSVKAQFKTSNSYTAGGLFKESWRGGGGDEGDTGRLTPSR | |
| | | SQILRIPSLGREGAHDKGAGRAAVSDELRQWYQRSTASHKEHSRLSHTSSTSSDSGSQYS | |
| | | TSSQSTFVAHSRVTRMPQMCKATSAALPQSQRSSTPSSEIGAITPSSPHHILTWQTGEAT | |
| | | ENSPILDGSESPPHQSTDE | |
| 31 | SPPL2A | MGPQRRLSPAGAALLWGFLLQLTAAQEAILHASGNGTTKDYCMLYNPYWTALPSTLENAT | ENA AJ345028.1 |
| | | SISLMNLTSTPLCNLSDIPPVGIKSKAVVVPWGSCHFLEKARIAQKGGAEAMLVVNNSVL | |
| | | FPPSGNRSEFPDVKILIAFISYKDFRDMNQTLGDNITVKMYSPSWPNFDYTMVVIFVIAV | |
| | | FTVALGGYWSGLVELENLKAVTTEDREMRKKKEEYLTFSPLTVVIFVVICCVMMVLLYFF | |
| | | YKWLVYVMIAIFCIASAMSLYNCLAALIHKIPYGQCTIACRGKNMEVRLIFLSGLCIAVA | |
| | | VVWAVFRNEDRWAWILQDILGIAFCLNLIKTLKLPNFKSCVILLGLLLLYDVFFVFITPF | |
| | | ITKNGESIMVELAAGPFGNNEKLPVVIRVPKLIYFSVMSVCLMPVSILGFGDIIVPGLLI | |
| | | AYCRRFDVQTGSSYIYYVSSTVAYAIGMILTFVVLVLMKKGQPALLYLVPCTLITASVVA | |
| | | WRRKEMKKFWKGNSYQMMDHLDCATNEENPVISGEQIVQQ | |
| 32 | MTHFD1L | MGTRLPLVLRQLRRPPQPPGPPRRLRVPCRASSGGGGGGGGGREGLLGQRRPQDGQARSS | ENA AY374130.1 |
| | | CSPGGRTPAARDSIVREVIQNSKEVLSLLQEKNPAFKPVLAIIQAGDDNLMQEINQNLAE | |
| | | EAGLNITHICLPPDSSEAEIIDEILKINEDTRVHGLALQISENLFSNKVLNALKPEKDVD | |
| | | GVTDINLGKLVRGDAHECFVSPVAKAVIELLEKSGVNLDGKKILVVGAHGSLEAALQCLF | |
| | | QRKGSMTMSIQWKTRQLQSKLHEADIVVLGSPKPELIPLTWIQPGTTVLNCSHDFLSGKV | |
| | | GCGSPRIHFGGLIEEDDVILLAAALRIQNMVSSGRRWLREQQHRRWRLHCLKLQPLSPVP | |
| | | SDIEISRGQTPKAVDVLAKEIGLLADEIEIYGKSKAKVRLSVLERLKDQADGKYVLVAGI | |
| | | TPTPLGEGKSTVTIGLVQALTAHLNVNSFACLRQPSQGPTFGVKGGAAGGGYAQVIPMEE | |
| | | FNLHLTGDTHAITAANNLLAAATDTRILHENTQTDKALYNRLVPLVNGVREFSEIQLARL | |
| | | KKLGINKTDPSTLTEEEVSKFARLDIDPSTITWQRVLDTNDRFLRKITIGQGNTEKGHYR | |
| | | QAQFDIAVASEIMAVLALTDSLADMKARLGRMVVASDKSGQPVTADDLGVTGALTVLMKD | |
| | | AIKPNLMQTLEGTPVFVHAGPFANIAHGNSSVLADKIALKLVGEEGFVVTEAGFGADIGM | |
| | | EKFFNIKCRASGLVPNVVVLVATVRALKMHGGGPSVTAGVPLKKEYTEENIQLVADGCCN | |
| | | LQKQIQITQLFGVPVVVALNVFKTDTRAEIDLVCELAKRAGAFDAVPCYHWSVGGKGSVD | |
| | | LARAVREAASKRSRFQFLYDVQVPIVDKIRTIAQAVYGAKDIELSPEAQAKIDRYTQQGF | |
| | | GNLPICMAKTHLSLSHQPDKKGVPRDFILPISDVRASIGAGFIYPLVGTMSTMPGLPTRP | |
| | | CFYDIDLDTETEQVKGLF | |
| 33 | STK24 | MDSRAQLWGLALNKRRATLPHPGGSTNLKADPEELFTKLEKIGKGSFGEVEKGIDNRTQK | ENA AF024636.1 |
| | | VVAIKIIDLEEAEDEIEDIQQEITVLSQCDSPYVTKYYGSYLKDTKLWIIMEYLGGGSAL | |
| | | DLLEPGPLDETQIATILREILKGLDYLHSEKKIHRDIKAANVLLSEHGEVKLADFGVAGQ | |
| | | LTDTQIKRNTFVGTPFWMAPEVIKQSAYDSKADIWSLGITAIELARGEPPHSELHPMKVL | |
| | | FLIPKNNPPTLEGNYSKPLKEFVEACLNKEPSFRPTAKELLKHKFILRNAKKTSYLTELI | |
| | | DRYKRWKAEQSHDDSSSEDSDAETDGQASGGSDSGDWIFTIREKDPKNLENGALQPSDLD | |
| | | RNKMKDIPKRPFSQCLSTIISPLFAELKEKSQACGGNLGSIEELRGAIYLAEEACPGISD | |
| | | TMVAQLVQRLQRYSLSGGGTSSH | |
| 34 | DISC1 | MPGGGPQGAPAAAGGGGVSHRAGSRDCLPPAACFRRRRLARRPGYMRSSTGPGIGELSPA | ENA AF222980.1 |
| | | VGTLFRFPGGVSGEESHHSESRARQCGLDSRGLLVRSPVSKSAAAPTVTSVRGTSAHFGI | |
| | | QLRGGTRLPDRLSWPCGPGSAGWQQEFAAMDSSETLDASWEAACSDGARRVRAAGSLPSA | |
| | | ELSSNSCSPGCGPEVPPTPPGSHSAFTSSFSFIRLSLGSAGERGEAEGCPPSREAESHCQ | |
| | | SPQEMGAKAASLDGPHEDPRCLSRPFSLLATRVSADLAQAARNSSRPERDMHSLPDMDPG | |
| | | SSSSLDPSLAGCGGDGSSGSGDAHSWDTLLRKWEPVLRDCLLRNRRQMEVISLRLKLQKL | |
| | | QEDAVENDDYDKAETLQQRLEDLEQEKISLHFQLPSRQPALSSFLGHLAAQVQAALRRGA | |
| | | TQQASGDDTHTPLRMEPRLLEPTAQDSLHVSITRRDWLLQEKQQLQKEIEALQARMFVLE | |
| | | AKDQQLRREIEEQEQQLQWQGCDLTPLVGQLSLGQLQEVSKALQDTLASAGQIPFHAEPP | |
| | | ETIRSLQERIKSLNLSLKEITTKVCMSEKFCSTLRKKVNDIETQLPALLEAKMHAISGNH | |
| | | FWTAKDLTEEIRSLTSEREGLEGLLSKLLVLSSRNVKKLGSVKEDYNRLRREVEHQETAY | |
| | | ETSVKENTMKYMETLKNKLCSCKCPLLGKVWEADLEACRLLIQSLQLQEARGSLSVEDER | |
| | | QMDDLEGAAPPIPPRLHSEDKRKTPLKVLEEWKTHLIPSLHCAGGEQKEESYILSAELGE | |
| | | KCEDIGKKLLYLEDQLHTAIHSHDEDLIQSLRRELQMVKETLQAMILQLQPAKEAGEREA | |
| | | AASCMTAGVHEAQA | |
| 35 | MPZL1 | MAASAGAGAVIAAPDSRRWLWSVLAAALGLLTAGVSALEVYTPKEIFVANGTQGKLTCKF | ENA AF087020.1 |
| | | KSTSTTGGLTSVSWSFQPEGADTTVSFFHYSQGQVYLGNYPPFKDRISWAGDLDKKDASI | |
| | | NIENMQFIHNGTYICDVKNPPDIVVQPGHIRLYVVEKENLPVFPVWVVVGIVTAVVLGLT | |
| | | LLISMILAVLYRRKNSKRDYTGCSTSESLSPVKQAPRKSPSDTEGLVKSLPSGSHQGPVI | |
| | | YAQLDHSGGHHSDKINKSESVVYADIRKN | |
| 36 | SLC4A1AP | MLAPLRNAPGREGATSPSPPTDATGSLGEWDVDRNVKTEGWVSKERISKLHRLRMADILS | ENA AY028435.1 |
| | | QSETLASQDLSGDFKKPALPVSPAARSKAPASSSSNPEEVQKEGPTALQDSNSGEPDIPP | |
| | | PQPDCGDFRSLQEEQSRPPTAVSSPGGPARAPPYQEPPWGGPATAPYSLETLKGGTILGT | |
| | | RSLKGTSYCLFGRLSGCDVCLEHPSVSRYHAVLQHRASGPDGECDSNGPGFYLYDLGSTH | |
| | | GTFLNKTRIPPRTYCRVHVGHVVRFGGSTRLFILQGPEEDREAESELTVTQLKELRKQQQ | |
| | | ILLEKKMLGEDSDEEEEMDTSERKINAGSQDDEMGCTWGMGEDAVEDDAEENPIVLEFQQ | |
| | | EREAFYIKDPKKALQGFEDREGEELEYEEDEQGHSTWLCRVRLPVDDSTGKQLVAEAIHS | |
| | | GKKKEAMIQCSLEACRILDTLGLLRQEAVSRKRKAKNWEDEDFYDSDDDTFLDRTGLIEK | |
| | | KRLNRMKKAGKIDEKPETFESLVAKLNDAERELSEISERLKASSQVLSESPSQDSLDAFM | |
| | | SEMKSGSTLDGVSRKKLHLRTFELRKEQQRLKGLIKIVKPAEIPELKKTETQTTGAENKA | |
| | | KKLTLPLFGAMKGGSKFKLKTGTVGKLPPKRPELPPTLMRMKDEPEVEEEEEEEEEEEKE | |
| | | KEEHEKKKLEDGSLSRPQPEIEPEAAVQEMRPPTDLTHFKETQTHENMSQLSEEEQNKDY | |
| | | QDCSKTTSLCAGPSASKNEYEKSRGELKKKKTPGPGKLPPTLSSKYPEDDPDYCVWVPPE | |
| | | GQSGDGRTHLNDKYGY | |
| 37 | TRIP4 | MAVAGAVSGEPLVHWCTQQLRKTFGLDVSEEIIQYVLSIESAEEIREYVTDLLQGNEGKK | ENA AF168418.1 |
| | | GQFTEELITKWQKNDQELISDPLQQCFKKDEILDGQKSGDHLKRGRKKGRNRQEVPAFTE | |
| | | PDTTAEVKTPFDLAKAQENSNSVKKKTKFVNLYTREGQDRLAVLLPGRHPCDCLGQKHKL | |
| | | INNCLICGRIVCEQEGSGPCLFCGTLVCTHEEQDILQRDSNKSQKLLKKLMSGVENSGKV | |
| | | DISTKDLLPHQELRIKSGLEKAIKHKDKLLEFDRTSIRRTQVIDDESDYFASDSNQWLSK | |
| | | LERETLQKREEELRELRHASRLSKKVTIDFAGRKILEEENSLAEYHSRLDETIQATANGT | |
| | | LNQPLTKLDRSSEEPLGVLVNPNMYQSPPQWVDHTGAASQKKAFRSSGFGLEFNSFQHQL | |
| | | RIQDQEFQEGFDGGWCLSVHQPWASLLVRGIKRVEGRSWYTPHRGRLWIAATAKKPSPQE | |
| | | VSELQATYRLLRGKDVEFPNDYPSGCLLGCVDLIDCLSQKQFKEQFPDISQESDSPFVFI | |
| | | CKNPQEMVVKFPIKGNPKIWKLDSKIHQGAKKGLMKQNKAV | |
| 38 | MSRA | MLSATRRACQLLLLHSLFPVPRMGNSASNIVSPQEALPGRKEQTPVAAKHHVNGNRTVEP | ENA AJ242973.1 |
| | | FPEGTQMAVFGMGCFWGAERKFWVLKGVYSTQVGFAGGYTSNPTYKEVCSEKTGHAEVVR | |
| | | VVYQPEHMSFEELLKVFWENHDPTQGMRQGNDHGTQYRSAIYPTSAKQMEAALSSKENYQ | |
| | | KVLSEHGFGPITTDIREGQTFYYAEDYHQQYLSKNPNGYCGLGGTGVSCPVGIKK | |
| 39 | HS3ST1 | MAALLLGAVLLVAQPQLVPSRPAELGQQELLRKAGTLQDDVRDGVAPNGSAQQLPQTIII | ENA AF019386.1 |
| | | GVRKGGTRALLEMLSLHPDVAAAENEVHFEDWEEHYSHGLGWYLSQMPFSWPHQLTVEKT | |
| | | PAYETSPKVPERVYSMNPSIRLLLILRDPSERVLSDYTQVFYNHMQKHKPYPSIEEFLVR | |
| | | DGRLNVDYKALNRSLYHVHMQNWLRFFPLRHIHIVDGDRLIRDPFPEIQKVERFLKLSPQ | |
| | | INASNFYFNKTKGFYCLRDSGRDRCLHESKGRAHPQVDPKLLNKLHEYFHEPNKKFFELV | |
| | | GRTFDWH | |
| 40 | ZNF224 | MTTFKEAMTFKDVAVVFTEEELGLLDLAQRKLYRDVMLENFRNLLSVGHQAFHRDTFHFL | ENA AF187990.2 |
| | | REEKIWMMKTAIQREGNSGDKIQTEMETVSEAGTHQEWSFQQIWEKIASDLTRSQDLMIN | |
| | | SSQFSKEGDFPCQTEAGLSVIHTRQKSSQGNGYKPSFSDVSHFDFHQQLHSGEKSHTCDE | |
| | | CGKNFCYISALRIHQRVHMGEKCYKCDVCGKEFSQSSHLQTHQRVHTGEKPFKCVECGKG | |
| | | FSRRSALNVHHKLHTGEKPYNCEECGKAFIHDSQLQEHQRIHIGEKPFKCDICGKSFCGR | |
| | | SRLNRHSMVHTAEKPFRCDTCDKSFRQRSALNSHRMIHTGEKPYKCEECGKGFICRRDLY | |
| | | THHMVHTGEKPYNCKECGKSFRWASCLLKHQRVHSGEKPFKCEECGKGFYTNSQCYSHQR | |
| | | SHSGEKPYKCVECGKGYKRRLDLDFHQRVHTGEKLYNCKECGKSFSRAPCLLKHERLHSG | |
| | | EKPFQCEECGKRFTQNSHLHSHQRVHTGEKPYKCEKCGKGYNSKFNLDMHQKVHTGERPY | |
| | | NCKECGKSFGWASCLLKHQRLHSGEKPFKCEECGKRFTQNSQLHSHQRVHTGEKPYKCDE | |
| | | CGKGFSWSSTRLTHQRRHSRETPLKCEQHGKNIVQNSFSKVQEKVHSVEKPYKCEDCGKG | |
| | | YNRRLNLDMHQRVHMGEKTWKCRECDMCFSQASSLRLHQNVHVGEKP | |
| 41 | AP2A2 | MPAVSKGDGMRGLAVFISDIRNCKSKEAEIKRINKELANIRSKFKGDKALDGYSKKKYVC | ENA AB020706.2 |
| | | KLLFIFLLGHDIDFGHMEAVNLLSSNRYTEKQIGYLFISVLVNSNSELIRLINNAIKNDL | |
| | | ASRNPTFMGLALHCIASVGSREMAEAFAGEIPKVLVAGDTMDSVKQSAALCLLRLYRTSP | |
| | | DLVPMGDWTSRVVHLLNDQHLGVVTAATSLITTLAQKNPEEFKTSVSLAVSRLSRIVTSA | |
| | | STDLQDYTYYFVPAPWLSVKLLRLLQCYPPPDPAVRGRLTECLETILNKAQEPPKSKKVQ | |
| | | HSNAKNAVLFEAISLIIHHDSEPNLLVRACNQLGQFLQHRETNLRYLALESMCTLASSEF | |
| | | SHEAVKTHIETVINALKTERDVSVRQRAVDLLYAMCDRSNAPQIVAEMLSYLETADYSIR | |
| | | EEIVLKVAILAEKYAVDYTWYVDTILNLIRIAGDYVSEEVWYRVIQIVINRDDVQGYAAK | |
| | | TVFEALQAPACHENLVKVGGYILGEFGNLIAGDPRSSPLIQFHLLHSKFHLCSVPTRALL | |
| | | LSTYIKFVNLFPEVKPTIQDVLRSDSQLRNADVELQQRAVEYLRLSTVASTDILATVLEE | |
| | | MPPFPERESSILAKLKKKKGPSTVTDLEDTKRDRSVDVNGGPEPAPASTSAVSTPSPSAD | |
| | | LLGLGAAPPAPAGPPPSSGGSGLLVDVFSDSASVVAPLAPGSEDNFARFVCKNNGVLFEN | |
| | | QLLQIGLKSEFRQNLGRMFIFYGNKTSTQFLNFTPTLICSDDLQPNLNLQTKPVDPTVEG | |
| | | GAQVQQVVNIECVSDFTEAPVLNIQFRYGGTFQNVSVQLPITLNKFFQPTEMASQDFFQR | |
| | | WKQLSNPQQEVQNIFKAKHPMDTEVTKAKIIGFGSALLEEVDPNPANFVGAGIIHTKTTQ | |
| | | IGCLLRLEPNLQAQMYRLTLRTSKEAVSQRLCELLSAQF | |

Agents that elevate the expression and/or activity level of one or more of the foregoing proteins that may be used in conjunction with the compositions and methods of the disclosure include nucleic acids that encode the protein or plurality of proteins (e.g., nucleic acids capable of expression in macrophages or microglia). Such nucleic acid molecules may be provided to a patient (e.g., a patient having Alzheimer's disease) in the form, for example, of a population of cells, such as a population of cells (e.g., pluripotent cells, ESCs, iPSCs, multipotent cells, CD34+ cells, HSCs, MPCs, BLPCs, monocytes, macrophages, microglial progenitor cells, or microglia) that contain the nucleic acid molecules. Such cells may be modified ex vivo so as to express the nucleic acid molecule(s) of interest, for example, using transfection and transduction methods described herein. Additionally or alternatively, nucleic acid molecules encoding one or more of the proteins of interest may be provided to the patient in the form of one or more viral vectors that collectively encode the one or more proteins. Exemplary viral vectors that may be used in conjunction with the compositions and methods of the disclosure include Retroviridae family viral vectors, such as a lentivirus, alpharetrovirus, or gammaretrovirus, among others described herein. In some embodiments, the nucleic acid molecule(s) are administered directly to the patient. Additional agents that may be provided to a patient for the purpose of augmenting the level of one or more of the foregoing proteins include interfering RNA molecules, such as siRNA, shRNA, and miRNA molecules, as well as small molecule agents that modulate the expression of one or more of the above proteins, in addition to the one or more of the above proteins themselves.

### Parkinson's Disease

Parkinson's disease is a progressive disorder that affects movement, and it is recognized as the second most common neurodegenerative disease after Alzheimer's disease. Common symptoms of Parkinson's disease include resting tremor, rigidity, and bradykinesia, and non-motor symptoms, such as depression, constipation, pain, sleep disorders, genitourinary problems, cognitive decline, and olfactory dysfunction, are also increasingly being associated with this disorder. A key feature of Parkinson's disease is the death of dopaminergic neurons in the substantia nigra pars compacta, and, for that reason, most current treatments for PD focus on increasing dopamine. Another well-known neuropathological hallmark of Parkinson's disease is the presence of Lewy bodies containing α-synuclein in brain regions affected by PD, which are thought to contribute to the disease.

Parkinson's is thought to result from a combination of genetic and environmental risk factors. There is no single gene responsible for all Parkinson's disease cases, and the vast majority of Parkinson's disease cases seem to be sporadic and not directly inherited. Mutations in the genes encoding parkin, PTEN-induced putative kinase 1 (PINK1), leucine-rich repeat kinase 2 (LRRK2), and Parkinsonism-associated deglycase (DJ-1) have been found to be associated with Parkinson's disease, but they represent only a small subset of the total number of Parkinson's disease cases. Occupational exposure to some pesticides and herbicides has also been proposed as a risk factor for Parkinson's disease.

### Glucocerebrosidase-Associated Parkinson's Disease

Recent studies have shown a link between mutations in the GBA gene and increased risk of PD, with more severe mutations imparting higher levels of risk. Glucocerebrosidase is a lysosomal enzyme responsible for the metabolism of glucocerebroside (also known as glucosylceramide) to glucose and ceramide. It plays an important role in sphingolipid degradation, especially in the macrophage/monocyte cell lineage. Reduced GBA activity has been reported in the substantia nigra, cerebellum, and caudate of PD patients, although GBA activity has also been shown to decrease with age (see Alcalay et al., Brain 138:2648 (2015), incorporated herein by reference as it pertains to GBA activity in PD). Severely pathogenic mutations include c.84GGIns, IVS2+1 G>A, p.V394L, p.D409H, p.L444P and RecTL, which are linked to a 9.92 to 21.29 odds-ratio of developing PD. Mild GBA mutations p.N370S and p.R496H are linked to an odds-ratio of 2.84-4.94 of developing PD. The mutation p.E326K has also been identified as a PD risk factor. GBA mutations are discussed in in Barkhuizen et al., Neurochemistry International 93:6 (2016) and Sidransky and Lopez, Lancet Neurol. 11:986 (2012), the disclosures of which are incorporated herein by reference as they pertain to human GBA mutations. These mutations may also elicit a gain of toxic function by activating endoplasmic reticulum (ER) stress as the mutant protein is trapped in the ER. Markers of ER stress are elevated in PD brains with GBA mutations, and dysregulation of ER calcium stores have been reported in cell models containing GBA mutations associated with PD. Additionally, these mutants could increase the total burden of to-be-degraded misfolded polypeptides in neural cells resulting in altered cellular function due to a diversion of cellular resources. GBA mutations resulting in a gain of toxic function and/or altered cellular function due to a diversion of cellular resources are discussed in Gregg et al., Ann. Neurol. 72:455-463 (2012), Schondorf et al., Nat. Commun. 5:4028 (2014), Kilpatrick et al., Cell Calcium. 59:12-20 (2016), and Cullen et al., Ann. Neuro1.69:940-953 (2011), the disclosure of which are incorporated herein by reference as they pertain to human GBA mutations. Studies in rodent models of PD have also suggested a link between GBA activity and α-synuclein accumulation, as described in Rocha et al., Antioxidants & Redox Signaling 23: 550 (2015) and Rocha et al., Neurobiology of Disease 82:495 (2015), the disclosures of which are disclosed herein by reference as they relate to the relationship between GBA and a-synuclein.

### Therapeutic Agents

Using the compositions and methods of the disclosure, a patient having Parkinson's disease may be administered one or more agents that together augment the expression and/or activity of one or more proteins selected from FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1 B, NUCKS1, and ACMSD, such as one or more agents that together augment the expression and/or activity of one or more proteins selected from FCGR2A, SCAF11, DNAJC13, GCH1, LRRK2, GBA, GAK, FGF20, HLA-DQB1, and NOD2. Exemplary amino acid sequences of these proteins are set forth in Table 7, below. Also included in Table 7 are exemplary nucleic acid sequences of genes encoding each corresponding protein. Nucleic acid sequences are listed using ENA reference identification numbers.

TABLE 7
Exemplary amino acid and nucleic acid sequences of proteins that may be modulated
for the treatment of Parkinson's disease

| | | | Reference for |
|---|---|---|---|
| | | | Exemplary |
| | | | Nucleic Acid |
| SEQ | | | Sequence |
| ID | | | Encoding |
| NO. | Protein | Exemplary Amino Acid Sequence of Protein Product | Protein Product |
| 42 | VPS1 | MEALIPVINKLQDVFNTVGADIIQLPQIVVVGTQSSGKSSVLESLVGRDLLPRGTGIVTR | ENA AB006965.1 |
| | | RPLILQLVHVSQEDKRKTTGEENGVEAEEWGKFLHTKNKLYTDFDEIRQEIENETERISG | |
| | | NNKGVSPEPIHLKIFSPNVVNLTLVDLPGMTKVPVGDQPKDIELQIRELILRFISNPNSI | |
| | | ILAVTAANTDMATSEALKISREVDPDGRRTLAVITKLDLMDAGTDAMDVLMGRVIPVKLG | |
| | | IIGVVNRSQLDINNKKSVTDSIRDEYAFLQKKYPSLANRNGTKYLARTLNRLLMHHIRDC | |
| | | LPELKTRINVLAAQYQSLLNSYGEPVDDKSATLLQLITKFATEYCNTIEGTAKYIETSEL | |
| | | CGGARICYIFHETFGRTLESVDPLGGLNTIDILTAIRNATGPRPALFVPEVSFELLVKRQ | |
| | | IKRLEEPSLRCVELVHEEMQRIIQHCSNYSTQELLRFPKLHDAIVEVVTCLLRKRLPVIN | |
| | | EMVHNLVAIELAYINIKHPDFADACGLMNNNIEEQRRNRLARELPSAVSRDKSSKVPSAL | |
| | | APASQEPSPAASAEADGKLIQDSRRETKNVASGGGGVGDGVQEPTTGNWRGMLKTSKAEE | |
| | | LLAEEKSKPIPIMPASPQKGHAVNLLDVPVPVARKLSAREQRDCEVIERLIKSYFLIVRK | |
| | | NIQDSVPKAVMHFLVNHVKDTLQSELVGQLYKSSLLDDLLTESEDMAQRRKEAADMLKAL | |
| | | QGASQIIAEIRETHLW | |
| 43 | SCARB | MGRCCFYTAGTLSLLLLVTSVTLLVARVFQKAVDQSIEKKIVLRNGTEAFDSWEKPPLPV | ENA D12676.1 |
| | 2 | YTQFYFFNVTNPEEILRGETPRVEEVGPYTYRELRNKANIQFGDNGTTISAVSNKAYVFE | |
| | | RDQSVGDPKIDLIRTLNIPVLTVIEWSQVHFLREIIEAMLKAYQQKLFVTHTVDELLWGY | |
| | | KDEILSLIHVFRPDISPYEGLEYEKNGTNDGDYVELTGEDSYLNFTKIVEWNGKTSLDWW | |
| | | ITDKCNMINGTDGDSFHPLITKDEVLYVFPSDFCRSVYITFSDYESVQGLPAFRYKVPAE | |
| | | ILANTSDNAGFCIPEGNCLGSGVLNVSICKNGAPIIMSFPHFYQADERFVSAIEGMHPNQ | |
| | | EDHETFVDINPLTGIILKAAKRFQINIYVKKLDDEVETGDIRTMVFPVMYLNESVHIDKE | |
| | | TASRLKSMINTTLIITNIPYIIMALGVFFGLVFTWLACKGQGSMDEGTADERAPLIRT | |
| 44 | GPNMB | MECLYYFLGFLLLAARLPLDAAKRFHDVLGNERPSAYMREHNQLNGWSSDENDWNEKLYP | ENA X76534.1 |
| | | VWKRGDMRWKNSWKGGRVQAVLTSDSPALVGSNITFAVNLIFPRCQKEDANGNIVYEKNC | |
| | | RNEAGLSADPYVYNWTAWSEDSDGENGTGQSHHNVFPDGKPFPHHPGWRRWNFIYVFHTL | |
| | | GQYFQKLGRCSVRVSVNTANVTLGPQLMEVTVYRRHGRAYVPIAQVKDVYVVTDQIPVFV | |
| | | TMFQKNDRNSSDETFLKDLPIMFDVLIHDPSHFLNYSTINYKWSEGDNTGLEVSTNHTVN | |
| | | HTYVLNGTFSLNLTVKAAAPGPCPPPPPPPRPSKPTPSLATTLKSYDSNTPGPAGDNPLE | |
| | | LSRIPDENCQINRYGHFQATITIVEGILEVNIIQMTDVLMPVPWPESSLIDFVVTCQGSI | |
| | | PTEVCTIISDPTCEITQNTVCSPVDVDEMCLLTVRRTFNGSGTYCVNLTLGDDTSLALTS | |
| | | TLISVPDRDPASPLRMANSALISVGCLAIFVTVISLLVYKKHKEYNPIENSPGNVVRSKG | |
| | | LSVFLNRAKAVFFPGNQEKDPLLKNQEFKGVS | |
| 45 | VPS35 | MPTTQQSPQDEQEKLLDEAIQAVKVQSFQMKRCLDKNKLMDALKHASNMLGELRTSMLSP | ENA AF191298.2 |
| | | KSYYELYMAISDELHYLEVYLTDEFAKGRKVADLYELVQYAGNIIPRLYLLITVGVVYVK | |
| | | SFPQSRKDILKDLVEMCRGVQHPLRGLFLRNYLLQCTRNILPDEGEPTDEETTGDISDSM | |
| | | DFVLLNFAEMNKLWVRMQHQGHSRDREKRERERQELRILVGTNLVRLSQLEGVNVERYKQ | |
| | | IVLTGILEQVVNCRDALAQEYLMECIIQVFPDEFHLQTLNPFLRACAELHQNVNVKNIII | |
| | | ALIDRLALFAHREDGPGIPADIKLFDIFSQQVATVIQSRQDMPSEDVVSLQVSLINLAMK | |
| | | CYPDRVDYVDKVLETIVEIFNKLNLEHIATSSAVSKELTRLLKIPVDTYNNILTVLKLKH | |
| | | FHPLFEYFDYESRKSMSCYVLSNVLDYNTEIVSQDQVDSIMNLVSTLIQDQPDQPVEDPD | |
| | | PEDFADEQSLVGRFIHLLRSEDPDQQYLILNTARKHFGAGGNQRIRFTLPPLVFAAYQLA | |
| | | FRYKENSKVDDKWEKKCQKIFSFAHQTISALIKAELAELPLRLFLQGALAAGEIGFENHE | |
| | | TVAYEFMSQAFSLYEDEISDSKAQLAAITLIIGTFERMKCFSEENHEPLRTQCALAASKL | |
| | | LKKPDQGRAVSTCAHLFWSGRNTDKNGEELHGGKRVMECLKKALKIANQCMDPSLQVQLF | |
| | | IEILNRYIYFYEKENDAVTIQVLNQLIQKIREDLPNLESSEETEQINKHFHNTLEHLRLR | |
| | | RESPESEGPIYEGLIL | |
| 46 | FBXO7 | MRLRVRLLKRTWPLEVPETEPTLGHLRSHLRQSLLCTWGYSSNTRFTITLNYKDPLTGDE | ENA AF233225.1 |
| | | ETLASYGIVSGDLICLILQDDIPAPNIPSSTDSEHSSLQNNEQPSLATSSNQTSMQDEQP | |
| | | SDSFQGQAAQSGVWNDDSMLGPSQNFEAESIQDNAHMAEGTGFYPSEPMLCSESVEGQVP | |
| | | HSLETLYQSADCSDANDALIVLIHLLMLESGYIPQGTEAKALSMPEKWKLSGVYKLQYMH | |
| | | PLCEGSSATLTCVPLGNLIVVNATLKINNEIRSVKRLQLLPESFICKEKLGENVANIYKD | |
| | | LQKLSRLFKDQLVYPLLAFTRQALNLPDVFGLVVLPLELKLRIFRLLDVRSVLSLSAVCR | |
| | | DLFTASNDPLLWRFLYLRDFRDNTVRVQDTDWKELYRKRHIQRKESPKGRFVMLLPSSTH | |
| | | TIPFYPNPLHPRPFPSSRLPPGIIGGEYDQRPTLPYVGDPISSLIPGPGETPSQFPPLRP | |
| | | RFDPVGPLPGPNPILPGRGGPNDRFPFRPSRGRPTDGRLSFM | |
| 47 | PARK7 | MASKRALVILAKGAEEMETVIPVDVMRRAGIKVTVAGLAGKDPVQCSRDVVICPDASLED | ENA D613802 |
| | | AKKEGPYDVVVLPGGNLGAQNLSESAAVKEILKEQENRKGLIAAICAGPTALLAHEIGFG | |
| | | SKVTTHPLAKDKMMNGGHYTYSENRVEKDGLILTSRGPGTSFEFALAIVEALNGKEVAAQ | |
| | | VKAPLVLKD | |
| 48 | INPP5F | MELFQAKDHYILQQGERALWCSRADGGLQLRPATDLLLAWNPICLGLVEGVIGKIQLHSD | ENA BC052367.1 |
| | | LPWWLILIRQKALVGKLPGDHEVCKVTKIAVLSLSEMEPQDLELELCKKHHFGINKPEKI | |
| | | IPSPDDSKFLLKTFTHIKSNVSAPNKKKVKESKEKEKLERRLLEELLKMFMDSESFYYSL | |
| | | TYDLTNSVQRQSTGERDGRPLWQKVDDRFFWNKYMIQDLTEIGTPDVDFWIIPMIQGFVQ | |
| | | IEELVVNYTESSDDEKSSPETPPQESTCVDDIHPRFLVALISRRSRHRAGMRYKRRGVDK | |
| | | NGNVANYVETEQLIHVHNHTLSFVQTRGSVPVFWSQVGYRYNPRPRLDRSEKETVAYFCA | |
| | | HFEEQLNIYKKQVIINLVDQAGREKIIGDAYLKQVLLENNSHLTYVSEDFHEHCRGMKFE | |
| | | NVQTLTDAIYDIILDMKWCWVDEAGVICKQEGIFRVNCMDCLDRTNVVQAAIARVVMEQQ | |
| | | LKKLGVMPPEQPLPVKCNRIYQIMWANNGDSISRQYAGTAALKGDFTRTGERKLAGVMKD | |
| | | GVNSANRYYLNRFKDAYRQAVIDLMQGIPVTEDLYSIFTKEKEHEALHKENQRSHQELIS | |
| | | QLLQSYMKLLLPDDEKFHGGWALIDCDPSLIDATHRDVDVLLLLSNSAYYVAYYDDEVDK | |
| | | VNQYQRLSLENLEKIEIGPEPTLFGKPKESCMRLHYRYKEASGYFHTLRAVMRNPEEDGK | |
| | | DTLQCIAEMLQITKQAMGSDLPIIEKKLERKSSKPHEDIIGIRSQNQGSLAQGKNFLMSK | |
| | | FSSLNQKVKQTKSNVNIGNLRKLGNETKPEMKVNELKPNLKVNLWKSDSSLETMENTGVM | |
| | | DKVQAESDGDMSSDNDSYHSDEFLTNSKSDEDRQLANSLESVGPIDYVLPSCGIIASAPR | |
| | | LGSRSQSLSSTDSSVHAPSEITVAHGSGLGKGQESPLKKSPSAGDVHILTGFAKPMDIYC | |
| | | HRFVQDAQNKVTHLSETRSVSQQASQERNQMTNQVSNETQSESTEQTPSRPSQLDVSLSA | |
| | | TGPQFLSVEPAHSVASQKTPTSASSMLELETGLHVTPSPSESSSSRAVSPFAKIRSSMVQ | |
| | | VASITQAGLTHGINFAVSKVQKSPPEPEIINQVQQNELKKMFIQCQTRIIQI | |
| 49 | DNAJC | MNIIRENKDLACFYTTKHSWRGKYKRVFSVGTHAITTYNPNTLEVTNQWPYGDICSISPV | ENA AY779857.1 |
| | 13 | GKGQGTEFNLTFRKGSGKKSETLKFSTEHRTELLTEALRFRTDFSEGKITGRRYNCYKHH | |
| | | WSDSRKPVILEVTPGGFDQINPATNRVLCSYDYRNIEGFVDLSDYQGGFCILYGGFSRLH | |
| | | LFASEQREEIIKSAIDHAGNYIGISLRIRKEPLEFEQYLNLRFGKYSTDESITSLAEFVV | |
| | | QKISPRHSEPVKRVLALTETCLVERDPATYNIATLKPLGEVFALVCDSENPQLFTIEFIK | |
| | | GQVRKYSSTERDSLLASLLDGVRASGNRDVCVKMTPTHKGQRWGLLSMPVDEEVESLHLR | |
| | | FLATPPNGNFADAVERFNANISYSGVLHAVTQDGLFSENKEKLINNAITALLSQEGDVVA | |
| | | SNAELESQFQAVRRLVASKAGFLAFTQLPKFRERLGVKVVKALKRSNNGIIHAAVDMLCA | |
| | | LMCPMHDDYDLRQEQLNKASLLSSKKFLENLLEKENSHVDHGTGALVISSLLDFLTFALC | |
| | | APYSETTEGQQFDMLLEMVASNGRTLFKLFQHPSMAIIKGAGLVMKAIIEEGDKEIATKM | |
| | | QELALSEGALPRHLHTAMFTISSDQRMLTNRQLSRHLVGLWTADNATATNLLKRILPPGL | |
| | | LAYLESSDLVPEKDADRMHVRDNVKIAMDQYGKENKVPEWQRLAGKAAKEVEKFAKEKVD | |
| | | LVLMHWRDRMGIAQKENINQKPVVLRKRRQRIKIEANWDLFYYRFGQDHARSNLIWNFKT | |
| | | REELKDTLESEMRAFNIDRELGSANVISWNHHEFEVKYECLAEEIKIGDYYLRLLLEEDE | |
| | | NEESGSIKRSYEFFNELYHRFLLTPKVNMKCLCLQALAIVYGRCHEEIGPFTDTRYIIGM | |
| | | LERCTDKLERDRLILFLNKLILNKKNVKDLMDSNGIRILVDLLTLAHLHVSRATVPLQSN | |
| | | VIEAAPDMKRESEKEWYFGNADKERSGPYGFHEMQELWTKGMLNAKTRCWAQGMDGWRPL | |
| | | QSIPQLKWCLLASGQAVLNETDLATLILNMLITMCGYFPSRDQDNAIIRPLPKVKRLLSD | |
| | | STCLPHIIQLLLTFDPILVEKVAILLYHIMQDNPQLPRLYLSGVEFFIMMYTGSNVLPVA | |
| | | RELKYTHTKQAFKSEETKGQDIFQRSILGHILPEAMVCYLENYEPEKESEIFLGEFDTPE | |
| | | AIWSSEMRRLMIEKIAAHLADFTPRLQSNTRALYQYCPIPIINYPQLENELFCNIYYLKQ | |
| | | LCDTLRFPDWPIKDPVKLLKDTLDAWKKEVEKKPPMMSIDDAYEVLNLPQGQGPHDESKI | |
| | | RKAYFRLAQKYHPDKNPEGRDMFEKVNKAYEFLCTKSAKIVDGPDPENIILILKTQSILF | |
| | | NRHKEDLQPYKYAGYPMLIRTITMETSDDLLFSKESPLLPAATELAFHTVNCSALNAEEL | |
| | | RRENGLEVLQEAFSRCVAVLTRASKPSDMSVQVCGYISKCYSVAAQFEECREKITEMPSI | |
| | | IKDLCRVLYFGKSIPRVAALGVECVSSFAVDFWLQTHLFQAGILWYLLGFLFNYDYTLEE | |
| | | SGIQKSEETNQQEVANSLAKLSVHALSRLGGYLAEEQATPENPTIRKSLAGMLTPYVARK | |
| | | LAVASVTEILKMLNSNTESPYLIWNNSTRAELLEFLESQQENMIKKGDCDKTYGSEFVYS | |
| | | DHAKELIVGEIFVRVYNEVPTFQLEVPKAFAASLLDYIGSQAQYLHTFMAITHAAKVESE | |
| | | QHGDRLPRVEMALEALRNVIKYNPGSESECIGHFKLIFSLLRVHGAGQVQQLALEVVNIV | |
| | | TSNQDCVNNIAESMVLSSLLALLHSLPSSRQLVLETLYALTSSTKIIKEAMAKGALIYLL | |
| | | DMFCNSTHPQVRAQTAELFAKMTADKLIGPKVRITLMKFLPSVFMDAMRDNPEAAVHIFE | |
| | | GTHENPELIWNDNSRDKVSTTVREMMLEHEKNQQDNPEANWKLPEDFAVVEGEAEGELAV | |
| | | GGVFLRIFIAQPAWVLRKPREFLIALLEKLTELLEKNNPHGETLETLTMATVCLFSAQPQ | |
| | | LADQVPPLGHLPKVIQAMNHRNNAIPKSAIRVIHALSENELCVRAMASLETIGPLMNGMK | |
| | | KRADTVGLACEAINRMFQKEQSELVAQALKADLVPYLLKLLEGIGLENLDSPAATKAQIV | |
| | | KALKAMTRSLQYGEQVNEILCRSSVWSAFKDQKHDLFISESQTAGYLTGPGVAGYLTAGT | |
| | | STSVMSNLPPPVDHEAGDLGYQT | |
| 50 | GCH1 | MEKGPVRAPAEKPRGARCSNGFPERDPPRPGPSRPAEKPPRPEAKSAQPADGWKGERPRS | ENA S44049.1 |
| | | EEDNELNLPNLAAAYSSILSSLGENPQRQGLLKTPWRAASAMQFFTKGYQETISDVLNDA | |
| | | IFDEDHDEMVIVKDIDMFSMCEHHLVPFVGKVHIGYLPNKQVLGLSKLARIVEIYSRRLQ | |
| | | VQERLTKQIAVAITEALRPAGVGVVVEATHMCMVMRGVQKMNSKTVTSTMLGVFREDPKT | |
| | | REEFLTLIRS | |
| 51 | NMD3 | MEYMAESTDRSPGHILCCECGVPISPNPANICVACLRSKVDISQGIPKQVSISFCKQCQR | ENA AF132941.1 |
| | | YFQPPGTWIQCALESRELLALCLKKIKAPLSKVRLVDAGFVWTEPHSKRLKVKLTIQKEV | |
| | | MNGAILQQVFVVDYVVQSQMCGDCHRVEAKDFWKAVIQVRQKTLHKKTFYYLEQLILKYG | |
| | | MHQNTLRIKEIHDGLDFYYSSKQHAQKMVEFLQCTVPCRYKASQRLISQDIHSNTYNYKS | |
| | | TFSVEIVPICKDNVVCLSPKLAQSLGNMNQICVCIRVTSAIHLIDPNTLQVADIDGSTFW | |
| | | SHPFNSLCHPKQLEEFIVMECSIVQDIKRAAGAGMISKKHTLGEVWVQKTSEMNTDKQYF | |
| | | CRTHLGHLLNPGDLVLGFDLANCNLNDEHVNKMNSDRVPDVVLIKKSYDRTKRQRRRNWK | |
| | | LKELARERENMDTDDERQYQDFLEDLEEDEAIRKNVNIYRDSAIPVESDTDDEGAPRISL | |
| | | AEMLEDLHISQDATGEEGASMLT | |
| 52 | USP25 | MTVEQNVLQQSAAQKHQQTFLNQLREITGINDTQILQQALKDSNGNLELAVAFLTAKNAK | ENA AF170562.1 |
| | | TPQQEETTYYQTALPGNDRYISVGSQADTNVIDLTGDDKDDLQRAIALSLAESNRAFRET | |
| | | GITDEEQAISRVLEASIAENKACLKRTPTEVWRDSRNPYDRKRQDKAPVGLKNVGNTCWF | |
| | | SAVIQSLFNLLEFRRLVLNYKPPSNAQDLPRNQKEHRNLPFMRELRYLFALLVGTKRKYV | |
| | | DPSRAVEILKDAFKSNDSQQQDVSEFTHKLLDWLEDAFQMKAEEETDEEKPKNPMVELFY | |
| | | GRFLAVGVLEGKKFENTEMFGQYPLQVNGFKDLHECLEAAMIEGEIESLHSENSGKSGQE | |
| | | HWFTELPPVLTFELSRFEFNQALGRPEKIHNKLEFPQVLYLDRYMHRNREITRIKREETK | |
| | | RLKDYLTVLQQRLERYLSYGSGPKRFPLVDVLQYALEFASSKPVCTSPVDDIDASSPPSG | |
| | | SIPSQTLPSTTEQQGALSSELPSTSPSSVAAISSRSVIHKPFTQSRIPPDLPMHPAPRHI | |
| | | TEEELSVLESCLHRWRTEIENDTRDLQESISRIHRTIELMYSDKSMIQVPYRLHAVLVHE | |
| | | GQANAGHYWAYIFDHRESRWMKYNDIAVTKSSWEELVRDSFGGYRNASAYCLMYINDKAQ | |
| | | FLIQEEFNKETGQPLVGIETLPPDLRDFVEEDNQRFEKELEEWDAQLAQKALQEKLLASQ | |
| | | KLRESETSVTTAQAAGDPEYLEQPSRSDFSKHLKEETIQIITKASHEHEDKSPETVLQSA | |
| | | IKLEYARLVKLAQEDIPPETDYRLHHVVVYFIQNQAPKKIIEKTLLEQFGDRNLSFDERC | |
| | | HNIMKVAQAKLEMIKPEEVNLEEYEEWHQDYRKFRETTMYLIIGLENFQRESYIDSLLFL | |
| | | ICAYQNNKELLSKGLYRGHDEELISHYRRECLLKLNEQAAELFESGEDREVNNGLIIMNE | |
| | | FIVPFLPLLLVDEMEEKDILAVEDMRNRWCSYLGQEMEPHLQEKLTDFLPKLLDCSMEIK | |
| | | SFHEPPKLPSYSTHELCERFARIMLSLSRTPADGR | |
| 53 | RAB7L1 | MGSRDHLFKVLVVGDAAVGKTSLVQRYSQDSFSKHYKSTVGVDFALKVLQWSDYEIVRLQ | ENA D84488.1 |
| | | LWDIAGQERFTSMTRLYYRDASACVIMFDVTNATTFSNSQRWKQDLDSKLTLPNGEPVPC | |
| | | LLLANKCDLSPWAVSRDQIDRFSKENGFTGWTETSVKENKNINEAMRVLIEKMMRNSTED | |
| | | IMSLSTQGDYINLQTKSSSWSCC | |
| 54 | SIPAIL | MSDPRQSQEEKFIKLGRASSKFKDPPRIMQSDDYFARKFKAINGNMGPTTSLNASNSNETG | ENA AY168879.1 |
| | 2 | GGGPANGTPAVPKMGVRARVSEWPPKKDCSKELTCKALWESRSQTSYESITSVLQNGQSD | |
| | | QSEGQQDEQLDLDFVEAKYTIGDIFVHSPQRGLHPIRQRSNSDVTISDIDAEDVLDQNAV | |
| | | NPNTGAALHREYGSTSSIDRQGLSGENFFAMLRGYRVENYDHKAMVPFGFPEFFRCDPAI | |
| | | SPSLHAAAQISRGEFVRISGLDYVDSALLMGRDRDKPFKRRLKSESVETSLFRKLRTVKS | |
| | | EHETFKFTSELEESRLERGIRPWNCQRCFAHYDVQSILENINEAMATRANVGKRKNITTG | |
| | | ASAASQTQMPTGQTGNCESPLGSKEDLNSKENLDADEGDGKSNDLVLSCPYFRNETGGEG | |
| | | DRRIALSRANSSSFSSGESCSFESSLSSHCTNAGVSVLEVPRENQPIHREKVKRYIIEHI | |
| | | DLGAYYYRKFFYGKEHQNYFGIDENLGPVAVSIRREKVEDAKEKEGSQFNYRVAFRTSEL | |
| | | TTLRGAILEDAIPSTARHGTARGLPLKEVLEYVIPELSIQCLRQASNSPKVSEQLLKLDE | |
| | | QGLSFQHKIGILYCKAGQSTEEEMYNNETAGPAFEEFLDLLGQRVRLKGFSKYRAQLDNK | |
| | | TDSTGTHSLYTTYKDYELMFHVSTLLPYMPNNRQQLLRKRHIGNDIVTIVFQEPGALPFT | |
| | | PKSIRSHFQHVFVIVKVHNPCTENVCYSVGVSRSKDVPPFGPPIPKGVTFPKSAVFRDFL | |
| | | LAKVINAENAAHKSEKFRAMATRTRQEYLKDLAENFVTTATVDTSVKFSFITLGAKKKEK | |
| | | VKPRKDAHLFSIGAIMWHVIARDFGQSADIECLLGISNEFIMLIEKDSKNVVFNCSCRDV | |
| | | IGWTSGLVSIKVFYERGECVLLSSVDNCAEDIREIVQRLVIVIRGCETVEMTLARNGLGQ | |
| | | LGFHVNFEGIVADVEPFGFAWKAGLRQGSRLVEICKVAVATLTHEQMIDLLRTSVTVKVV | |
| | | IIQPHDDGSPRRGCSELCRIPMVEYKLDSEGTPCEYKTPFRRNTTWHRVPTPALQPLSRA | |
| | | SPIPGTPDRLPCQQLLQQAQAAIPRSTSFDRKLPDGTRSSPSNQSSSSDPGPGGSGPWRP | |
| | | QVGYDGCQSPLLLEHQGSGPLECDGAREREDTMEASRHPETKWHGPPSKVLGSYKERALQ | |
| | | KDGSCKDSPNKLSHIGDKSCSSHSSSNTLSSNTSSNSDDKHFGSGDLMDPELLGLTYIKG | |
| | | ASTDSGIDTAPCMPATILGPVHLAGSRSLIHSRAEQWADAADVSGPDDEPAKLYSVHGYA | |
| | | STISAGSAAEGSMGDLSEISSHSSGSHHSGSPSAHCSKSSGSLDSSKVYIVSHSSGQQVP | |
| | | GSMSKPYHRQGAVNKYVIGWKKSEGSPPPEEPEVTECPGMYSEMDVMSTATQHQTVVGDA | |
| | | VAETQHVLSKEDFLKLMLPDSPLVEEGRRKFSFYGNLSPRRSLYRTLSDESICSNRRGSS | |
| | | FGSSRSSVLDQALPNDILFSTTPPYHSTLPPRAHPAPSMGSLRNEFWFSDGSLSDKSKCA | |
| | | DPGLMPLPDTATGLDWTHLVDAARAFEGLDSDEELGLLCHHTSYLDQRVASFCTLTDMQH | |
| | | GQDLEGAQELPLCVDPGSGKEFMDTTGERSPSPLTGKVNQLELILRQLQTDLRKEKQDKA | |
| | | VLQAEVQHLRQDNMRLQEESQTATAQLRKFTEWFFTTIDKKS | |
| 55 | MCCC1 | MAAASAVSVLLVAAERNRWHRLPSLLLPPRTWVWRQRTMKYTTATGRNITKVLIANRGEI | ENA AF310972.1 |
| | | ACRVMRTAKKLGVQTVAVYSEADRNSMHVDMADEAYSIGPAPSQQSYLSMEKIIQVAKTS | |
| | | AAQAIHPGCGFLSENMEFAELCKQEGIIFIGPPPSAIRDMGIKSTSKSIMAAAGVPVVEG | |
| | | YHGEDQSDQCLKEHARRIGYPVMIKAVRGGGGKGMRIVRSEQEFQEQLESARREAKKSFN | |
| | | DDAMLIEKFVDTPRHVEVQVFGDHHGNAVYLFERDCSVQRRHQKIIEEAPAPGIKSEVRK | |
| | | KLGEAAVRAAKAVNYVGAGTVEFIMDSKHNFCFMEMNTRLQVEHPVTEMITGTDLVEWQL | |
| | | RIAAGEKIPLSQEEITLQGHAFEARIYAEDPSNNFMPVAGPLVHLSTPRADPSTRIETGV | |
| | | RQGDEVSVHYDPMIAKLVVWAADRQAALTKLRYSLRQYNIVGLHTNIDFLLNLSGHPEFE | |
| | | AGNVHTDFIPQHHKQLLLSRKAAAKESLCQAALGLILKEKAMTDTFTLQAHDQFSPFSSS | |
| | | SGRRLNISYTRNMTLKDGKNNVAIAVTYNHDGSYSMQIEDKTFQVLGNLYSEGDCTYLKC | |
| | | SVNGVASKAKLIILENTIYLFSKEGSIEIDIPVPKYLSSVSSQETQGGPLAPMTGTIEKV | |
| | | FVKAGDKVKAGDSLMVMIAMKMEHTIKSPKDGTVKKVFYREGAQANRHTPLVEFEEEESD | |
| | | KRESE | |
| 56 | SYNJ1 | MAFSKGFRIYHKLDPPPFSLIVETRHKEECLMFESGAVAVLSSAEKEAIKGTYSKVLDAY | ENA AF009039.1 |
| | | GLLGVLRLNLGDTMLHYLVLVTGCMSVGKIQESEVFRVTSTEFISLRIDSSDEDRISEVR | |
| | | KVLNSGNFYFAWSASGISLDLSLNAHRSMQEQTTDNRFFWNQSLHLHLKHYGVNCDDWLL | |
| | | RLMCGGVEIRTIYAAHKQAKACLISRLSCERAGTRFNVRGTNDDGHVANFVETEQVVYLD | |
| | | DSVSSFIQIRGSVPLFWEQPGLQVGSHRVRMSRGFEANAPAFDRHFRTLKNLYGKQIIVN | |
| | | LLGSKEGEHMLSKAFQSHLKASEHAADIQMVNEDYHQMVKGGKAEKLHSVLKPQVQKFLD | |
| | | YGFFYFNGSEVQRCQSGTVRTNCLDCLDRTNSVQAFLGLEMLAKQLEALGLAEKPQLVIR | |
| | | FQEVERSMWSVNGDSISKIYAGTGALEGKAKLKDGARSVTRTIQNNFFDSSKQEAIDVLL | |
| | | LGNTLNSDLADKARALLTTGSLRVSEQTLQSASSKVLKSMCENFYKYSKPKKIRVCVGIW | |
| | | NVNGGKQFRSIAFKNQTLTDWLLDAPKLAGIQEFQDKRSKPTDIFAIGFEEMVELNAGNI | |
| | | VSASTTNQKLWAVELQKTISRDNKYVLLASEQLVGVCLFVFIRPQHAPFIRDVAVDTVKT | |
| | | GMGGATGNKGAVAIRMLFHTTSLCFVCSHFAAGQSQVKERNEDFIEIARKLSFPMGRMLF | |
| | | SHDYVFWCGDFNYRIDLPNEEVKELIRQQNWDSLIAGDQLINQKNAGQVFRGFLEGKVTF | |
| | | APTYKYDLFSDDYDTSEKCRTPAWTDRVLWRRRKWPFDRSAEDLDLLNASFQDESKILYT | |
| | | WTPGTLLHYGRAELKTSDHRPVVALIDIDIFEVEAEERQNIYKEVIAVQGPPDGTVLVSI | |
| | | KSSLPENNFFDDALIDELLQQFASFGEVILIRFVEDKMWVTFLEGSSALNVLSLNGKELL | |
| | | NRTITIALKSPOWIKNLEEEMSLEKISIALPSSTSSTLLGEDAEVAADFDMEGDVDDYSA | |
| | | EVEELLPQHLQPSSSSGLGTSPSSSPRTSPCQSPTISEGPVPSLPIRESRAPSRTPGPPS | |
| | | AQSSPIDAQPATPLPQKDPAQPLEPKRPPPPRPVAPPTRPAPPQRPPPPSGARSPAPTRK | |
| | | EFGGIGAPPSPGVARREMEAPKSPGTTRKDNIGRSQPSPQAGLAGPGPAGYSTARPTIPP | |
| | | RAGVISAPQSHARASAGRLTPESQSKTSETSKGSTFLPEPLKPQAAFPPQSSLPPPAQRL | |
| | | QEPLVPVAAPMPQSGPQPNLETPPQPPPRSRSSHSLPSEASSQPQVKTNGISDGKRESPL | |
| | | KIDPFEDLSFNLLAVSKAQLSVQTSPVPTPDPKRLIQLPSATQSNVLSSVSCMPTMPPIP | |
| | | ARSQSQENMRSSPNPFITGLTRTNPFSDRTAAPGNPFRAKSEESEATSWFSKEEPVTISP | |
| | | FPSLQPLGHNKSRASSSLDGFKDSFDLQGQSTLKISNPKGWVIFEEEEDFGVKGKSKSAC | |
| | | SDLLGNQPSSFSGSNLTLNDDWNKGTNVSFCVLPSRRPPPPPVPLLPPGTSPPVDPFTTL | |
| | | ASKASPTLDFTER | |
| 57 | LRRK2 | MASGSCQGCEEDEETLKKLIVRLNNVQEGKQIETLVQILEDLLVFTYSERASKLFQGKNI | ENA AY792511.1 |
| | | HVPLLIVLDSYMRVASVQQVGWSLLCKLIEVCPGTMQSLMGPQDVGNDWEVLGVHQLILK | |
| | | MLTVHNASVNLSVIGLKTLDLLLTSGKITLLILDEESDIFMLIFDAMHSFPANDEVQKLG | |
| | | CKALHVLFERVSEEQLTEFVENKDYMILLSALTNFKDEEEIVLHVLHCLHSLAIPCNNVE | |
| | | VLMSGNVRCYNIVVEAMKAFPMSERIQEVSCCLLHRLTLGNFFNILVLNEVHEFVVKAVQ | |
| | | QYPENAALQISALSCLALLTETIFLNQDLEEKNENQENDDEGEEDKLFWLEACYKALTWH | |
| | | RKNKHVQEAACWALNNLLMYQNSLHEKIGDEDGHFPAHREVMLSMLMHSSSKEVFQASAN | |
| | | ALSTLLEQNVNFRKILLSKGIHLNVLELMQKHIHSPEVAESGCKMLNHLFEGSNTSLDIM | |
| | | AAVVPKILTVMKRHETSLPVQLEALRAILHFIVPGMPEESREDTEFHHKLNMVKKQCFKN | |
| | | DIHKLVLAALNRFIGNPGIQKCGLKVISSIVHFPDALEMLSLEGAMDSVLHTLQMYPDDQ | |
| | | EIQCLGLSLIGYLITKKNVFIGTGHLLAKILVSSLYRFKDVAEIQTKGFQTILAILKLSA | |
| | | SFSKLLVHHSFDLVIFHQMSSNIMEQKDQQFLNLCCKCFAKVAMDDYLKNVMLERACDQN | |
| | | NSIMVECLLLLGADANQAKEGSSLICQVCEKESSPKLVELLLNSGSREQDVRKALTISIG | |
| | | KGDSQIISLLLRRLALDVANNSICLGGFCIGKVEPSWLGPLFPDKTSNLRKQTNIASTLA | |
| | | RMVIRYQMKSAVEEGTASGSDGNFSEDVLSKFDEWTFIPDSSMDSVFAQSDDLDSEGSEG | |
| | | SFLVKKKSNSISVGEFYRDAVLQRCSPNLQRHSNSLGPIFDHEDLLKRKRKILSSDDSLR | |
| | | SSKLQSHMRHSDSISSLASEREYITSLDLSANELRDIDALSQKCCISVHLEHLEKLELHQ | |
| | | NALTSFPQQLCETLKSLTHLDLHSNKFTSEPSYLLKMSCIANLDVSRNDIGPSVVLDPTV | |
| | | KCPTLKQFNLSYNQLSFVPENLTDVVEKLEQLILEGNKISGICSPLRLKELKILNLSKNH | |
| | | ISSLSENFLEACPKVESFSARMNFLAAMPFLPPSMTILKLSQNKFSCIPEATLNLPHLRS | |
| | | LDMSSNDIQYLPGPAHWKSLNLRELLFSHNQISILDLSEKAYLWSRVEKLHLSHNKLKEI | |
| | | PPEIGCLENLTSLDVSYNLELRSFPNEMGKLSKIWDLPLDELHLNFDFKHIGCKAKDIIR | |
| | | FLQQRLKKAVPYNRMKLMIVGNTGSGKTTLLQQLMKTKKSDLGMQSATVGIDVKDWPIQI | |
| | | RDKRKRDLVLNVWDFAGREEFYSTHPHFMTQRALYLAVYDLSKGQAEVDAMKPWLFNIKA | |
| | | RASSSPVILVGTHLDVSDEKQRKACMSKITKELLNKRGFPAIRDYHFVNATEESDALAKL | |
| | | RKTIINESLNFKIRDQLVVGQLIPDCYVELEKIILSERKNVPIEFPVIDRKRLLQLVREN | |
| | | QLQLDENELPHAVHFLNESGVLLHFQDPALQLSDLYFVEPKWLCKIMAQILTVKVEGCPK | |
| | | HPKGIISRRDVEKFLSKKRKFPKNYMSQYFKLLEKFQIALPIGEEYLLVPSSLSDHRPVI | |
| | | ELPHCENSEIIIRLYEMPYFPMGFWSRLINRLLEISPYMLSGRERALRPNRMYWRQGIYL | |
| | | NWSPEAYCLVGSEVLDNHPESFLKITVPSCRKGCILLGQVVDHIDSLMEEWFPGLLEIDI | |
| | | CGEGETLLKKWALYSFNDGEEHQKILLDDLMKKAEEGDLLVNPDQPRLTIPISQIAPDLI | |
| | | LADLPRNIMLNNDELEFEQAPEFLLGDGSFGSVYRAAYEGEEVAVKIFNKHTSLRLLRQE | |
| | | LVVLCHLHHPSLISLLAAGIRPRMLVMELASKGSLDRLLQQDKASLTRTLQHRIALHVAD | |
| | | GLRYLHSAMIIYRDLKPHNVLLFTLYPNAAIIAKIADYGIAQYCCRMGIKTSEGTPGFRA | |
| | | PEVARGNVIYNQQADVYSFGLLLYDILTTGGRIVEGLKFPNEFDELEIQGKLPDPVKEYG | |
| | | CAPWPMVEKLIKQCLKENPQERPTSAQVFDILNSAELVCLTRRILLPKNVIVECMVATHH | |
| | | NSRNASIWLGCGHTDRGQLSFLDLNTEGYTSEEVADSRILCLALVHLPVEKESWIVSGIQ | |
| | | SGTLLVINTEDGKKRHTLEKMTDSVTCLYCNSFSKQSKQKNFLLVGTADGKLAIFEDKTV | |
| | | KLKGAAPLKILNIGNVSTPLMCLSESINSTERNVMWGGCGTKIFSFSNDFTIQKLIETRT | |
| | | SQLFSYAAFSDSNIITVVVDTALYIAKQNSPVVEVWDKKTEKLCGLIDCVHFLREVMVKE | |
| | | NKESKHKMSYSGRVKTLCLQKNTALWIGTGGGHILLLDLSTRRLIRVIYNFCNSVRVMMT | |
| | | AQLGSLKNVMLVLGYNRKNTEGTQKQKEIQSCLTVWDINLPHEVQNLEKHIEVRKELAEK | |
| | | MRRTSVE | |
| 58 | SNCA | MDVFMKGLSKAKEGVVAAAEKTKQGVAEAAGKTKEGVLYVGSKTKEGVVHGVATVAEKIK | ENA L08850.1 |
| | | EQVINVGGAVVTGVTAVAQKTVEGAGSIAAATGFVKKDQLGKNEEGAPQEGILEDMPVDP | |
| | | DNEAYEMPSEEGYQDYEPEA | |
| 59 | PTRHD | MHRGVGPAFRVVRKMAASGAEPQVLVQYLVLRKDLSQAPFSWPAGALVAQACHAATAALH | ENA BC073803.1 |
| | 1 | THRDHPHTAAYLQELGRMRKVVLEAPDETTLKELAETLQQKNIDHMLWLEQPENIATCIA | |
| | | LRPYPKEEVGQYLKKFRLFK | |
| 60 | PINK1 | MAVRQALGRGLQLGRALLLRFTGKPGRAYGLGRPGPAAGCVRGERPGWAAGPGAEPRRVG | ENA AB053323.1 |
| | | LGLPNRLRFFRQSVAGLAARLQRQFVVRAWGCAGPCGRAVFLAFGLGLGLIEEKQAESRR | |
| | | AVSACQEIQAIFTQKSKPGPDPLDTRRLQGFRLEEYLIGQSIGKGCSAAVYEATMPTLPQ | |
| | | NLEVIKSTGLLPGRGPGTSAPGEGQERAPGARAFPLAIKMMWNISAGSSSEAILNTMSQE | |
| | | LVPASRVALAGEYGAVTYRKSKRGPKQLAPHPNIIRVLRAFTSSVPLLPGALVDYPDVLP | |
| | | SRLHPEGLGHGRTLFLVMKNYPCTLRQYLCVNTPSPRLAAMMLLQLLEGVDHLVQQGIAH | |
| | | RDLKSDNILVELDPDGCPWLVIADEGCCLADESIGLQLPFSSWYVDRGGNGCLMAPEVST | |
| | | ARPGPRAVIDYSKADAWAVGAIAYEIFGLVNPFYGQGKAHLESRSYQEAQLPALPESVPP | |
| | | DVRQLVRALLQREASKRPSARVAANVLHLSLWGEHILALKNLKLDKMVGWLLQQSAATLL | |
| | | ANRLTEKCCVETKMKMLFLANLECETLCQAALLLCSWRAAL | |
| 104 | GBA | MEFSSPSREECPKPLSRVSIMAGSLTGLLLLQAVSWASGARPCIPKSFGYSSVVCVCNAT | ENA M16328.1 |
| | | YCDSFDPPTFPALGTFSRYESTRSGRRMELSMGPIQANHTGTGLLLTLQPEQKFQKVKGF | |
| | | GGAMTDAAALNILALSPPAQNLLLKSYFSEEGIGYNIIRVPMASCDFSIRTYTYADTPDD | |
| | | FQLHNFSLPEEDTKLKIPLIHRALQLAQRPVSLLASPWTSPTWLKTNGAVNGKGSLKGQP | |
| | | GDIYHQTWARYEVKFLDAYAEHKLQFWAVTAENEPSAGLLSGYPEQCLGETPEHQRDFIA | |
| | | RDLGPTLANSTHHNVRLLMLDDQRLLLPHWAKVVLTDPEAAKYVHGIAVHWYLDFLAPAK | |
| | | ATLGETHRLFPNTMLFASEACVGSKFWEQSVRLGSWDRGMQYSHSIITNLLYHVVGWTDW | |
| | | NLALNPEGGPNWVRNFVDSPIIVDITKDTFYKQPMFYHLGHFSKFIPEGSQRVGLVASQK | |
| | | NDLDAVALMHPDGSAVVVVLNRSSKDVPLTIKDPAVGFLETISPGYSIHTYLWRRQ | |
| 61 | TMEM1 | MEPAAGIQRRSSQGPTVPPPPRGHAPPAAAPGPAPLSSPVREPPQLEEERQVRISESGQF | ENA AL122044.1 |
| | 63 | SDGLEDRGLLESSWESTTRLKPHEAQNYRKKALWVSIVTLALAVAAFTVSVMRYSASAFG | |
| | | FAFDAILDVLSSAIVLWRYSNAAAVHSAHREYIACVILGVIFLLSSICIVVKAIHDLSTR | |
| | | LLPEVDDFLFSVSILSGILCSILAVLKFMLGKVLTSRALITDGENSLVGGVMGFSILLSA | |
| | | EVFKHDSAVWYLDGSIGVLIGLTIFAYGVKLLIDMVPRVRQTRHYEMFE | |
| 62 | GAK | MSLLQSALDFLAGPGSLGGASGRDQSDFVGQTVELGELRLRVRRVLAEGGFAFVYEAQDV | ENA D88435.1 |
| | | GSGREYALKRLLSNEEEKNRAIIQEVCFMKKLSGHPNIVQFCSAASIGKEESDTGQAEFL | |
| | | LLTELCKGQLVEFLKKMESRGPLSCDTVLKIFYQTCRAVQHMHRQKPPIIHRDLKVENLL | |
| | | LSNQGTIKLCDFGSATTISHYPDYSWSAQRRALVEEEITRNTTPMYRTPEIIDLYSNFPI | |
| | | GEKQDIWALGCILYLLCFRQHPFEDGAKLRIVNGKYSIPPHDTQYTVFHSLIRAMLQVNP | |
| | | EERLSIAEVVHQLQEIAAARNVNPKSPITELLEQNGGYGSATLSRGPPPPVGPAGSGYSG | |
| | | GLALAEYDQPYGGFLDILRGGTERLFTNLKDTSSKVIQSVANYAKGDLDISYITSRIAVM | |
| | | SEPAEGVESALKNNIEDVRLFLDSKHPGHYAVYNLSPRTYRPHRFHNRVSECGWAARRAP | |
| | | HLHTLYNICRNMHAWLRQDHKNVCVVHCMDGRAASAVAVCSFLCFCRLFSTAEAAVYMFS | |
| | | MKRCPPGIWPSHKRYIEYMCDMVAEEPITPHSKPILVRAVVMTPVPLFSKQRSGCRPFCE | |
| | | VYVGDERVASTSQEYDKMRDFKIEDGKAVIPLGVTVQGDVLIVIYHARSTLGGRLQAKMA | |
| | | SMKMFQIQFHTGFVPRNATTVKFAKYDLDACDIQEKYPDLFQVNLEVEVEPRDRPSREAP | |
| | | PWENSSMRGLNPKILFSSREEQQDILSKFGKPELPRQPGSTAQYDAGAGSPEAEPTDSDS | |
| | | PPSSSADASRFLHTLDWQEEKEAETGAENASSKESESALMEDRDESEVSDEGGSPISSEG | |
| | | QEPRADPEPPGLAAGLVQQDLVFEVETPAVLPEPVPQEDGVDLLGLHSEVGAGPAVPPQA | |
| | | CKAPSSNTDLLSCLLGPPEAASQGPPEDLLSEDPLLLASPAPPLSVQSTPRGGPPAAADP | |
| | | FGPLLPSSGNNSQPCSNPDLFGEFLNSDSVTVPPSFPSAHSAPPPSCSADFLHLGDLPGE | |
| | | PSKMTASSSNPDLLGGWAAWTETAASAVAPTPATEGPLFSPGGQPAPCGSQASWTKSQNP | |
| | | DPFADLGDLSSGLQGSPAGFPPGGFIPKTATTPKGSSSWQTSRPPAQGASWPPQAKPPPK | |
| | | ACTQPRPNYASNFSVIGAREERGVRAPSFAQKPKVSENDFEDLLSNQGFSSRSDKKGPKT | |
| | | IAEMRKQDLAKDTDPLKLKLLDWIEGKERNIRALLSTLHTVLWDGESRWTPVGMADLVAP | |
| | | EQVKKHYRRAVLAVHPDKAAGQPYEQHAKMIFMELNDAWSEFENQGSRPLF | |
| 63 | FGF20 | MAPLAEVGGFLGGLEGLGQQVGSHELLPPAGERPPLLGERRSAAERSARGGPGAAQLAHL | ENA AB044277.1 |
| | | HGILRRRQLYCRTGFHLQILPDGSVQGTRQDHSLFGILEFISVAVGLVSIRGVDSGLYLG | |
| | | MNDKGELYGSEKLTSECIFREQFEENWYNTYSSNIYKHGDTGRRYFVALNKDGTPRDGAR | |
| | | SKRHQKFTHFLPRPVDPERVPELYKDLLMYT | |
| 64 | DLG2 | MFFACYCALRTNVKKYRYQDEDAPHDHSLPRLTHEVRGPELVHVSEKNLSQIENVHGYVL | ENA U32376.1 |
| | | QSHISPLKASPAPIIVNTDTLDTIPYVNGTEIEYEFEEITLERGNSGLGFSIAGGTDNPH | |
| | | IGDDPGIFITKIIPGGAAAEDGRLRVNDCILRVNEVDVSEVSHSKAVEALKEAGSIVRLY | |
| | | VRRRRPILETVVEIKLFKGPKGLGFSIAGGVGNQHIPGDNSIYVTKIIDGGAAQKDGRLQ | |
| | | VGDRLLMVNNYSLEEVTHEEAVAILKNTSEVVYLKVGKPTTIYMTDPYGPPDITHSYSPP | |
| | | MENHLLSGNNGTLEYKTSLPPISPGRYSPIPKHMLVDDDYTRPPEPVYSTVNKLCDKPAS | |
| | | PRHYSPVECDKSFLLSAPYSHYHLGLLPDSEMTSHSQHSTATRQPSMTLQRAVSLEGEPR | |
| | | KVVLHKGSTGLGFNIVGGEDGEGIFVSFILAGGPADLSGELQRGDQILSVNGIDLRGASH | |
| | | EQAAAALKGAGQTVTIIAQYQPEDYARFEAKIHDLREQMMNHSMSSGSGSLRTNQKRSLY | |
| | | VRAMFDYDKSKDSGLPSQGLSFKYGDILHVINASDDEWWQARRVMLEGDSEEMGVIPSKR | |
| | | RVERKERARLKTVKFNAKPGVIDSKGSFNDKRKKSFIFSRKFPFYKNKEQSEQETSDPER | |
| | | GQEDLILSYEPVTRQEINYTRPVIILGPMKDRINDDLISEFPDKEGSCVPHTTRPKRDYE | |
| | | VDGRDYHFVISREQMEKDIQEHKFIEAGQYNDNLYGTSVQSVRFVAERGKHCILDVSGNA | |
| | | IKRLQVAQLYPIAIFIKPRSLEPLMEMNKRLTEEQAKKTYDRAIKLEQEFGEYFTAIVQG | |
| | | DTLEDIYNQCKLVIEEQSGPFIWIPSKEKL | |
| 65 | DDRGK | MVAPVWYLVAAALLVGFILFLTRSRGRAASAGQEPLHNEELAGAGRVAQPGPLEPEEPRA | ENABC000643.1 |
| | 1 | GGRPRRRRDLGSRLQAQRRAQRVAWAEADENEEEAVILAQEEEGVEKPAETHLSGKIGAK | |
| | | KLRKLEEKQARKAQREAEEAEREERKRLESQREAEWKKEEERLRLEEEQKEEEERKAREE | |
| | | QAQREHEEYLKLKEAFVVEEEGVGETMTEEQSQSFLTEFINYIKQSKVVLLEDLASQVGL | |
| | | RTQDTINRIQDLLAEGTITGVIDDRGKFIYITPEELAAVANFIRQRGRVSIAELAQASNS | |
| | | LIAWGRESPAQAPA | |
| 66 | SREBF | MDEPPFSEAALEQALGEPCDLDAALLTDIEDMLQLINNQDSDFPGLFDPPYAGSGAGGTD | ENA U00968.1 |
| | | PASPDTSSPGSLSPPPATLSSSLEAFLSGPQAAPSPLSPPQPAPTPLKMYPSMPAFSPGP | |
| | | GIKEESVPLSILQTPTPQPLPGALLPQSFPAPAPPQFSSTPVLGYPSPPGGFSTGSPPGN | |
| | | TQQPLPGLPLASPPGVPPVSLHTQVQSVVPQQLLTVTAAPTAAPVTTTVTSQIQQVPVLL | |
| | | QPHFIKADSLLLTAMKTDGATVKAAGLSPLVSGTTVQTGPLPTLVSGGTILATVPLVVDA | |
| | | EKLPINRLAAGSKAPASAQSRGEKRTAHNAIEKRYRSSINDKIIELKDLVVGTEAKLNKS | |
| | | AVLRKAIDYIRFLQHSNQKLKQENLSLRTAVEKSKSLKDLVSACGSGGNTDVLMEGVKTE | |
| | | VEDTLTPPPSDAGSPFQSSPLSLGSRGSGSGGSGSDSEPDSPVFEDSKAKPEQRPSLHSR | |
| | | GMLDRSRLALCTLVFLCLSCNPLASLLGARGLPSPSDTTSVYHSPGRNVLGTESRDGPGW | |
| | | AQWLLPPVVWLLNGLLVLVSLVLLFVYGEPVTRPHSGPAVYFWRHRKQADLDLARGDFAQ | |
| | | AAQQLWLALRALGRPLPTSHLDLACSLLWNLIRHLLQRLWVGRWLAGRAGGLQQDCALRV | |
| | | DASASARDAALVYHKLHQLHTMGKHTGGHLTATNLALSALNLAECAGDAVSVATLAEIYV | |
| | | AAALRVKTSLPRALHFLTRFFLSSARQACLAQSGSVPPAMQWLCHPVGHRFFVDGDWSVL | |
| | | STPWESLYSLAGNPVDPLAQVTQLFREHLLERALNCVTQPNPSPGSADGDKEFSDALGYL | |
| | | QLLNSCSDAAGAPAYSFSISSSMATTIGVDPVAKWWASLTAVVIHWLRRDEEAAERLCPL | |
| | | VEHLPRVLQESERPLPRAALHSFKAARALLGCAKAESGPASLTICEKASGYLQDSLATTP | |
| | | ASSSIDKAVQLFLCDLLLVVRTSLWRQQQPPAPAPAAQGTSSRPQASALELRGFQRDLSS | |
| | | LRRLAQSFRPAMRRVFLHEATARLMAGASPTRTHQLLDRSLRRRAGPGGKGGAVAELEPR | |
| | | PTRREHAEALLLASCYLPPGFLSAPGQRVGMLAEAARTLEKLGDRRLLHDCQQMLMRLGG | |
| | | GTTVTSS | |
| 67 | BCKDK | MILASVLRSGPGGGLPLRPLLGPALALRARSTSATDTHHVEMARERSKTVTSFYNQSAID | ENA AF026548.1 |
| | | AAAEKPSVRLTPTMMLYAGRSQDGSHLLKSARYLQQELPVRIAHRIKGFRCLPFIIGCNP | |
| | | TILHVHELYIRAFQKLTDFPPIKDQADEAQYCQLVRQLLDDHKDVVTLLAEGLRESRKHI | |
| | | EDEKLVRYFLDKTLTSRLGIRMLATHHLALHEDKPDFVGIICTRLSPKKIIEKWVDFARR | |
| | | LCEHKYGNAPRVRINGHVAARFPFIPMPLDYILPELLKNAMRATMESHLDTPYNVPDVVI | |
| | | TIANNDVDLIIRISDRGGGIAHKDLDRVMDYHFTTAEASTQDPRISPLFGHLDMHSGAQS | |
| | | GPMHGEGFGLPTSRAYAEYLGGSLQLQSLQGIGTDVYLRLRHIDGREESFRI | |
| 68 | PARK2 | MIVEVRENSSHGFPVEVDSDTSIFQLKEVVAKRQGVPADQLRVIFAGKELRNDWTVQNCD | ENA AB009973.1 |
| | | LDQQSIVHIVQRPWRKGQEMNATGGDDPRNAAGGCEREPQSLTRVDLSSSVLPGDSVGLA | |
| | | VILHTDSRKDSPPAGSPAGRSIYNSFYVYCKGPCQRVQPGKLRVQCSTCRQATLTLTQGP | |
| | | SCWDDVLIPNRMSGECQSPHCPGTSAEFFFKCGAHPTSDKETSVALHLIATNSRNITCIT | |
| | | CTDVRSPVLVFQCNSRHVICLDCFHLYCVTRLNDRQFVHDPQLGYSLPCVAGCPNSLIKE | |
| | | LHHFRILGEEQYNRYQQYGAEECVLQMGGVLCPRPGCGAGLLPEPDQRKVTCEGGNGLGC | |
| | | GFAFCRECKEAYHEGECSAVFEASGTTTQAYRVDERAAEQARWEAASKETIKKTTKPCPR | |
| | | CHVPVEKNGGCMHMKCPQPQCRLEWCWNCGCEWNRVCMGDHWFDV | |
| 69 | RAB39B | MEAIWLYQFRLIVIGDSTVGKSCLIRRFTEGRFAQVSDPTVGVDFFSRLVEIEPGKRIKL | ENA AY052478.1 |
| | | QIWDTAGQERFRSITRAYYRNSVGGLLLFDITNRRSFQNVHEWLEETKVHVQPYQIVFVL | |
| | | VGHKCDLDTQRQVTRHEAEKLAAAYGMKYIETSARDAINVEKAFTDLTRDIYELVKRGEI | |
| | | TIQEGWEGVKSGFVPNVVHSSFEVVKSERRCLC | |
| 70 | DNAJC | MKDSENKGASSPDMEPSYGGGLFDMVKGGAGRLFSNLKDNLKDTLKDTSSRVIQSVTSYT | ENA AB007942.1 |
| | 6 | KGDLDFTYVTSRIIVMSFPLDNVDIGFRNQVDDIRSFLDSRHLDHYTVYNLSPKSYRTAK | |
| | | FHSRVSECSWPIRQAPSLHNLFAVCRNMYNWLLQNPKNVCVVHCLDGRAASSILVGAMFI | |
| | | FCNLYSTPGPAIRLLYAKRPGIGLSPSHRRYLGYMCDLLADKPYRPHFKPLTIKSITVSP | |
| | | IPFFNKQRNGCRPYCDVLIGETKIYSICTDFERMKEYRVQDGKIFIPLNITVQGDVVVSM | |
| | | YHLRSTIGSRLQAKVTNTQIFQLQFHTGFIPLDTTVLKFTKPELDACDVPEKYPQLFQVT | |
| | | LDVELQPHDKVIDLTPPWEHYCTKDVNPSILESSHQEHQDTLALGGQAPIDIPPDNPRHY | |
| | | GQSGEFASLCWQDQKSEKSECEEDHAALVNQESEQSDDELLTLSSPHGNANGDKPHGVKK | |
| | | PSKKQQEPAAPPPPEDVDLLGLEGSAMSNSFSPPAAPPTNSELLSDLFGGGGAAGPTQAG | |
| | | QSGVEDVFHPSGPASTQSTPRRSATSTSASPTLRVGEGATFDPFGAPSKPSGQDLLGSFL | |
| | | NTSSASSDPFLQPTRSPSPTVHASSTPAVNIQPDVSGGWDWHAKPGGFGMGSKSAATSPT | |
| | | GSSHGTPTHQSKPQTLDPFADLGTLGSSSFASKPTTPTGLGGGFPPLSSPQKASPQPMGG | |
| | | GWQQGGAYNWQQPQPKPQPSMPHSSPQNRPNYNVSFSAMPGGQNERGKGSSNLEGKQKAA | |
| | | DFEDLLSGQGFNAHKDKKGPRTIAEMRKEEMAKEMDPEKLKILEWIEGKERNIRALLSTM | |
| | | HTVLWAGETKWKPVGMADLVTPEQVKKVYRKAVLVVHPDKATGQPYEQYAKMIFMELNDA | |
| | | WSEFENQGQKPLY | |
| 71 | SMPD1 | MPRYGASLRQSCPRSGREQGQDGTAGAPGLLWMGLVLALALALALALALSDSRVLWAPAE | ENA M59916.1 |
| | | AHPLSPQGHPARLHRIVPRLRDVFGWGNLTCPICKGLFTAINLGLKKEPNVARVGSVAIK | |
| | | LCNLLKIAPPAVCQSIVHLFEDDMVEVWRRSVLSPSEACGLLLGSTCGHWDIFSSWNISL | |
| | | PTVPKPPPKPPSPPAPGAPVSRILFLTDLHWDHDYLEGTDPDCADPLCCRRGSGLPPASR | |
| | | PGAGYWGEYSKCDLPLRTLESLLSGLGPAGPFDMVYWTGDIPAHDVWHQTRQDQLRALTT | |
| | | VTALVRKFLGPVPVYPAVGNHESTPVNSFPPPFIEGNHSSRWLYEAMAKAWEPWLPAEAL | |
| | | RTLRIGGFYALSPYPGLRLISLNMNFCSRENFWLLINSTDPAGQLQWLVGELQAAEDRGD | |
| | | KVHIIGHIPPGHCLKSWSWNYYRIVARYENTLAAQFFGHTHVDEFEVFYDEETLSRPLAV | |
| | | AFLAPSATTYIGLNPGYRVYQIDGNYSGSSHVVLDHETYILNLTQANIPGAIPHWQLLYR | |
| | | ARETYGLPNTLPTAWHNLVYRMRGDMQLFQTFWFLYHKGHPPSEPCGTPCRLATLCAQLS | |
| | | ARADSPALCRHLMPDGSLPEAQSLWPRPLFC | |
| 72 | TMEM1 | MSQPRTPEQALDTPGDCPPGRRDEDAGEGIQCSQRMLSFSDALLSIIATVMILPVTHTEI | ENA AL834199.1 |
| | 75 | SPEQQFDRSVQRLLATRIAVYLMTFLIVTVAWAAHTRLFQVVGKTDDTLALLNLACMMTI | |
| | | TFLPYTFSLMVTFPDVPLGIFLFCVCVIAIGVVQALIVGYAFHFPHLLSPQIQRSAHRAL | |
| | | YRRHVLGIVLQGPALCFAAAIFSLFFVPLSYLLMVTVILLPYVSKVTGWCRDRLLGHREP | |
| | | SAHPVEVFSFDLHEPLSKERVEAFSDGVYAIVATLLILDICEDNVPDPKDVKERFSGSLV | |
| | | AALSATGPRFLAYFGSFATVGLLWFAHHSLFLHVRKATRAMGLLNTLSLAFVGGLPLAYQ | |
| | | QTSAFARQPRDELERVRVSCTIIFLASIFQLAMWITALLHQAETLQPSVWFGGREHVLMF | |
| | | AKLALYPCASLLAFASTCLLSRFSVGIFHLMQIAVPCAFLLLRLLVGLALATLRVLRGLA | |
| | | RPEHPPPAPTGQDDPQSQLLPAPC | |
| 73 | STK39 | MAEPSGSPVHVQLPQQAAPVTAAAAAAPAAATAAPAPAAPAAPAPAPAPAAQAVGWPICR | ENA AF099989.1 |
| | | DAYELQEVIGSGATAVVQAALCKPRQERVAIKRINLEKCQTSMDELLKEIQAMSQCSHPN | |
| | | VVTYYTSFVVKDELWLVMKLLSGGSMLDIIKYIVNRGEHKNGVLEEAIIATILKEVLEGL | |
| | | DYLHRNGQIHRDLKAGNILLGEDGSVQIADFGVSAFLATGGDVTRNKVRKTFVGTPCWMA | |
| | | PEVMEQVRGYDFKADMWSFGITAIELATGAAPYHKYPPMKVLMLTLQNDPPTLETGVEDK | |
| | | EMMKKYGKSFRKLLSLCLQKDPSKRPTAAELLKCKFFQKAKNREYLIEKLLTRTPDIAQR | |
| | | AKKVRRVPGSSGHLHKTEDGDWEWSDDEMDEKSEEGKAAFSQEKSRRVKEENPEIAVSAS | |
| | | TIPEQIQSLSVEDSQGPPNANEDYREASSCAVNLVLRLRNSRKELNDIRFEFTPGRDTAD | |
| | | GVSQELFSAGLVDGHDVVIVAANLQKIVDDPKALKTLTFKLASGCDGSEIPDEVKLIGRA | |
| | | QLSVS | |
| 74 | BST1 | MAAQGCAASRLLQLLLQLLLLLLLLAAGGARARWRGEGTSAHLRDIFLGRCAEYRALLSP | ENA D21878.1 |
| | | EQRNKNCTAIWEAFKVALDKDPCSVLPSDYDLFINLSRHSIPRDKSLFWENSHLLVNSFA | |
| | | DNTRRFMPLSDVLYGRVADFLSWCRQKNDSGLDYQSCPTSEDCENNPVDSFWKRASIQYS | |
| | | KDSSGVIHVMLNGSEPTGAYPIKGFFADYEIPNLQKEKITRIEIWVMHEIGGPNVESCGE | |
| | | GSMKVLEKRLKDMGFQYSCINDYRPVKLLQCVDHSTHPDCALKSAAAATQRKAPSLYTEQ | |
| | | RAGLIIPLFLVLASRIQL | |
| 75 | MMP16 | MILLITSTGRRLDFVHHSGVFFLQTLLWILCATVCGTEQYFNVEVWLQKYGYLPPTDPRM | ENA AB009303.1 |
| | | SVLRSAETMQSALAAMQQFYGINMTGKVDRNTIDWMKKPRCGVPDQTRGSSKFHIRRKRY | |
| | | ALTGQKWQHKHITYSIKNVTPKVGDPETRKAIRRAFDVWQNVTPLTFEEVPYSELENGKR | |
| | | DVDITIIFASGFHGDSSPFDGEGGFLAHAYFPGPGIGGDTHFDSDEPWTLGNPNHDGNDL | |
| | | FLVAVHELGHALGLEHSNDPTAIMAPFYQYMETDNFKLPNDDLQGIQKIYGPPDKIPPPT | |
| | | RPLPTVPPHRSIPPADPRKNDRPKPPRPPTGRPSYPGAKPNICDGNFNTLAILRREMFVF | |
| | | KDQWFWRVRNNRVMDGYPMQITYFWRGLPPSIDAVYENSDGNFVFFKGNKYWVFKDTTLQ | |
| | | PGYPHDLITLGSGIPPHGIDSAIWWEDVGKTYFFKGDRYWRYSEEMKTMDPGYPKPITVW | |
| | | KGIPESPQGAFVHKENGFTYFYKGKEYWKFNNQILKVEPGYPRSILKDFMGCDGPTDRVK | |
| | | EGHSPPDDVDIVIKLDNTASTVKAIAIVIPCILALCLLVLVYTVFQFKRKGTPRHILYCK | |
| | | RSMQEWV | |
| 76 | RIT2 | MEVENEASCSPGSASGGSREYKVVMLGAGGVGKSAMTMQFISHQFPDYHDPTIEDAYKTQ | ENA U71204.1 |
| | | VRIDNEPAYLDILDTAGQAEFTAMREQYMRGGEGFIICYSVTDRQSFQEAAKFKELIFQV | |
| | | RHTYEIPLVLVGNKIDLEQFRQVSTEEGLSLAQEYNCGFFETSAALRFCIDDAFHGLVRE | |
| | | IRKKESMPSLMEKKLKRKDSLWKKLKGSLKKKRENMT | |
| 77 | FAM47 | MADRRRRLRPGTLAPVREGVNCRSRCFTKHKNGLKFPTSLHSRQLVFPRKGLDDFRKGCP | ENA AK092277.1 |
| | E | PCTGLVTQVPVEGFLPQIYHRAPQLAPKKRQIKLLKEADVLSKLSPAQQARKAFLEDVEA | |
| | | HLTPHPLALYLNLEEAMPIELLSKVLEVLDPDRKLEDTWAYCQDTRKGMKEPTKLLKKHS | |
| | | TQVYLGPSKKTSVSNAGQWLYEEKPHKMDLLHENGPRPGLHENGISDIDEEFILKQFDID | |
| | | YETKPSHDALHTMKLNQVPLELKRSVGLSKLQETEFFQKLGYERKLQKPQNPYKPKWVKM | |
| | | RYGAWYLNPKLWKKQRVDEPLVDPEVSHKAQEENFKKELQEQEELLADLHGTVAFKDFIL | |
| | | SRGYRTPRFLENMYIGKECKRACNKTPIKRTQA | |
| 78 | CCDC6 | MNPPAAFLAGRQNIGSEVEISTTEKQRKELQLLIGELKDRDKELNDMVAVHQQQLLSWEE | ENA AY254201.1 |
| | 2 | DRQKVLTLEERCSKLEGELHKRTEIIRSLTKKVKALESNQMECQTALQKTQLQLQEMAQK | |
| | | ATHSSLLSEDLEARNETLSNTLVELSAQVGQLQAREQALTTMIKLKDKDIIEAVNHTADC | |
| | | SGKFKMLEHALRDAKMAETCIVKEKQDYKQKLKALKIEVNKLKEDLNEKTTENNEQREEI | |
| | | IRLKQEKSCLHDELLFTVEREKRKDELLNIAKSKQERINSELHNLRQIYVKQQSDLQFLN | |
| | | FNVENSQELIQMYDSKMEESKALDSSRDMCLSDLENNHPKVDIKREKNQKSLFKDQKFEA | |
| | | MLVQQNRSDKSSCDECKEKKQQIDTVFGEKSVITLSSIFTKDLVEKHNLPWSLGGKTQIE | |
| | | PENKITLCKIHTKSPKCHGTGVQNEGKQPSETPTLSDEKQWHDVSVYLGLTNCPSSKHPE | |
| | | KLDVECQDQMERSEISCCQKNEACLGESGMCDSKCCHPSNFIIEAPGHMSDVEWMSIFKP | |
| | | SKMQRIVRLKSGCTCSESICGTQHDSPASELIAIQDSHSLGSSKSALREDETESSSNKKN | |
| | | SPTSLLIYKDAPAFNEKASTVLPSQDDFSPTSKLQRLLAESRQMVTDLELSTLLPISHEN | |
| | | LTGSATNKSEVPEESAQKNTFVSY | |
| 79 | HLA- | MSWKKALRIPGGLRAATVTLMLAMLSTPVAEGRDSPEDFVYQFKAMCYFTNGTERVRYVT | ENA X03068.1 |
| | DQB1 | RYIYNREEYARFDSDVEVYRAVTPLGPPDAEYWNSQKEVLERTRAELDTVCRHNYQLELR | |
| | | TTLQRRVEPTVTISPSRTEALNHHNLLVCSVTDFYPAQIKVRWFRNDQEETTGVVSTPLI | |
| | | RNGDWTFQILVMLEMTPQHGDVYTCHVEHPSLQNPITVEWRAQSESAQSKMLSGIGGFVL | |
| | | GLIFLGLGLIIHHRSQKGLLH | |
| 8O | TMEM2 | MASAEPLTALSRWYLYAIHGYFCEVMFTAAWEFVVNLNWKFPGVTSVWALFIYGTSILIV | ENA AK090706.1 |
| | 29B | ERMYLRLRGRCPLLLRCLIYTLWTYLWEFTTGFILRQFNACPWDYSQFDFDFMGLITLEY | |
| | | AVPWFCGALIMEQFIIRNTLRLRFDKDAEPGEPSGALALANGHVKTD | |
| 81 | MAPT | MAEPRQEFEVMEDHAGTYGLGDRKDQGGYTMHQDQEGDTDAGLKESPLQTPTEDGSEEPG | ENA J03778.1 |
| | | SETSDAKSTPTAEDVTAPLVDEGAPGKQAAAQPHTEIPEGTTAEEAGIGDTPSLEDEAAG | |
| | | HVTQEPESGKVVQEGFLREPGPPGLSHQLMSGMPGAPLLPEGPREATRQPSGTGPEDTEG | |
| | | GRHAPELLKHQLLGDLHQEGPPLKGAGGKERPGSKEEVDEDRDVDESSPQDSPPSKASPA | |
| | | QDGRPPQTAAREATSIPGFPAEGAIPLPVDFLSKVSTEIPASEPDGPSVGRAKGQDAPLE | |
| | | FTFHVEITPNVQKEQAHSEEHLGRAAFPGAPGEGPEARGPSLGEDTKEADLPEPSEKQPA | |
| | | AAPRGKPVSRVPQLKARMVSKSKDGTGSDDKKAKTSTRSSAKTLKNRECLSPKHPTPGSS | |
| | | DPLIQPSSPAVCPEPPSSPKYVSSVTSRTGSSGAKEMKLKGADGKTKIATPRGAAPPGQK | |
| | | GQANATRIPAKTPPAPKTPPSSGEPPKSGDRSGYSSPGSPGTPGSRSRTPSLPTPPTREF | |
| | | KKVAVVRTPPKSPSSAKSRLQTAPVPMPDLKNVKSKIGSTENLKHQPGGGKVQIINKKLD | |
| | | LSNVQSKCGSKDNIKHVPGGGSVQIVYKPVDLSKVTSKCGSLGNIHHKPGGGQVEVKSEK | |
| | | LDFKDRVQSKIGSLDNITHVPGGGNKKIETHKLTFRENAKAKTDHGAEIVYKSPVVSGDT | |
| | | SPRHLSNVSSTGSIDMVDSPQLATLADEVSASLAKQGL | |
| 82 | SPPL2B | MAAAVAAALARLLAAFLLLAAQVACEYGMVHVVSQAGGPEGKDYCILYNPQWAHLPHDLS | ENA AJ345027.1 |
| | | KASFLQLRNWTASLLCSAADLPARGFSNQIPLVARGNCTFYEKVRLAQGSGARGLLIVSR | |
| | | ERLVPPGGNKTQYDEIGIPVALLSYKDMLDIFTRFGRTVRAALYAPKEPVLDYNMVIIFI | |
| | | MAVGTVAIGGYWAGSRDVKKRYMKHKRDDGPEKQEDEAVDVTPVMTOVFVVMCCSMLVLL | |
| | | YYFYDLLVYVVIGIFCLASATGLYSCLAPCVRRLPFGKCRIPNNSLPYFHKRPQARMLLL | |
| | | ALFCVAVSVVWGVFRNEDQWAWVLQDALGIAFCLYMLKTIRLPTFKACTLLLLVLFLYDI | |
| | | FFVFITPFLTKSGSSIMVEVATGPSDSATREKLPMVLKVPRLNSSPLALCDREFSLLGFG | |
| | | DILVPGLLVAYCHRFDIQVQSSRVYFVACTIAYGVGLLVTFVALALMQRGQPALLYLVPC | |
| | | TLVTSCAVALWRRELGVFWTGSGFAKVLPPSPWAPAPADGPQPPKDSATPLSPQPPSEEP | |
| | | ATSPWPAEQSPKSRTSEEMGAGAPMREPGSPAESEGRDQAQPSPVTQPGASA | |
| 83 | ITGAB | MSPGASRGPRGSQAPLIAPLCCAAAALGMLLWSPACQAFNLDVEKLTVYSGPKGSYFGYA | ENA L36531.1 |
| | | VDFHIPDARTASVLVGAPKANTSQPDIVEGGAVYYCPWPAEGSAQCRQIPFDTTNNRKIR | |
| | | VNGTKEPIEFKSNQWFGATVKAHKGKVVACAPLYHWRTLKPTPEKDPVGTCYVAIQNFSA | |
| | | YAEFSPCRNSNADPEGQGYCQAGFSLDFYKNGDLIVGGPGSFYWQGQVITASVADIIANY | |
| | | SFKDILRKLAGEKQTEVAPASYDDSYLGYSVAAGEFTGDSQQELVAGIPRGAQNFGYVSI | |
| | | INSTDMTFIQNFTGEQMASYFGYTVVVSDVNSDGLDDVLVGAPLFMEREFESNPREVGQI | |
| | | YLYLQVSSLLFRDPQILTGTETEGRFGSAMAHLGDLNQDGYNDIAIGVPFAGKDQRGKVL | |
| | | IYNGNKDGLNTKPSQVLQGVWASHAVPSGEGFTLRGDSDIDKNDYPDLIVGAFGTGKVAV | |
| | | YRARPVVTVDAQLLLHPMIINLENKTCQVPDSMTSAACFSLRVCASVTGQSIANTIVLMA | |
| | | EVQLDSLKQKGAIKRTLFLDNHQAHRVFPLVIKRQKSHQCQDFIVYLRDETEFRDKLSPI | |
| | | NISLNYSLDESTFKEGLEVKPILNYYRENIVSEQAHILVDCGEDNLCVPDLKLSARPDKH | |
| | | QVIIGDENHLMLIINARNEGEGAYEAELFVMIPEEADYVGIERNNKGFRPLSCEYKMENV | |
| | | TRMVVCDLGNPMVSGTNYSLGLRFAVPRLEKTNMSINFDLQIRSSNKDNPDSNFVSLQIN | |
| | | ITAVAQVEIRGVSHPPQIVLPIHNWEPEEEPHKEEEVGPLVEHIYELHNIGPSTISDTIL | |
| | | EVGWPFSARDEFLLYIFHIQTLGPLQCQPNPNINPQDIKPAASPEDTPELSAFLRNSTIP | |
| | | HLVRKRDVHVVEFHRQSPAKILNCTNIECLQISCAVGRLEGGESAVLKVRSRLWAHTFLQ | |
| | | RKNDPYALASLVSFEVKKMPYTDQPAKLPEGSIVIKTSVIWATPNVSFSIPLWVIILAIL | |
| | | LGLLVLAILTLALWKCGFFDRARPPQEDMTDREQLTNDKTPEA | |
| 84 | ATP13A | MSADSSPLVGSTPTGYGTLTIGTSIDPLSSSVSSVRLSGYCGSPWRVIGYHVVVWMMAGI | ENA AL354615.1 |
| | 2 | PLLLFRWKPLWGVRLRLRPCNLAHAETLVIEIRDKEDSSWQLFTVQVQTEAIGEGSLEPS | |
| | | PQSQAEDGRSQAAVGAVPEGAWKDTAQLHKSEEAVSVGQKRVLRYYLFQGQRYIWIETQQ | |
| | | AFYQVSLLDHGRSCDDVHRSRHGLSLQDQMVRKAIYGPNVISIPVKSYPQLLVDEALNPY | |
| | | YGFQAFSIALWLADHYYWYALCIFLISSISICLSLYKTRKQSQTLRDMVKLSMRVCVCRP | |
| | | GGEEEWVDSSELVPGDCLVLPQEGGLMPCDAALVAGECMVNESSLTGESIPVLKTALPEG | |
| | | LGPYCAETHRRHTLFCGTLILQARAYVGPHVLAVVTRTGFCTAKGGLVSSILHPRPINFK | |
| | | FYKHSMKFVAALSVLALLGTIYSIFILYRNRVPLNEIVIRALDLVTVVVPPALPAAMTVC | |
| | | TLYAQSRLRRQGIFCIHPLRINLGGKLQLVCFDKTGTLTEDGLDVMGVVPLKGQAFLPLV | |
| | | PEPRRLPVGPLLRALATCHALSRLQDTPVGDPMDLKMVESTGWVLEEEPAADSAFGTQVL | |
| | | AVMRPPLWEPQLQAMEEPPVPVSVLHRFPFSSALQRMSVVVAWPGATQPEAYVKGSPELV | |
| | | AGLCNPETVPTDFAQMLQSYTAAGYRVVALASKPLPTVPSLEAAQQLTRDTVEGDLSLLG | |
| | | LLVMRNLLKPQTTPVIQALRRTRIRAVMVTGDNLQTAVTVARGCGMVAPQEHLIIVHATH | |
| | | PERGQPASLEFLPMESPTAVNGVKDPDQAASYTVEPDPRSRHLALSGPTEGIIVKHFPKL | |
| | | LPKVLVQGTVFARMAPEQKTELVCELQKLQYCVGMCGDGANDCGALKAADVGISLSQAEA | |
| | | SVVSPFTSSMASIECVPMVIREGRCSLDTSFSVFKYMALYSLTQFISVLILYTINTNLGD | |
| | | LQFLAIDLVITTTVAVLMSRTGPALVLGRVRPPGALLSVPVLSSLLLQMVLVTGVQLGGY | |
| | | FLTLAQPWFVPLNRTVAAPDNLPNYENTVVFSLSSFQYLILAAAVSKGAPFRRPLYTNVP | |
| | | FLVALALLSSVLVGLVLVPGLLQGPLALRNITDTGFKLLLLGLVTLNFVGAFMLESVLDQ | |
| | | CLPACLRRLRPKRASKKRFKQLERELAEQPWPPLPAGPLR | |
| 85 | DGKQ | MAAAAEPGARAWLGGGSPRPGSPACSPVLGSGGRARPGPGPGPGPERAGVRAPGPAAAPG | ENA L38707.1 |
| | | HSFRKVTLTKPTFCHLCSDFINGLAGFLCDVCNFMSHEKCLKHVRIPCTSVAPSLVRVPV | |
| | | AHCFGPRGLHKRKFCAVCRKVLEAPALHCEVCELHLHPDCVPFACSDCRQCHQDGHQDHD | |
| | | THHHHWREGNLPSGARCEVCRKTCGSSDVLAGVRCEWCGVQAHSLCSAALAPECGFGRLR | |
| | | SLVLPPACVRLLPGGPSKTQSFRIVEAAEPGEGGDGADGSAAVGPGRETQATPESGKQTL | |
| | | KIFDGDDAVRRSQFRLVTVSRLAGAEEVLEAALRAHHIPEDPGHLELCRLPPSSQACDAW | |
| | | AGGKAGSAVISEEGRSPGSGEATPEAWVIRALPRAQEVLKIYPGWLKVGVAYVSVRVTPK | |
| | | STARSVVLEVLPLLGRQAESPESFQLVEVAMGCRHVQRTMLMDEQPLLDRLQDIRQMSVR | |
| | | QVSQTREYVAESRDVAPHVSLEVGGLPPGLSPEEYSSLLHEAGATKATVVSVSHIYSSQG | |
| | | AVVLDVACFAEAERLYMLLKDMAVRGRLLTALVLPDLLHAKLPPDSCPLLVEVNPKSGGL | |
| | | KGRDLLCSFRKLLNPHQVFDLTNGGPLPGLHLFSQVPCFRVLVCGGDGTVGWVLGALEET | |
| | | RYRLACPEPSVAILPLGTGNDLGRVLRWGAGYSGEDPFSVLLSVDEADAVLMDRWTILLD | |
| | | AHEAGSAENDTADAEPPKIVQMSNYCGIGIDAELSLDFHQAREEEPGKFTSRLHNKGVYV | |
| | | RVGLQKISHSRSLHKQIRLQVERQEVELPSIEGLIFINIPSWGSGADLWGSDSDTRFEKP | |
| | | RMDDGLLEVVGVTGVVHMGQVQGGLRSGIRIAQGSYFRVTLLKATPVQVDGEPWVQAPGH | |
| | | MIISAAGPKVHMLRKAKQKPRRAGTTRDARADAAPAPESDPR | |
| 86 | STX1B | MKDRTQELRSAKDSDDEEEVVHVDRDHEMDEFFEQVEEIRGCIEKLSEDVEQVKKQHSAI | ENA AY995211.1 |
| | | LAAPNPDEKTKQELEDLTADIKKTANKVRSKLKAIEQSIEQEEGLNRSSADLRIRKTQHS | |
| | | TLSRKFVEVMTEYNATQSKYRDRCKDRIQRQLEITGRTTTNEELEDMLESGKLAIFTDDI | |
| | | KMDSQMTKQALNEIETRHNEIIKLETSIRELHDMFVDMAMLVESQGEMIDRIEYNVEHSV | |
| | | DYVERAVSDTKKAVKYQSKARRKKIMIIICCVVLGVVLASSIGGTLGL | |
| 87 | NUCKS | MSRPVRNRKVVDYSQFQESDDADEDYGRDSGPPTKKIRSSPREAKNKRRSGKNSQEDSED | ENA AJ012584.1 |
| | 1 | SEDKDVKTKKDDSHSAEDSEDEKEDHKNVRQQRQAASKAASKQREMLMEDVGSEEEQEEE | |
| | | DEAPFQEKDSGSDEDFLMEDDDDSDYGSSKKKNKKMVKKSKPERKEKKMPKPRLKATVTP | |
| | | SPVKGKGKVGRPTASKASKEKTPSPKEEDEEPESPPEKKTSTSPPPEKSGDEGSEDEAPS | |
| | | GED | |
| 88 | ACMSD | MKIDIHSHILPKEWPDLKKRFGYGGWVQLQHHSKGEAKLLKDGKVFRVVRENCWDPEVRI | ENA AB071418.1 |
| | | REMDQKGVTVQALSTVPVMFSYWAKPEDTLNLCQLLNNDLASTVVSYPRRFVGLGTLPMQ | |
| | | APELAVKEMERCVKELGFPGVQIGTHVNEWDLNAQELFPVYAAAERLKCSLFVHPWDMQM | |
| | | DGRMAKYWLPWLVGMPAETTIAICSMIMGGVFEKFPKLKVCFAHGGGAFPFTVGRISHGF | |
| | | SMRPDLCAQDNPMNPKKYLGSFYTDALVHDPLSLKLLTDVIGKDKVILGTDYPFPLGELE | |
| | | PGKLIESMEEFDEETKNKLKAGNALAFLGLERKQFE | |

Agents that elevate the expression and/or activity level of one or more of the foregoing proteins that may be used in conjunction with the compositions and methods of the disclosure include nucleic acids that encode the protein or plurality of proteins (e.g., nucleic acids capable of expression in macrophages or microglia). Such nucleic acid molecules may be provided to a patient (e.g., a patient having Alzheimer's disease) in the form, for example, of a population of cells, such as a population of cells (e.g., pluripotent cells, ESCs, iPSCs, multipotent cells, CD34+ cells, HSCs, MPCs, BLPCs, monocytes, macrophages, microglial progenitor cells, or microglia) that contain the nucleic acid molecules. Such cells may be modified ex vivo so as to express the nucleic acid molecule(s) of interest, for example, using transfection and transduction methods described herein. Additionally or alternatively, nucleic acid molecules encoding one or more of the proteins of interest may be provided to the patient in the form of one or more viral vectors that collectively encode the one or more proteins. Exemplary viral vectors that may be used in conjunction with the compositions and methods of the disclosure include Retroviridae family viral vectors, such as a lentivirus, alpharetrovirus, or gammaretrovirus, among others described herein. In some embodiments, the nucleic acid molecule(s) are administered directly to the patient. Additional agents that may be provided to a patient for the purpose of augmenting the level of one or more of the foregoing proteins include interfering RNA molecules, such as siRNA, shRNA, and miRNA molecules, as well as small molecule agents that modulate the expression of one or more of the above proteins, in addition to the one or more of the above proteins themselves.

### Frontotemporal Lobar Degeneration

FTLD is a clinical syndrome characterized by progressive neurodegeneration in the frontal and temporal lobes of the cerebral cortex. The manifestation of FTLD is complex and heterogeneous, and may present as one of three clinically distinct variants including: 1) behavioral-variant frontotemporal dementia (BVFTD), characterized by changes in behavior and personality, apathy, social withdrawal, perseverative behaviors, attentional deficits, disinhibition, and a pronounced degeneration of the frontal lobe; 2) semantic dementia (SD), characterized by fluent, anomic aphasia, progressive loss of semantic knowledge of words, objects, and concepts and a pronounced degeneration of the anterior temporal lobes. Furthermore, SD variant of FTLD exhibit a flat affect, social deficits, perseverative behaviors, and disinhibition; or 3) progressive nonfluent aphasia (PNA); characterized by motor deficits in speech production, reduced language expression, and pronounced degeneration of the perisylvian cortex. Neuronal loss in brains of FTLD patients is associated with one of three distinct neuropathologies: 1) the presence of tau-positive neuronal and glial inclusions; 2) ubiquitin (ub)-positive and TAR DNA-binding protein 43 (TDP43)-positive, but tau-negative inclusions; or 3) ub and fused in sarcoma (FUS)-positive, but tau and TDP-43-negative inclusions. These neuropathologies are considered to be important in the etiology of FTLD.

Nearly half of FTLD patients have a first-degree family member with dementia, ALS, or Parkinson's disease, suggesting a strong genetic link to the cause of the disease. A number of mutations in chromosome 17q21 have been linked to FTLD presentation.

### Progranulin-Associated Frontotemporal Lobar Degeneration

Studies investigating the link between chromosome 17q21 and FTLD have found a number of FTLD-related mutations in the PGRN gene. These mutations often result in aggregation and accumulation of ub-positive, TDP43-positive, tau-negative neuropathological inclusions in brains of FTLD patients. PGRN is a secreted precursor peptide to a number of mature GRN proteins and is thought to function primarily as a neurotrophic growth factor, promoting neuronal differentiation and survival. PGRN has also been demonstrated to serve anti-inflammatory and neuroprotective functions. PGRN Is expressed ubiquitously, but as a result of its association with FTLD, significant attention has been directed to the central nervous system (CNS) where it is expressed in multiple cell types including neuronal, glial, and endothelial cells. Over 70 loss-of-function mutations in the PGRN gene have been identified in FTLD, the vast majority of which result in haploinsufficiency and a reduction in serum PGRN levels by more than a 50%. PGRN mutations are described in Gijselinck et al., Human Mutation 29: 1373-86 (2008), the disclosures of which are incorporated herein by reference as they relate to human PGRN mutations. The effects of PGRN mutations are dose dependent as homozygous patients completely lacking functional PGRN protein develop a lysosomal storage disease known as CLN11 neuronal ceroid lipofuscinosis (NCL), suggesting an additional role for this protein in normal lysosomal function. Neurodegeneration, dementia, and premature cognitive decline are also a hallmark of NCL symptomology.

Clinical management of FTLD has primarily employed selective serotonin reuptake inhibitors (SSRIs) and antipsychotics to manage the changes in affect and behavior that accompany FTLD. This strategy, however, is targeted at ameliorating the symptoms of the disease without addressing its development and progression. Unlike these treatments, the compositions and methods described herein provide the benefit of treating a different biochemical phenomenon that can underlie the development of FTLD.

### Therapeutic Agents

Using the compositions and methods of the disclosure, a patient having a FTLD may be administered one or more agents that together augment the expression and/or activity of one or more proteins selected from HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT, such as one or more agents that together augment the expression and/or activity of one or more proteins selected from HLA-DRA, HLA-DRB5, C9ORF72, SQSTM1, TBK1, PSEN1, GRN, and CTSF. Exemplary amino acid sequences of these proteins are set forth in Table 8, below. Also included in Table 8 are exemplary nucleic acid sequences of genes encoding each corresponding protein. Nucleic acid sequences are listed using ENA reference identification numbers.

TABLE 8
Exemplary amino acid and nucleic acid sequences of proteins that may be modulated for
the treatment of a FTLD

| | | | Reference for |
|---|---|---|---|
| | | | Exemplary |
| | | | Nucleic Acid |
| SEQ | | | Sequence |
| ID | | | Encoding |
| NO. | Protein | Exemplary Amino Acid Sequence of Protein Product | Protein Product |
| 89 | HLA- | MAISGVPVLGFFIIAVLMSAQESWAIKEEHVIIQAEFYLNPDQSGEFMFDFDGDEIFHVD | ENA J00194.1 |
| | DRA | MAKKETVWRLEEFGRFASFEAQGALANIAVDKANLEIMTKRSNYTPITNVPPEVTVLTNS | |
| | | PVELREPNVLICFIDKFTPPVVNVTWLRNGKPVTTGVSETVFLPREDHLFRKFHYLPFLP | |
| | | STEDVYDCRVEHWGLDEPLLKHWEFDAPSPLPETTENVVCALGLTVGLVGIIIGTIFIIK | |
| | | GVRKSNAAERRGPL | |
| 90 | C9ORF | MSTLCPPPSPAVAKTEIALSGKSPLLAATFAYWDNILGPRVRHIWAPKTEQVLLSDGEIT | ENA JN681271.1 |
| | 72 | FLANHTLNGEILRNAESGAIDVKFFVLSEKGVIIVSLIFDGNWNGDRSTYGLSIILPQTE | |
| | | LSFYLPLHRVCVDRLTHIIRKGRIWMHKERQENVQKIILEGTERMEDQGQSIIPMLTGEV | |
| | | IPVMELLSSMKSHSVPEEIDIADTVLNDDDIGDSCHEGFLLNAISSHLQTCGCSVVVGSS | |
| | | AEKVNKIVRTLCLFLTPAERKCSRLCEAESSFKYESGLFVQGLLKDSTGSFVLPFRQVMY | |
| | | APYPTTHIDVDVNTVKQMPPCHEHIYNQRRYMRSELTAFWRATSEEDMAQDTIIYTDESF | |
| | | TPDLNIFQDVLHRDTLVKAFLDQVFQLKPGLSLRSTFLAQFLLVLHRKALTLIKYIEDDT | |
| | | QKGKKPFKSLRNLKIDLDLTAEGDLNIIMALAEKIKPGLHSFIFGRPFYTSVQERDVLMT | |
| | | F | |
| 91 | SQSTM | MASLTVKAYLLGKEDAAREIRRFSFCCSPEPEAEAEAAAGPGPCERLLSRVAALFPALRP | ENA U41806.1 |
| | 1 | GGFQAHYRDEDGDLVAFSSDEELTMAMSYVKDDIFRIYIKEKKECRRDHRPPCAQEAPRN | |
| | | MVHPNVICDGCNGPVVGTRYKCSVCPDYDLCSVCEGKGLHRGHTKLAFPSPFGHLSEGFS | |
| | | HSRWLRKVKHGHFGWPGWEMGPPGNWSPRPPRAGEARPGPTAESASGPSEDPSVNFLKNV | |
| | | GESVAAALSPLGIEVDIDVEHGGKRSRLTPVSPESSSTEEKSSSQPSSCCSDPSKPGGNV | |
| | | EGATQSLAEQMRKIALESEGRPEEQMESDNCSGGDDDWTHLSSKEVDPSTGELQSLQMPE | |
| | | SEGPSSLDPSQEGPTGLKEAALYPHLPPEADPRLIESLSQMLSMGFSDEGGWLTRLLQTK | |
| | | NYDIGAALDTIQYSKHPPPL | |
| 92 | TARDB | MSEYIRVTEDENDEPIEIPSEDDGTVLLSTVTAQFPGACGLRYRNPVSQCMRGVRLVEGI | ENA U23731.1 |
| | P | LHAPDAGWGNLVYVVNYPKDNKRKMDETDASSAVKVKRAVQKTSDLIVLGLPWKITEQDL | |
| | | KEYFSTFGEVLMVQVKKDLKTGHSKGEGFVRFTEYETQVKVMSQRHMIDGRWCDCKLPNS | |
| | | KQSQDEPLRSRKVFVGRCTEDMTEDELREFFSQYGDVMDVFIPKPFRAFAFVTFADDQIA | |
| | | QSLCGEDLIIKGISVHISNAEPKHNSNRQLERSGRFGGNPGGFGNQGGFGNSRGGGAGLG | |
| | | NNQGSNMGGGMNFGAFSINPAMMAAAQAALQSSWGMMGMLASQQNQSGPSGNNQNQGNMQ | |
| | | REPNQAFGSGNNSYSGSNSGAAIGWGSASNAGSGSGFNGGFGSSMDSKSSGWGM | |
| 93 | TBK1 | MQSTSNHLWLLSDILGQGATANVERGRHKKTGDLFAIKVFNNISFLRPVDVQMREFEVLK | ENA AF191838.1 |
| | | KLNHKNIVKLEAIEEETTTRHKVLIMEFCPCGSLYTVLEEPSNAYGLPESEFLIVLRDVV | |
| | | GGMNHLRENGIVHRDIKPGNIMRVIGEDGQSVYKLTDFGAARELEDDEQFVSLYGTEEYL | |
| | | HPDMYERAVLRKDHQKKYGATVDLWSIGVTFYHAATGSLPFRPFEGPRRNKEVMYKIITG | |
| | | KPSGAISGVQKAENGPIDWSGDMPVSCSLSRGLQVLLTPVLANILEADQEKCWGFDQFFA | |
| | | ETSDILHRMVIHVFSLQQMTAHKIYIHSYNTATIFHELVYKQTKIISSNQELIYEGRRLV | |
| | | LEPGRLAQHFPKTTEENPIFVVSREPLNTIGLIYEKISLPKVHPRYDLDGDASMAKAITG | |
| | | VVCYACRIASTLLLYQELMRKGIRWLIELIKDDYNETVHKKTEVVITLDFCIRNIEKTVK | |
| | | VYEKLMKINLEAAELGEISDIHTKLLRLSSSQGTIETSLQDIDSRLSPGGSLADAWAHQE | |
| | | GTHPKDRNVEKLQVLLNCMTEIYYQFKKDKAERRLAYNEEQIHKFDKQKLYYHATKAMTH | |
| | | FTDECVKKYEAFLNKSEEWIRKMLHLRKQLLSLTNQCFDIEEEVSKYQEYTNELQETLPQ | |
| | | KMFTASSGIKHTMTPIYPSSNTLVEMTLGMKKLKEEMEGVVKELAENNHILERFGSLTMD | |
| | | GGLRNVDCL | |
| 94 | VCP | MASGADSKGDDLSTAILKQKNRPNRLIVDEAINEDNSVVSLSQPKMDELQLFRGDTVLLK | ENA AF1007521 |
| | | GKKRREAVCIVLSDDTCSDEKIRMNRVVRNNLRVRLGDVISIQPCPDVKYGKRIHVLPID | |
| | | DTVEGITGNLFEVYLKPYFLEAYRPIRKGDIFLVRGGMRAVEFKVVETDPSPYCIVAPDT | |
| | | VIHCEGEPIKREDEEESLNEVGYDDIGGCRKQLAQIKEMVELPLRHPALFKAIGVKPPRG | |
| | | ILLYGPPGTGKTLIARAVANETGAFFFLINGPEIMSKLAGESESNLRKAFEEAEKNAPAI | |
| | | IFIDELDAIAPKREKTHGEVERRIVSQLLTLMDGLKQRAHVIVMAATNRPNSIDPALRRF | |
| | | GRFDREVDIGIPDATGRLEILQIHTKNMKLADDVDLEQVANETHGHVGADLAALCSEAAL | |
| | | QAIRKKMDLIDLEDETIDAEVMNSLAVTMDDFRWALSQSNPSALRETVVEVPQVTWEDIG | |
| | | GLEDVKRELQELVQYPVEHPDKFLKFGMTPSKGVLFYGPPGCGKTLLAKAIANECQANFI | |
| | | SIKGPELLTMWFGESEANVREIFDKARQAAPCVLFFDELDSIAKARGGNIGDGGGAADRV | |
| | | INQILTEMDGMSTKKNVFIIGATNRPDIIDPAILRPGRLDQLIYIPLPDEKSRVAILKAN | |
| | | LRKSPVAKDVDLEFLAKMTNGFSGADLTEICQRACKLAIRESIESEIRRERERQTNPSAM | |
| | | EVEEDDPVPEIRRDHFEEAMRFARRSVSDNDIRKYEMFAQTLQQSRGFGSFRFPSGNQGG | |
| | | AGPSQGSGGGTGGSVYTEDNDDDLYG | |
| 2 | PSEN1 | MTELPAPLSYFQNAQMSEDNHLSNTVRSQNDNRERQEHNDRRSLGHPEPLSNGRPQGNSR | ENA U40379.1 |
| | | QVVEQDEEEDEELTLKYGAKHVIMLFVPVTLCMVVVVATIKSVSFYTRKDGQLIYTPFTE | |
| | | DTETVGQRALHSILNAAIMISVIVVMTILLVVLYKYRCYKVIHAWLIISSLLLLFFFSFI | |
| | | YLGEVFKTYNVAVDYITVALLIWNFGVVGMISIHWKGPLRLQQAYLIMISALMALVFIKY | |
| | | LPEWTAWLILAVISVYDLVAVLCPKGPLRMLVETAQERNETLFPALIYSSTMVWLVNMAE | |
| | | GDPEAQRRVSKNSKYNAESTERESQDTVAENDDGGFSEEWEAQRDSHLGPHRSTPESRAA | |
| | | VQELSSSILAGEDPEERGVKLGLGDFIFYSVLVGKASATASGDWNTTIACFVAILIGLCL | |
| | | TLLLLAIFKKALPALPISITFGLVFYFATDYLVQPFMDQLAFHQFYI | |
| 95 | FUS | MASNDYTQQATQSYGAYPTQPGQGYSQQSSQPYGQQSYSGYSQSTDTSGYGQSSYSSYGQ | ENA S62140.1 |
| | | SQNTGYGTQSTPQGYGSTGGYGSSQSSQSSYGQQSSYPGYGQQPAPSSTSGSYGSSSQSS | |
| | | SYGQPQSGSYSQQPSYGGQQQSYGQQQSYNPPQGYGQQNQYNSSSGGGGGGGGGGNYGQD | |
| | | QSSMSSGGGSGGGYGNQDQSGGGGSGGYGQQDRGGRGRGGSGGGGGGGGGGYNRSSGGYE | |
| | | PRGRGGGRGGRGGMGGSDRGGFNKFGGPRDQGSRHDSEQDNSDNNTIFVQGLGENVTIES | |
| | | VADYFKQIGIIKTNKKTGQPMINLYTDRETGKLKGEATVSFDDPPSAKAAIDWFDGKEFS | |
| | | GNPIKVSFATRRADFNRGGGNGRGGRGRGGPMGRGGYGGGGSGGGGRGGFPSGGGGGGGQ | |
| | | QRAGDWKCPNPTCENMNFSWRNECNQCKAPKPDGPGGGPGGSHMGGNYGDDRRGGRGGYD | |
| | | RGGYRGRGGDRGGFRGGRGGGDRGGFGPGKMDSRGEHRQDRRERPY | |
| 96 | CHMP2 | NASLFKKKTVDDVIKEQNRELRGTQRATIRDRAALEKQEKQLELEIKKMAKIGNKEACKV | ENA AF151842.2 |
| | B | LAKQLVHLRKQKTRTFAVSSKVTSMSTQTKVMNSQMKMAGAMSTTAKTMQAVNKKMDPQK | |
| | | TLQTMQNFQKENMKMEMTEEMINDTLDDIFOGSDDEEESQDIVNQVLDEIGIElSGKMAK | |
| | | APSAARSLPSASTSKATISDEEIERQLKALGVD | |
| 97 | UBQLN | MAENGESSGPPRPSRGPAAAQGSAAAPAEPKIIKVTVKTPKEKEEFAVPENSSVQQFKEA | ENA AF189009.1 |
| | 2 | ISKRFKSQTDQLVLIFAGKILKDQDTLIQHGIHDGLTVHLVIKSQNRPQGQSTQPSNAAG | |
| | | TNTTSASTPRSNSTPISTNSNPFGLGSLGGLAGLSSLGLSSTNFSELQSQMQQQLMASPE | |
| | | MMIQIMENPFVQSMLSNPDLMRQLIMANPQMQQLIQRNPEISHLLNNPDIMRQTLETARN | |
| | | PAMMQEMMRNQDLALSNLESIPGGYNALRRMYTDIQEPMLNAAQEQFGGNPFASVGSSSS | |
| | | SGEGTQPSRTENRDPLPNPWAPPPATQSSATTSTTTSTGSGSGNSSSNATGNTVAAANYV | |
| | | ASIFSTPGMQSLLQQITENPQLIQNMLSAPYMRSMMQSLSQNPDLAAQMMLNSPLFTANP | |
| | | QLQEQMRPQLPAFLQQMQNPDTLSAMSNPRAMQALMQIQQGLQTLATEAPGLIPSFTPGV | |
| | | GVGVLGTAIGPVGPVTPIGPIGPIVPFTPIGPIGPIGPTGPAAPPGSTGSGGPTGPTVSS | |
| | | AAPSETTSPTSESGPNQQFIQQMVQALAGANAPQLPNPEVREQQQLEQLNAMGELNREAN | |
| | | LQALIATGGDINAAIERLLGSQPS | |
| 98 | CHCHD | MPRGSRSAASRPASRPAAPSAHPPAHPPPSAAAPAPAPSGQPGLMAQMATTAAGVAVGSA | ENA AK289560.1 |
| | 10 | VGHVMGSALTGAFSGGSSEPSQPAVQQAPTPAAPQPLQMGPCAYEIRQFLDCSTTQSDLS | |
| | | LCEGFSEALKQCKYYHGLSSLP | |
| 106 | GRN | MWTLVSWVALTAGLVAGTRCPDGQFCPVACCLDPGGASYSCCRPLLDKWPTTLSRHLGGP | ENA BC000324.2 |
| | | CQVDAHCSAGHSCIFTVSGTSSCCPFPEAVACGDGHHCCPRGFHCSADGRSCFQRSGNNS | |
| | | VGAIQCPDSQFECPDFSTCCVMVDGSWGCCPMPQASCCEDRVHCCPHGAFCDLVHTRCIT | |
| | | PTGTHPLAKKLPAQRTNRAVALSSSVMCPDARSRCPDGSTCCELPSGKYGCCPMPNATCC | |
| | | SDHLHCCPQDTVCDLIQSKCLSKENATTDLLTKLPAHTVGDVKCDMEVSCPDGYTCCRLQ | |
| | | SGAWGCCPFTQAVCCEDHIHCCPAGFTCDTQKGTCEQGPHQVPWMEKAPAHLSLPDPQAL | |
| | | KRDVPCDNVSSCPSSDTCCQLTSGEWGCCPIPEAVCCSDHQHCCPQGYTCVAEGQCQRGS | |
| | | EIVAGLEKMPARRASLSHPRDIGCDQHTSCPVGQTCCPSLGGSWACCQLPHAVCCEDRQH | |
| | | CCPAGYTCNVKARSCEKEVVSAQPATFLARSPHVGVKDVECGEGHFCHDNQTCCRDNRQG | |
| | | WACCPYRQGVCCADRRHCCPAGFRCAARGTKCLRREAPRWDAPLRDPALRQLL | |
| 99 | RAB38 | MQAPHKEHLYKLLVIGDLGVGKTSIIKRYVHQNFSSHYRATIGVDFALKVLHWDPETVVR | ENA AF235022.1 |
| | | LQLWDIAGQERFGNMTRVYYREAMGAFIVEDVTRPATFEAVAKWKNDLDSKLSLPNGKPV | |
| | | SVVLLANKCDQGKDVLMNNGLKMDQFCKEHGFVGWFETSAKENINIDEASRCLVKHILAN | |
| | | ECDLMESIEPDVVKPHLTSTKVASCSGCAKS | |
| 100 | CTSF | MAPWLQLLSLLGLLPGAVAAPAQPRAASFQAWGPPSPELLAPTRFALEMENRGRAAGTRA | ENA AJ007331.1 |
| | | VLGLVRGRVRRAGQGSLYSLEATLEEPPCNDPMVCRLPVSKKTLLCSFQVLDELGRHVLL | |
| | | RKDCGPVDTKVPGAGEPKSAFTQGSAMISSLSQNHPDNRNETFSSVISLLNEDPLSQDLP | |
| | | VKMASIFKNFVITYNRTYESKEEARWRLSVFVNNMVRAQKIQALDRGTAQYGVTKFSDLT | |
| | | EEEFRTIYLNTLLRKEPGNKMKQAKSVGDLAPPEWDWRSKGAVTKVKDQGMCGSCWAFSV | |
| | | TGNVEGQWFLNQGTLLSLSEQELLDCDKMDKACMGGLPSNAYSAIKNLGGLETEDDYSYQ | |
| | | GHMQSCNFSAEKAKVYINDSVELSQNEQKLAAWLAKRGPISVAINAFGMQFYRHGISRPL | |
| | | RPLCSPWLIDHAVLLVGYGNRSDVPFWAIKNSWGTDWGEKGYYYLHRGSGACGVNTMASS | |
| | | AVVD | |
| 3 | PSEN2 | MLTFMASDSEEEVCDERTSLMSAESPTPRSCQEGRQGPEDGENTAQWRSQENEEDGEEDP | ENA L43964.1 |
| | | DRYVCSGVPGRPPGLEEELTLKYGAKHVIMLFVPVTLCMIVVVATIKSVRFYTEKNGQLI | |
| | | YTPFTFDTPSVGQRLLNSVLNTLIMISVIVVMTIFLVVLYKYRCYKFIHGWLIMSSLMLL | |
| | | FLFTYIYLGEVLKTYNVAMDYPTLLLTVWNFGAVGMVCIHWKGPLVLQQAYLIMISALMA | |
| | | LVFIKYLPEWSAWVILGAISVYDLVAVLCPKGPLRMLVETAQERNEPIFPALIYSSAMVW | |
| | | TVGMAKLDPSSQGALQLPYDPEMEEDSYDSFGEPSYPEVFEPPLTGYPGEELEEEEERGV | |
| | | KLGLGDFIFYSVLVGKAAATGSGDWNTTLACFVAILIGLCLTLLLLAVFKKALPALP1ST | |
| | | TFGLIFYFSTDNLVRPFMDTLASHQLYI | |
| 101 | CYP27A | MAALGCARLRWALRGAGRGLCPHGARAKAAIPAALPSDKATGAPGAGPGVRRRQRSLEEI | ENA M62401.1 |
| | 1 | PRLGQLRFFFQLFVQGYALQLHQLQVLYKAKYGPMWMSYLGPQMHVNLASAPLLEQVMRQ | |
| | | EGKYPVRNDMELWKEHRDQHDLTYGPFTTEGHHWYQLRQALNQRLLKPAEAALYTDAFNE | |
| | | VIDDFMTRLDQLRAESASGNQVSDMAQLFYYFALEAICYILFEKRIGCLQRSIPEDTVTF | |
| | | VRSIGLMFQNSLYATFLPKWTRPVLPFWKRYLDGWNAIFSFGKKLIDEKLEDMEAQLQAA | |
| | | GPDGIQVSGYLHFLLASGQLSPREAMGSLPELLMAGVDTTSNTLTWALYHLSKDPEIQEA | |
| | | LHEEVVGVVPAGQVPQHKDFAHMPLLKAVLKETLRLYPVVPTNSRIIEKEIEVDGFLFPK | |
| | | NTQFVFCHYVVSRDPTAFSEPESFQPHRWLRNSQPATPRIQHPFGSVPFGYGVRACLGRR | |
| | | IAELEMQLLLARLIQKYKVVLAPETGELKSVARIVLVPNKKVGLQFLQRQC | |
| 102 | BTNL2 | MVDFPGYNLSGAVASFLFILLTMKQSEDFRVIGPAHPILAGVGEDALLTCQLLPKRTTMH | ENA BC119668.1 |
| | | VEVRWYRSEPSTPVFVHRDGVEVTEMQMEEYRGWVEWIENGIAKGNVALKIHNIQPSDNG | |
| | | QYWCHFQDGNYCGETSLLLKVAGLGSAPSIHMEGPGESGVQLVCTARGWFPEPQVYWEDI | |
| | | RGEKLLAVSEHRIQDKDGLFYAEATLVVRNASAESVSCLVHNPVLTEEKGSVISLPEKLQ | |
| | | TELASLKVNGPSQPILVRVGEDIQLTCYLSPKANAQSMEVRWDRSHRYPAVHVYMDGDHV | |
| | | AGEQMAEYRGRTVLVSDAIDEGRLTLQILSARPSDDGQYRCLFEKDDVYQEASLDLKVVS | |
| | | LGSSPLITVEGQEDGEMQPMCSSDGWFPQPHVPWRDMEGKTIPSSSQALTQGSHGLFHVQ | |
| | | TLLRVTNISAVDVTCSISIPFLGEEKIATFSLSGW | |
| 81 | MAPT | MAEPRQEFEVMEDHAGTYGLGDRKDQGGYTMHQDQEGDTDAGLKESPLQTPTEDGSEEPG | ENA J03778.1 |
| | | SETSDAKSTPTAEDVTAPLVDEGAPGKQAAAQPHTEIPEGTTAEEAGIGDTPSLEDEAAG | |
| | | HVTQEPESGKVVQEGFLREPGPPGLSHQLMSGMPGAPLLPEGPREATRQPSGTGPEDTEG | |
| | | GRHAPELLKHQLLGDLHQEGPPLKGAGGKERPGSKEEVDEDRDVDESSPQDSPPSKASPA | |
| | | QDGRPPQTAAREATSIPGFPAEGAIPLPVDFLSKVSTEIPASEPDGPSVGRAKGQDAPLE | |
| | | FTFHVEITPNVQKEQAHSEEHLGRAAFPGAPGEGPEARGPSLGEDTKEADLPEPSEKQPA | |
| | | AAPRGKPVSRVPQLKARMVSKSKDGTGSDDKKAKTSTRSSAKILKNRPCLSPKHPTPGSS | |
| | | DPLIQPSSPAVCPEPPSSPKYVSSVTSRTGSSGAKEMKLKGADGKTKIATPRGAAPPGQK | |
| | | GQANATRIPAKTPPAPKTPPSSGEPPKSGDRSGYSSPGSPGTPGSRSRTPSLPTPPTREP | |
| | | KKVAVVRTPPKSPSSAKSRLQTAPVPMPDLKNVKSKIGSTENLKHQPGGGKVQIINKKLD | |
| | | LSNVQSKCGSKDNIKHVPGGGSVQIVYKPVDLSKVTSKCGSLGNIHHKPGGGQVEVKSEK | |
| | | LDFKDRVQSKIGSLDNITHVPGGGNKKIETHKLTFRENAKAKTDHGAEIVYKSPVVSGDT | |
| | | SPRHLSNVSSTGSIDMVDSPQLATLADEVSASLAKQGL | |
| 12 | HLA- | MVCLKLPGGSYMAKLIVTLMVLSSPLALAGDTRPRFLQQDKYECHFFNGTERVRFLHRDI | ENA M20429.1 |
| | DRB5 | YNQEEDLRFDSDVGEYRAVTELGRPDAEYWNSQKDFLEDRRAAVDTYCRHNYGVGESFTV | |
| | | QRRVEPKVTVYPARTQTLQHHNLLVCSVNGFYPGSIEVRWFRNSQEEKAGVVSTGLIQNG | |
| | | DWTFQTLVMLETVPRSGEVYTCQVEHPSVTSPLTVEWRAQSESAQSKMLSGVGGFVLGLL | |
| | | FLGAGLFIYFKNQKGHSGLHPTGLVS | |

Agents that elevate the expression and/or activity level of one or more of the foregoing proteins that may be used in conjunction with the compostons and methods of the disclosure include nucleic acids that encode the protein or plurality of proteins (e.g., nucleic acids capable of expression in macrophages or microglia). Such nucleic acid molecules may be provided to a patient (e.g., a patient having a FTLD) in the form, for example, of a population of cells, such as a population of cells (e.g., pluripotent cells, ESCs, iPSCs, multipotent cells, CD34+ cells, HSCs, MPCs, BLPCs, monocytes, macrophages, microglial progenitor cells, or microglia) that contain the nucleic acid molecules. Such cells may be modified ex vivo so as to express the nucleic acid molecule(s) of interest, for example, using transfection and transduction methods described herein. Additionally or alternatively, nucleic acid molecules encoding one or more of the proteins of interest may be provided to the patient in the form of one or more viral vectors that collectively encode the one or more proteins. Exemplary viral vectors that may be used in conjunction with the compositions and methods of the disclosure include Retroviridae family viral vectors, such as a lentivirus, alpharetrovirus, or gammaretrovirus, among others described herein. In some embodiments, the nucleic acid molecule(s) are administered directly to the patient. Additional agents that may be provided to a patient for the purpose of augmenting the level of one or more of the foregoing proteins include interfering RNA molecules, such as siRNA, shRNA, and miRNA molecules, as well as small molecule agents that modulate the expression of one or more of the above proteins, in addition to the one or more of the above proteins themselves.

Furthermore, the compositions and methods of the present disclosure can be used for treatment of two or more disorders or conditions when such disorders or conditions are associated with the same or overlapping genetic risk loci (e.g., mutation(s) in a single gene may be associated with more than one disease or condition). In a particular example, the compositions and methods described herein may be advantageously used to treat a patient having any one of Alzheimer's disease, Parkinson disease, or a FTLD by administering one or more agents that together augment the expression and/or activity of one or more APP, PSEN1, PSEN2, APOE, TOMM40, GAB2, APOC1, TREM2, ABI3, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1L, STK24, DISC1, MPZL1, SLC4A1AP, TRIP4, MSRA, HS3ST1, ZNF224, AP2A2, FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1L2, MCC01, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1B, NUCKS1, ACMSD, HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT.

Agents that elevate the expression and/or activity level of one or more of the foregoing proteins that may be used in conjunction with the compositions and methods of the disclosure include nucleic acids that encode the protein or plurality of proteins. Such nucleic acid molecules may be provided to a patient (e.g., a patient having Alzheimer's disease, Parkinson disease, or a FTLD) in the form, for example, of a population of cells, such as a population of cells (e.g., pluripotent cells, ESCs, iPSCs, multipotent cells, CD34+ cells, HSCs, MPCs, BLPCs, monocytes, macrophages, microglial progenitor cells, or microglia) that contain the nucleic acid molecules. Such cells may be modified ex vivo so as to express the nucleic acid molecule(s) of interest, for example, using transfection and transduction methods described herein. Additionally or alternatively, nucleic acid molecules encoding one or more of the proteins of interest may be provided to the patient in the form of one or more viral vectors that collectively encode the one or more proteins. Exemplary viral vectors that may be used in conjunction with the compositions and methods of the disclosure include Retroviridae family viral vectors, such as a lentivirus, alpharetrovirus, or gammaretrovirus, among others described herein. In some embodiments, the nucleic acid molecule(s) are administered directly to the patient. Additional agents that may be provided to a patient for the purpose of augmenting the level of one or more of the foregoing proteins include interfering RNA molecules, such as siRNA, shRNA, and miRNA molecules, as well as small molecule agents that modulate the expression of one or more of the above proteins, in addition to the one or more of the above proteins themselves.

### Therapeutic Cells

Cells that may be used in conjunction with the compositions and methods described herein include cells that are capable of undergoing further differentiation (e.g., pluripotent cells, ESCs, iPSCs, CD34+ cells, HSCs, MPCs, BLPCs, monocytes, or microglial progenitor cells) or differentiated cells (e.g., macrophages or microglia). For example, one type of cell that can be used in conjunction with the compositions and methods described herein is a pluripotent cell. A pluripotent cell is a cell that possesses the ability to develop into more than one differentiated cell type. Examples of pluripotent cells are ESCs and iPSCs. ESCs and iPSCs have the ability to differentiate into cells of the ectoderm, which gives rise to the skin and nervous system, endoderm, which forms the gastrointestinal and respiratory tracts, endocrine glands, liver, and pancreas, and mesoderm, which forms bone, cartilage, muscles, connective tissue, and most of the circulatory system. Another type of cell that can be used in conjunction with the compositions and methods described herein is a multipotent cell. A multipotent cell is a cell that possesses the ability to differentiate into multiple, but not all cell types. A non-limiting example of a multipotent cell is a CD34+ cell (e.g., HSCs or MPC).

Cells that may be used in conjunction with the compositions and methods described herein include HSCs and MPCs. HSCs are immature blood cells that have the capacity to self-renew and to differentiate into mature blood cells including diverse lineages including but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Human HSCs are CD34+. In addition, HSCs also refer to long term repopulating HSC (LT-HSC) and short-term repopulating HSC (ST-HSC). Any of these HSCs can be used in conjunction with the compositions and methods described herein.

HSCs can differentiate into myeloid progenitor cells, which are also CD34+. Myeloid progenitors can further differentiate into granulocytes (e.g., promyelocytes, neutrophils, eosinophils, and basophils), erythrocytes (e.g., reticulocytes, erythrocytes), thrombocytes (e.g., megakaryoblasts, platelet producing megakaryocytes, and platelets), monocytes (e.g., monocytes and macrophages), dendritic cells, and microglia. Common myeloid progenitors can be characterized by cell surface molecules and are known to be lin−, SCA1−, c-kit+, CD34+, and CD16/32^{mid}.

HSCs and myeloid progenitors can be obtained from blood products. A blood product is a product obtained from the body or an organ of the body containing cells of hematopoietic origin. Such sources include unfractionated bone marrow, umbilical cord, placenta, peripheral blood, or mobilized peripheral blood. All of the aforementioned crude or unfractionated blood products can be enriched for cells having HSC or myeloid progenitor cell characteristics in a number of ways. For example, the more mature, differentiated cells can be selected against based on cell surface molecules they express. The blood product may be fractionated by positively selecting for CD34+ cells, which include a subpopulation of hematopoietic stem cells capable of self-renewal, multi-potency, and that can be re-introduced into a transplant recipient whereupon they home to the hematopoietic stem cell niche and reestablish productive and sustained hematopoiesis. Such selection is accomplished using, for example, commercially available magnetic anti-CD34 beads (Dynal, Lake Success, N.Y.). Myeloid progenitor cells can also be isolated based on the markers they express. Unfractionated blood products can be obtained directly from a donor or retrieved from cryopreservative storage. HSCs and myeloid progenitor cells can also be obtained from by differentiation of ES cells, iPS cells or other reprogrammed mature cells types.

Cells that may be used in conjunction with the compositions and methods described herein include allogeneic cells and autologous cells. All of the aforementioned cell types are capable of differentiating into microglia. Cells described herein may also differentiate into microglial progenitors or microglial stem cells. Differentiation may occur ex vivo or in vivo. Methods for ex vivo differentiation of human ESCs and iPSCs are known by those of skill in the art and are described in Muffat et al., Nature Medicine 22:1358-1367 (2016) and Pandya et al., Nature Neuroscience (2017) epub ahead of print, the disclosures of which are incorporated herein by reference as they pertain to methods of differentiating cells into microglia.

### Microglia

Cells that may be used in conjunction with the compositions and methods described herein include microglial cells and those that are capable of differentiating into microglial cells or cells that are differentiated microglial cells. Microglia are myeloid-derived cells that serve as the immune cells, or resident macrophages, of the central nervous system. Microglia are highly similar to macrophages, both genetically and functionally, and share the ability to shift dynamically between pro-inflammatory and anti-inflammatory states. The pro-inflammatory state is known as classical activation, or Ml, and the anti-inflammatory state is called alternative activation, or M2. Microglia can be made to shift between the two states by extracellular signals, e.g., signals from neighboring neurons or astrocytes, cell debris, toxins, infection, ischemia, and traumatic injury, among others. M1 microglia are often observed in the diseased brain, particularly in diseases involving neuroinflammation, such as AD. Classically activated M1 phenotypes have also been observed in mouse models of AD, such as the double transgenic APP/PS1 mouse. It is unclear whether M1 microglia are a cause or consequence of neuroinflammation, but once microglia are classically activated, they can secrete pro-inflammatory cytokines, e.g., TNF-α, IL-1β, and IL-6, chemokines, and nitric oxide, which can lead to sustained inflammation, neuronal damage, and further activation of M1 microglia. This positive feedback loop can be harmful to brain tissue; therefore, methods of reducing M1 activation and/or increasing M2 activation may help patients with diseases featuring neuroinflammation.

### Expression of Therapeutic Proteins in Host Cells

The present disclosure includes compositions and methods for expressing one or more therapeutic proteins, such as a therapeutic protein set forth in any one of Tables 1-4, herein, in a host cell, such as a mammalian (e.g., human) pluripotent cell, ESC, iPSC, multipotent cell, CD34+ cell, HSCs, MPC, BLPC, monocyte, macrophage, microglial progenitor cell, or microglial cell) . Exemplary methods that can be used for effectuating the expression of one or more therapeutic proteins in a host cell are described in further detail in the sections that follow.

### Polynucleotides Encoding Therapeutic Proteins of the Disclosure

One platform that can be used to achieve therapeutically effective intracellular concentrations of one or more proteins described herein in mammalian cells (e.g., pluripotent cells, ESCs, iPSCs, multipotent cells, CD34+ cells, HSCs, MPCs, BLPCs, monocytes, macrophages, microglial progenitor cells, or microglia) is via the stable expression of genes encoding these agents (e.g., by integration into the nuclear or mitochondrial genome of a mammalian cell). These genes are polynucleotides that encode the primary amino acid sequence of the corresponding protein. In order to introduce such exogenous genes into a mammalian cell, these genes can be incorporated into a vector. Vectors can be introduced into a cell by a variety of methods, including transformation, transfection, direct uptake, projectile bombardment, and by encapsulation of the vector in a liposome. Examples of suitable methods of transfecting or transforming cells are calcium phosphate precipitation, electroporation, microinjection, infection, lipofection, and direct uptake. Such methods are described in more detail, for example, in Green et al., Molecular Cloning: A Laboratory Manual, Fourth Edition (Cold Spring Harbor University Press, New York (2014)); and Ausubel et al., Current Protocols in Molecular Biology (John Wiley & Sons, New York (2015)), the disclosures of each of which are incorporated herein by reference.

Genes encoding therapeutic proteins of the disclosure can also be introduced into mammalian cells by targeting a vector containing a gene encoding such an agent to cell membrane phospholipids. For example, vectors can be targeted to the phospholipids on the extracellular surface of the cell membrane by linking the vector molecule to a VSV-G protein, a viral protein with affinity for all cell membrane phospholipids. Such, a construct can be produced using methods well known to those of skill in the field.

Recognition and binding of the polynucleotide encoding one or more therapeutic proteins of the disclosure by mammalian RNA polymerase is important for gene expression. As such, one may include sequence elements within the polynucleotide that exhibit a high affinity for transcription factors that recruit RNA polymerase and promote the assembly of the transcription complex at the transcription initiation site. Such sequence elements include, e.g., a mammalian promoter, the sequence of which can be recognized and bound by specific transcription initiation factors and ultimately RNA polymerase. Examples of mammalian promoters have been described in Smith et al., Mol. Sys. Biol., 3:73, online publication, the disclosure of which is incorporated herein by reference.

Polynucleotides suitable for use with the compositions and methods described herein also include those that encode a therapeutic protein of the disclosure operably linked to (e.g., downstream of) a mammalian promoter. Promoters that are useful for the expression of a therapeutic protein described herein in mammalian cells include, e.g., elongation factor 1-alpha (EF1α) promoter, phosphoglycerate kinase 1 (PGK) promoter, CD68 molecule (CD68) promoter (see Dahl et al., Molecular Therapy 23:835 (2015), incorporated herein by reference as it pertains to the use of PGK and CD68 promoters to modulate gene expression), C—X3-C motif chemokine receptor 1 (CX3CR1) promoter, CD1 1 b promoter, allograft inflammatory factor 1 (AIF1) promoter, purinergic receptor P2Y12 (P2Y12) promoter, transmembrane protein 119 (TMEM119) promoter, and colony stimulating factor 1 receptor (CSF1 R) promoter. Alternatively, promoters derived from viral genomes can also be used for the stable expression of these agents in mammalian cells. Examples of functional viral promoters that can be used to promote mammalian expression of these agents are adenovirus late promoter, vaccinia virus 7.5K promoter, simian virus 40 (SV40) promoter, cytomegalovirus promoter, tk promoter of herpes simplex virus (HSV), mouse mammary tumor virus (MMTV) promoter, long terminal repeat (LTR) promoter of human immunodeficiency virus (HIV), promoter of moloney virus, Epstein barr virus (EBV), Rous sarcoma virus (RSV), and the cytomegalovirus (CMV) promoter. Additionally or alternatively, synthetic promoters optimized for use in mammalian cells can be employed for stable expression of one or more therapeutic proteins described herein.

Once a polynucleotide encoding one or more therapeutic proteins has been incorporated into the nuclear DNA of a mammalian cell, the transcription of this polynucleotide can be induced by methods known in the art. For example, expression can be induced by exposing the mammalian cell to an external chemical reagent, such as an agent that modulates the binding of a transcription factor and/or RNA polymerase to the mammalian promoter and thus regulates gene expression. The chemical reagent can serve to facilitate the binding of RNA polymerase and/or transcription factors to the mammalian promoter, e.g., by removing a repressor protein that has bound the promoter. Alternatively, the chemical reagent can serve to enhance the affinity of the mammalian promoter for RNA polymerase and/or transcription factors such that the rate of transcription of the gene located downstream of the promoter is increased in the presence of the chemical reagent. Examples of chemical reagents that potentiate polynucleotide transcription by the above mechanisms are tetracycline and doxycycline. These reagents are commercially available (Life Technologies, Carlsbad, CA) and can be administered to a mammalian cell in order to promote gene expression according to established protocols.

Other DNA sequence elements that may be included in polynucleotides for use in the compositions and methods described herein are enhancer sequences. Enhancers represent another class of regulatory elements that induce a conformational change in the polynucleotide containing the gene of interest such that the DNA adopts a three-dimensional orientation that is favorable for binding of transcription factors and RNA polymerase at the transcription initiation site. Thus, polynucleotides for use in the compositions and methods described herein include those that encode one or more therapeutic proteins and additionally include a mammalian enhancer sequence. Many enhancer sequences are now known from mammalian genes, and examples are enhancers from the genes that encode mammalian globin, elastase, albumin, a-fetoprotein, and insulin. Enhancers for use in the compositions and methods described herein also include those that are derived from the genetic material of a virus capable of infecting a eukaryotic cell. Examples are the SV40 enhancer on the late side of the replication origin (bp 100-270), the cytomegalovirus early promoter enhancer, the polyoma enhancer on the late side of the replication origin, and adenovirus enhancers. Additional enhancer sequences that induce activation of eukaryotic gene transcription are disclosed in Yaniv et al., Nature 297:17 (1982).

### Cell-Specific Gene Expression

Interfering RNA (RNAi) are widely used to knock down the expression of endogenous genes by delivering small interfering RNA (siRNA) into cells triggering the degradation of complementary mRNA. An additional application is to utilize the diversity of endogenous micro RNAs (miRNA) to negatively regulate the expression of exogenously introduced transgenes tagged with artificial miRNA target sequences. These miRNA target tagged transgenes can be negatively regulated according to the activity of a given miRNA which can be tissue, lineage, activation, or differentiation stage specific. These artificial miRNA target sequences (miRTs) can be recognized as targets by a specific miRNA thus inducing post-transcriptional gene silencing. While robust transgene expression in targeted cells can have beneficial therapeutic results, off-target expression, such as the ectopic or non-regulated transgene expression in HSPCs or other progenitor cells, can have cytotoxic effects, which can result in counter-selection of transgene-containing cells leading to altered cellular behavior and reduced therapeutic efficacy. The incorporation of miRNA target sequences (miRTs) for miRNAs widely expressed in HSPCs and progenitors, but absent in cells of the myeloid lineage can allow for repressed transgene expression in HSPCs and other progenitor cells allowing for silent, long-term reservoir transgene-containing hematopoietic progeny, while allowing for robust transgene expression in differentiated, mature target cells. miR-126 is highly expressed in HSPCs, other progenitor cells, and cells of the erythroid lineage, but absent from those of the myeloid lineage (e.g., macrophages and microglia) (Gentner et al., Science Translational Medicine. 2:58ra34 (2010)). A miR-126 targeting sequence, for example, incorporated within a transgene can allow for targeted expression of the transgene in cells of the myeloid lineage and repressed expression in HSPCs and other progenitor cells, thus minimizing off-target cytotoxic effects. In some embodiments, a transgene encoding one or more therapeutic proteins of the disclosure includes a miR-126 targeting sequence.

### ApoE Tag for Blood-Brain Barrier Penetrance

In some embodiments, one or more therapeutic proteins of the disclosure is modified to enhance penetration of the blood-brain barrier (BBB). Exemplary modifications for this purpose are the use of tags containing a receptor-binding (Rb) domain of apolipoprotein E (ApoE). The complete ApoE amino acid sequence is shown below.

(SEQ ID NO: 105)
MKVLWAALLVTFLAGCQAKVEQAVETEPEPELRQQTEWQSGQRWELALGR
FWDYLRWVQTLSEQVQEELLSSQVTQELRALMDETMKELKAYKSELEEQL
TPVAEETRARLSKELQAAQARLGADMEDVCGRLVQYRGEVQAMLGQSTEE
LRVRLASHLRKLRKRLLRDADDLQKRLAVYQAGAREGAERGLSAIRERLG
PLVEQGRVRAATVGSLAGQPLQERAQAWGERLRARMEEMGSRTRDRLDEV
KEQVAEVRAKLEEQAQQIRLQAEAFQARLKSWFEPLVEDMQRQWAGLVEK
VQAAVGTSAAPVPSDNH

ApoE is an important protein involved in lipid transport, and its cellular internalization is mediated by several members of the low-density lipoprotein (LDL) receptor gene family, including the LDL receptor, very low-density lipoprotein receptor (VLDLR), and LDL receptor-related proteins (LRPs, including LRP1, LRP2, and LRP8). The LDL receptor is found to be highly expressed in brain capillary endothelial cells (BCECs), with down-regulated expression observed in peripheral vessels. Restricted expressions of LRPs and VLDLR have also been shown prominently in the liver and brain when they have been detected in BCECs, neurons, and glial cells. Several members of the low-density lipoprotein receptor family (LDLRf) proteins, including LRP1 and VLDLR, but not LDLR, are highly expressed in BBB-forming BCECs. These proteins can bind ApoE to facilitate their transcytosis into the abluminal side of the BBB.

In addition, receptor-associated protein (RAP), an antagonist as well as a ligand for both LRP1 and VLDLR, has been shown to have higher permeability across the BBB than transferrin in vivo and in vitro (Pan et al., J. Cell Sci. 117:5071-8 (2004)), indicating that these lipoprotein receptors (LDLRf) can represent efficient BBB delivery targets despite their lower expression than the transferrin receptor. As described herein, a Rb peptide derived from ApoE, when incorporated into a fusion protein containing a therapeutic protein of the disclosure, can effectuate the translocation of the therapeutic protein across the BBB and into the brain. The use of ApoE Rb peptides thus represents a strategy for selectively opening the BBB for therapeutic agents (e.g., one or more therapeutic proteins of the disclosure) when incorporated into a fusion construct. ApoE Rb peptides can be readily attached to therapeutic agents without jeopardizing their biological functions or interfering with the important biological functions of ApoE due to the utilization of the Rb domain of ApoE, rather than the entire ApoE protein. This pathway is also an alternative uptake pathway that can facilitate further/secondary distribution within the brain after the agents reach the CNS due to the widespread expression of LDLRf members in brain parenchyma. Regardless of application strategies, e.g., enzyme replacement therapy or cell-based, gene-based therapy, both the quantity and distribution of therapeutics within the brain parenchyma will have a significant impact on the clinical outcome of disease treatment. The development of and a detailed description of the use of the Rb domain of ApoE in targeted delivery of proteins across the BBB can be found in U.S. Publication No. 20140219974, which is hereby incorporated by reference in its entirety.

In some embodiments, a therapeutic protein of the disclosure contains the LDLRf Rb domain of SEQ ID NO: 105, or a fragment, variant, or oligomer thereof. An exemplary Rb domain can be found in the N-terminus of ApoE, for example, between amino acid residues 1 to 191 of SEQ ID NO: 105, between amino acid residues 25 to 185 of SEQ ID NO: 105, between amino acid residues 50 to 180 of SEQ ID NO: 105, between amino acid residues 75 to 175 of SEQ ID NO: 105, between amino acid residues 100 to 170 of SEQ ID NO: 105, or between amino acid residues 125 to 165 of SEQ ID NO: 105. An exemplary receptor-binding domain has the amino acid sequence of residues 159 to 167 of SEQ ID NO: 105.

In some embodiments, the peptide sequence containing the receptor-binding domain of ApoE can include at least one amino acid mutation, deletion, addition, or substitution. In some embodiments, the amino acid substitutions can be a combination of two or more mutations, deletions, additions, or substitutions. In some embodiments, the at least one substation is a conservative substitution. In some embodiments, the at least one amino acid addition includes addition of a selected sequence already found in the Rb domain of ApoE. A person of ordinary skill in the art will recognize suitable modifications that can be made to the sequence while retaining the biochemical activity for transport across the BBB.

### Vectors for the Expression of Therapeutic Proteins

In addition to achieving high rates of transcription and translation, stable expression of an exogenous gene in a mammalian cell (e.g., pluripotent cell, ESC, iPSC, multipotent cell, CD34+ cell, HSC, MPC, BLPC, monocyte, macrophage, microglial progenitor cell, or microglial cell) can be achieved by integration of the polynucleotide containing the gene into the nuclear genome of the mammalian cell. A variety of vectors for the delivery and integration of polynucleotides encoding exogenous proteins into the nuclear DNA of a mammalian cell have been developed. Examples of expression vectors are disclosed in, e.g., WO 1994/011026 and are incorporated herein by reference. Expression vectors for use in the compositions and methods described herein may contain one or more polynucleotides encoding one or more therapeutic proteins of the disclosure, and may further include, for example, nucleic acid elements used to regulate the expression of these agents and/or the integration of such polynucleotides into the genome of a mammalian cell. Certain vectors that can be used for the expression of one or more therapeutic proteins described herein include plasmids that contain regulatory sequences, such as promoter and enhancer regions, which direct gene transcription. Other useful vectors for expression of one or more therapeutic proteins of the disclosure contain polynucleotide sequences that enhance the rate of translation of these genes or improve the stability or nuclear export of the mRNA that results from gene transcription. These sequence elements include, e.g., 5′ and 3′ untranslated regions, an IRES, and polyadenylation signal site in order to direct efficient transcription of the gene carried on the expression vector. The expression vectors suitable for use with the compositions and methods described herein may also contain a polynucleotide encoding a marker for selection of cells that contain such a vector. Examples of a suitable marker are genes that encode resistance to antibiotics, such as ampicillin, chloramphenicol, kanamycin, nourseothricin, among others.

### Viral Vectors for Expression of Therapeutic Proteins

Viral genomes provide a rich source of vectors that can be used for the efficient delivery of exogenous genes into a mammalian cell (e.g., pluripotent cell, ESC, iPSC, multipotent cell, CD34+ cell, HSC, MPC, BLPC, monocyte, macrophage, microglial progenitor cell, or microglial cell). Viral genomes are particularly useful vectors for gene delivery as the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors are a retrovirus (e.g., Retroviridae family viral vector), adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses are: avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology, Third Edition (Lippincott-Raven, Philadelphia, (1996))). Other examples are murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in McVey et al., (U.S. Pat. No. 5,801,030), the teachings of which are incorporated herein by reference.

### Retroviral Vectors

The delivery vector used in the methods and compositions described herein may be a retroviral vector. One type of retroviral vector that may be used in the methods and compositions described herein is a lentiviral vector. Lentiviral vectors (LVs), a subset of retroviruses, transduce a wide range of dividing and non-dividing cell types with high efficiency, conferring stable, long-term expression of the transgene. An overview of optimization strategies for packaging and transducing LVs is provided in Delenda, The Journal of Gene Medicine 6: S125 (2004), the disclosure of which is incorporated herein by reference.

The use of lentivirus-based gene transfer techniques relies on the in vitro production of recombinant lentiviral particles carrying a highly deleted viral genome in which the transgene of interest is accommodated. In particular, the recombinant lentivirus are recovered through the in trans coexpression in a permissive cell line of (1) the packaging constructs, i.e., a vector expressing the Gag-Pol precursors together with Rev (alternatively expressed in trans); (2) a vector expressing an envelope receptor, generally of an heterologous nature; and (3) the transfer vector, consisting in the viral cDNA deprived of all open reading frames, but maintaining the sequences required for replication, incapsidation, and expression, in which the sequences to be expressed are inserted.

A LV used in the methods and compositions described herein may include one or more of a 5′-Long terminal repeat (LTR), HIV signal sequence, HIV Psi signal 5′-splice site (SD), delta-GAG element, Rev Responsive Element (RRE), 3′-splice site (SA), elongation factor (EF) 1-alpha promoter and 3′-self inactivating LTR (SIN-LTR). The lentiviral vector optionally includes a central polypurine tract (cPPT) and a woodchuck hepatitis virus post-transcriptional regulatory element (WPRE), as described in U.S. Pat. No. 6,136,597, the disclosure of which is incorporated herein by reference as it pertains to WPRE. The lentiviral vector may further include a pHR' backbone, which may include for example as provided below.

The Lentigen LV described in Lu et al., Journal of Gene Medicine 6:963 (2004) may be used to express the DNA molecules and/or transduce cells. A LV used in the methods and compositions described herein may a 5′-Long terminal repeat (LTR), HIV signal sequence, HIV Psi signal 5′-splice site (SD), delta-GAG element, Rev Responsive Element (RRE), 3′-splice site (SA), elongation factor (EF) 1-alpha promoter and 3′-self inactivating L TR (SIN-LTR). It will be readily apparent to one skilled in the art that optionally one or more of these regions is substituted with another region performing a similar function.

Enhancer elements can be used to increase expression of modified DNA molecules or increase the lentiviral integration efficiency. The LV used in the methods and compositions described herein may include a nef sequence. The LV used in the methods and compositions described herein may include a cPPT sequence which enhances vector integration. The cPPT acts as a second origin of the (+)-strand DNA synthesis and introduces a partial strand overlap in the middle of its native HIV genome. The introduction of the cPPT sequence in the transfer vector backbone strongly increased the nuclear transport and the total amount of genome integrated into the DNA of target cells. The LV used in the methods and compositions described herein may include a Woodchuck Posttranscriptional Regulatory Element (WPRE). The WPRE acts at the transcriptional level, by promoting nuclear export of transcripts and/or by increasing the efficiency of polyadenylation of the nascent transcript, thus increasing the total amount of mRNA in the cells. The addition of the WPRE to LV results in a substantial improvement in the level of transgene expression from several different promoters, both in vitro and in vivo. The LV used in the methods and compositions described herein may include both a cPPT sequence and WPRE sequence. The vector may also include an IRES sequence that permits the expression of multiple polypeptides from a single promoter.

In addition to IRES sequences, other elements which permit expression of multiple polypeptides are useful. The vector used in the methods and compositions described herein may include multiple promoters that permit expression more than one polypeptide. The vector used in the methods and compositions described herein may include a protein cleavage site that allows expression of more than one polypeptide. Examples of protein cleavage sites that allow expression of more than one polypeptide are described in Klump et al., Gene Ther. 8:811 (2001), Osborn et al., Molecular Therapy 12:569 (2005), Szymczak and Vignali, Expert Opin Biol Ther. 5:627 (2005), and Szymczak et al., Nat Biotechnol. 22:589 (2004), the disclosures of which are incorporated herein by reference as they pertain to protein cleavage sites that allow expression of more than one polypeptide. It will be readily apparent to one skilled in the art that other elements that permit expression of multiple polypeptides identified in the future are useful and may be utilized in the vectors suitable for use with the compositions and methods described herein.

The vector used in the methods and compositions described herein may, be a clinical grade vector.

### Adeno-Associated Viral Vectors

Nucleic acids of the compositions and methods described herein may be incorporated into rAAV vectors and/or virions in order to facilitate their introduction into a cell (e.g., pluripotent cell, ESC, iPSC, multipotent cell, CD34+ cell, HSC, MPC, BLPC, monocyte, macrophage, microglial progenitor cell, or microglial cell). AAV vectors can be used in the central nervous system, and appropriate promoters and serotypes are discussed in Pignataro et al., J Neural Transm (2017), epub ahead of print, the disclosure of which is incorporated herein by reference as it pertains to promoters and AAV serotypes useful in CNS gene therapy. rAAV vectors useful in the compositions and methods described herein are recombinant nucleic acid constructs (e.g., nucleic acids capable of expression in macrophages or microglia) that include (1) a heterologous sequence to be expressed and (2) viral sequences that facilitate integration and expression of the heterologous genes. The viral sequences may include those sequences of AAV that are required in cis for replication and packaging (e.g., functional ITRs) of the DNA into a virion. Such rAAV vectors may also contain marker or reporter genes. Useful rAAV vectors have one or more of the AAV WT genes deleted in whole or in part but retain functional flanking ITR sequences. The AAV ITRs may be of any serotype suitable for a particular application. Methods for using rAAV vectors are described, for example, in Tai et al., J. Biomed. Sci. 7:279 (2000), and Monahan and Samulski, Gene Delivery 7:24 (2000), the disclosures of each of which are incorporated herein by reference as they pertain to AAV vectors for gene delivery.

The nucleic acids and vectors described herein can be incorporated into a rAAV virion in order to facilitate introduction of the nucleic acid or vector into a cell. The capsid proteins of AAV compose the exterior, non-nucleic acid portion of the virion and are encoded by the AAV cap gene. The cap gene encodes three viral coat proteins, VP1, VP2, and VP3, which are required for virion assembly. The construction of rAAV virions has been described, for example, in U.S. Pat. No. 5,173,414; U.S. Pat. No. 5,139,941; U.S. Pat. No. 5,863,541; U.S. Pat. No. 5,869,305; U.S. Pat. No. 6,057,152; and U.S. Pat. No. 6,376,237; as well as in Rabinowitz et al., J. Virol. 76:791 (2002) and Bowles et al., J. Virol. 77:423 (2003), the disclosures of each of which are incorporated herein by reference as they pertain to AAV vectors for gene delivery.

rAAV virions useful in conjunction with the compositions and methods described herein include those derived from a variety of AAV serotypes including AAV 1, 2, 3, 4, 5, 6, 7, 8, 9, 10 and rh74. For targeting cells located in or delivered to the central nervous system, AAV2, AAV9, and AAV10 may be particularly useful. Construction and use of AAV vectors and AAV proteins of different serotypes are described, for example, in Chao et al., Mol. Ther. 2:619 (2000); Davidson et al., Proc. Natl. Acad. Sci. USA 97:3428 (2000); Xiao et al., J. Virol. 72:2224 (1998); Halbert et al., J. Virol. 74:1524 (2000); Halbert et al., J. Virol. 75:6615 (2001); and Auricchio et al., Hum. Molec. Genet. 10:3075 (2001), the disclosures of each of which are incorporated herein by reference as they pertain to AAV vectors for gene delivery.

Also useful in conjunction with the compositions and methods described herein are pseudotyped rAAV vectors. Pseudotyped vectors include AAV vectors of a given serotype pseudotyped with a capsid gene derived from a serotype other than the given serotype (e.g., AAV1, AAV2, AAV3, AAV4, AAVS, AAV6, AAV7, AAV8, AAV9, and AAV10, among others). Techniques involving the construction and use of pseudotyped rAAV virions are known in the art and are described, for example, in Duan et al., J. Virol. 75:7662 (2001); Halbert et al., J. Virol. 74:1524 (2000); Zolotukhin et al., Methods, 28:158 (2002); and Auricchio et al., Hum. Molec. Genet. 10:3075 (2001).

AAV virions that have mutations within the virion capsid may be used to infect particular cell types more effectively than non-mutated capsid virions. For example, suitable AAV mutants may have ligand insertion mutations for the facilitation of targeting AAV to specific cell types. The construction and characterization of AAV capsid mutants including insertion mutants, alanine screening mutants, and epitope tag mutants is described in Wu et al., J. Virol. 74:8635 (2000). Other rAAV virions that can be used in methods described herein include those capsid hybrids that are generated by molecular breeding of viruses as well as by exon shuffling. See, e.g., Soong et al., Nat. Genet., 25:436 (2000) and Kolman and Stemmer, Nat. Biotechnol. 19:423 (2001).

### Methods for the Delivery of Exogenous Nucleic Acids to Target Cells

Techniques that can be used to introduce a polynucleotide, such as codon-optimized DNA or RNA (e.g., mRNA, tRNA, siRNA, miRNA, shRNA, chemically modified RNA) into a mammalian cell (e.g., pluripotent cell, ESC, iPSC, multipotent cell, CD34+ cell, HSC, MPC, BLPC, monocyte, macrophage, microglial progenitor cell, or microglial cell) are well known in the art. For example, electroporation can be used to permeabilize mammalian cells (e.g., human target cells) by the application of an electrostatic potential to the cell of interest. Mammalian cells, such as human cells, subjected to an external electric field in this manner are subsequently predisposed to the uptake of exogenous nucleic acids (e.g., nucleic acids capable of expression in macrophages or microglia). Electroporation of mammalian cells is described in detail, e.g., in Chu et al., Nucleic Acids Research 15:1311 (1987), the disclosure of which is incorporated herein by reference. A similar technique, Nucleofection™, utilizes an applied electric field in order to stimulate the uptake of exogenous polynucleotides into the nucleus of a eukaryotic cell. Nucleofection™ and protocols useful for performing this technique are described in detail, e.g., in Distler et al., Experimental Dermatology 14:315 (2005), as well as in US 2010/0317114, the disclosures of each of which are incorporated herein by reference.

Additional techniques useful for the transfection of target cells are the squeeze-poration methodology. This technique induces the rapid mechanical deformation of cells in order to stimulate the uptake of exogenous DNA through membranous pores that form in response to the applied stress. This technology is advantageous in that a vector is not required for delivery of nucleic acids into a cell, such as a human target cell. Squeeze-poration is described in detail, e.g., in Sharei et al., Journal of Visualized Experiments 81:e50980 (2013), the disclosure of which is incorporated herein by reference.

Lipofection represents another technique useful for transfection of target cells. This method involves the loading of nucleic acids into a liposome, which often presents cationic functional groups, such as quaternary or protonated amines, towards the liposome exterior. This promotes electrostatic interactions between the liposome and a cell due to the anionic nature of the cell membrane, which ultimately leads to uptake of the exogenous nucleic acids, for example, by direct fusion of the liposome with the cell membrane or by endocytosis of the complex. Lipofection is described in detail, for example, in U.S. Pat. No. 7,442,386, the disclosure of which is incorporated herein by reference. Similar techniques that exploit ionic interactions with the cell membrane to provoke the uptake of foreign nucleic acids are contacting a cell with a cationic polymer-nucleic acid complex. Exemplary cationic molecules that associate with polynucleotides so as to impart a positive charge favorable for interaction with the cell membrane are activated dendrimers (described, e.g., in Dennig, Topics in Current Chemistry 228:227 (2003), the disclosure of which is incorporated herein by reference) polyethylenimine, and diethylaminoethyl (DEAE)-dextran, the use of which as a transfection agent is described in detail, for example, in Gulick et al., Current Protocols in Molecular Biology 40:1:9.2:9.2.1 (1997), the disclosure of which is incorporated herein by reference. Magnetic beads are another tool that can be used to transfect target cells in a mild and efficient manner, as this methodology utilizes an applied magnetic field in order to direct the uptake of nucleic acids. This technology is described in detail, for example, in US 2010/0227406, the disclosure of which is incorporated herein by reference.

Another useful tool for inducing the uptake of exogenous nucleic acids by target cells is laserfection, also called optical transfection, a technique that involves exposing a cell to electromagnetic radiation of a particular wavelength in order to gently permeabilize the cells and allow polynucleotides to penetrate the cell membrane. The bioactivity of this technique is similar to, and in some cases found superior to, electroporation.

Impalefection is another technique that can be used to deliver genetic material to target cells. It relies on the use of nanomaterials, such as carbon nanofibers, carbon nanotubes, and nanowires. Needle-like nanostructures are synthesized perpendicular to the surface of a substrate. DNA containing the gene, intended for intracellular delivery, is attached to the nanostructure surface. A chip with arrays of these needles is then pressed against cells or tissue. Cells that are impaled by nanostructures can express the delivered gene(s). An example of this technique is described in Shalek et al., PNAS 107:25 1870 (2010), the disclosure of which is incorporated herein by reference.

Magnetofection can also be used to deliver nucleic acids to target cells. The magnetofection principle is to associate nucleic acids with cationic magnetic nanoparticles. The magnetic nanoparticles are made of iron oxide, which is fully biodegradable, and coated with specific cationic proprietary molecules varying upon the applications. Their association with the gene vectors (DNA, siRNA, viral vector, etc.) is achieved by salt-induced colloidal aggregation and electrostatic interaction. The magnetic particles are then concentrated on the target cells by the influence of an external magnetic field generated by magnets. This technique is described in detail in Scherer et al., Gene Therapy 9:102 (2002), the disclosure of which is incorporated herein by reference.

Another useful tool for inducing the uptake of exogenous nucleic acids by target cells is sonoporation, a technique that involves the use of sound (typically ultrasonic frequencies) for modifying the permeability of the cell plasma membrane permeabilize the cells and allow polynucleotides to penetrate the cell membrane. This technique is described in detail, e.g., in Rhodes et al., Methods in Cell Biology 82:309 (2007), the disclosure of which is incorporated herein by reference.

Microvesicles represent another potential vehicle that can be used to modify the genome of a target cell according to the methods described herein. For example, microvesicles that have been induced by the co-overexpression of the glycoprotein VSV-G with, e.g., a genome-modifying protein, such as a nuclease, can be used to efficiently deliver proteins into a cell that subsequently catalyze the site-specific cleavage of an endogenous polynucleotide sequence so as to prepare the genome of the cell for the covalent incorporation of a polynucleotide of interest, such as a gene or regulatory sequence. The use of such vesicles, also referred to as Gesicles, for the genetic modification of eukaryotic cells is described in detail, e.g., in Quinn et al., Genetic Modification of Target Cells by Direct Delivery of Active Protein [abstract]. In: Methylation changes in early embryonic genes in cancer [abstract], in: Proceedings of the 18th Annual Meeting of the American Society of Gene and Cell Therapy; 2015 May 13, Abstract No. 122.

### Modulation of Gene Expression Using Gene Editing Techniques

### Disruption of Endogenous Genes

In some embodiments, endogenous expression of a protein described herein is disrupted (e.g., in a patient undergoing treatment, such as in a population of neurons in a patient undergoing treatment). This may be done, for example, in order to suppress expression of an allelic variant of a gene that harbors a deleterious mutation before providing the patient with a functional form of the gene or its protein product. Exemplary methods for disrupting endogenous gene expression are those in which an inhibitory RNA molecule is administered to the patient or contacted with a population of neurons in the patient or the population of cells to be administered to the patient. The inhibitory RNA molecule may function to disrupt endogenous gene expression, for example, act by way of the RNA interference (RNAi) pathway. An inhibitory RNA molecule can decrease the expression level (e.g., protein level or mRNA level) of one or more endogenous genes. For example, an inhibitory RNA molecule may include a short interfering RNA, short hairpin RNA, and/or a miRNA that targets one or more endogenous genes corresponding to a therapeutic protein described herein but harboring a deleterious mutation, such as a mutation that gives rise to, or is associated with the risk of developing an NCD (e.g., Alzheimer's disease, Parkinson's disease, or FTLD). A siRNA is a double-stranded RNA molecule that typically has a length of about 19-25 base pairs. A shRNA is a RNA molecule including a hairpin turn that decreases expression of target genes via RNAi. shRNAs can be delivered to cells in the form of plasmids, e.g., viral or bacterial vectors, e.g., by transfection, electroporation, or transduction). A miRNA is a non-coding RNA molecule that typically has a length of about 22 nucleotides. miRNAs bind to target sites on mRNA molecules and silence the mRNA, e.g., by causing cleavage of the mRNA, destabilization of the mRNA, or inhibition of translation of the mRNA. An inhibitory RNA molecule can be modified, e.g., to contain modified nucleotides, e.g., 2′-fluoro, 2′-o-methyl, 2′-deoxy, unlocked nucleic acid, 2′-hydroxy, phosphorothioate, 2′-thiouridine, 4′-thiouridine, 2′-deoxyuridine. Without being bound by theory, it is believed that certain modification can increase nuclease resistance and/or serum stability or decrease immunogenicity.

In some embodiments, the inhibitory RNA molecule decreases the level and/or activity or function of an endogenous gene, such as an endogenous gene corresponding to a therapeutic protein of the disclosure but harboring one or more deleterious mutations. In some embodiments, the inhibitory RNA molecule inhibits expression of the endogenous gene. In some embodiments, the inhibitory RNA molecule increases degradation of the endogenous gene and/or decreases the stability of the endogenous gene. The inhibitory RNA molecule can be chemically synthesized or transcribed in vitro.

The preparation and use of inhibitory therapeutic agents based on non-coding RNA, such as ribozymes, RNAse P, siRNAs, and miRNAs, are described, for example, in Sioud, RNA Therapeutics: Function, Design, and Delivery (Methods in Molecular Biology). Humana Press 2010, the disclosure of which is incorporated herein by reference.

### Nuclease-Mediated Gene Regulation

Another useful tool for the disruption and/or integration of target genes into the genome of a cell (e.g., pluripotent cell, ESC, iPSC, multipotent cell, CD34+ cell, HSC, MPC, BLPC, monocyte, macrophage, microglial progenitor cell, or microglial cell) is the clustered regularly interspaced short palindromic repeats (CRISPR)/Cas system, a system that originally evolved as an adaptive defense mechanism in bacteria and archaea against viral infection. The CRISPR/Cas system includes palindromic repeat sequences within plasmid DNA and a CRISPR-associated protein (Cas; e.g., Cas9 or Cas12a). This ensemble of DNA and protein directs site specific DNA cleavage of a target sequence by first incorporating foreign DNA into CRISPR loci. Polynucleotides containing these foreign sequences and the repeat-spacer elements of the CRISPR locus are in turn transcribed in a host cell to create a guide RNA, which can subsequently anneal to a target sequence and localize the Cas nuclease to this site. In this manner, highly site-specific Cas-mediated DNA cleavage can be engendered in a foreign polynucleotide because the interaction that brings Cas within close proximity of the target DNA molecule is governed by RNA: DNA hybridization. As a result, one can theoretically design a CRISPR/Cas system to cleave any target DNA molecule of interest. This technique has been exploited in order to edit eukaryotic genomes (Hwang et al. Nature Biotechnology 31:227 (2013), the disclosure of which is incorporated herein by reference) and can be used as an efficient means of site-specifically editing cell genomes in order to cleave DNA prior to the incorporation of a gene encoding a target gene. The use of CRISPR/Cas to modulate gene expression has been described in, e.g., U.S. Pat. No. 8,697,359, the disclosure of which is incorporated herein by reference. Alternative methods for disruption of a target DNS by site-specifically cleaving genomic DNA prior to the incorporation of a gene of interest in a cell include the use of zinc finger nucleases (ZFNs) and transcription activator-like effector nucleases (TALENs). Unlike the CRISPR/Cas system, these enzymes do not contain a guiding polynucleotide to localize to a specific target sequence. Target specificity is instead controlled by DNA binding domains within these enzymes. The use of ZFNs and TALENs in genome editing applications is described, e.g., in Urnov et al. Nature Reviews Genetics 11:636 (201 0); and in Joung et al. Nature Reviews Molecular Cell Biology 14:49 (2013), the disclosures of each of which are incorporated herein by reference. In some embodiments, an endogenous gene is disrupted, e.g., in a cell, using the gene editing techniques described above.

### Transposon-Mediated Gene Regulation

In addition to viral vectors, a variety of additional tools have been developed that can be used for the incorporation of exogenous genes into cells (e.g., pluripotent cells, ESC, iPSC, multipotent cell, CD34+ cell, HSC, MPC, BLPC, monocyte, macrophage, microglial progenitor cell, or microglial cell). One such method that can be used for incorporating polynucleotides encoding target genes into cells involves the use of transposons. Transposons are polynucleotides that encode transposase enzymes and contain a polynucleotide sequence or gene of interest flanked by 5′ and 3′ excision sites. Once a transposon has been delivered into a cell, expression of the transposase gene commences and results in active enzymes that cleave the gene of interest from the transposon. This activity is mediated by the site-specific recognition of transposon excision sites by the transposase. In certain cases, these excision sites may be terminal repeats or inverted terminal repeats. Once excised from the transposon, the gene of interest can be integrated into the genome of a mammalian cell by transposase-catalyzed cleavage of similar excision sites that exist within the nuclear genome of the cell. This allows the gene of interest to be inserted into the cleaved nuclear DNA at the complementary excision sites, and subsequent covalent ligation of the phosphodiester bonds that join the gene of interest to the DNA of the mammalian cell genome completes the incorporation process. In certain cases, the transposon may be a retrotransposon, such that the gene encoding the target gene is first transcribed to an RNA product and then reverse-transcribed to DNA before incorporation in the mammalian cell genome. Transposon systems include the piggybac transposon (described in detail in, e.g., WO 2010/085699) and the sleeping beauty transposon (described in detail in, e.g., US 2005/0112764), the disclosures of each of which are incorporated herein by reference.

### Methods of Diagnosis

### Methods of Diagnosing Alzheimer's Disease

Patients may be diagnosed as having Alzheimer's disease using methods well-known in the art, such as, e.g., the methods described in The Diagnostic and Statistical Manual of Mental Disorders, Fifth Edition and the International Classification of Diseases, 11th Revision. For example, diagnosis of Alzheimer's disease in a patient may be guided by neuropsychological testing to assess the degree of cognitive impairment in a patient. The patient's cognitive function may be assessed by performing cognitive tests that evaluate performance across one or more cognitive domains including but not limited to complex attention, executive function, learning and memory, language, perceptual-motor function, and social cognition. Comparison of cognitive function in the patient relative to a norm appropriate for the patients age, medical history, education, socioeconomic status, and lifestyle (e.g., a reference population, such as, e.g., a general population) may be done to determine the diagnosis of Alzheimer's disease in the patient. The patient may be diagnosed as having major Alzheimer's disease or mild Alzheimer's disease. Major Alzheimer's disease is characterized by significant cognitive decline that interferes with personal independence and normal daily functioning and is not due to delirium or other mental disorder. Mild Alzheimer's disease is characterized by moderate cognitive decline that does not interfere with personal independence and normal daily functioning and is not due to delirium or other mental disorder. Major Alzheimer's disease can be characterized by a score obtained on a cognitive test by a patient that is more than two standard deviations away from the mean score of a reference population (e.g., the mean score of a general population) or a score that is in the third percentile of the distribution of scores of the reference population. Mild Alzheimer's disease can be characterized by a score obtained on a cognitive test by a patient that is between one to two standard deviations away from the mean score of a reference population (e.g., the mean score of a general population) or a score that is between the 3^{rd} and 16^{th} percentile of the distribution of scores of the reference population. Non-limiting examples of cognitive tests include Eight-item Informant Interview to Differentiate Aging and Dementia (AD8), Annual Wellness Visit (AWV), General Practitioner Assessment of Cognition (GPCOG), Health Risk Assessment (HRA), Memory Impairment Screen (MIS), Mini Mental Status Exam (MMSE), Montreal Cognitive Assessment (MoCA), St. Louis University Mental Status Exam (SLUMS), and Short Informant Questionnaire on Cognitive Decline in the Elderly (Short IQCODE). Additionally or alternatively, the use of F18-fluorodeoxyglucose PET scans or MRI scans may be used to determine the presence of neurodegeneration in a patient with Alzheimer's disease.

Furthermore, the patient may be tested for the presence of biomarkers specific to Alzheimer's disease. For example, a patient may be tested for the presence of biomarkers that indicate that the patient has Alzheimer's disease, such as the presence of Aβ plaques or NFTs of hyperphosphorylated tau proteins in the forebrain of the patient, presence of mutations in the APP, PSEN1, PSEN2, and/or TREM2 genes in the patient, as well as variations in the ε4 allele of APOE.

### Methods of Diagnosing Parkinson Disease

Patients may be diagnosed as having Parkinson disease using methods well-known in the art, such as, e.g., the methods described in The Diagnostic and Statistical Manual of Mental Disorders, Fifth Edition and the International Classification of Diseases, 11th Revision. For example, diagnosis of Parkinson disease in a patient may be guided by neuropsychological testing to assess the degree of cognitive impairment in a patient. The patient's cognitive function may be assessed by performing cognitive tests that evaluate performance across one or more cognitive domains including but not limited to complex attention, executive function, learning and memory, language, perceptual-motor function, and social cognition. Comparison of cognitive function in the patient relative to a norm appropriate for the patients age, medical history, education, socioeconomic status, and lifestyle (e.g., a reference population, such as, e.g., a general population) may be done to determine the diagnosis of Parkinson disease in the patient. The patient may be diagnosed as having major Parkinson disease or mild Parkinson disease. Major Parkinson disease is characterized by significant cognitive decline that interferes with personal independence and normal daily functioning and is not due to delirium or other mental disorder. Mild Parkinson disease is characterized by moderate cognitive decline that does not interfere with personal independence and normal daily functioning and is not due to delirium or other mental disorder. Major Parkinson disease can be characterized by a score obtained on a cognitive test by a patient that is more than two standard deviations away from the mean score of a reference population (e.g., the mean score of a general population) or a score that is in the third percentile of the distribution of scores of the reference population. Mild Parkinson disease can be characterized by a score obtained on a cognitive test by a patient that is between one to two standard deviations away from the mean score of a reference population (e.g., the mean score of a general population) or a score that is between the 3rd and 16th percentile of the distribution of scores of the reference population. Non-limiting examples of cognitive tests include AD8, AWV, GPCOG, HRA, MIS, MMSE, MoCA, SLUMS, and Short IQCODE. Additionally or alternatively, the use of F18-fluorodeoxyglucose PET scans or MRI scans may be used to determine the presence of neurodegeneration in a patient with Parkinson disease.

Furthermore, the patient may be tested for the presence of biomarkers specific to Parkinson disease. For example, a patient may be tested for the presence of biomarkers that indicate that the patient has Parkinson disease, such as, e.g., the presence of dopaminergic neuron death, presence of Lewy bodies containing a-synuclein in the brain, and/or mutations in the glucocerebrocidase (GBA), parkin, PTEN-induced putative kinase 1 (PINK1), leucine-rich repeat kinase 2 (LRRK2), and Parkinsonism-associated deglycase (DJ-1) genes described herein to determine whether the patient has Parkinson disease.

### Methods of Diagnosing Frontotemporal Lobar Degeneration

Patients may be diagnosed as having a FTLD using methods well-known in the art, such as, e.g., the methods described in The Diagnostic and Statistical Manual of Mental Disorders, Fifth Edition and the International Classification of Diseases, 11th Revision. For example, diagnosis of FTLD in a patient may be guided by neuropsychological testing to assess the degree of cognitive impairment in a patient. The patient's cognitive function may be assessed by performing cognitive tests that evaluate performance across one or more cognitive domains including but not limited to complex attention, executive function, learning and memory, language, perceptual-motor function, and social cognition. Comparison of cognitive function in the patient relative to a norm appropriate for the patients age, medical history, education, socioeconomic status, and lifestyle (e.g., a reference population, such as, e.g., a general population) may be done to determine the diagnosis of FTLD in the patient. The patient may be diagnosed as having major FTLD or mild FTLD. Major FTLD is characterized by significant cognitive decline that interferes with personal independence and normal daily functioning and is not due to delirium or other mental disorder. Mild FTLD is characterized by moderate cognitive decline that does not interfere with personal independence and normal daily functioning and is not due to delirium or other mental disorder. Major FTLD can be characterized by a score obtained on a cognitive test by a patient that is more than two standard deviations away from the mean score of a reference population (e.g., the mean score of a general population) or a score that is in the third percentile of the distribution of scores of the reference population. Mild FTLD can be characterized by a score obtained on a cognitive test by a patient that is between one to two standard deviations away from the mean score of a reference population (e.g., the mean score of a general population) or a score that is between the 3^{rd} and 16^{th} percentile of the distribution of scores of the reference population. Non-limiting examples of cognitive tests include AD8, AWV, GPCOG, HRA, MIS, MMSE, MoCA, SLUMS, and Short IQCODE. Additionally or alternatively, the use of F18-fluorodeoxyglucose PET scans or MRI scans may be used to determine the presence of neurodegeneration in a patient with FTLD.

Furthermore, the patient may be tested for the presence of biomarkers specific to Parkinson disease. For example, a patient may be tested for the presence of biomarkers that indicate that the patient has FTLD, such as, e.g., the presence of tau-positive neuronal and glial inclusions, ub-positive and TDP43-positive but tau-negative inclusions, ub and FUS-positive but tau-negative inclusions, mutations in the PGRN gene disclosed herein and/or mutations on chromosome 17q21 described herein.

### Methods of Treatment

### Routes of Administration

The compositions described herein may be administered to a patient (e.g., a patient having an NCD such as, e.g., Alzheimer's disease, Parkinson's disease, or a FTLD) by one or more of a variety of routes, such as intracerebroventricularly, intrathecally, intraparenchymally, stereotactically, intravenously, intraosseously, or by means of a bone marrow transplant. In some embodiments, the compositions described herein may be administered to a patient systemically (e.g., intravenously), directly to the central nervous system (CNS) (e.g., intracerebroventricularly, directly to the cerebrospinal fluid (such as intrathecally), intraparenchymally, or stereotactically), or directly into the bone marrow (e.g., intraosseously). In some embodiments, the compositions described herein are administered to a patient intracerebroventricularly into the cerebral lateral ventricles (a description of this method can be found in Capotondo et al., Science Advances 3:e1701211 (2017), the disclosure of which is incorporated herein by reference as it pertains to intracerebroventricular injection methods). The most suitable route for administration in any given case may depend on the particular composition administered, the patient, pharmaceutical formulation methods, administration methods (e.g., administration time and administration route), the patient's age, body weight, sex, severity of the diseases being treated, the patients diet, and the patient's excretion rate. Multiple routes of administration may be used to treat a single patient, e.g., intracerebroventricular or stereotactic injection and intravenous injection, intracerebroventricular or stereotactic injection and intraosseous injection, intracerebroventricular or stereotactic injection and bone marrow transplant, intracerebroventricular or stereotactic injection and intraparenchymal injection, intrathecal injection and intravenous injection, intrathecal injection and intraosseous injection, intrathecal injection and bone marrow transplant, intrathecal injection and intraparenchymal injection, intraparenchymal injection and intravenous injection, intraparenchymal injection and intraosseous injection, or intraparenchymal injection and bone marrow transplant. Multiple routes of administration may be used to treat a single patient at one time, or the patient may receive treatment via one route of administration first, and receive treatment via another route of administration during a second appointment, e.g., 1 week later, 2 weeks later, 1 month later, 6 months later, or 1 year later. Compositions may be administered to a patient once, or cells may be administered one or more times (e.g., 2-10 times) per week, month, or year.

### Conditioning

Prior to administration of a composition of the disclosure to a patient (e.g., a patient having an NCD such as, e.g., Alzheimer's disease, Parkinson's disease, or a FTLD), it may be advantageous to deplete or ablate endogenous microglia and/or hematopoietic stem and progenitor cells. Microglia and/or hematopoietic stem and progenitor cells can be ablated through the use of chemical agents (e.g., busulfan, treosulfan, PLX3397, PLX647, PLX5622, or clodronate liposomes), irradiation, or a combination thereof. The agents used for cell ablation may be BBB-penetrating (e.g., busulfan) or may lack the ability to cross the BBB (e.g., treosulfan). Exemplary microglia and/or hematopoietic stem and progenitor cells ablating agents are busulfan (Capotondo et al., PNAS 109:15018 (2012), the disclosure of which is incorporated by reference as it pertains to the use of busulfan to ablate microglia), treosulfan, PLX3397, PLX647, PLX5622, or clodronate liposomes. Other agents for the depletion of endogenous microglia and/or hematopoietic stem and progenitor cells include cytotoxins covalently conjugated to antibodies or antigen binding fragments thereof capable of binding antigens expressed by hematopoietic stem cells so as to form an antibody-drug conjugate. Cytotoxins suitable for antibody drug conjugates include DNA-intercalating agents, (e.g., anthracyclines), agents capable of disrupting the mitotic spindle apparatus (e.g., vinca alkaloids, maytansine, maytansinoids, and derivatives thereof), RNA polymerase inhibitors (e.g., an amatoxin, such as a-amanitin and derivatives thereof), agents capable of disrupting protein biosynthesis (e.g., agents that exhibit rRNA N-glycosidase activity, such as saporin and ricin A-chain), among others known in the art.

Ablation may eliminate all microglia and/or hematopoietic stem and progenitor cells, or it may reduce microglia and/or hematopoietic stem and progenitor cells numbers by at least, e.g., 5% (e.g., at least 5%, 10%, 20%, 30%, 40%, 50%, 60%, 70%, 80%, 90%, 95%, or more). In some embodiments, one or more agents to ablate microglia and/or hematopoietic stem and progenitor cells are administered at least one week (e.g., 1, 2, 3, 4, 5, or 6 weeks or more) before administration of a composition described herein. Cells administered in accordance with the methods described herein may replace the ablated microglia and/or hematopoietic stem and progenitor cells, and may repopulate the brain following intracerebroventricular, stereotactic, intravenous, or intraosseous injection, or following bone marrow transplant. Cells administered intravenously, intraosseously, or by bone marrow transplant may cross the blood brain barrier to enter the brain and differentiate into microglia. Cells administered to the brain, e.g., cells administered intracerebroventricularly or stereotactically, can differentiate into microglia in vivo or can be differentiated into microglia ex vivo.

### Stem Cell Rescue

The methods described herein may include administering to a patient a population of cells (e.g., ESCs, iPSCs, or CD34+ cells). In some embodiments, these cells are cells that have not been modified to contain a transgene encoding one or more therapeutic proteins of the disclosure. Instead, these cells may first be modified so as to disrupt endogenous expression of a protein of interest before administration of the cells to the patient. The cells may be administered using any route of administration described herein, such as systemically (e.g., intravenously), or by bone marrow transplantation to reconstitute the bone marrow compartment following conditioning as described herein. For example, these cells may migrate to a stem cell niche and increase the quantity of cells of the hematopoietic lineage at such a site by, for example, 1%, 2%, 3%, 4%, 5%, 6%, 7%, 8%, 9%, 10%, 11%, 12%, 13%, 14%, 15%, 16%, 35 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or more. Administration may occur prior to, during, or following administration of a therapeutic composition described herein.

### Selection of Donor Cells

In some embodiments, the patient undergoing treatment is the donor that provides cells (e.g., ESCs, iPSCs, or CD34+ cells) which are subsequently modified to contain nucleic acids encoding one or more therapeutic proteins of the disclosure (e.g., nucleic acids capable of expression in macrophages or microglia) before being re-administered to the patient. In such cases, withdrawn cells (e.g., hematopoietic stem or progenitor cells) may be re-infused into the patient following, for example, incorporation of a transgene encoding one or more therapeutic proteins of the disclosure, and/or disruption of an allelic variant harboring a deleterious mutation), such that the cells may subsequently home to hematopoietic tissue and establish productive hematopoiesis, thereby populating or repopulating a line of cells that is defective or deficient in the patient (e.g., a population of microglia). In cases in which the patient undergoing treatment also serves as the cell donor, the transplanted cells (e.g., hematopoietic stem or progenitor cells) are less likely to undergo graft rejection. This stems from the fact that the infused cells are derived from the patient and express the same HLA class I and class II antigens as expressed by the patient. Alternatively, the patient and the donor may be distinct. In some embodiments, the patient and the donor are related, and may, for example, be HLA-matched. As described herein, HLA-matched donor-recipient pairs have a decreased risk of graft rejection, as endogenous T cells and NK cells within the transplant recipient are less likely to recognize the incoming hematopoietic stem or progenitor cell graft as foreign and are thus less likely to mount an immune response against the transplant. Exemplary HLA-matched donor-recipient pairs are donors and recipients that are genetically related, such as familial donor-recipient pairs (e.g., sibling donor-recipient pairs). In some embodiments, the patient and the donor are HLA-mismatched, which occurs when at least one HLA antigen, in particular with respect to HLA-A, HLA-B and HLA-DR, is mismatched between the donor and recipient. To reduce the likelihood of graft rejection, for example, one haplotype may be matched between the donor and recipient, and the other may be mismatched.

Pharmaceutical Compositions and Dosing

In cases in which a patient is administered a population of cells (e.g., pluripotent cells, ESCs, iPSCs, multipotent cells, CD34+ cells, HSCs, MPCs, BLPCs, monocytes, macrophages, microglial progenitor cells, or microglia) that together contain nucleic acids encoding one or more therapeutic proteins of the disclosure (e.g., nucleic acids capable of expression in macrophages or microglia), the number of cells administered may depend, for example, on the expression level of the desired protein(s), the patient, pharmaceutical formulation methods, administration methods (e.g., administration time and administration route), the patient's age, body weight, sex, severity of the disease being treated, and whether or not the patient has been treated with agents to ablate endogenous pluripotent cells (e.g., endogenous CD34+ cells, hematopoietic stem or progenitor cells, or microglia, among others). The number of cells administered may be, for example, from 1×10⁶ cells/kg to 1×10¹² cells/kg, or more (e.g., 1×10⁷ cells/kg, 1×10⁸ cells/kg, 1×10⁹ cells/kg, 1×10¹⁰ cells/kg, 1×10¹¹ cells/kg, 1×10¹² cells/kg, or more). Cells may be administered in an undifferentiated state, or after partial or complete differentiation into microglia. The number of cells may be administered in any suitable dosage form.

### EXAMPLES

The following examples are put forth so as to provide those of ordinary skill in the art with a description of how the compositions and methods described herein may be used, made, and evaluated, and are intended to be purely exemplary of the disclosure and are not intended to limit the scope of what the inventors regard as their disclosure.

### Example 1

### Generation of a Cell Containing a Transgene Encoding One or More Therapeutic Proteins Useful for the Treatment of Alzheimer's Disease

An exemplary method for making cells (e.g., pluripotent cells (e.g., embryonic stem cells (ESCs) or induced pluripotent stem cells (ISPCs)), multipotent cells (e.g., CD34+ cells such as, e.g., hematopoietic stem cells (HSCs) or myeloid precursor cells (MPCs)), blood lineage progenitor cells (BLPCS; e.g., monocytes), macrophages, microglial progenitor cells, or microglia) that contain nucleic acids encoding one or more therapeutic proteins useful for the treatment of Alzheimer's disease (e.g., nucleic acids capable of expression in macrophages or microglia), such as one or more proteins selected from APP, PSEN1, PSEN2, APOE, TOMM40, GAB2, APOC1, TREM2, ABI3, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1 L, STK24, DISC1, MPZL1, SLC4A1AP, TRIP4, MSRA, HS3ST1, ZNF224, and AP2A2, is by way of transduction. Retroviral vectors (e.g., a lentiviral vector, alpharetroviral vector, or gammaretroviral vector) containing, e.g., a microglia-specific promoter, such as the CD68 promoter, and a polynucleotide encoding one or more proteins of interest can be engineered using standard techniques known in the art. After the retroviral vector is engineered, the retrovirus can be used to transduce cells to generate a population of cells that contain nucleic acids encoding the therapeutic protein(s).

Additional exemplary methods for making cells that contain nucleic acids encoding such proteins for use in the treatment of Alzheimer's disease are transfection techniques. Using molecular biology procedures described herein and known in the art, plasmid DNA containing a promoter, such as a microglia-specific promoter, (e.g., the CD68 promoter), and a polynucleotide encoding one or more therapeutic proteins can be produced. For example, a therapeutic transgene may be amplified from a human cell line using PCR-based techniques known in the art, or the transgene may be synthesized, for example, using solid-phase polynucleotide synthesis procedures. The transgene and promoter can then be ligated into a plasmid of interest, for example, using suitable restriction endonuclease-mediated cleavage and ligation protocols. After the plasmid DNA is engineered, the plasmid can be used to transfect the cell using, for example, electroporation or another transfection technique described herein to generate a population of cells that contain nucleic acids encoding the protein(s). In both exemplary methods described herein, each of the one or more therapeutic proteins may be expressed as a fusion protein. The fusion protein may contain a receptor-binding (Rb) domain of Apolipoprotein E (ApoE), such as an Rb domain described herein, so as to allow for the penetration of the blood-brain barrier by the desired therapeutic protein(s).

### Example 2

### Generation of a Cell Containing a Transgene Encoding One or More Therapeutic Proteins Useful for the Treatment of Parkinson's Disease

An exemplary method for making cells (e.g., pluripotent cells, ESCs, iPSCs, multipotent cells, CD34+ cells, HSCs, MPCs, BLPCs, monocytes, macrophages, microglial progenitor cells, or microglia) that contain nucleic acids encoding one or more therapeutic proteins useful for the treatment of Parkinson's disease (e.g., nucleic acids capable of expression in macrophages or microglia), such as one or more proteins selected from FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1 B, NUCKS1, and ACMSD, is by way of transduction. Retroviral vectors (e.g., a lentiviral vector, alpharetroviral vector, or gammaretroviral vector) containing, e.g., a microglia-specific promoter, such as the CD68 promoter, and a polynucleotide encoding one or more proteins of interest can be engineered using standard techniques known in the art. After the retroviral vector is engineered, the retrovirus can be used to transduce cells to generate a population of cells that contain nucleic acids encoding the therapeutic protein(s).

Additional exemplary methods for making cells that contain nucleic acids encoding such proteins for use in the treatment of Parkinson's disease are transfection techniques. Using molecular biology procedures described herein and known in the art, plasmid DNA containing a promoter, such as a microglia-specific promoter, (e.g., the CD68 promoter), and a polynucleotide encoding one or more therapeutic proteins can be produced. For example, a therapeutic transgene may be amplified from a human cell line using PCR-based techniques known in the art, or the transgene may be synthesized, for example, using solid-phase polynucleotide synthesis procedures. The transgene and promoter can then be ligated into a plasmid of interest, for example, using suitable restriction endonuclease-mediated cleavage and ligation protocols. After the plasmid DNA is engineered, the plasmid can be used to transfect the cells using, for example, electroporation or another transfection technique described herein to generate a population of cells that contain nucleic acids encoding the protein(s). In both exemplary methods described herein, each of the one or more therapeutic proteins may be expressed as a fusion protein. The fusion protein may contain a Rb domain of ApoE, such as an Rb domain described herein, so as to allow for the penetration of the blood-brain barrier by the desired therapeutic protein(s).

### Example 3

### Generation of a Cell Containing a Transgene One or More Therapeutic Proteins Useful for the Treatment of a Frontotemporal Lobar Degeneration

An exemplary method for making cells (e.g., pluripotent cells, ESCs, iPSCs, multipotent cells, CD34+ cells, HSCs, MPCs, BLPCs, monocytes, macrophages, microglial progenitor cells, or microglia) that contain nucleic acids encoding one or more therapeutic proteins useful for the treatment of a FTLD (e.g., nucleic acids capable of expression in macrophages or microglia), such as one or more proteins selected from HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT, is by way of transduction. Retroviral vectors (e.g., a lentiviral vector, alpharetroviral vector, or gammaretroviral vector) containing, e.g., a microglia-specific promoter, such as the CD68 promoter, and a polynucleotide encoding one or more proteins of interest can be engineered using standard techniques known in the art. After the retroviral vector is engineered, the retrovirus can be used to transduce cells to generate a population of cells that contain nucleic acids encoding the therapeutic protein(s).

Additional exemplary methods for making cells that contain nucleic acids encoding such proteins for use in the treatment of a FTLD, such as frontotemporal dementia, semantic dementia, or progressive nonfluent aphasia, are transfection techniques. Using molecular biology procedures described herein and known in the art, plasmid DNA containing a promoter, such as a microglia-specific promoter, (e.g., the CD68 promoter), and a polynucleotide encoding one or more therapeutic proteins can be produced. For example, a therapeutic transgene may be amplified from a human cell line using PCR-based techniques known in the art, or the transgene may be synthesized, for example, using solid-phase polynucleotide synthesis procedures. The transgene and promoter can then be ligated into a plasmid of interest, for example, using suitable restriction endonuclease-mediated cleavage and ligation protocols. After the plasmid DNA is engineered, the plasmid can be used to transfect the cells using, for example, electroporation or another transfection technique described herein to generate a population of cells that contain nucleic acids encoding the protein(s). In both exemplary methods described herein, each of the one or more therapeutic proteins may be expressed as a fusion protein. The fusion protein may contain a Rb domain of ApoE, such as an Rb domain described herein, so as to allow for the penetration of the blood-brain barrier by the desired therapeutic protein(s).

### Example 4

### Generation of a Cell Containing a Transgene Encoding One or More Therapeutic Proteins Useful for the Treatment of Alzheimer's Disease, Parkinson Disease, or a Frontotemporal Lobar Degeneration

An exemplary method for making cells (e.g., pluripotent cells, ESCs, iPSCs, multipotent cells, CD34+ cells, HSCs, MPCs, BLPCs, monocytes, macrophages, microglial progenitor cells, or microglia) that contain nucleic acids encoding one or more therapeutic proteins useful for the treatment of Alzheimer's disease, Parkinson disease, or a FTLD, such as one or more proteins selected from APP, PSEN1, PSEN2, APOE, TOMM40, GAB2, APOC1, TREM2, ABI3, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1 L, STK24, DISCI , MPZL1, SLC4A1AP, TRIP4, MSRA, HS3ST1, ZNF224, AP2A2, FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1 L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1B, NUCKS1, ACMSD, HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT, is by way of transduction. Retroviral vectors (e.g., a lentiviral vector, alpharetroviral vector, or gammaretroviral vector) containing, e.g., a microglia-specific promoter, such as the CD68 promoter, and a polynucleotide encoding one or more proteins of interest can be engineered using standard techniques known in the art. After the retroviral vector is engineered, the retrovirus can be used to transduce cells to generate a population of cells that contain nucleic acids encoding the therapeutic protein(s).

Additional exemplary methods for making cells that contain nucleic acids encoding such proteins for use in the treatment of Alzheimer's disease, Parkinson disease, or a FTLD, such as frontotemporal dementia, semantic dementia, or progressive nonfluent aphasia, are transfection techniques. Using molecular biology procedures described herein and known in the art, plasmid DNA containing a promoter, such as a microglia-specific promoter, (e.g., the CD68 promoter), and a polynucleotide encoding one or more therapeutic proteins can be produced. For example, a therapeutic transgene may be amplified from a human cell line using PCR-based techniques known in the art, or the transgene may be synthesized, for example, using solid-phase polynucleotide synthesis procedures. The transgene and promoter can then be ligated into a plasmid of interest, for example, using suitable restriction endonuclease-mediated cleavage and ligation protocols. After the plasmid DNA is engineered, the plasmid can be used to transfect the cells using, for example, electroporation or another transfection technique described herein to generate a population of cells that contain nucleic acids encoding the protein(s). In both exemplary methods described herein, each of the one or more therapeutic proteins may be expressed as a fusion protein. The fusion protein may contain a Rb domain of ApoE, such as an Rb domain described herein, so as to allow for the penetration of the blood-brain barrier by the desired therapeutic protein(s).

### Example 5

### Administration of a Therapeutic Composition to a Patient Suffering from Alzheimer's Disease

According to the methods disclosed herein, a patient, such as a human patient, can be treated so as to reduce or alleviate symptoms of Alzheimer's disease and/or so as to target an underlying biochemical etiology of the disease. To this end, the patient may be administered, for example, a population of cells (e.g., pluripotent cells, ESCs, iPSCs, multipotent cells, CD34+ cells, HSCs, MPCs, BLPCs, monocytes, macrophages, microglial progenitor cells, or microglia) expressing one or more therapeutic proteins selected from APP, PSEN1, PSEN2, APOE, TOMM40, GAB2, APOC1, TREM2, ABI3, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1 L, STK24, DISC1, MPZL1, SLC4A1AP, TRIP4, MSRA, HS3ST1, ZNF224, and AP2A2. The population of cells may be administered to the patient, for example, systemically (e.g., intravenously), directly to the CNS (e.g., intracerebroventricularly or stereotactically), or directly into the bone marrow (e.g., intraosseously). The cells can also be administered to the patient by multiple routes of administration, for example, intravenously and intracerebroventricularly. The cells are administered in a therapeutically effective amount, such as from 1×10⁶ cells/kg to 1×10¹² cells/kg or more (e.g., 1×10⁷ cells/kg, 1×10⁸ cells/kg, 1×10⁹ cells/kg, 1×10¹⁰ cells/kg, 1×10¹¹ cells/kg, 1×10¹² cells/kg, or more).

Before the population of cells is administered to the patient, one or more agents may be administered to the patient to ablate the patient's endogenous microglia and/or hematopoietic stem and progenitor cells, such as, busulfan, treosulfan, PLX3397, PLX647, PLX5622, and/or clodronate liposomes. Other methods of cell ablation may also be used, such as irradiation, which may be performed alone or in combination with one or more of the aforementioned agents to ablate the patient's microglia and/or hematopoietic stem and progenitor cells. These agents and/or treatments may ablate endogenous microglia and/or hematopoietic stem and progenitor cells by at least 5% (e.g., at least 5%, 10%, 15%, 20%, 25%, 30%, 35%, 40%, 45%, 50%, 60%, 70%, 80%, 90%, 99%, or more), as assessed by PET imaging techniques known in the art. If the population of cells is administered to the patient after ablation, the cells may have an improved rate or repopulation of the brain, where they may differentiate, e.g., into microglia. The population of cells can be administered to the patient from, for example, 1 week to 1 month (e.g., 1 week, 2 weeks, 3 weeks, 4, weeks) or more after ablation.

Additionally or alternatively, the patient may be administered, for example, one or more other agents that collectively elevate the expression and/or activity level of one or more of the foregoing proteins. Such agents include viral vectors that collectively encode the one or more proteins. Exemplary viral vectors are Retroviridae family viral vectors, such as a lentivirus, alpharetrovirus, or gammaretrovirus, among others described herein. Additional agents that may be provided to a patient for the purpose of augmenting the level of one or more of the foregoing proteins include interfering RNA molecules, such as siRNA, shRNA, and miRNA molecules, as well as small molecule agents that modulate the expression of one or more of the above proteins, in addition to the one or more of the above proteins themselves.

### Example 6

### Administration of a Therapeutic Composition to a Patient Suffering from Parkinson's Disease

According to the methods disclosed herein, a patient, such as a human patient, can be treated so as to reduce or alleviate symptoms of Parkinson's disease and/or so as to target an underlying biochemical etiology of the disease. To this end, the patient may be administered, for example, a population of cells (e.g., pluripotent cells, ESCs, iPSCs, multipotent cells, CD34+ cells, HSCs, MPCs, BLPCs, monocytes, macrophages, microglial progenitor cells, or microglia) expressing one or more therapeutic proteins selected from FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1 B, NUCKS1, and ACMSD. The population of cells may be administered to the patient, for example, systemically (e.g., intravenously), directly to the CNS (e.g., intracerebroventricularly or stereotactically), or directly into the bone marrow (e.g., intraosseously). The cells can also be administered to the patient by multiple routes of administration, for example, intravenously and intracerebroventricularly. The cells are administered in a therapeutically effective amount, such as from 1×10⁶ cells/kg to 1×10¹² cells/kg or more (e.g., 1×10⁷ cells/kg, 1×10⁸ cells/kg, 1×10⁹ cells/kg, 1×10¹⁰ cells/kg, 1×10¹¹ cells/kg, 1×10¹² cells/kg, or more).

Before the population of cells is administered to the patient, one or more agents may be administered to the patient to ablate the patient's endogenous microglia and/or hematopoietic stem and progenitor cells, such as, busulfan, treosulfan, PLX3397, PLX647, PLX5622, and/or clodronate liposomes. Other methods of cell ablation may also be used, such as irradiation, which may be performed alone or in combination with one or more of the aforementioned agents to ablate the patient's microglia and/or hematopoietic stem and progenitor cells. These agents and/or treatments may ablate endogenous microglia and/or hematopoietic stem and progenitor cells by at least 5% (e.g., at least 5%, 10%, 15%, 20%, 25%, 30%, 35%, 40%, 45%, 50%, 60%, 70%, 80%, 90%, 99%, or more), as assessed by PET imaging techniques known in the art. If the population of cells is administered to the patient after ablation, the cells may have an improved rate or repopulation of the brain, where they may differentiate, e.g., into microglia. The population of cells can be administered to the patient from, for example, 1 week to 1 month (e.g., 1 week, 2 weeks, 3 weeks, 4, weeks) or more after ablation.

Additionally or alternatively, the patient may be administered, for example, one or more other agents that collectively elevate the expression and/or activity level of one or more of the foregoing proteins. Such agents include viral vectors that collectively encode the one or more proteins. Exemplary viral vectors are Retroviridae family viral vectors, such as a lentivirus, alpharetrovirus, or gammaretrovirus, among others described herein. Additional agents that may be provided to a patient for the purpose of augmenting the level of one or more of the foregoing proteins include interfering RNA molecules, such as siRNA, shRNA, and miRNA molecules, as well as small molecule agents that modulate the expression of one or more of the above proteins, in addition to the one or more of the above proteins themselves.

### Example 7

### Administration of a Therapeutic Composition to a Patient Suffering from a Frontotemporal Lobar Degeneration

According to the methods disclosed herein, a patient, such as a human patient, can be treated so as to reduce or alleviate symptoms of a FTLD and/or so as to target an underlying biochemical etiology of this class of disease. To this end, the patient may be administered, for example, a population of cells (e.g., pluripotent cells, ESCs, iPSCs, multipotent cells, CD34+ cells, HSCs, MPCs, BLPCs, monocytes, macrophages, microglial progenitor cells, or microglia) expressing one or more therapeutic proteins selected from HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT. The population of cells may be administered to the patient, for example, systemically (e.g., intravenously), directly to the CNS (e.g., intracerebroventricularly or stereotactically), or directly into the bone marrow (e.g., intraosseously). The cells can also be administered to the patient by multiple routes of administration, for example, intravenously and intracerebroventricularly. The cells are administered in a therapeutically effective amount, such as from 1×10⁶ cells/kg to 1×10¹² cells/kg or more (e.g., 1×10⁷ cells/kg, 1×10⁸ cells/kg, 1×10⁹ cells/kg, 1×10¹⁰ cells/kg, 1×10¹¹ cells/kg, 1×10¹² cells/kg, or more).

Before the population of cells is administered to the patient, one or more agents may be administered to the patient to ablate the patient's endogenous microglia and/or hematopoietic stem and progenitor cells, such as, busulfan, treosulfan, PLX3397, PLX647, PLX5622, and/or clodronate liposomes. Other methods of cell ablation may also be used, such as irradiation, which may be performed alone or in combination with one or more of the aforementioned agents to ablate the patient's microglia and/or hematopoietic stem and progenitor cells. These agents and/or treatments may ablate endogenous microglia and/or hematopoietic stem and progenitor cells by at least 5% (e.g., at least 5%, 10%, 15%, 20%, 25%, 30%, 35%, 40%, 45%, 50%, 60%, 70%, 80%, 90%, 99%, or more), as assessed by PET imaging techniques known in the art. If the population of cells is administered to the patient after ablation, the cells may have an improved rate or repopulation of the brain, where they may differentiate, e.g., into microglia. The population of cells can be administered to the patient from, for example, 1 week to 1 month (e.g., 1 week, 2 weeks, 3 weeks, 4, weeks) or more after ablation.

Additionally or alternatively, the patient may be administered, for example, one or more other agents that collectively elevate the expression and/or activity level of one or more of the foregoing proteins. Such agents include viral vectors that collectively encode the one or more proteins. Exemplary viral vectors are Retroviridae family viral vectors, such as a lentivirus, alpharetrovirus, or gammaretrovirus, among others described herein. Additional agents that may be provided to a patient for the purpose of augmenting the level of one or more of the foregoing proteins include interfering RNA molecules, such as siRNA, shRNA, and miRNA molecules, as well as small molecule agents that modulate the expression of one or more of the above proteins, in addition to the one or more of the above proteins themselves.

### Example 8

### Administration of a Therapeutic Composition to a Patient Suffering from Alzheimer's Disease, Parkinson Disease, or a Frontotemporal Lobar Degeneration

According to the methods disclosed herein, a patient, such as a human patient, can be treated so as to reduce or alleviate symptoms of Alzheimer's disease, Parkinson disease, or a FTLD and/or so as to target an underlying biochemical etiology of this class of disease. To this end, the patient may be administered, for example, a population of cells (e.g., pluripotent cells, ESCs, iPSCs, multipotent cells, CD34+ cells, HSCs, MPCs, BLPCs, monocytes, macrophages, microglial progenitor cells, or microglia) expressing one or more therapeutic proteins selected from APP, PSEN1, PSEN2, APOE, TOMM40, GAB2, APOC1, TREM2, ABI3, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1L, STK24, DISCI , MPZL1, SLC4A1AP, TRIP4, MSRA, HS3ST1, ZNF224, AP2A2, FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1B, NUCKS1, ACMSD, HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT. The population of cells may be administered to the patient, for example, systemically (e.g., intravenously), directly to the CNS (e.g., intracerebroventricularly or stereotactically), or directly into the bone marrow (e.g., intraosseously). The cells can also be administered to the patient by multiple routes of administration, for example, intravenously and intracerebroventricularly. The cells are administered in a therapeutically effective amount, such as from 1×10⁶ cells/kg to 1×10¹² cells/kg or more (e.g., 1×10⁷ cells/kg, 1×10⁸ cells/kg, 1×10⁹ cells/kg, 1×10¹⁰ cells/kg, 1×10¹¹ cells/kg, 1×10¹² cells/kg, or more).

Before the population of cells is administered to the patient, one or more agents may be administered to the patient to ablate the patient's endogenous microglia and/or hematopoietic stem and progenitor cells, such as, busulfan, treosulfan, PLX3397, PLX647, PLX5622, and/or clodronate liposomes. Other methods of cell ablation may also be used, such as irradiation, which may be performed alone or in combination with one or more of the aforementioned agents to ablate the patient's microglia and/or hematopoietic stem and progenitor cells. These agents and/or treatments may ablate endogenous microglia and/or hematopoietic stem and progenitor cells by at least 5% (e.g., at least 5%, 10%, 15%, 20%, 25%, 30%, 35%, 40%, 45%, 50%, 60%, 70%, 80%, 90%, 99%, or more), as assessed by PET imaging techniques known in the art. If the population of cells is administered to the patient after ablation, the cells may have an improved rate or repopulation of the brain, where they may differentiate, e.g., into microglia. The population of cells can be administered to the patient from, for example, 1 week to 1 month (e.g., 1 week, 2 weeks, 3 weeks, 4, weeks) or more after ablation.

Additionally or alternatively, the patient may be administered, for example, one or more other agents that collectively elevate the expression and/or activity level of one or more of the foregoing proteins. Such agents include viral vectors that collectively encode the one or more proteins. Exemplary viral vectors are Retroviridae family viral vectors, such as a lentivirus, alpharetrovirus, or gammaretrovirus, among others described herein. Additional agents that may be provided to a patient for the purpose of augmenting the level of one or more of the foregoing proteins include interfering RNA molecules, such as siRNA, shRNA, and miRNA molecules, as well as small molecule agents that modulate the expression of one or more of the above proteins, in addition to the one or more of the above proteins themselves.

### Example 9

### Generation of Mammalian Cell Lines Expressing TREM2

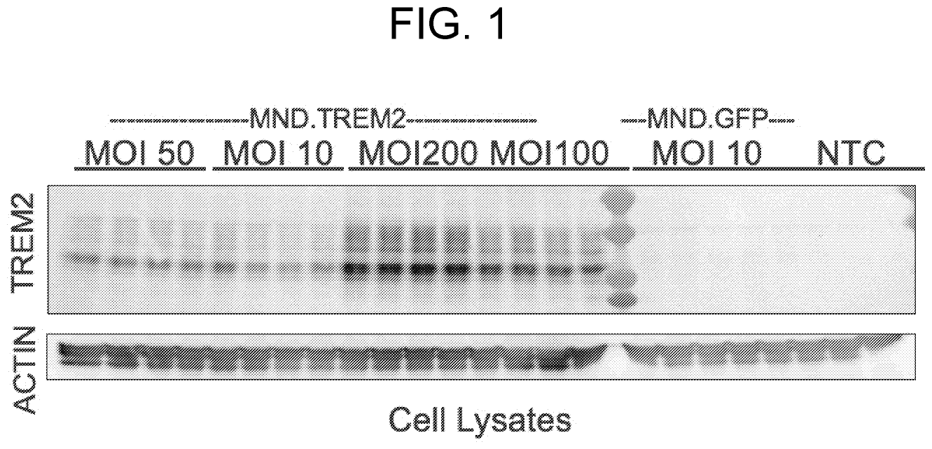

To assess the ability of lentivirally-encoded, codon-optimized TREM2 transgenes to stably express in mammalian cell lines, murine RAW macrophage cell lines, murine primary microglia, and murine lineage negative (Lin-) negative cells were transduced in vitro. In a first experiment, murine RAW macrophage cells were either transduced with a lentiviral vector carrying a transgene encoding the human TREM2 protein (MND.TREM2) or GFP (MND.GFP) at a multiplicity of infection (MOI) of 10, 50, 100, or 200. A separate set of control cells were not transduced (NTC). TREM2 expression was assessed using an antibody raised against human TREM2. Stable expression of human TREM2 was observed in murine macrophages (FIG. 1).

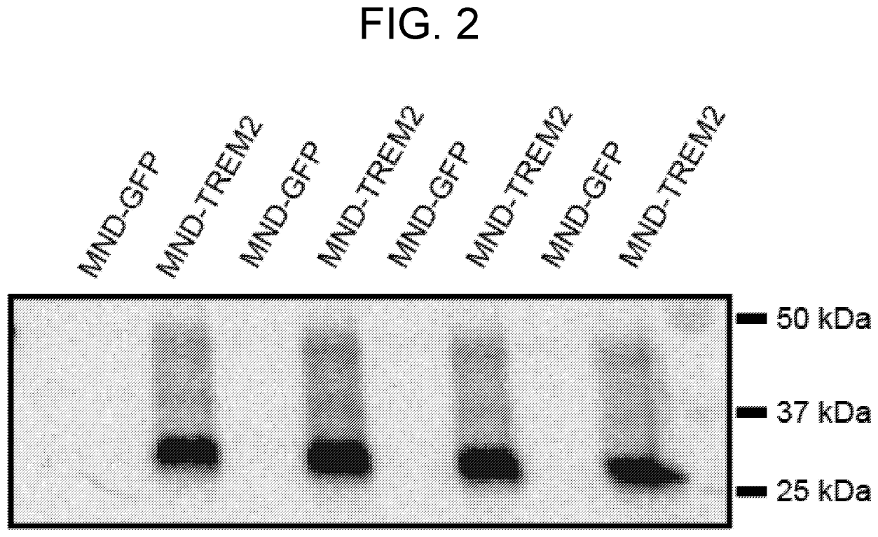

In a separate experiment, murine primary microglia were either transduced with a lentiviral vector carrying a transgene encoding the human TREM2 protein (MND-TREM2) or GFP (MND-GFP). A separate set of control cells were not transduced (NT). TREM2 expression was assessed using an antibody raised against human TREM2. Stable expression of human TREM2 was observed in murine primary microglia (FIG. 2).

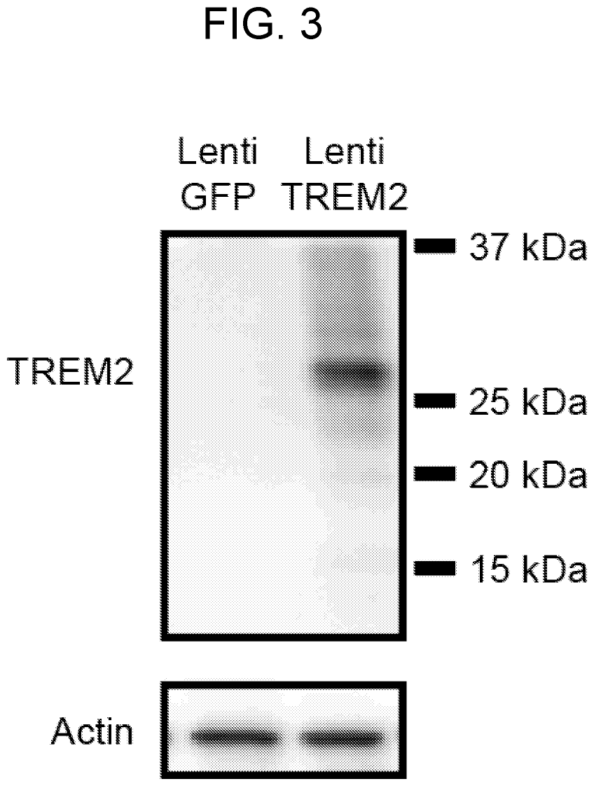

In another experiment, murine Lin− cells were either transduced with a lentiviral vector carrying a transgene encoding the human TREM2 protein (Lenti TREM2) or GFP (Lenti GFP). TREM2 expression was assessed using an antibody raised against human TREM2. Stable expression of human TREM2 was observed in murine Lin− cells. (FIG. 3).

Combined, the above results demonstrate that stable expression of codon-optimized human TREM2 protein can be achieved in vitro using lentiviral vectors, resulting in increased levels of TREM2 in immortalized murine macrophages, primary microglia, and Lin− cells in which human TREM2 is normally absent. These findings demonstrate a potential therapeutic approach for diseases caused by or associated with mutations in the TREM2 gene.

### Example 10

### Generation of Mammalian Cell Lines Expressing Progranulin

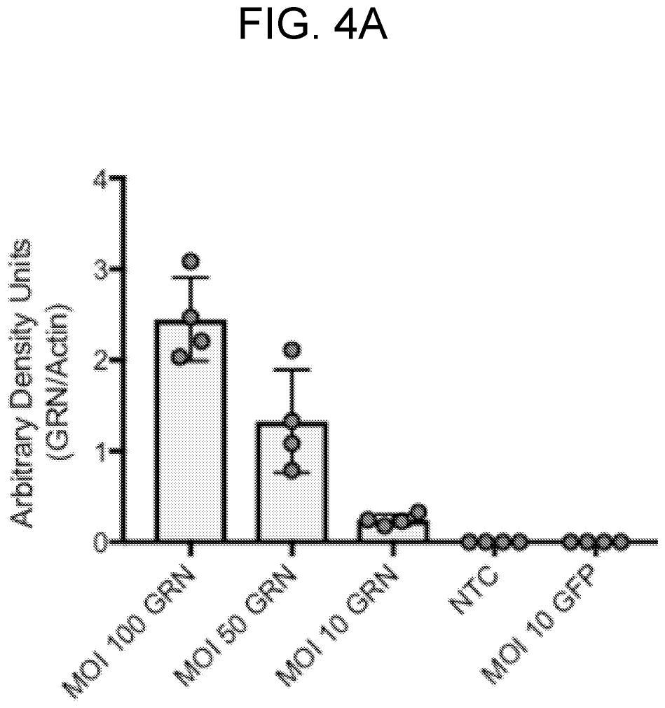

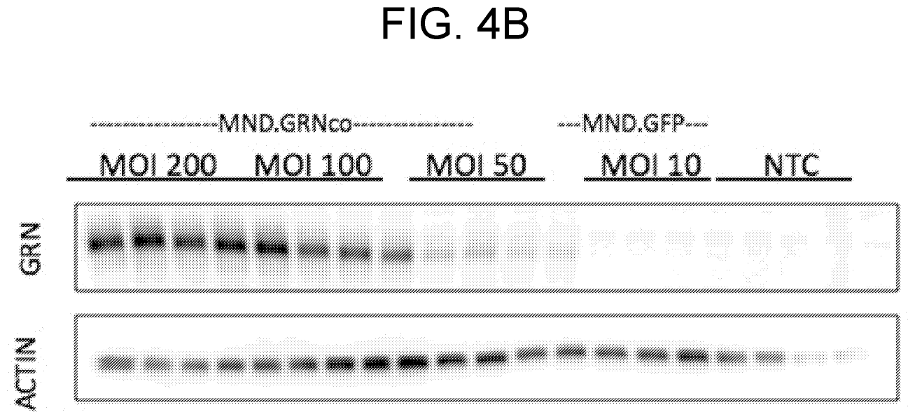

To assess the ability of lentivirally-encoded, codon-optimized PGRN transgenes to stably express in mammalian cell lines, human and murine cells were transduced in vitro. In a first experiment, human 239T cells were transduced with a lentiviral vector containing a transgene encoding a human PGRN protein (MND.GRN) or green fluorescent protein (GFP; MND.GFP) at a multiplicity of infection (MOI) of 10, 50, 100, or 200. A separate set of control cells were not transduced (NTC). Densitometry was used to quantify PGRN levels over actin (FIG. 4A). Western blots were performed using an antibody raised against human PGRN protein, demonstrating stable expression of human PGRN in human cells, with the highest expression observed at MOI 200 (FIG. 4B).

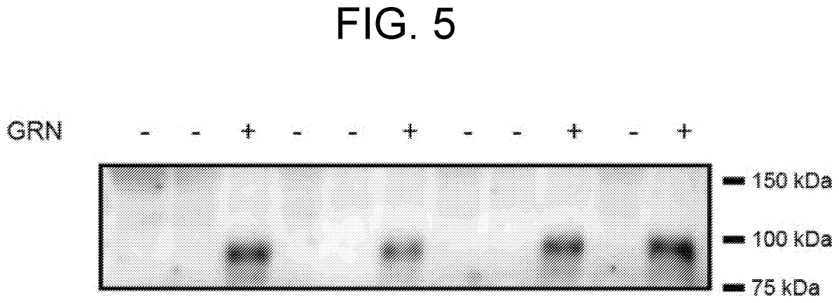

In a separate experiment, murine lineage negative (Lin−) cells were transduced with a lentiviral vector containing a transgene encoding human PGRN protein (i.e., a MND.GRN vector). Conditioned media generated from Lin− mouse cells non-transduced or transduced with MND.GRN lentiviral vector were analyzed using Western blot with an antibody raised against human PGRN protein, showing release of human PGRN protein into the growth media by the transduced cells (FIG. 5).

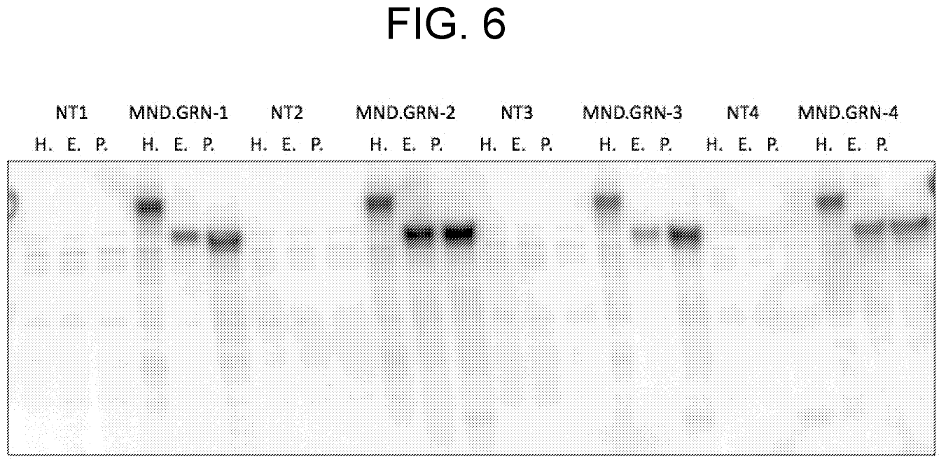

In another experiment, human 239T cells were transduced with a lentiviral vector containing a transgene encoding a human PGRN protein in four independent rounds of transduction. Cell lysates were generated from 239T non-transduced cells or cell lines transduced with a lentiviral vector encoding human PG RN. Cell lysates were then enzymatically digested with EndoH or PNGase enzymes, or heated, and analyzed using Western blot with an antibody raised against human PGRN protein (FIG. 6). Enzymatic digestion by EndoH and PNGase indicate that the human PGRN protein produced by the transduced cells is N-linked glycosylated.

Combined together, the above results show that lentivirally-mediated transduction of human and murine cells with transgenes encoding a human PGRN protein achieves stable PGRN expression in cells in which PGRN expression is otherwise absent. Transduction of murine primary Lin− cells with lentivirally-encoded PGRN results in the release of PGRN protein into the growth media. Furthermore, the PGRN protein produced by the lentiviral vector described above is N-linked glycosylated. These findings demonstrate that lentiviral transduction with the PGRN-encoding vector described above increases PGRN levels and enables the release of PGRN by hematopoietic cells, thereby suggesting a potential therapeutic approach for diseases caused by or linked to mutations in the PGRN gene.

### Other Embodiments

Various modifications and variations of the described disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure that are obvious to those skilled in the art are intended to be within the scope of the disclosure.

Other embodiments are in the claims.

1. A method of treating a patient diagnosed as having a neurocognitive disorder (NCD), the method comprising providing to the patient one or more agents that collectively increase expression and/or activity of two or more proteins selected from APP, PSEN1, PSEN2, APOE, TOMM40, GAB2, APOC1, TREM2, ABI3, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1L, STK24, DISCI , MPZL1, SLC4A1AP, TRIP4, MSRA, HS3ST1, ZNF224, and AP2A2.

2. The method of claim 1, wherein the proteins are selected from PSEN1, GAB2, APOC1, TREM2, ABI3, BIN1, HLA-DRB5, HLA-DRB1, CD2AP, PTK2B, INPP5D, MEF2C, CD33, MS4A4A, RIN3, PICALM, CASS4, SORL1, PLCG2, SCIMP, FRMD4A, SPPL2A, MTHFD1L, DISC1, TRIP4, and HS3ST1, optionally wherein the proteins comprise a panel set forth in Table 1.

3. A method of treating a patient diagnosed as having an NCD, the method comprising providing to the patient one or more agents that collectively increase expression and/or activity of two or more proteins selected from FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1B, NUCKS1, and ACMSD.

4. The method of claim 3, wherein the proteins are selected from FCGR2A, SCAF11, DNAJC13, GCH1, LRRK2, GBA, GAK, FGF20, HLA-DQB1, and NOD2, optionally wherein the proteins comprise a panel set forth in Table 2.

5. A method of treating a patient diagnosed as having an NCD, the method comprising providing to the patient one or more agents that collectively increase expression and/or activity of two or more proteins selected from HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT.

6. The method of claim 5, wherein the proteins are selected from HLA-DRA, HLA-DRB5, C9ORF72, SQSTM1, TBK1, PSEN1, GRN, and CTSF, optionally wherein the proteins comprise a panel set forth in Table 3.

7. A method of treating a patient diagnosed as having an NCD, the method comprising providing to the patient one or more agents that collectively increase expression and/or activity of two or more proteins selected from APP, PSEN1, PSEN2, APOE, TOMM40, GAB2, APOC1, TREM2, ABI3, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1L, STK24, DISCI , MPZL1, SLC4A1AP, TRIP4, MSRA, HS3ST1, ZNF224, AP2A2, FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1B, NUCKS1, ACMSD, HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT.

8. The method of any one of claims 1-7, wherein the NCD is a major NCD.

9. The method of claim 8, wherein the major NCD interferes with the patient's independence and/or normal daily functioning.

10. The method of claim 8 or 9, wherein the major NCD is associated with a score obtained by the patient on a cognitive test that is at least two standard deviations away from the mean score of a reference population.

11. The method of any one of claims 1-7, wherein the NCD is a mild NCD.

12. The method of claim 11, wherein the mild NCD does not interfere with the patient's independence and/or normal daily functioning.

13. The method of claim 11 or 12, wherein the mild NCD is associated with a score obtained by the patient on a cognitive test that is between one to two standard deviations away from the mean score of a reference population.

14. The method of claim 10 or 13, wherein the reference population is a general population.

15. The method of claim 10, 13, or 14, wherein the cognitive test is selected from the group consisting of ADB, AWV, GPCOG, HRA, MIS, MMSE, MoCA, SLUMS, and Short IQCODE.

16. The method of any one of claims 1-15, wherein the NCD is associated with impairment in one or more of complex attention, executive function, learning and memory, language, perceptual-motor function, and social cognition.

17. The method of any one of claims 1-16, wherein the NCD is not due to delirium or other mental disorder.

18. The method of any one of claim 1, 2 or 7, wherein the NCD is Alzheimer's disease.

19. The method of any one of claim 3, 4, or 7, wherein the NCD is a movement disorder.

20. The method of claim 18, wherein the movement disorder is Parkinson disease.

21. The method of any one of claims 5-7 wherein the NCD is a frontotemporal NCD.

22. The method of claim 21, wherein the frontotemporal NCD is frontotemporal lobar degeneration (FTLD).

23. The method of claim 22, wherein the FTLD is behavioral-variant frontotemporal dementia.

24. The method of claim 22, wherein the FTLD is semantic dementia.

25. The method of claim 22, wherein the FTLD is progressive nonfluent aphasia.

26. The method of any one of claims 1-25, wherein the one or more agents collectively increase expression and/or activity of three or more of the proteins, optionally wherein the one or more agents collectively increase expression and/or activity of four or more of the proteins, or optionally wherein the one or more agents collectively increase expression and/or activity of five or more of the proteins.

27. The method of any one of claim 1, 2, or 7-18, wherein the one or more agents collectively increase expression and/or activity of from five to 20 of the proteins, optionally wherein the one or more agents collectively increase expression and/or activity of from eight to 18 of the proteins, or optionally wherein the one or more agents collectively increase expression and/or activity of from 10 to 15 of the proteins.

28. The method of any one of claim 3, 4, 7-17, 19, or 20, wherein the one or more agents collectively increase expression and/or activity of from three to 10 of the proteins, optionally wherein the one or more agents collectively increase expression and/or activity of from four to eight of the proteins, or optionally wherein the one or more agents collectively increase expression and/or activity of from five to seven of the proteins.

29. The method of any one of claim 5-17, or 21-25, wherein the one or more agents collectively increase expression and/or activity of from two to seven of the proteins, optionally wherein the one or more agents collectively increase expression and/or activity of from three to six of the proteins, or optionally wherein the one or more agents collectively increase expression and/or activity of four or five of the proteins.

30. The method of any one of claims 1-29, wherein the one or more agents comprise (i) one or more nucleic acid molecules that collectively encode the two or more proteins, (ii) one or more interfering RNA molecules that collectively increase expression and/or activity of the two or more proteins, (iii) one or more nucleic acid molecules encoding the one or more interfering RNA molecules, (iv) two or more of the proteins, and/or (v) one or more small molecules that collectively increase expression and/or activity of the two or more proteins.

31. The method of claim 29, wherein the one or more interfering RNA molecules comprise short interfering RNA (siRNA), short hairpin RNA (shRNA), and/or micro RNA (miRNA).

32. The method of any one of claims 1-31, wherein the one or more agents comprise one or more nucleic acid molecules that collectively encode the two or more proteins, optionally wherein the one or more nucleic acid molecules collectively encode three or more of the protein, optionally wherein the one or more nucleic acid molecules collectively encode four or more of the proteins, or optionally wherein the one or more nucleic acid molecules collectively encode five or more of the proteins.

33. The method of any one of claim 1, 2, or 7-18, wherein the one or more agents comprise one or more nucleic acid molecules that collectively encode from five to 20 of the proteins, optionally wherein the one or more nucleic acid molecules collectively encode from eight to 18 of the proteins, or optionally wherein the one or more nucleic acid molecules collectively encode from 10 to 15 of the proteins.

34. The method of any one of claim 3, 4, 7-17, 19, or 20, wherein the one or more agents comprise one or more nucleic acid molecules that collectively encode from three to 10 of the proteins, optionally wherein the one or more nucleic acid molecules collectively encode from four to eight of the proteins, optionally wherein the one or more nucleic acid molecules collectively encode from five to seven of the proteins.

35. The method of any one of claim 5-17, or 21-25, wherein the one or more agents comprise one or more nucleic acid molecules that collectively encode from two to seven of the proteins, optionally wherein the one or more nucleic acid molecules collectively encode from three to six of the proteins, optionally wherein the one or more nucleic acid molecules collectively encode four or five of the proteins.

36. The method of any one of claims 32-35, wherein the one or more nucleic acid molecules are provided to the patient by administering to the patient a composition comprising a population of cells that together contain nucleic acids encoding the proteins.

37. The method of claim 36, wherein the population is a uniform population of cells that contain nucleic acids encoding the proteins or a heterogeneous population of cells that together contain nucleic acids encoding the proteins.

38. The method of claim 36 or 37, wherein the cells are pluripotent cells or multipotent cells.

39. The method of claim 38, wherein the multipotent cells are CD34+ cells.

40. The method of claim 39, wherein the CD34+ cells are HSCs or MPCs.

41. The method of claim 38, wherein the pluripotent cells are ESCs or iPSCs,

42. The method of claim 36 or 37, wherein the cells are BLPCs, microglial progenitor cells, monocytes, macrophages, or microglia.

43. The method of claim 42, wherein the BLPCs are monocytes.

44. The method of any one of claims 1-43, wherein the composition is administered to the subject by way of systemic administration, by way of direct administration to the central nervous system of the subject, by way of direct administration to the bone marrow of the subject, or by way of bone marrow transplant comprising the composition.

45. The method of any one of claims 36-44, wherein the cells are autologous cells or allogeneic cells.

46. The method of any one of claims 36-45, wherein the cells are transfected or transduced ex vivo to express the proteins.

47. The method of claim 46, wherein the cells are transduced with a viral vector selected from the group consisting of an adeno-associated virus (AAV), an adenovirus, a parvovirus, a coronavirus, a rhabdovirus, a paramyxovirus, a picornavirus, an alphavirus, a herpes virus, a poxvirus, and a Retroviridae family virus.

48. The method of claim 46, wherein the cells are transfected using: a) an agent selected from the group consisting of a cationic polymer, diethylaminoethyldextran, polyethylenimine, a cationic lipid, a liposome, calcium phosphate, an activated dendrimer, and a magnetic bead; or b) a technique selected from the group consisting of electroporation, Nucleofection, squeeze-poration, sonoporation, optical transfection, Magnetofection, and impalefection.

49. The method of any one of claims 30-35, wherein the one or more nucleic acid molecules are provided to the patient by administering to the patient one or more viral vectors that together comprise the one or more nucleic acid molecules.

50. The method of claim 49, wherein the patient is administered a plurality of viral vectors that together comprise the one or more nucleic acid molecules.

51. The method of claim 49, wherein the patient is administered a plurality of viral vectors that each individually comprise the one or more nucleic acid molecules.

52. The method of any one of claims 49-51, wherein the one or more viral vectors are administered systemically to the patient or directly to the central nervous system of the patient,

53. The method of any one of claims 47-52, wherein the viral vector is a Retroviridae family viral vector.

54. The method of claim 53, wherein the Retroviridae family viral vector is a lentiviral vector, alpharetroviral vector, or gamma retroviral vector.

55. The method of any one of claim 53 or 54, wherein the Retroviridae family viral vector comprises a central polypurine tract, a woodchuck hepatitis virus post-transcriptional regulatory element, a 5′-LTR, HIV signal sequence, HIV Psi signal 5′-splice site, delta-GAG element, 3′-splice site, and a 3′-self inactivating LTR.

56. The method of any one of claims 47-52, wherein the viral vector is an AAV selected from the group consisting of AAV1, AAV2, AAV3, AAV4, AAV5, AAV6, AAV7, AAV8, AAV9, AAV10, and AAVrh74.

57. The method of any one of claims 47-56, wherein the viral vector is a pseudotyped viral vector.

58. The method of claim 57, wherein the pseudotyped viral vector selected from the group consisting of a pseudotyped AAV, a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus.

59. The method of any one of claims 30-58, wherein one or more of the nucleic acid molecules comprises a transgene encoding one or more of the proteins operably linked to a ubiquitous promoter, a cell lineage-specific promoter, or a synthetic promoter.

60. The method of claim 59, wherein the ubiquitous promoter is selected from the group consisting of an elongation factor 1-alpha promoter and a phosphoglycerate kinase 1 promoter.

61. The method of claim 59, wherein the cell lineage-specific promoter is selected from the group consisting of a PGRN promoter, CD11 b promoter, CD68 promoter, a C—X3-C motif chemokine receptor 1 promoter, an allograft inflammatory factor 1 promoter, a purinergic receptor P2Y12 promoter, a transmembrane protein 119 promoter, and a colony stimulating factor 1 receptor promoter.

62. The method of any one of claims 32-61, wherein one or more of the proteins further comprises a receptor-binding (Rb) domain of apolipoprotein E (ApoE).

63. The method of claim 62, wherein the Rb domain comprises a portion of ApoE having the amino acid sequence of residues 25-185, 50-180, 75-175, 100-170, 125-160, or 130-150 of SEQ ID NO: 105.

64. The method of claim 62 or 63, wherein the Rb domain comprises a region having at least 70% sequence identity to the amino acid sequence of residues 159-167 of SEQ ID NO: 105.

65. The method of any one of claims 30-64, wherein the one or more nucleic acid molecules comprise a micro RNA (miRNA)-126 (miR-126) targeting sequence in the 3′-UTR.

66. The method of any one of claims 30-65, wherein upon providing the one or more nucleic acid molecules to the patient, the proteins penetrate the blood-brain barrier in the patient.

67. The method of any one of claims 30-66, wherein a population of endogenous microglia in the patient has been ablated prior to providing the patient with the one or more nucleic acid molecules.

68. The method of any one of claims 30-67, the method comprising ablating a population of endogenous microglia in the patient prior to providing the patient with the one or more nucleic acid molecules.

69. The method of claim 67 or 68, wherein the microglia are ablated using an agent selected from the group consisting of busulfan, PLX3397, PLX647, PLX5622, treosulfan, and clodronate liposomes, by radiation therapy, or a combination thereof.

70. The method of any one of claims 30-69, wherein, prior to providing the patient with the one or more nucleic acid molecules, endogenous expression of one or more of the proteins is disrupted in the cells, in the patient, or in a population of neurons in the patient.

71. The method of claim 70, wherein the endogenous expression is disrupted by contacting the cells with a nuclease that catalyzes cleavage of an endogenous gene encoding one of the proteins.

72. The method of claim 71, wherein the nuclease is a CRISPR associated protein 9 (Cas9), CRISPR-associated protein 12a (Cas12a), a transcription activator-like effector nuclease, a meganuclease, or a zinc finger nuclease.

73. The method of any one of claims 70-72, wherein endogenous expression of one or more of the proteins is disrupted by administering an inhibitory RNA molecule to the cells, the patient, or the population of neurons.

74. The method of claim 73, wherein the inhibitory RNA molecule is a siRNA, a shRNA, or a miRNA.

75. A pharmaceutical composition comprising a population of cells that together contain nucleic acids encoding two or more proteins selected from APP, PSEN1, PSEN2, APOE, TOMM40, GAB2, APOC1, TREM2, ABI3, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1L, STK24, DISCI , MPZL1, SLC4A1AP, TRIP4, MSRA, HS3ST1, ZNF224, and AP2A2.

76. The pharmaceutical composition of claim 75, wherein the proteins are selected from PSEN1, GAB2, APOC1, TREM2, ABI3, BIN1, HLA-DRB5, HLA-DRB1, CD2AP, PTK2B, INPP5D, MEF2C, CD33, MS4A4A, RIN3, PICALM, CASS4, SORL1, PLCG2, SCIMP, FRMD4A, SPPL2A, MTHFD1 L, DISCI , TRIP4, and HS3ST1.

77. A pharmaceutical composition comprising a population of cells that together contain nucleic acids encoding two or more proteins selected from FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1B, NUCKS1, and ACMSD.

78. The pharmaceutical composition of claim 77, wherein the proteins are selected from FCGR2A, SCAF11, DNAJC13, GCH1, LRRK2, GBA, GAK, FGF20, HLA-DQB1, and NOD2.

79. A pharmaceutical composition comprising a population of cells that together contain nucleic acids encoding two or more proteins selected from HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT.

80. The pharmaceutical composition of claim 79, wherein the proteins are selected from HLA-DRA, HLA-DRB5, C9ORF72, SQSTM1, TBK1, PSEN1, GRN, and CTSF.

81. A pharmaceutical composition comprising a population of cells that together contain nucleic acids encoding two or more proteins selected from APP, PSEN1, PSEN2, APOE, TOMM40, GAB2, APOC1, TREM2, ABI3, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1L, STK24, DISCI , MPZL1, SLC4A1AP, TRIP4, MSRA, HS3ST1, ZNF224, AP2A2, FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1 L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1B, NUCKS1, ACMSD, HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT.

82. The pharmaceutical composition of any one of claims 75-81, wherein the cells together contain nucleic acids encoding three or more of the proteins, optionally wherein the cells together contain nucleic acids encoding four or more of the proteins, or optionally wherein the cells together contain nucleic acids encoding five or more of the proteins.

83. The pharmaceutical composition of claim 75 or 76, wherein the cells together contain nucleic acids encoding from five to 20 of the proteins, optionally wherein the cells together contain nucleic acids encoding from eight to 18 of the proteins, or optionally wherein the cells together contain nucleic acids encoding from 10 to 15 of the proteins.

84. The pharmaceutical composition of claim 77 or 78, wherein the cells together contain nucleic acids encoding from three to 10 of the proteins, optionally wherein the cells together contain nucleic acids encoding from four to eight of the proteins, or optionally wherein the cells together contain nucleic acids encoding from five to seven of the proteins.

85. The pharmaceutical composition of claim 79 or 80, wherein the cells together contain nucleic acids encoding from two to seven of the proteins, optionally wherein the cells together contain nucleic acids encoding from three to six of the proteins, optionally wherein the cells together contain nucleic acids encoding four or five of the proteins.

86. The pharmaceutical composition of any one of claims 75-85, wherein the population is a uniform population of cells or a heterogenous population of cells that contain nucleic acids encoding the proteins.

87. The composition of any one of claims 75-86, wherein the cells are pluripotent cells or multipotent cells.

88. The composition of claim 87, wherein the multipotent cells are CD34+ cells.

89. The composition of claim 88, wherein the CD34+ cells are HSCs or MPCs.

90. The composition of claim 87, wherein the pluripotent cells are ESCs or iPSCs.

91. The composition of any one of claims 75-86, wherein the cells are BLPCs, microglial progenitor cells, macrophages, or microglia.

92. The composition of claim 91, wherein the BLPCs are monocytes.

93. The pharmaceutical composition of any one of claims 75-92, wherein the cells are autologous cells or allogeneic cells.

94. The pharmaceutical composition of any one of claims 75-93, wherein the cells comprise a transgene encoding one or more of the proteins operably linked to a ubiquitous promoter, a cell-lineage specific promoter, or a synthetic promoter

95. The pharmaceutical composition of claim 94, wherein the ubiquitous promoter is selected from the group consisting of an elongation factor 1-alpha promoter and a phosphoglycerate kinase 1 promoter.

96. The pharmaceutical composition of claim 94, wherein the cell lineage-specific promoter is selected from the group consisting of a PGRN promoter, CD11 b promoter, CD68 promoter, a C—X3-C motif chemokine receptor 1 promoter, an allograft inflammatory factor 1 promoter, a purinergic receptor P2Y12 promoter, a transmembrane protein 119 promoter, and a colony stimulating factor 1 receptor promoter.

97. The pharmaceutical composition of any one of claims 75-96, wherein one or more of the proteins further comprises an Rb domain of ApoE.

98. The pharmaceutical composition of claim 97, wherein the Rb domain comprises a portion of ApoE having the amino acid sequence of residues 25-185, 50-180, 75-175, 100-170, 125-160, or 130-150 of SEQ ID NO: 105.

99. The pharmaceutical composition of claim 97 or 98, wherein the Rb domain comprises a region having at least 70% sequence identity to the amino acid sequence of residues 159-167 of SEQ ID NO: 105.

100. A pharmaceutical composition comprising a population of viral vectors that together encode two or more proteins selected from APP, PSEN1, PSEN2, APOE, TOMM40, GAB2, APOC1, TREM2, ABI3, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1 L, STK24, DISCI , MPZL1, SLC4A1AP, TRIP4, MSRA, HS3ST1, ZNF224, and AP2A2.

101. The pharmaceutical composition of claim 100, wherein the proteins are selected from PSEN1, GAB2, APOC1, TREM2, ABI3, BIN1, HLA-DRB5, HLA-DRB1, CD2AP, PTK2B, INPP5D, MEF2C, CD33, MS4A4A, RIN3, PICALM, CASS4, SORL1, PLCG2, SCIMP, FRMD4A, SPPL2A, MTHFD1 L, DISCI , TRIP4, and HS3ST1.

102. A pharmaceutical composition comprising a population of viral vectors that together encode two or more proteins selected from FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1 B, NUCKS1, and ACMSD.

103. The pharmaceutical composition of claim 102, wherein the proteins are selected from FCGR2A, SCAF11, DNAJC13, GCH1, LRRK2, GBA, GAK, FGF20, HLA-DQB1, and NOD2.

104. A pharmaceutical composition comprising a population of viral vectors that together encode two or more proteins selected from HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT.

105. The pharmaceutical composition of claim 104, wherein the proteins are selected from HLA-DRA, HLA-DRB5, C9ORF72, SQSTM1, TBK1, PSEN1, GRN, and CTSF.

106. A pharmaceutical composition comprising a population of viral vectors that together encode wo or more proteins selected from APP, PSEN1, PSEN2, APOE, TOMM40, GAB2, APOC1, TREM2, ABI3, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1L, STK24, DISCI , MPZL1, SLC4A1AP, TRIP4, MSRA, HS3ST1, ZNF224, AP2A2, FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1 B, NUCKS1, ACMSD, HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT.

107. The pharmaceutical composition of any one of claims 100-106, wherein the viral vectors together encode three or more of the proteins, optionally wherein the viral vectors together encode four or more of the proteins, optionally wherein the viral vectors together encode five or more of the proteins.

108. The pharmaceutical composition of claim 100, 101 or 106, wherein the viral vectors together encode from five to 20 of the proteins, optionally wherein the viral vectors together encode from eight to 18 of the proteins, optionally wherein the viral vectors together encode from 10 to 15 of the proteins.

109. The pharmaceutical composition of claim 102, 103, or 106, wherein the viral vectors together encode from three to 10 of the proteins, optionally wherein the viral vectors together encode from four to eight of the proteins, optionally wherein the viral vectors together encode from five to seven of the proteins.

110. The pharmaceutical composition of claim 104, 105, or 106, wherein the viral vectors together encode from two to seven of the proteins, optionally wherein the viral vectors together encode from three to six of the proteins, optionally wherein the viral vectors together encode four or five of the proteins.

111. The pharmaceutical composition of any one of claims 100-110, wherein the viral vectors comprise an AAV, an adenovirus, a parvovirus, a coronavirus, a rhabdovirus, a paramyxovirus, a picornavirus, an alphavirus, a herpes virus, a poxvirus, and/or a Retroviridae family virus.

112. The pharmaceutical composition of claim 111, wherein the viral vectors comprise a Retroviridae family viral vector.

113. The composition of claim 112, wherein the Retroviridae family viral vector is a lentiviral vector, alpharetroviral vector, or gamma retroviral vector.

114. The pharmaceutical composition of any one of claims 111-113, wherein the Retroviridae family viral vector comprises a central polypurine tract, a woodchuck hepatitis virus post-transcriptional regulatory element, a 5′-LTR, HIV signal sequence, HIV Psi signal 5′-splice site, delta-GAG element, 3′-splice site, and a 3′-self inactivating LTR.

115. The pharmaceutical composition of claim 111, wherein the viral vector is an AAV selected from the group consisting of AAV1, AAV2, AAV3, AAV4, AAVS, AAV6, AAV7, AAV8, AAV9, AAV10, and AAVrh74.

116. The pharmaceutical composition of any one of claims 100-115, wherein the viral vectors comprise a pseudotyped viral vector.

117. The pharmaceutical composition of claim 116, wherein the pseudotyped viral vector selected from the group consisting of a pseudotyped AAV, a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus.

118. The pharmaceutical composition of any one of claims 100-117, wherein one or more of the viral vectors comprises a transgene encoding one or more of the proteins operably linked to a ubiquitous promoter, a cell-lineage specific promoter, or a synthetic promoter.

119. The pharmaceutical composition of claim 118, wherein the ubiquitous promoter is selected from the group consisting of an elongation factor 1-alpha promoter and a phosphoglycerate kinase 1 promoter.

120. The pharmaceutical composition of claim 118, wherein the cell lineage-specific promoter is selected from the group consisting of a PGRN promoter, CD11b promoter, CD68 promoter, a C—X3-C motif chemokine receptor 1 promoter, an allograft inflammatory factor 1 promoter, a purinergic receptor P2Y12 promoter, a transmembrane protein 119 promoter, and a colony stimulating factor 1 receptor promoter.

121. The pharmaceutical composition of any one of claims 100-120, wherein one or more of the proteins further comprises an Rb domain of ApoE.

122. The pharmaceutical composition of claim 121, wherein the Rb domain comprises a portion of ApoE having the amino acid sequence of residues 25-185, 50-180, 75-175, 100-170, 125-160, or 130-150 of SEQ ID NO: 105.

123. The pharmaceutical composition of claim 121 or 122, wherein the Rb domain comprises a region having at least 70% sequence identity to the amino acid sequence of residues 159-167 of SEQ ID NO: 105.

124. The pharmaceutical composition of any one of claims 100-123, wherein one or more of the viral vectors comprises a transgene encoding one or more of the proteins, and wherein the transgene further encodes a miR-126 targeting sequence in the 3′-UTR.

125. A kit comprising the pharmaceutical composition of any one of claim 100, 101, 108, or 111-124, wherein the kit further comprises a package insert instructing a user of the kit to administer the pharmaceutical composition to a human patient having an NCD.

126. A kit comprising the pharmaceutical composition of any one of claim 102, 103, 109, or 111-124, wherein the kit further comprises a package insert instructing a user of the kit to administer the pharmaceutical composition to a human patient having an NCD.

127. A kit comprising the pharmaceutical composition of any one of claim 104. 105, 111, or 111-124, wherein the kit further comprises a package insert instructing a user of the kit to administer the pharmaceutical composition to a human patient having an NCD.

128. A kit comprising the pharmaceutical composition of any one of claims APP, PSEN1, PSEN2, APOE, TOMM40, GAB2, APOC1, TREM2, ABI3, BIN1, CR1, ABCA7, FERMT2, HLA-DRB5, HLA-DRB1, CD2AP, PTK2B, CELF1, INPP5D, MEF2C, ZCWPW1, CD33, MS4A4A, RIN3, EPHA1, PICALM, CASS4, CLU, SORL1, PLCG2, SCIMP, FRMD4A, SPPL2A, MTHFD1L, STK24, DISC1, MPZL1, SLC4A1AP, TRIP4, MSRA, HS3ST1, ZNF224, AP2A2, FCGR2A, SCAF11, HLA-DQB1, NOD2, VPS1, SCARB2, GPNMB, VPS35, FBXO7, PARK7, INPP5F, DNAJC13, GCH1, NMD3, USP25, RAB7L1, SIPA1L2, MCCC1, SYNJ1, LRRK2, SNCA, PTRHD1, PINK1, GBA, TMEM163, GAK, FGF20, DLG2, DDRGK1, SREBF, BCKDK, PARK2, RAB39B, DNAJC6, SMPD1, TMEM175, STK39, BST1, MMP16, RIT2, FAM47E, CCDC62, TMEM229B, MAPT, SPPL2B, ITGA8, ATP13A2, DGKQ, STX1B, NUCKS1, ACMSD, HLA-DRA, HLA-DRB5, C9ORF72, SQSTM1, TARDBP, TBK1, VCP, PSEN1, FUS, CHMP2B, UBQLN2, CHCHD10, GRN, RAB38, CTSF, PSEN2, CYP27A1, BTNL2, and MAPT, wherein the kit further comprises a package insert instructing a user of the kit to administer the pharmaceutical composition to a human patient having an NCD.

129. The kit of any one of claims 125-128, wherein the NCD is a major NCD.

130. The kit of claim 129, wherein the major NCD interferes with the patient's independence and/or normal daily functioning.

131. The kit of claim 129 or 130, wherein the major NCD is associated with a score obtained by the patient on a cognitive test that is at least two standard deviations away from the mean score of a reference population.

132. The kit of any one of claims 125-128, wherein the NCD is a mild NCD.

133. The kit of claim 132, wherein the mild NCD does not interfere with the patient's independence and/or normal daily functioning.

134. The kit of claim 132 or 133, wherein the mild NCD is associated with a score obtained by the patient on a cognitive test that is between one to two standard deviations away from the mean score of a reference population.

135. The kit of claim 131 or 134, wherein the reference population is a general population.

136. The kit of claim 131, 134, or 135, wherein the cognitive test is selected from the group consisting of ADB, AWV, GPCOG, HRA, MIS, MMSE, MoCA, SLUMS, and Short IQCODE.

137. The kit of claim 125 or 128, wherein the NCD is Alzheimer's disease.

138. The kit of claim 126 or 128, wherein the NCD is a movement disorder.

139. The kit of claim 138, wherein the movement disorder is Parkinson disease.

140. The kit of claim 127 or 128, wherein the NCD is a frontotemporal NCD.

141. The kit of claim 140, wherein the frontotemporal NCD is FTLD.

142. The kit of claim 141, wherein the FTLD is behavioral-variant frontotemporal dementia.

143. The kit of claim 141, wherein the FTLD is semantic dementia.

144. The kit of claim 141, wherein the FTLD is progressive nonfluent aphasia.


SEQUENCE LISTING (106 sequences)

SEQ ID NO: 1 - protein, 770 aa, Artificial Sequence
  Synthetic Construct
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 2 - protein, 467 aa, Artificial Sequence
  Synthetic Construct
MTELPAPLSYFQNAQMSEDNHLSNTVRSQNDNRERQEHNDRRSLGHPEPLSNGRPQGNSRQVVEQDEEEDEELTLKYGAKHVIMLFVPVTLCMVVVVATIKSVSFYTRKDGQLIYTPFTEDTETVGQRALHSILNAAIMISVIVVMTILLVVLYKYRCYKVIHAWLIISSLLLLFFFSFIYLGEVFKTYNVAVDYITVALLIWNFGVVGMISIHWKGPLRLQQAYLIMISALMALVFIKYLPEWTAWLILAVISVYDLVAVLCPKGPLRMLVETAQERNETLFPALIYSSTMVWLVNMAEGDPEAQRRVSKNSKYNAESTERESQDTVAENDDGGFSEEWEAQRDSHLGPHRSTPESRAAVQELSSSILAGEDPEERGVKLGLGDFIFYSVLVGKASATASGDWNTTIACFVAILIGLCLTLLLLAIFKKALPALPISITFGLVFYFATDYLVQPFMDQLAFHQFYI

SEQ ID NO: 3 - protein, 448 aa, Artificial Sequence
  Synthetic Construct
MLTFMASDSEEEVCDERTSLMSAESPTPRSCQEGRQGPEDGENTAQWRSQENEEDGEEDPDRYVCSGVPGRPPGLEEELTLKYGAKHVIMLFVPVTLCMIVVVATIKSVRFYTEKNGQLIYTPFTEDTPSVGQRLLNSVLNTLIMISVIVVMTIFLVVLYKYRCYKFIHGWLIMSSLMLLFLFTYIYLGEVLKTYNVAMDYPTLLLTVWNFGAVGMVCIHWKGPLVLQQAYLIMISALMALVFIKYLPEWSAWVILGAISVYDLVAVLCPKGPLRMLVETAQERNEPIFPALIYSSAMVWTVGMAKLDPSSQGALQLPYDPEMEEDSYDSFGEPSYPEVFEPPLTGYPGEELEEEEERGVKLGLGDFIFYSVLVGKAAATGSGDWNTTLACFVAILIGLCLTLLLLAVFKKALPALPISITFGLIFYFSTDNLVRPFMDTLASHQLYI

SEQ ID NO: 4 - protein, 361 aa, Artificial Sequence
  Synthetic Construct
MGNVLAASSPPAGPPPPPAPALVGLPPPPPSPPGFTLPPLGGSLGAGTSTSRSSERTPGAATASASGAAEDGACGCLPNPGTFEECHRKCKELFPIQMEGVKLTVNKGLSNHFQVNHTVALSTIGESNYHFGVTYVGTKQLSPTEAFPVLVGDMDNSGSLNAQVIHQLGPGLRSKMAIQTQQSKFVNWQVDGEYRGSDFTAAVTLGNPDVLVGSGILVAHYLQSITPCLALGGELVYHRRPGEEGTVMSLAGKYTLNNWLATVTLGQAGMHATYYHKASDQLQVGVEFEASTRMQDTSVSFGYQLDLPKANLLFKGSVDSNWIVGATLEKKLPPLPLTLALGAFLNHRKNKFQCGFGLTIG

SEQ ID NO: 5 - protein, 676 aa, Artificial Sequence
  Synthetic Construct
MSGGGDVVCTGWLRKSPPEKKLRRYAWKKRWFILRSGRMSGDPDVLEYYKNDHSKKPLRIINLNFCEQVDAGLTFNKKELQDSFVFDIKTSERTFYLVAETEEDMNKWVQSICQICGFNQAEESTDSLRNVSSAGHGPRSSPAELSSSSQHLLRERKSSAPSHSSQPTLFTFEPPVSNHMQPTLSTSAPQEYLYLHQCISRRAENARSASFSQGTRASFLMRSDTAVQKLAQGNGHCVNGISGQVHGFYSLPKPSRHNTEFRDSTYDLPRSLASHGHTKGSLTGSETDNEDVYTFKTPSNTLCREFGDLLVDNMDVPATPLSAYQIPRTFTLDKNHNAMTVATPGDSAIAPPPRPPKPSQAETPRWGSPQQRPPISENSRSVAATIPRRNTLPAMDNSRLHRASSCETYEYPQRGGESAGRSAESMSDGVGSFLPGKMIVGRSDSTNSEDNYVPMNPGSSTLLAMERAGDNSQSVYIPMSPGAHHFDSLGYPSTTLPVHRGPSRGSEIQPPPVNRNLKPDRKAKPTPLDLRNNTVIDELPFKSPITKSWSRANHTFNSSSSQYCRPISTQSITSTDSGDSEENYVPMQNPVSASPVPSGTNSPAPKKSTGSVDYLALDFQPSSPSPHRKPSTSSVTSDEKVDYVQVDKEKTQALQNTMQEWTDVRQSSEPSKGAKL

SEQ ID NO: 6 - protein, 83 aa, Artificial Sequence
  Synthetic Construct
MRLFLSLPVLVVVLSIVLEGPAPAQGTPDVSSALDKLKEFGNTLEDKARELISRIKQSELSAKMREWFSETFQKVKEKLKIDS

SEQ ID NO: 7 - protein, 366 aa, Artificial Sequence
  Synthetic Construct
MAELQQLQEFEIPTGREALRGNHSALLRVADYCEDNYVQATDKRKALEETMAFTTQALASVAYQVGNLAGHTLRMLDLQGAALRQVEARVSTLGQMVNMHMEKVARREIGTLATVQRLPPGQKVIAPENLPPLTPYCRRPLNFGCLDDIGHGIKDLSTQLSRTGTLSRKSIKAPATPASATLGRPPRIPEPVHLPVVPDGRLSAASSAFSLASAGSAEGVGGAPTPKGQAAPPAPPLPSSLDPPPPPAAVEVFQRPPTLEELSPPPPDEELPLPLDLPPPPPLDGDELGLPPPPPGFGPDEPSWVPASYLEKVVTLYPYTSQKDNELSFSEGTVICVTRRYSDGWCEGVSSEGTGFFPGNYVEPSC

SEQ ID NO: 8 - protein, 593 aa, Artificial Sequence
  Synthetic Construct
MAEMGSKGVTAGKIASNVQKKLTRAQEKVLQKLGKADETKDEQFEQCVQNFNKQLTEGTRLQKDLRTYLASVKAMHEASKKLNECLQEVYEPDWPGRDEANKIAENNDLLWMDYHQKLVDQALLTMDTYLGQFPDIKSRIAKRGRKLVDYDSARHHYESLQTAKKKDEAKIAKPVSLLEKAAPQWCQGKLQAHLVAQTNLLRNQAEEELIKAQKVFEEMNVDLQEELPSLWNSRVGFYVNTFQSIAGLEENFHKEMSKLNQNLNDVLVGLEKQHGSNTFTVKAQPSDNAPAKGNKSPSPPDGSPAATPEIRVNHEPEPAGGATPGATLPKSPSQLRKGPPVPPPPKHTPSKEVKQEQILSLFEDTFVPEISVTTPSQFEAPGPFSEQASLLDLDFDPLPPVTSPVKAPTPSGQSIPWDLWEPTESPAGSLPSGEPSAAEGTFAVSWPSQTAEPGPAQPAEASEVAGGTQPAAGAQEPGETAASEAASSSLPAVVVETFPATVNGTVEGGSGAGRLDLPPGFMFKVQAQHDYTATDTDELQLKAGDVVLVIPFQNPEEQDEGWLMGVKESDWNQHKELEKCRGVFPENFTERVP

SEQ ID NO: 9 - protein, 2039 aa, Artificial Sequence
  Synthetic Construct
MGASSPRSPEPVGPPAPGLPFCCGGSLLAVVVLLALPVAWGQCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPEILHGEHTPSHQDNFSPGQEVFYSCEPGYDLRGAASLHCTPQGDWSPEAPRCAVKSCDDFLGQLPHGRVLFPLNLQLGAKVSFVCDEGFRLKGSSVSHCVLVGMRSLWNNSVPVCEHIFCPNPPAILNGRHTGTPSGDIPYGKEISYTCDPHPDRGMTFNLIGESTIRCTSDPHGNGVWSSPAPRCELSVRAGHCKTPEQFPFASPTIPINDFEFPVGTSLNYECRPGYFGKMFSISCLENLVWSSVEDNCRRKSCGPPPEPFNGMVHINTDTQFGSTVNYSCNEGFRLIGSPSTTCLVSGNNVTWDKKAPICEIISCEPPPTISNGDFYSNNRTSFHNGTVVTYQCHTGPDGEQLFELVGERSIYCTSKDDQVGVWSSPPPRCISTNKCTAPEVENAIRVPGNRSFFSLTEIIRFRCQPGFVMVGSHTVQCQTNGRWGPKLPHCSRVCQPPPEILHGEHTLSHQDNFSPGQEVFYSCEPSYDLRGAASLHCTPQGDWSPEAPRCTVKSCDDFLGQLPHGRVLLPLNLQLGAKVSFVCDEGFRLKGRSASHCVLAGMKALWNSSVPVCEQIFCPNPPAILNGRHTGTPFGDIPYGKEISYACDTHPDRGMTFNLIGESSIRCTSDPQGNGVWSSPAPRCELSVPAACPHPPKIQNGHYIGGHVSLYLPGMTISYICDPGYLLVGKGFIFCTDQGIWSQLDHYCKEVNCSFPLFMNGISKELEMKKVYHYGDYVTLKCEDGYTLEGSPWSQCQADDRWDPPLAKCTSRTHDALIVGTLSGTIFFILLIIFLSWIILKHRKGNNAHENPKEVAIHLHSQGGSSVHPRTLQTNEENSRVLP

SEQ ID NO: 10 - protein, 2146 aa, Artificial Sequence
  Synthetic Construct
MAFWTQLMLLLWKNFMYRRRQPVQLLVELLWPLFLFFILVAVRHSHPPLEHHECHFPNKPLPSAGTVPWLQGLICNVNNTCFPQLTPGEEPGRLSNFNDSLVSRLLADARTVLGGASAHRTLAGLGKLIATLRAARSTAQPQPTKQSPLEPPMLDVAELLTSLLRTESLGLALGQAQEPLHSLLEAAEDLAQELLALRSLVELRALLQRPRGTSGPLELLSEALCSVRGPSSTVGPSLNWYEASDLMELVGQEPESALPDSSLSPACSELIGALDSHPLSRLLWRRLKPLILGKLLFAPDTPFTRKLMAQVNRTFEELTLLRDVREVWEMLGPRIFTFMNDSSNVAMLQRLLQMQDEGRRQPRPGGRDHMEALRSFLDPGSGGYSWQDAHADVGHLVGTLGRVTECLSLDKLEAAPSEAALVSRALQLLAEHRFWAGVVFLGPEDSSDPTEHPTPDLGPGHVRIKIRMDIDVVTRTNKIRDRFWDPGPAADPLTDLRYVWGGFVYLQDLVERAAVRVLSGANPRAGLYLQQMPYPCYVDDVFLRVLSRSLPLFLTLAWIYSVTLTVKAVVREKETRLRDTMRAMGLSRAVLWLGWFLSCLGPFLLSAALLVLVLKLGDILPYSHPGVVFLFLAAFAVATVTQSFLLSAFFSRANLAAACGGLAYFSLYLPYVLCVAWRDRLPAGGRVAASLLSPVAFGFGCESLALLEEQGEGAQWHNVGTRPTADVFSLAQVSGLLLLDAALYGLATWYLEAVCPGQYGIPEPWNFPFRRSYWCGPRPPKSPAPCPTPLDPKVLVEEAPPGLSPGVSVRSLEKRFPGSPQPALRGLSLDFYQGHITAFLGHNGAGKTTTLSILSGLFPPSGGSAFILGHDVRSSMAAIRPHLGVCPQYNVLFDMLTVDEHVWFYGRLKGLSAAVVGPEQDRLLQDVGLVSKQSVQTRHLSGGMQRKLSVAIAFVGGSQVVILDEPTAGVDPASRRGIWELLLKYREGRTLILSTHHLDEAELLGDRVAVVAGGRLCCCGSPLFLRRHLGSGYYLTLVKARLPLTTNEKADTDMEGSVDTRQEKKNGSQGSRVGTPQLLALVQHWVPGARLVEELPHELVLVLPYTGAHDGSFATLFRELDTRLAELRLTGYGISDTSLEEIFLKVVEECAADTDMEDGSCGQHLCTGIAGLDVTLRLKMPPQETALENGEPAGSAPETDQGSGPDAVGRVQGWALTRQQLQALLLKRFLLARRSRRGLFAQIVLPALFVGLALVFSLIVPPFGHYPALRLSPTMYGAQVSFFSEDAPGDPGRARLLEALLQEAGLEEPPVQHSSHRFSAPEVPAEVAKVLASGNWTPESPSPACQCSRPGARRLLPDCPAAAGGPPPPQAVTGSGEVVQNLTGRNLSDFLVKTYPRLVRQGLKTKKWVNEVRYGGFSLGGRDPGLPSGQELGRSVEELWALLSPLPGGALDRVLKNLTAWAHSLDAQDSLKIWFNNKGWHSMVAFVNRASNAILRAHLPPGPARHAHSITTLNHPLNLTKEQLSEGALMASSVDVLVSICVVFAMSFVPASFTLVLIEERVTRAKHLQLMGGLSPTLYWLGNFLWDMCNYLVPACIVVLIFLAFQQRAYVAPANLPALLLLLLLYGWSITPLMYPASFFFSVPSTAYVVLTCINLFIGINGSMATFVLELFSDQKLQEVSRILKQVFLIFPHFCLGRGLIDMVRNQAMADAFERLGDRQFQSPLRWEVVGKNLLAMVIQGPLFLLFTLLLQHRSQLLPQPRVRSLPLLGEEDEDVARERERVVQGATQGDVLVLRNLTKVYRGQRMPAVDRLCLGIPPGECFGLLGVNGAGKTSTFRMVTGDTLASRGEAVLAGHSVAREPSAAHLSMGYCPQSDAIFELLTGREHLELLARLRGVPEAQVAQTAGSGLARLGLSWYADRPAGTYSGGNKRKLATALALVGDPAVVFLDEPTTGMDPSARRFLWNSLLAVVREGRSVMLTSHSMEECEALCSRLAIMVNGRFRCLGSPQHLKGRFAAGHTLTLRVPAARSQPAAAFVAAEFPGAELREAHGGRLRFQLPPGGRCALARVFGELAVHGAEHGVEDFSVSQTMLEEVFLYFSKDQGKDEDTEEQKEAGVGVDPAPGLQHPKRVSQFLDDPSTAETVL

SEQ ID NO: 11 - protein, 680 aa, Artificial Sequence
  Synthetic Construct
MALDGIRMPDGCYADGTWELSVHVTDLNRDVTLRVTGEVHIGGVMLKLVEKLDVKKDWSDHALWWEKKRTWLLKTHWTLDKYGIQADAKLQFTPQHKLLRLQLPNMKYVKVKVNFSDRVFKAVSDICKTFNIRHPEELSLLKKPRDPTKKKKKKLDDQSEDEALELEGPLITPGSGSIYSSPGLYSKTMTPTYDAHDGSPLSPTSAWFGDSALSEGNPGILAVSQPITSPEILAKMFKPQALLDKAKINQGWLDSSRSLMEQDVKENEALLLRFKYYSFFDLNPKYDAIRINQLYEQAKWAILLEEIECTEEEMMMFAALQYHINKLSIMTSENHLNNSDKEVDEVDAALSDLEITLEGGKTSTILGDITSIPELADYIKVFKPKKLTLKGYKQYWCTFKDTSISCYKSKEESSGTPAHQMNLRGCEVTPDVNISGQKFNIKLLIPVAEGMNEIWLRCDNEKQYAHWMAACRLASKGKTMADSSYNLEVQNILSFLKMQHLNPDPQLIPEQITTDITPECLVSPRYLKKYKNKQITARILEAHQNVAQMSLIEAKMRFIQAWQSLPEFGITHFIARFQGGKKEELIGIAYNRLIRMDASTGDAIKTWRFSNMKQWNVNWEIKMVTVEFADEVRLSFICTEVDCKVVHEFIGGYIFLSTRAKDQNESLDEEMFYKLTSGWV

SEQ ID NO: 12 - protein, 266 aa, Artificial Sequence
  Synthetic Construct
MVCLKLPGGSYMAKLTVTLMVLSSPLALAGDTRPRFLQQDKYECHFFNGTERVRFLHRDIYNQEEDLRFDSDVGEYRAVTELGRPDAEYWNSQKDFLEDRRAAVDTYCRHNYGVGESFTVQRRVEPKVTVYPARTQTLQHHNLLVCSVNGFYPGSIEVRWFRNSQEEKAGVVSTGLIQNGDWTFQTLVMLETVPRSGEVYTCQVEHPSVTSPLTVEWRAQSESAQSKMLSGVGGFVLGLLFLGAGLFIYFKNQKGHSGLHPTGLVS

SEQ ID NO: 13 - protein, 266 aa, Artificial Sequence
  Synthetic Construct
MVCLKLPGGSCMTALTVTLMVLSSPLALAGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSKMLSGVGGFVLGLLFLGAGLFIYFRNQKGHSGLQPTGFLS

SEQ ID NO: 14 - protein, 639 aa, Artificial Sequence
  Synthetic Construct
MVDYIVEYDYDAVHDDELTIRVGEIIRNVKKLQEEGWLEGELNGRRGMFPDNFVKEIKRETEFKDDSLPIKRERHGNVASLVQRISTYGLPAGGIQPHPQTKNIKKKTKKRQCKVLFEYIPQNEDELELKVGDIIDINEEVEEGWWSGTLNNKLGLFPSNFVKELEVTDDGETHEAQDDSETVLAGPTSPIPSLGNVSETASGSVTQPKKIRGIGFGDIFKEGSVKLRTRTSSSETEEKKPEKPLILQSLGPKTQSVEITKTDTEGKIKAKEYCRTLFAYEGTNEDELTFKEGEIIHLISKETGEAGWWRGELNGKEGVFPDNFAVQINELDKDFPKPKKPPPPAKAPAPKPELIAAEKKYFSLKPEEKDEKSTLEQKPSKPAAPQVPPKKPTPPTKASNLLRSSGTVYPKRPEKPVPPPPPIAKINGEVSSISSKFETEPVSKLKLDSEQLPLRPKSVDFDSLTVRTSKETDVVNFDDIASSENLLHLTANRPKMPGRRLPGRFNGGHSPTHSPEKILKLPKEEDSANLKPSELKKDTCYSPKPSVYLSTPSSASKANTTAFLTPLEIKAKVETDDVKKNSLDELRAQIIELLCIVEALKKDHGKELEKLRKDLEEEKTMRSNLEMEIEKLKKAVLSS

SEQ ID NO: 15 - protein, 1009 aa, Artificial Sequence
  Synthetic Construct
MSGVSEPLSRVKLGTLRRPEGPAEPMVVVPVDVEKEDVRILKVCFYSNSFNPGKNFKLVKCTVQTEIREIITSILLSGRIGPNIRLAECYGLRLKHMKSDEIHWLHPQMTVGEVQDKYECLHVEAEWRYDLQIRYLPEDFMESLKEDRTTLLYFYQQLRNDYMQRYASKVSEGMALQLGCLELRRFFKDMPHNALDKKSNFELLEKEVGLDLFFPKQMQENLKPKQFRKMIQQTFQQYASLREEECVMKFFNTLAGFANIDQETYRCELIQGWNITVDLVIGPKGIRQLTSQDAKPTCLAEFKQIRSIRCLPLEEGQAVLQLGIEGAPQALSIKTSSLAEAENMADLIDGYCRLQGEHQGSLIIHPRKDGEKRNSLPQIPMLNLEARRSHLSESCSIESDIYAEIPDETLRRPGGPQYGIAREDVVLNRILGEGFFGEVYEGVYTNHKGEKINVAVKTCKKDCTLDNKEKFMSEAVIMKNLDHPHIVKLIGIIEEEPTWIIMELYPYGELGHYLERNKNSLKVLTLVLYSLQICKAMAYLESINCVHRDIAVRNILVASPECVKLGDFGLSRYIEDEDYYKASVTRLPIKWMSPESINFRRFTTASDVWMFAVCMWEILSFGKQPFFWLENKDVIGVLEKGDRLPKPDLCPPVLYTLMTRCWDYDPSDRPRFTELVCSLSDVYQMEKDIAMEQERNARYRTPKILEPTAFQEPPPKPSRPKYRPPPQTNLLAPKLQFQVPEGLCASSPTLTSPMEYPSPVNSLHTPPLHRHNVFKRHSMREEDFIQPSSREEAQQLWEAEKVKMRQILDKQQKQMVEDYQWLRQEEKSLDPMVYMNDKSPLTPEKEVGYLEFTGPPQKPPRLGAQSIQPTANLDRTDDLVYLNVMELVRAVLELKNELCQLPPEGYVVVVKNVGLTLRKLIGSVDDLLPSLPSSSRTEIEGTQKLLNKDLAELINKMRLAQQNAVTSLSEECKRQMLTASHTLAVDAKNLLDAVDQAKVLANLAHPPAE

SEQ ID NO: 16 - protein, 486 aa, Artificial Sequence
  Synthetic Construct
MNGTLDHPDQPDLDAIKMFVGQVPRTWSEKDLRELFEQYGAVYEINVLRDRSQNPPQSKGCCFVTFYTRKAALEAQNALHNMKVLPGMHHPIQMKPADSEKNNAVEDRKLFIGMISKKCTENDIRVMFSSFGQIEECRILRGPDGLSRGCAFVTFTTRAMAQTAIKAMHQAQTMEGCSSPMVVKFADTQKDKEQKRMAQQLQQQMQQISAASVWGNLAGLNTLGPQYLALYLQLLQQTASSGNLNTLSSLHPMGGLNAMQLQNLAALAAAASAAQNTPSGTNALTTSSSPLSVLTSSGSSPSSSSSNSVNPIASLGALQTLAGATAGLNVGSLAGMAALNGGLGSSGLSNGTGSTMEALTQAYSGIQQYAAAALPTLYNQNLLTQQSIGAAGSQKEGPEGANLFIYHLPQEFGDQDLLQMFMPFGNVVSAKVFIDKQTNLSKCFGFVSYDNPVSAQAAIQSMNGFQIGMKRLKVQLKRSKNDSKPY

SEQ ID NO: 17 - protein, 1189 aa, Artificial Sequence
  Synthetic Construct
MVPCWNHGNITRSKAEELLSRTGKDGSFLVRASESISRAYALCVLYRNCVYTYRILPNEDDKFTVQASEGVSMRFFTKLDQLIEFYKKENMGLVTHLQYPVPLEEEDTGDDPEEDTVESVVSPPELPPRNIPLTASSCEAKEVPFSNENPRATETSRPSLSETLFQRLQSMDTSGLPEEHLKAIQDYLSTQLAQDSEFVKTGSSSLPHLKKLTTLLCKELYGEVIRTLPSLESLQRLFDQQLSPGLRPRPQVPGEANPINMVSKLSQLTSLLSSIEDKVKALLHEGPESPHRPSLIPPVTFEVKAESLGIPQKMQLKVDVESGKLIIKKSKDGSEDKFYSHKKILQLIKSQKFLNKLVILVETEKEKILRKEYVFADSKKREGFCQLLQQMKNKHSEQPEPDMITIFIGTWNMGNAPPPKKITSWFLSKGQGKTRDDSADYIPHDIYVIGTQEDPLSEKEWLEILKHSLQEITSVTFKTVAIHTLWNIRIVVLAKPEHENRISHICTDNVKTGIANTLGNKGAVGVSFMFNGTSLGFVNSHLTSGSEKKLRRNQNYMNILRFLALGDKKLSPFNITHRFTHLFWFGDLNYRVDLPTWEAETIIQKIKQQQYADLLSHDQLLTERREQKVFLHFEEEEITFAPTYRFERLTRDKYAYTKQKATGMKYNLPSWCDRVLWKSYPLVHVVCQSYGSTSDIMTSDHSPVFATFEAGVTSQFVSKNGPGTVDSQGQIEFLRCYATLKTKSQTKFYLEFHSSCLESFVKSQEGENEEGSEGELVVKFGETLPKLKPIISDPEYLLDQHILISIKSSDSDESYGEGCIALRLEATETQLPIYTPLTHHGELTGHFQGEIKLQTSQGKTREKLYDFVKTERDESSGPKTLKSLTSHDPMKQWEVTSRAPPCSGSSITEIINPNYMGVGPFGPPMPLHVKQTLSPDQQPTAWSYDQPPKDSPLGPCRGESPPTPPGQPPISPKKFLPSTANRGLPPRTQESRPSDLGKNAGDTLPQEDLPLTKPEMFENPLYGSLSSFPKPAPRKDQESPKMPRKEPPPCPEPGILSPSIVLTKAQEADRGEGPGKQVPAPRLRSFTCSSSAEGRAAGGDKSQGKPKTPVSSQAPVPAKRPIKPSRSEINQQTPPTPTPRPPLPVKSPAVLHLQHSKGRDYRDNTELPHHGKHRPEEGPPGPLGRTAMQ

SEQ ID NO: 18 - protein, 473 aa, Artificial Sequence
  Synthetic Construct
MGRKKIQITRIMDERNRQVTFTKRKFGLMKKAYELSVLCDCEIALIIFNSTNKLFQYASTDMDKVLLKYTEYNEPHESRTNSDIVETLRKKGLNGCDSPDPDADDSVGHSPESEDKYRKINEDIDLMISRQRLCAVPPPNFEMPVSIPVSSHNSLVYSNPVSSLGNPNLLPLAHPSLQRNSMSPGVTHRPPSAGNTGGLMGGDLTSGAGTSAGNGYGNPRNSPGLLVSPGNLNKNMQAKSPPPMNLGMNNRKPDLRVLIPPGSKNTMPSVSEDVDLLLNQRINNSQSAQSLATPVVSVATPTLPGQGMGGYPSAISTTYGTEYSLSSADLSSLSGFNTASALHLGSVTGWQQQHLHNMPPSALSQLGACTSTHLSQSSNLSLPSTQSLNIKSEPVSPPRDRTTTPSRYPQHTRHEAGRSPVDSLSSCSSSYDGSDREDHRNEFHSPIGLTRPSPDERESPSVKRMRLSEGWAT

SEQ ID NO: 19 - protein, 648 aa, Artificial Sequence
  Synthetic Construct
MMTTLQNKEECGKGPKRIFAPPAQKSYSLLPCSPNSPKEETPGISSPETEARISLPKASLKKKEEKATMKNVPSREQEKKRKAQINKQAEKKEKEKSSLTNAEFEEIVQIVLQKSLQECLGMGSGLDFAETSCAQPVVSTQSDKEPGITASATDTDNANGEEVPHTQEISVSWEGEAAPEIRTSKLGQPDPAPSKKKSNRLTLSKRKKEAHEKVEKTQGGHEHRQEDRLKKTVQDHSQIRDQQKGEISGFGQCLVWVQCSFPNCGKWRRLCGNIDPSVLPDNWSCDQNTDVQYNRCDIPEETWTGLESDVAYASYIPGSIIWAKQYGYPWWPGMIESDPDLGEYFLFTSHLDSLPSKYHVTFFGETVSRAWIPVNMLKNFQELSLELSVMKKRRNDCSQKLGVALMMAQEAEQISIQERVNLFGFWSRFNGSNSNGERKDLQLSGLNSPGSCLEKKEKEEELEKEEGEKTDPILPIRKRVKIQTQKTKPRGLGGDAGTADGRGRTLQRKIMKRSLGRKSTAPPAPRMGRKEGQGNSDSDQPGPKKKFKAPQSKALAASFSEGKEVRTVPKNLGLSACKGACPSSAKEEPRHREPLTQEAGSVPLEDEASSDLDLEQLMEDVGRELGQSGELQHSNSDGEDFPVALFGK

SEQ ID NO: 20 - protein, 364 aa, Artificial Sequence
  Synthetic Construct
MPLLLLLPLLWAGALAMDPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 21 - protein, 239 aa, Artificial Sequence [sequence omitted]

SEQ ID NO: 22 - protein, 985 aa, Artificial Sequence
  Synthetic Construct
MIRHAGAPARGDPTGPVPVVGKGEEEEEEDGMRLCLPANPKNCLPHRRGISILEKLIKTCPVWLQLSLGQAEVARILHRVVAGMFLVRRDSSSKQLVLCVHFPSLNESSAEVLEYTIKEEKSILYLEGSALVFEDIFRLIAFYCVSRDLLPFTLRLPQAILEASSFTDLETIANLGLGFWDSSLNPPQERGKPAEPPRDRAPGFPLVSSLRPTAHDANCACEIELSVGNDRLWFVNPIFIEDCSSALPTDQPPLGNCPARPLPPTSDATSPTSRWAPRRPPPPPPVLPLQPCSPAQPPVLPALAPAPACPLPTSPPVPAPHVTPHAPGPPDHPNQPPMMTCERLPCPTAGLGPLREEAMKPGAASSPLQQVPAPPLPAKKNLPTAPPRRRVSERVSLEDQSPGMAAEGDQLSLPPQGTSDGPEDTPRESTEQGQDTEVKASDPHSMPELPRTAKQPPVPPPRKKRISRQLASTLPAPLENAELCTQAMALETPTPGPPREGQSPASQAGTQHPPAQATAHSQSSPEFKGSLASLSDSLGVSVMATDQDSYSTSSTEEELEQFSSPSVKKKPSMILGKARHRLSFASFSSMFHAFLSNNRKLYKKVVELAQDKGSYFGSLVQDYKVYSLEMMARQTSSTEMLQEIRTMMTQLKSYLLQSTELKALVDPALHSEEELEAIVESALYKCVLKPLKEAINSCLHQIHSKDGSLQQLKENQLVILATTTTDLGVTTSVPEVPMMEKILQKFTSMHKAYSPEKKISILLKTCKLIYDSMALGNPGKPYGADDFLPVLMYVLARSNLTEMLLNVEYMMELMDPALQLGEGSYYLTTTYGALEHIKSYDKITVTRQLSVEVQDSIHRWERRRTLNKARASRSSVQDFICVSYLEPEQQARTLASRADTQAQALCAQCAEKFAVERPQAHRLFVLVDGRCFQLADDALPHCIKGYLLRSEPKRDFHFVYRPLDGGGGGGGGSPPCLVVREPNFL

SEQ ID NO: 23 - protein, 976 aa, Artificial Sequence
  Synthetic Construct
MERRWPLGLGLVLLLCAPLPPGARAKEVTLMDTSKAQGELGWLLDPPKDGWSEQQQILNGTPLYMYQDCPMQGRRDTDHWLRSNWIYRGEEASRVHVELQFTVRDCKSFPGGAGPLGCKETFNLLYMESDQDVGIQLRRPLFQKVTTVAADQSFTIRDLVSGSVKLNVERCSLGRLTRRGLYLAFHNPGACVALVSVRVFYQRCPETLNGLAQFPDTLPGPAGLVEVAGTCLPHARASPRPSGAPRMHCSPDGEWLVPVGRCHCEPGYEEGGSGEACVACPSGSYRMDMDTPHCLTCPQQSTAESEGATICTCESGHYRAPGEGPQVACTGPPSAPRNLSFSASGTQLSLRWEPPADTGGRQDVRYSVRCSQCQGTAQDGGPCQPCGVGVHFSPGARGLTTPAVHVNGLEPYANYTFNVEAQNGVSGLGSSGHASTSVSISMGHAESLSGLSLRLVKKEPRQLELTWAGSRPRSPGANLTYELHVLNQDEERYQMVLEPRVLLTELQPDTTYIVRVRMLTPLGPGPFSPDHEFRTSPPVSRGLTGGEIVAVIFGLLLGAALLLGILVFRSRRAQRQRQQRQRDRATDVDREDKLWLKPYVDLQAYEDPAQGALDFTRELDPAWLMVDTVIGEGEFGEVYRGTLRLPSQDCKTVAIKTLKDTSPGGQWWNFLREATIMGQFSHPHILHLEGVVTKRKPIMIITEFMENGALDAFLREREDQLVPGQLVAMLQGIASGMNYLSNHNYVHRDLAARNILVNQNLCCKVSDFGLTRLLDDFDGTYETQGGKIPIRWTAPEAIAHRIFTTASDVWSFGIVMWEVLSFGDKPYGEMSNQEVMKSIEDGYRLPPPVDCPAPLYELMKNCWAYDRARRPHFQKLQAHLEQLLANPHSLRTIANFDPRMTLRLPSLSGSDGIPYRTVSEWLESIRMKRYILHFHSAGLDTMECVLELTAEDLTQMGITLPGHQKRILCSIQGFKD

SEQ ID NO: 24 - protein, 652 aa, Artificial Sequence
  Synthetic Construct
MSGQSLTDRITAAQHSVTGSAVSKTVCKATTHEIMGPKKKHLDYLIQCTNEMNVNIPQLADSLFERTTNSSWVVVFKSLITTHHLMVYGNERFIQYLASRNTLFNLSNFLDKSGLQGYDMSTFIRRYSRYLNEKAVSYRQVAFDFTKVKRGADGVMRTMNTEKLLKTVPIIQNQMDALLDFNVNSNELTNGVINAAFMLLFKDAIRLFAAYNEGIINLLEKYFDMKKNQCKEGLDIYKKFLTRMTRISEFLKVAEQVGIDRGDIPDLSQAPSSLLDALEQHLASLEGKKIKDSTAASRATTLSNAVSSLASTGLSLTKVDEREKQAALEEEQARLKALKEQRLKELAKKPHTSLTTAASPVSTSAGGIMTAPAIDIFSTPSSSNSTSKLPNDLLDLQQPTFHPSVHPMSTASQVASTWGDPFSATVDAVDDAIPSLNPFLTKSSGDVHLSISSDVSTFTTRTPTHEMFVGFTPSPVAQPHPSAGLNVDFESVFGNKSTNVIVDSGGFDELGGLLKPTVASQNQNLPVAKLPPSKLVSDDLDSSLANLVGNLGIGNGTTKNDVNWSQPGEKKLTGGSNWQPKVAPTTAWNAATMAPPVMAYPATTPTGMIGYGIPPQMGSVPVMTQPTLIYSQPVMRPPNPFGPVSGAQIQFM

SEQ ID NO: 25 - protein, 786 aa, Artificial Sequence
  Synthetic Construct
MKGTGIMDCAPKALLARALYDNCPDCSDELAFSRGDILTILEQHVPESEGWWKCLLHGRQGLAPANRLQILTEVAADRPCPPFLRGLEEAPASSEETYQVPTLPRPPTPGPVYEQMRSWAEGPQPPTAQVYEFPDPPTSARIICEKTLSFPKQAILTLPRPVRASLPTLPSQVYDVPTQHRGPVVLKEPEKQQLYDIPASPKKAGLHPPDSQASGQGVPLISVTTLRRGGYSTLPNPQKSEWIYDTPVSPGKASVRNTPLTSFAEESRPHALPSSSSTFYNPPSGRSRSLTPQLNNNVPMQKKLSLPEIPSYGFLVPRGTFPLDEDVSYKVPSSFLIPRVEQQNTKPNIYDIPKATSSVSQAGKELEKAKEVSENSAGHNSSWFSRRTTSPSPEPDRLSGSSSDSRASIVSSCSTTSTDDSSSSSSEESAKELSLDLDVAKETVMALQHKVVSSVAGLMLFVSRKWRFRDYLEANIDAIHRSTDHIEESVREFLDFARGVHGTACNLTDSNLQNRIRDQMQTISNSYRILLETKESLDNRNWPLEVLVTDSVQNSPDDLERFVMVARMLPEDIKRFASIVIANGRLLFKRNCEKEETVQLTPNAEFKCEKYIQPPQRETESHQKSTPSTKQREDEHSSELLKKNRANICGQNPGPLIPQPSSQQTPERKPRLSEHCRLYFGALFKAISAFHGSLSSSQPAEIITQSKLVIMVGQKLVDTLCMETQERDVRNEILRGSSHLCSLLKDVALATKNAVLTYPSPAALGHLQAEAEKLEQHTRQFRGTLG

SEQ ID NO: 26 - protein, 449 aa, Artificial Sequence
  Synthetic Construct
MMKTLLLFVGLLLTWESGQVLGDQTVSDNELQEMSNQGSKYVNKEIQNAVNGVKQIKTLIEKTNEERKTLLSNLEEAKKKKEDALNETRESETKLKELPGVCNETMMALWEECKPCLKQTCMKFYARVCRSGSGLVGRQLEEFLNQSSPFYFWMNGDRIDSLLENDRQQTHMLDVMQDHFSRASSIIDELFQDRFFTREPQDTYHYLPFSLPHRRPHFFFPKSRIVRSLMPFSPYEPLNFHAMFQPFLEMIHEAQQAMDIHFHSPAFQHPPTEFIREGDDDRTVCREIRHNSTGCLRMKDQCDKCREILSVDCSTNNPSQAKLRRELDESLQVAERLTRKYNELLKSYQWKMLNTSSLLEQLNEQFNWVSRLANLTQGEDQYYLRVTTVASHTSDSDVPSGVTEVVVKLFDSDPITVTVPVEVSRKNPKFMETVAEKALQEYRKKHREE

SEQ ID NO: 27 - protein, 2214 aa, Artificial Sequence
  Synthetic Construct
MATRSSRRESRLPFLFTLVALLPPGALCEVWTQRLHGGSAPLPQDRGFLVVQGDPRELRLWARGDARGASRADEKPLRRKRSAALQPEPIKVYGQVSLNDSHNQMVVHWAGEKSNVIVALARDSLALARPKSSDVYVSYDYGKSFKKISDKLNFGLGNRSEAVIAQFYHSPADNKRYIFADAYAQYLWITFDFCNTLQGFSIPFRAADLLLHSKASNLLLGFDRSHPNKQLWKSDDFGQTWIMIQEHVKSFSWGIDPYDKPNTIYIERHEPSGYSTVFRSTDFFQSRENQEVILEEVRDFQLRDKYMFATKVVHLLGSEQQSSVQLWVSFGRKPMRAAQFVTRHPINEYYIADASEDQVFVCVSHSNNRTNLYISEAEGLKFSLSLENVLYYSPGGAGSDTLVRYFANEPFADFHRVEGLQGVYIATLINGSMNEENMRSVITFDKGGTWEFLQAPAFTGYGEKINCELSQGCSLHLAQRLSQLLNLQLRRMPILSKESAPGLIIATGSVGKNLASKTNVYISSSAGARWREALPGPHYYTWGDHGGIITAIAQGMETNELKYSTNEGETWKTFIFSEKPVFVYGLLTEPGEKSTVFTIFGSNKENVHSWLILQVNATDALGVPCTENDYKLWSPSDERGNECLLGHKTVFKRRTPHATCFNGEDFDRPVVVSNCSCTREDYECDFGFKMSEDLSLEVCVPDPEFSGKSYSPPVPCPVGSTYRRTRGYRKISGDTCSGGDVEARLEGELVPCPLAEENEFILYAVRKSIYRYDLASGATEQLPLTGLRAAVALDFDYEHNCLYWSDLALDVIQRLCLNGSTGQEVIINSGLETVEALAFEPLSQLLYWVDAGFKKIEVANPDGDFRLTIVNSSVLDRPRALVLVPQEGVMFWTDWGDLKPGIYRSNMDGSAAYHLVSEDVKWPNGISVDDQWIYWTDAYLECIERITFSGQQRSVILDNLPHPYAIAVFKNEIYWDDWSQLSIFRASKYSGSQMEILANQLTGLMDMKIFYKGKNTGSNACVPRPCSLLCLPKANNSRSCRCPEDVSSSVLPSGDLMCDCPQGYQLKNNTCVKQENTCLRNQYRCSNGNCINSIWWCDFDNDCGDMSDERNCPTTICDLDTQFRCQESGTCIPLSYKCDLEDDCGDNSDESHCEMHQCRSDEYNCSSGMCIRSSWVCDGDNDCRDWSDEANCTAIYHTCEASNFQCRNGHCIPQRWACDGDTDCQDGSDEDPVNCEKKCNGFRCPNGTCIPSSKHCDGLRDCSDGSDEQHCEPLCTHFMDFVCKNRQQCLFHSMVCDGIIQCRDGSDEDAAFAGCSQDPEFHKVCDEFGFQCQNGVCISLIWKCDGMDDCGDYSDEANCENPTEAPNCSRYFQFRCENGHCIPNRWKCDRENDCGDWSDEKDCGDSHILPFSTPGPSTCLPNYYRCSSGTCVMDTWVCDGYRDCADGSDEEACPLLANVTAASTPTQLGRCDRFEFECHQPKTCIPNWKRCDGHQDCQDGRDEANCPTHSTLTCMSREFQCEDGEACIVLSERCDGFLDCSDESDEKACSDELTVYKVQNLQWTADFSGDVTLTWMRPKKMPSASCVYNVYYRVVGESIWKTLETHSNKTNTVLKVLKPDTTYQVKVQVQCLSKAHNTNDFVTLRTPEGLPDAPRNLQLSLPREAEGVIVGHWAPPIHTHGLIREYIVEYSRSGSKMWASQRAASNFTEIKNLLVNTLYTVRVAAVTSRGIGNWSDSKSITTIKGKVIPPPDIHIDSYGENYLSFTLTMESDIKVNGYVVNLFWAFDTHKQERRTLNFRGSILSHKVGNLTAHTSYEISAWAKTDLGDSPLAFEHVMTRGVRPPAPSLKAKAINQTAVECTWTGPRNVVYGIFYATSFLDLYRNPKSLTTSLHNKTVIVSKDEQYLFLVRVVVPYQGPSSDYVVVKMIPDSRLPPRHLHVVHTGKTSVVIKWESPYDSPDQDLLYAVAVKDLIRKTDRSYKVKSRNSTVEYTLNKLEPGGKYHIIVQLGNMSKDSSIKITTVSLSAPDALKIITENDHVLLFWKSLALKEKHFNESRGYEIHMFDSAMNITAYLGNTTDNFFKISNLKMGHNYTFTVQARCLFGNQICGEPAILLYDELGSGADASATQAARSTDVAAVVVPILFLILLSLGVGFAILYTKHRRLQSSFTAFANSHYSSRLGSAIFSSGDDLGEDDEDAPMITGFSDDVPMVIA

SEQ ID NO: 28 - protein, 1265 aa, Artificial Sequence
  Synthetic Construct
MSTTVNVDSLAEYEKSQIKRALELGTVMTVFSFRKSTPERRTVQVIMETRQVAWSKTADKIEGFLDIMEIKEIRPGKNSKDFERAKAVRQKEDCCFTILYGTQFVLSTLSLAADSKEDAVNWLSGLKILHQEAMNASTPTIIESWLRKQIYSVDQTRRNSISLRELKTILPLINFKVSSAKFLKDKFVEIGAHKDELSFEQFHLFYKKLMFEQQKSILDEFKKDSSVFILGNTDRPDASAVYLHDFQRFLIHEQQEHWAQDLNKVRERMTKFIDDTMRETAEPFLFVDEFLTYLFSRENSIWDEKYDAVDMQDMNNPLSHYWISSSHNTYLTGDQLRSESSPEAYIRCLRMGCRCIELDCWDGPDGKPVIYHGWTRTTKIKFDDVVQAIKDHAFVTSSFPVILSIEEHCSVEQQRHMAKAFKEVFGDLLLTKPTEASADQLPSPSQLREKIIIKHKKLGPRGDVDVNMEDKKDEHKQQGELYMWDSIDQKWTRHYCAIADAKLSFSDDIEQTMEEEVPQDIPPTELHFGEKWFHKKVEKRTSAEKLLQEYCMETGGKDGTFLVRESETFPNDYTLSFWRSGRVQHCRIRSTMEGGTLKYYLTDNLTFSSIYALIQHYRETHLRCAEFELRLTDPVPNPNPHESKPWYYDSLSRGEAEDMLMRIPRDGAFLIRKREGSDSYAITFRARGKVKHCRINRDGRHFVLGTSAYFESLVELVSYYEKHSLYRKMRLRYPVTPELLERYNMERDINSLYDVSRMYVDPSEINPSMPQRTVKALYDYKAKRSDELSFCRGALIHNVSKEPGGWWKGDYGTRIQQYFPSNYVEDISTADFEELEKQIIEDNPLGSLCRGILDLNTYNVVKAPQGKNQKSFVFILEPKQQGDPPVEFATDRVEELFEWFQSIREITWKIDTKENNMKYWEKNQSIAIELSDLVVYCKPTSKTKDNLENPDFREIRSFVETKADSIIRQKPVDLLKYNQKGLTRVYPKGQRVDSSNYDPFRLWLCGSQMVALNFQTADKYMQMNHALFSLNGRTGYVLQPESMRTEKYDPMPPESQRKILMTLTVKVLGARHLPKLGRSIACPFVEVEICGAEYDNNKFKTTVVNDNGLSPIWAPTQEKVTFEIYDPNLAFLRFVVYEEDMFSDPNFLAHATYPIKAVKSGFRSVPLKNGYSEDIELASLLVFCEMRPVLESEEELYSSCRQLRRRQEELNNQLFLYDTHQNLRNANRDALVKEFSVNENQLQLYQEKCNKRLREKRVSNSKFYS

SEQ ID NO: 29 - protein, 145 aa, Artificial Sequence
  Synthetic Construct
MDTFTVQDSTAMSWWRNNFWIILAVAIIVVSVGLGLILYCVCKWQLRRGKKWEIAKPLKHKQVDEEKMYENVLNESPVQLPPLPPRNWPSLEDSSPQEAPSQPPATYSLVNKVKNKKTVSIPSYIEPEDDYDDVEIPANTEKASF

SEQ ID NO: 30 - protein, 1039 aa, Artificial Sequence
  Synthetic Construct
MAVQLVPDSALGLLMMTEGRRCQVHLLDDRKLELLVQPKLLAKELLDLVASHFNLKEKEYFGIAFTDETGHLNWLQLDRRVLEHDFPKKSGPVVLYFCVRFYIESISYLKDNATIELFFLNAKSCIYKELIDVDSEVVFELASYILQEAKGDFSSNEVVRSDLKKLPALPTQALKEHPSLAYCEDRVIEHYKKLNGQTRGQAIVNYMSIVESLPTYGVHYYAVKDKQGIPWWLGLSYKGIFQYDYHDKVKPRKIFQWRQLENLYFREKKFSVEVHDPRRASVTRRTFGHSGIAVHTWYACPALIKSIWAMAISQHQFYLDRKQSKSKIHAARSLSEIAIDLTETGTLKTSKLANMGSKGKIISGSSGSLLSSGSQESDSSQSAKKDMLAALKSRQEALEETLRQRLEELKKLCLREAELTGKLPVEYPLDPGEEPPIVRRRIGTAFKLDEQKILPKGEEAELERLEREFAIQSQITEAARRLASDPNVSKKLKKQRKTSYLNALKKLQEIENAINENRIKSGKKPTQRASLIIDDGNIASEDSSLSDALVLEDEDSQVTSTISPLHSPHKGLPPRPPSHNRPPPPQSLEGLRQMHYHRNDYDKSPIKPKMWSESSLDEPYEKVKKRSSHSHSSSHKRFPSTGSCAEAGGGSNSLQNSPIRGLPHWNSQSSMPSTPDLRVRSPHYVHSTRSVDISPTRLHSLALHFRHRSSSLESQGKLLGSENDTGSPDFYTPRTRSSNGSDPMDDCSSCTSHSSSEHYYPAQMNANYSTLAEDSPSKARQRQRQRQRAAGALGSASSGSMPNLAARGGAGGAGGAGGGVYLHSQSQPSSQYRIKEYPLYIEGGATPVVVRSLESDQEGHYSVKAQFKTSNSYTAGGLFKESWRGGGGDEGDTGRLTPSRSQILRTPSLGREGAHDKGAGRAAVSDELRQWYQRSTASHKEHSRLSHTSSTSSDSGSQYSTSSQSTFVAHSRVTRMPQMCKATSAALPQSQRSSTPSSEIGATPPSSPHHILTWQTGEATENSPILDGSESPPHQSTDE

SEQ ID NO: 31 - protein, 520 aa, Artificial Sequence
  Synthetic Construct
MGPQRRLSPAGAALLWGFLLQLTAAQEAILHASGNGTTKDYCMLYNPYWTALPSTLENATSISLMNLTSTPLCNLSDIPPVGIKSKAVVVPWGSCHFLEKARIAQKGGAEAMLVVNNSVLFPPSGNRSEFPDVKILIAFISYKDFRDMNQTLGDNITVKMYSPSWPNFDYTMVVIFVIAVFTVALGGYWSGLVELENLKAVTTEDREMRKKKEEYLTFSPLTVVIFVVICCVMMVLLYFFYKWLVYVMIAIFCIASAMSLYNCLAALIHKIPYGQCTIACRGKNMEVRLIFLSGLCIAVAVVWAVFRNEDRWAWILQDILGIAFCLNLIKTLKLPNFKSCVILLGLLLLYDVFFVFITPFITKNGESIMVELAAGPFGNNEKLPVVIRVPKLIYFSVMSVCLMPVSILGFGDIIVPGLLIAYCRRFDVQTGSSYIYYVSSTVAYAIGMILTFVVLVLMKKGQPALLYLVPCTLITASVVAWRRKEMKKFWKGNSYQMMDHLDCATNEENPVISGEQIVQQ

SEQ ID NO: 32 - protein, 978 aa, Artificial Sequence
  Synthetic Construct
MGTRLPLVLRQLRRPPQPPGPPRRLRVPCRASSGGGGGGGGGREGLLGQRRPQDGQARSSCSPGGRTPAARDSIVREVIQNSKEVLSLLQEKNPAFKPVLAIIQAGDDNLMQEINQNLAEEAGLNITHICLPPDSSEAEIIDEILKINEDTRVHGLALQISENLFSNKVLNALKPEKDVDGVTDINLGKLVRGDAHECFVSPVAKAVIELLEKSGVNLDGKKILVVGAHGSLEAALQCLFQRKGSMTMSIQWKTRQLQSKLHEADIVVLGSPKPEEIPLTWIQPGTTVLNCSHDFLSGKVGCGSPRIHFGGLIEEDDVILLAAALRIQNMVSSGRRWLREQQHRRWRLHCLKLQPLSPVPSDIEISRGQTPKAVDVLAKEIGLLADEIEIYGKSKAKVRLSVLERLKDQADGKYVLVAGITPTPLGEGKSTVTIGLVQALTAHLNVNSFACLRQPSQGPTFGVKGGAAGGGYAQVIPMEEFNLHLTGDIHAITAANNLLAAAIDTRILHENTQTDKALYNRLVPLVNGVREFSEIQLARLKKLGINKTDPSTLTEEEVSKFARLDIDPSTITWQRVLDTNDRFLRKITIGQGNTEKGHYRQAQFDIAVASEIMAVLALTDSLADMKARLGRMVVASDKSGQPVTADDLGVTGALTVLMKDAIKPNLMQTLEGTPVFVHAGPFANIAHGNSSVLADKIALKLVGEEGFVVTEAGFGADIGMEKFFNIKCRASGLVPNVVVLVATVRALKMHGGGPSVTAGVPLKKEYTEENIQLVADGCCNLQKQIQITQLFGVPVVVALNVFKTDTRAEIDLVCELAKRAGAFDAVPCYHWSVGGKGSVDLARAVREAASKRSRFQFLYDVQVPIVDKIRTIAQAVYGAKDIELSPEAQAKIDRYTQQGFGNLPICMAKTHLSLSHQPDKKGVPRDFILPISDVRASIGAGFIYPLVGTMSTMPGLPTRPCFYDIDLDTETEQVKGLF

SEQ ID NO: 33 - protein, 443 aa, Artificial Sequence
  Synthetic Construct
MDSRAQLWGLALNKRRATLPHPGGSTNLKADPEELFTKLEKIGKGSFGEVFKGIDNRTQKVVAIKIIDLEEAEDEIEDIQQEITVLSQCDSPYVTKYYGSYLKDTKLWIIMEYLGGGSALDLLEPGPLDETQIATILREILKGLDYLHSEKKIHRDIKAANVLLSEHGEVKLADFGVAGQLTDTQIKRNTFVGTPFWMAPEVIKQSAYDSKADIWSLGITAIELARGEPPHSELHPMKVLFLIPKNNPPTLEGNYSKPLKEFVEACLNKEPSFRPTAKELLKHKFILRNAKKTSYLTELIDRYKRWKAEQSHDDSSSEDSDAETDGQASGGSDSGDWIFTIREKDPKNLENGALQPSDLDRNKMKDIPKRPFSQCLSTIISPLFAELKEKSQACGGNLGSIEELRGAIYLAEEACPGISDTMVAQLVQRLQRYSLSGGGTSSH

SEQ ID NO: 34 - protein, 854 aa, Artificial Sequence
  Synthetic Construct
MPGGGPQGAPAAAGGGGVSHRAGSRDCLPPAACFRRRRLARRPGYMRSSTGPGIGFLSPAVGTLFRFPGGVSGEESHHSESRARQCGLDSRGLLVRSPVSKSAAAPTVTSVRGTSAHFGIQLRGGTRLPDRLSWPCGPGSAGWQQEFAAMDSSETLDASWEAACSDGARRVRAAGSLPSAELSSNSCSPGCGPEVPPTPPGSHSAFTSSFSFIRLSLGSAGERGEAEGCPPSREAESHCQSPQEMGAKAASLDGPHEDPRCLSRPFSLLATRVSADLAQAARNSSRPERDMHSLPDMDPGSSSSLDPSLAGCGGDGSSGSGDAHSWDTLLRKWEPVLRDCLLRNRRQMEVISLRLKLQKLQEDAVENDDYDKAETLQQRLEDLEQEKISLHFQLPSRQPALSSFLGHLAAQVQAALRRGATQQASGDDTHTPLRMEPRLLEPTAQDSLHVSITRRDWLLQEKQQLQKEIEALQARMFVLEAKDQQLRREIEEQEQQLQWQGCDLTPLVGQLSLGQLQEVSKALQDTLASAGQIPFHAEPPETIRSLQERIKSLNLSLKEITTKVCMSEKFCSTLRKKVNDIETQLPALLEAKMHAISGNHFWTAKDLTEEIRSLTSEREGLEGLLSKLLVLSSRNVKKLGSVKEDYNRLRREVEHQETAYETSVKENTMKYMETLKNKLCSCKCPLLGKVWEADLEACRLLIQSLQLQEARGSLSVEDERQMDDLEGAAPPIPPRLHSEDKRKTPLKVLEEWKTHLIPSLHCAGGEQKEESYILSAELGEKCEDIGKKLLYLEDQLHTAIHSHDEDLIQSLRRELQMVKETLQAMILQLQPAKEAGEREAAASCMTAGVHEAQA

SEQ ID NO: 35 - protein, 269 aa, Artificial Sequence
  Synthetic Construct
MAASAGAGAVIAAPDSRRWLWSVLAAALGLLTAGVSALEVYTPKEIFVANGTQGKLTCKFKSTSTTGGLTSVSWSFQPEGADTTVSFFHYSQGQVYLGNYPPFKDRISWAGDLDKKDASINIENMQFIHNGTYICDVKNPPDIVVQPGHIRLYVVEKENLPVFPVWVVVGIVTAVVLGLTLLISMILAVLYRRKNSKRDYTGCSTSESLSPVKQAPRKSPSDTEGLVKSLPSGSHQGPVIYAQLDHSGGHHSDKINKSESVVYADIRKN

SEQ ID NO: 36 - protein, 796 aa, Artificial Sequence
  Synthetic Construct
MLAPLRNAPGREGATSPSPPTDATGSLGEWDVDRNVKTEGWVSKERISKLHRLRMADILSQSETLASQDLSGDFKKPALPVSPAARSKAPASSSSNPEEVQKEGPTALQDSNSGEPDIPPPQPDCGDFRSLQEEQSRPPTAVSSPGGPARAPPYQEPPWGGPATAPYSLETLKGGTILGTRSLKGTSYCLFGRLSGCDVCLEHPSVSRYHAVLQHRASGPDGECDSNGPGFYLYDLGSTHGTFLNKTRIPPRTYCRVHVGHVVRFGGSTRLFILQGPEEDREAESELTVTQLKELRKQQQILLEKKMLGEDSDEEEEMDTSERKINAGSQDDEMGCTWGMGEDAVEDDAEENPIVLEFQQEREAFYIKDPKKALQGFFDREGEELEYEFDEQGHSTWLCRVRLPVDDSTGKQLVAEAIHSGKKKEAMIQCSLEACRILDTLGLLRQEAVSRKRKAKNWEDEDFYDSDDDTFLDRTGLIEKKRLNRMKKAGKIDEKPETFESLVAKLNDAERELSEISERLKASSQVLSESPSQDSLDAFMSEMKSGSTLDGVSRKKLHLRTFELRKEQQRLKGLIKIVKPAEIPELKKTETQTTGAENKAKKLTLPLFGAMKGGSKFKLKTGTVGKLPPKRPELPPTLMRMKDEPEVEEEEEEEEEEEKEKEEHEKKKLEDGSLSRPQPEIEPEAAVQEMRPPTDLTHFKETQTHENMSQLSEEEQNKDYQDCSKTTSLCAGPSASKNEYEKSRGELKKKKTPGPGKLPPTLSSKYPEDDPDYCVWVPPEGQSGDGRTHLNDKYGY

SEQ ID NO: 37 - protein, 581 aa, Artificial Sequence
  Synthetic Construct
MAVAGAVSGEPLVHWCTQQLRKTFGLDVSEEIIQYVLSIESAEEIREYVTDLLQGNEGKKGQFIEELITKWQKNDQELISDPLQQCFKKDEILDGQKSGDHLKRGRKKGRNRQEVPAFTEPDTTAEVKTPFDLAKAQENSNSVKKKTKFVNLYTREGQDRLAVLLPGRHPCDCLGQKHKLINNCLICGRIVCEQEGSGPCLFCGTLVCTHEEQDILQRDSNKSQKLLKKLMSGVENSGKVDISTKDLLPHQELRIKSGLEKAIKHKDKLLEFDRTSIRRTQVIDDESDYFASDSNQWLSKLERETLQKREEELRELRHASRLSKKVTIDFAGRKILEEENSLAEYHSRLDETIQAIANGTLNQPLTKLDRSSEEPLGVLVNPNMYQSPPQWVDHTGAASQKKAFRSSGFGLEFNSFQHQLRIQDQEFQEGFDGGWCLSVHQPWASLLVRGIKRVEGRSWYTPHRGRLWIAATAKKPSPQEVSELQATYRLLRGKDVEFPNDYPSGCLLGCVDLIDCLSQKQFKEQFPDISQESDSPFVFICKNPQEMVVKFPIKGNPKIWKLDSKIHQGAKKGLMKQNKAV

SEQ ID NO: 38 - protein, 235 aa, Artificial Sequence
  Synthetic Construct
MLSATRRACQLLLLHSLFPVPRMGNSASNIVSPQEALPGRKEQTPVAAKHHVNGNRTVEPFPEGTQMAVFGMGCFWGAERKFWVLKGVYSTQVGFAGGYTSNPTYKEVCSEKTGHAEVVRVVYQPEHMSFEELLKVFWENHDPTQGMRQGNDHGTQYRSAIYPTSAKQMEAALSSKENYQKVLSEHGFGPITTDIREGQTFYYAEDYHQQYLSKNPNGYCGLGGTGVSCPVGIKK

SEQ ID NO: 39 - protein, 307 aa, Artificial Sequence
  Synthetic Construct
MAALLLGAVLLVAQPQLVPSRPAELGQQELLRKAGTLQDDVRDGVAPNGSAQQLPQTIIIGVRKGGTRALLEMLSLHPDVAAAENEVHFFDWEEHYSHGLGWYLSQMPFSWPHQLTVEKTPAYFTSPKVPERVYSMNPSIRLLLILRDPSERVLSDYTQVFYNHMQKHKPYPSIEEFLVRDGRLNVDYKALNRSLYHVHMQNWLRFFPLRHIHIVDGDRLIRDPFPEIQKVERFLKLSPQINASNFYFNKTKGFYCLRDSGRDRCLHESKGRAHPQVDPKLLNKLHEYFHEPNKKFFELVGRTFDWH

SEQ ID NO: 40 - protein, 707 aa, Artificial Sequence
  Synthetic Construct
MTTFKEAMTFKDVAVVFTEEELGLLDLAQRKLYRDVMLENFRNLLSVGHQAFHRDTFHFLREEKIWMMKTAIQREGNSGDKIQTEMETVSEAGTHQEWSFQQIWEKIASDLTRSQDLMINSSQFSKEGDFPCQTEAGLSVIHTRQKSSQGNGYKPSFSDVSHFDFHQQLHSGEKSHTCDECGKNFCYISALRIHQRVHMGEKCYKCDVCGKEFSQSSHLQTHQRVHTGEKPFKCVECGKGFSRRSALNVHHKLHTGEKPYNCEECGKAFIHDSQLQEHQRIHTGEKPFKCDICGKSFCGRSRLNRHSMVHTAEKPFRCDTCDKSFRQRSALNSHRMIHTGEKPYKCEECGKGFICRRDLYTHHMVHTGEKPYNCKECGKSFRWASCLLKHQRVHSGEKPFKCEECGKGFYTNSQCYSHQRSHSGEKPYKCVECGKGYKRRLDLDFHQRVHTGEKLYNCKECGKSFSRAPCLLKHERLHSGEKPFQCEECGKRFTQNSHLHSHQRVHTGEKPYKCEKCGKGYNSKFNLDMHQKVHTGERPYNCKECGKSFGWASCLLKHQRLHSGEKPFKCEECGKRFTQNSQLHSHQRVHTGEKPYKCDECGKGFSWSSTRLTHQRRHSRETPLKCEQHGKNIVQNSFSKVQEKVHSVEKPYKCEDCGKGYNRRLNLDMHQRVHMGEKTWKCRECDMCFSQASSLRLHQNVHVGEKP

SEQ ID NO: 41 - protein, 939 aa, Artificial Sequence
  Synthetic Construct
MPAVSKGDGMRGLAVFISDIRNCKSKEAEIKRINKELANIRSKFKGDKALDGYSKKKYVCKLLFIFLLGHDIDFGHMEAVNLLSSNRYTEKQIGYLFISVLVNSNSELIRLINNAIKNDLASRNPTFMGLALHCIASVGSREMAEAFAGEIPKVLVAGDTMDSVKQSAALCLLRLYRTSPDLVPMGDWTSRVVHLLNDQHLGVVTAATSLITTLAQKNPEEFKTSVSLAVSRLSRIVTSASTDLQDYTYYFVPAPWLSVKLLRLLQCYPPPDPAVRGRLTECLETILNKAQEPPKSKKVQHSNAKNAVLFEAISLIIHHDSEPNLLVRACNQLGQFLQHRETNLRYLALESMCTLASSEFSHEAVKTHIETVINALKTERDVSVRQRAVDLLYAMCDRSNAPQIVAEMLSYLETADYSIREEIVLKVAILAEKYAVDYTWYVDTILNLIRIAGDYVSEEVWYRVIQIVINRDDVQGYAAKTVFEALQAPACHENLVKVGGYILGEFGNLIAGDPRSSPLIQFHLLHSKFHLCSVPTRALLLSTYIKFVNLFPEVKPTIQDVLRSDSQLRNADVELQQRAVEYLRLSTVASTDILATVLEEMPPFPERESSILAKLKKKKGPSTVTDLEDTKRDRSVDVNGGPEPAPASTSAVSTPSPSADLLGLGAAPPAPAGPPPSSGGSGLLVDVFSDSASVVAPLAPGSEDNFARFVCKNNGVLFENQLLQIGLKSEFRQNLGRMFIFYGNKTSTQFLNFTPTLICSDDLQPNLNLQTKPVDPTVEGGAQVQQVVNIECVSDFTEAPVLNIQFRYGGTFQNVSVQLPITLNKFFQPTEMASQDFFQRWKQLSNPQQEVQNIFKAKHPMDTEVTKAKIIGFGSALLEEVDPNPANFVGAGIIHTKTTQIGCLLRLEPNLQAQMYRLTLRTSKEAVSQRLCELLSAQF

SEQ ID NO: 42 - protein, 736 aa, Artificial Sequence
  Synthetic Construct
MEALIPVINKLQDVFNTVGADIIQLPQIVVVGTQSSGKSSVLESLVGRDLLPRGTGIVTRRPLILQLVHVSQEDKRKTTGEENGVEAEEWGKFLHTKNKLYTDFDEIRQEIENETERISGNNKGVSPEPIHLKIFSPNVVNLTLVDLPGMTKVPVGDQPKDIELQIRELILRFISNPNSIILAVTAANTDMATSEALKISREVDPDGRRTLAVITKLDLMDAGTDAMDVLMGRVIPVKLGIIGVVNRSQLDINNKKSVTDSIRDEYAFLQKKYPSLANRNGTKYLARTLNRLLMHHIRDCLPELKTRINVLAAQYQSLLNSYGEPVDDKSATLLQLITKFATEYCNTIEGTAKYIETSELCGGARICYIFHETFGRTLESVDPLGGLNTIDILTAIRNATGPRPALFVPEVSFELLVKRQIKRLEEPSLRCVELVHEEMQRIIQHCSNYSTQELLRFPKLHDAIVEVVTCLLRKRLPVTNEMVHNLVAIELAYINTKHPDFADACGLMNNNIEEQRRNRLARELPSAVSRDKSSKVPSALAPASQEPSPAASAEADGKLIQDSRRETKNVASGGGGVGDGVQEPTTGNWRGMLKTSKAEELLAEEKSKPIPIMPASPQKGHAVNLLDVPVPVARKLSAREQRDCEVIERLIKSYFLIVRKNIQDSVPKAVMHFLVNHVKDTLQSELVGQLYKSSLLDDLLTESEDMAQRRKEAADMLKALQGASQIIAEIRETHLW

SEQ ID NO: 43 - protein, 478 aa, Artificial Sequence
  Synthetic Construct
MGRCCFYTAGTLSLLLLVTSVTLLVARVFQKAVDQSIEKKIVLRNGTEAFDSWEKPPLPVYTQFYFFNVTNPEEILRGETPRVEEVGPYTYRELRNKANIQFGDNGTTISAVSNKAYVFERDQSVGDPKIDLIRTLNIPVLTVIEWSQVHFLREIIEAMLKAYQQKLFVTHTVDELLWGYKDEILSLIHVFRPDISPYFGLFYEKNGTNDGDYVFLTGEDSYLNFTKIVEWNGKTSLDWWITDKCNMINGTDGDSFHPLITKDEVLYVFPSDFCRSVYITFSDYESVQGLPAFRYKVPAEILANTSDNAGFCIPEGNCLGSGVLNVSICKNGAPIIMSFPHFYQADERFVSAIEGMHPNQEDHETFVDINPLTGIILKAAKRFQINIYVKKLDDFVETGDIRTMVFPVMYLNESVHIDKETASRLKSMINTTLIITNIPYIIMALGVFFGLVFTWLACKGQGSMDEGTADERAPLIRT

SEQ ID NO: 44 - protein, 572 aa, Artificial Sequence
  Synthetic Construct
MECLYYFLGFLLLAARLPLDAAKRFHDVLGNERPSAYMREHNQLNGWSSDENDWNEKLYPVWKRGDMRWKNSWKGGRVQAVLTSDSPALVGSNITFAVNLIFPRCQKEDANGNIVYEKNCRNEAGLSADPYVYNWTAWSEDSDGENGTGQSHHNVFPDGKPFPHHPGWRRWNFIYVFHTLGQYFQKLGRCSVRVSVNTANVTLGPQLMEVTVYRRHGRAYVPIAQVKDVYVVTDQIPVFVTMFQKNDRNSSDETFLKDLPIMFDVLIHDPSHFLNYSTINYKWSFGDNTGLFVSTNHTVNHTYVLNGTFSLNLTVKAAAPGPCPPPPPPPRPSKPTPSLATTLKSYDSNTPGPAGDNPLELSRIPDENCQINRYGHFQATITIVEGILEVNIIQMTDVLMPVPWPESSLIDFVVTCQGSIPTEVCTIISDPTCEITQNTVCSPVDVDEMCLLTVRRTFNGSGTYCVNLTLGDDTSLALTSTLISVPDRDPASPLRMANSALISVGCLAIFVTVISLLVYKKHKEYNPIENSPGNVVRSKGLSVFLNRAKAVFFPGNQEKDPLLKNQEFKGVS

SEQ ID NO: 45 - protein, 796 aa, Artificial Sequence
  Synthetic Construct
MPTTQQSPQDEQEKLLDEAIQAVKVQSFQMKRCLDKNKLMDALKHASNMLGELRTSMLSPKSYYELYMAISDELHYLEVYLTDEFAKGRKVADLYELVQYAGNIIPRLYLLITVGVVYVKSFPQSRKDILKDLVEMCRGVQHPLRGLFLRNYLLQCTRNILPDEGEPTDEETTGDISDSMDFVLLNFAEMNKLWVRMQHQGHSRDREKRERERQELRILVGTNLVRLSQLEGVNVERYKQIVLTGILEQVVNCRDALAQEYLMECIIQVFPDEFHLQTLNPFLRACAELHQNVNVKNIIIALIDRLALFAHREDGPGIPADIKLFDIFSQQVATVIQSRQDMPSEDVVSLQVSLINLAMKCYPDRVDYVDKVLETTVEIFNKLNLEHIATSSAVSKELTRLLKIPVDTYNNILTVLKLKHFHPLFEYFDYESRKSMSCYVLSNVLDYNTEIVSQDQVDSIMNLVSTLIQDQPDQPVEDPDPEDFADEQSLVGRFIHLLRSEDPDQQYLILNTARKHFGAGGNQRIRFTLPPLVFAAYQLAFRYKENSKVDDKWEKKCQKIFSFAHQTISALIKAELAELPLRLFLQGALAAGEIGFENHETVAYEFMSQAFSLYEDEISDSKAQLAAITLIIGTFERMKCFSEENHEPLRTQCALAASKLLKKPDQGRAVSTCAHLFWSGRNTDKNGEELHGGKRVMECLKKALKIANQCMDPSLQVQLFIEILNRYIYFYEKENDAVTIQVLNQLIQKIREDLPNLESSEETEQINKHFHNTLEHLRLRRESPESEGPIYEGLIL

SEQ ID NO: 46 - protein, 522 aa, Artificial Sequence
  Synthetic Construct
MRLRVRLLKRTWPLEVPETEPTLGHLRSHLRQSLLCTWGYSSNTRFTITLNYKDPLTGDEETLASYGIVSGDLICLILQDDIPAPNIPSSTDSEHSSLQNNEQPSLATSSNQTSMQDEQPSDSFQGQAAQSGVWNDDSMLGPSQNFEAESIQDNAHMAEGTGFYPSEPMLCSESVEGQVPHSLETLYQSADCSDANDALIVLIHLLMLESGYIPQGTEAKALSMPEKWKLSGVYKLQYMHPLCEGSSATLTCVPLGNLIVVNATLKINNEIRSVKRLQLLPESFICKEKLGENVANIYKDLQKLSRLFKDQLVYPLLAFTRQALNLPDVFGLVVLPLELKLRIFRLLDVRSVLSLSAVCRDLFTASNDPLLWRFLYLRDFRDNTVRVQDTDWKELYRKRHIQRKESPKGRFVMLLPSSTHTIPFYPNPLHPRPFPSSRLPPGIIGGEYDQRPTLPYVGDPISSLIPGPGETPSQFPPLRPRFDPVGPLPGPNPILPGRGGPNDRFPFRPSRGRPTDGRLSFM

SEQ ID NO: 47 - protein, 189 aa, Artificial Sequence
  Synthetic Construct
MASKRALVILAKGAEEMETVIPVDVMRRAGIKVTVAGLAGKDPVQCSRDVVICPDASLEDAKKEGPYDVVVLPGGNLGAQNLSESAAVKEILKEQENRKGLIAAICAGPTALLAHEIGFGSKVTTHPLAKDKMMNGGHYTYSENRVEKDGLILTSRGPGTSFEFALAIVEALNGKEVAAQVKAPLVLKD

SEQ ID NO: 48 - protein, 1132 aa, Artificial Sequence
  Synthetic Construct
MELFQAKDHYILQQGERALWCSRRDGGLQLRPATDLLLAWNPICLGLVEGVIGKIQLHSDLPWWLILIRQKALVGKLPGDHEVCKVTKIAVLSLSEMEPQDLELELCKKHHFGINKPEKIIPSPDDSKFLLKTFTHIKSNVSAPNKKKVKESKEKEKLERRLLEELLKMFMDSESFYYSLTYDLTNSVQRQSTGERDGRPLWQKVDDRFFWNKYMIQDLTEIGTPDVDFWIIPMIQGFVQIEELVVNYTESSDDEKSSPETPPQESTCVDDIHPRFLVALISRRSRHRAGMRYKRRGVDKNGNVANYVETEQLIHVHNHTLSFVQTRGSVPVFWSQVGYRYNPRPRLDRSEKETVAYFCAHFEEQLNIYKKQVIINLVDQAGREKIIGDAYLKQVLLFNNSHLTYVSFDFHEHCRGMKFENVQTLTDAIYDIILDMKWCWVDEAGVICKQEGIFRVNCMDCLDRTNVVQAAIARVVMEQQLKKLGVMPPEQPLPVKCNRIYQIMWANNGDSISRQYAGTAALKGDFTRTGERKLAGVMKDGVNSANRYYLNRFKDAYRQAVIDLMQGIPVTEDLYSIFTKEKEHEALHKENQRSHQELISQLLQSYMKLLLPDDEKFHGGWALIDCDPSLIDATHRDVDVLLLLSNSAYYVAYYDDEVDKVNQYQRLSLENLEKIEIGPEPTLFGKPKFSCMRLHYRYKEASGYFHTLRAVMRNPEEDGKDTLQCIAEMLQITKQAMGSDLPIIEKKLERKSSKPHEDIIGIRSQNQGSLAQGKNFLMSKFSSLNQKVKQTKSNVNIGNLRKLGNFTKPEMKVNFLKPNLKVNLWKSDSSLETMENTGVMDKVQAESDGDMSSDNDSYHSDEFLTNSKSDEDRQLANSLESVGPIDYVLPSCGIIASAPRLGSRSQSLSSTDSSVHAPSEITVAHGSGLGKGQESPLKKSPSAGDVHILTGFAKPMDIYCHRFVQDAQNKVTHLSETRSVSQQASQERNQMTNQVSNETQSESTEQTPSRPSQLDVSLSATGPQFLSVEPAHSVASQKTPTSASSMLELETGLHVTPSPSESSSSRAVSPFAKIRSSMVQVASITQAGLTHGINFAVSKVQKSPPEPEIINQVQQNELKKMFIQCQTRIIQI

SEQ ID NO: 49 - protein, 2243 aa, Artificial Sequence
  Synthetic Construct
MNIIRENKDLACFYTTKHSWRGKYKRVFSVGTHAITTYNPNTLEVTNQWPYGDICSISPVGKGQGTEFNLTFRKGSGKKSETLKFSTEHRTELLTEALRFRTDFSEGKITGRRYNCYKHHWSDSRKPVILEVTPGGFDQINPATNRVLCSYDYRNIEGFVDLSDYQGGFCILYGGFSRLHLFASEQREEIIKSAIDHAGNYIGISLRIRKEPLEFEQYLNLRFGKYSTDESITSLAEFVVQKISPRHSEPVKRVLALTETCLVERDPATYNIATLKPLGEVFALVCDSENPQLFTIEFIKGQVRKYSSTERDSLLASLLDGVRASGNRDVCVKMTPTHKGQRWGLLSMPVDEEVESLHLRFLATPPNGNFADAVFRFNANISYSGVLHAVTQDGLFSENKEKLINNAITALLSQEGDVVASNAELESQFQAVRRLVASKAGFLAFTQLPKFRERLGVKVVKALKRSNNGIIHAAVDMLCALMCPMHDDYDLRQEQLNKASLLSSKKFLENLLEKFNSHVDHGTGALVISSLLDFLTFALCAPYSETTEGQQFDMLLEMVASNGRTLFKLFQHPSMAIIKGAGLVMKAIIEEGDKEIATKMQELALSEGALPRHLHTAMFTISSDQRMLTNRQLSRHLVGLWTADNATATNLLKRILPPGLLAYLESSDLVPEKDADRMHVRDNVKIAMDQYGKFNKVPEWQRLAGKAAKEVEKFAKEKVDLVLMHWRDRMGIAQKENINQKPVVLRKRRQRIKIEANWDLFYYRFGQDHARSNLIWNFKTREELKDTLESEMRAFNIDRELGSANVISWNHHEFEVKYECLAEEIKIGDYYLRLLLEEDENEESGSIKRSYEFFNELYHRFLLTPKVNMKCLCLQALAIVYGRCHEEIGPFTDTRYIIGMLERCTDKLERDRLILFLNKLILNKKNVKDLMDSNGIRILVDLLTLAHLHVSRATVPLQSNVIEAAPDMKRESEKEWYFGNADKERSGPYGFHEMQELWTKGMLNAKTRCWAQGMDGWRPLQSIPQLKWCLLASGQAVLNETDLATLILNMLITMCGYFPSRDQDNAIIRPLPKVKRLLSDSTCLPHIIQLLLTFDPILVEKVAILLYHIMQDNPQLPRLYLSGVFFFIMMYTGSNVLPVARFLKYTHTKQAFKSEETKGQDIFQRSILGHILPEAMVCYLENYEPEKFSEIFLGEFDTPEAIWSSEMRRLMIEKIAAHLADFTPRLQSNTRALYQYCPIPIINYPQLENELFCNIYYLKQLCDTLRFPDWPIKDPVKLLKDTLDAWKKEVEKKPPMMSIDDAYEVLNLPQGQGPHDESKIRKAYFRLAQKYHPDKNPEGRDMFEKVNKAYEFLCTKSAKIVDGPDPENIILILKTQSILFNRHKEDLQPYKYAGYPMLIRTITMETSDDLLFSKESPLLPAATELAFHTVNCSALNAEELRRENGLEVLQEAFSRCVAVLTRASKPSDMSVQVCGYISKCYSVAAQFEECREKITEMPSIIKDLCRVLYFGKSIPRVAALGVECVSSFAVDFWLQTHLFQAGILWYLLGFLFNYDYTLEESGIQKSEETNQQEVANSLAKLSVHALSRLGGYLAEEQATPENPTIRKSLAGMLTPYVARKLAVASVTEILKMLNSNTESPYLIWNNSTRAELLEFLESQQENMIKKGDCDKTYGSEFVYSDHAKELIVGEIFVRVYNEVPTFQLEVPKAFAASLLDYIGSQAQYLHTFMAITHAAKVESEQHGDRLPRVEMALEALRNVIKYNPGSESECIGHFKLIFSLLRVHGAGQVQQLALEVVNIVTSNQDCVNNIAESMVLSSLLALLHSLPSSRQLVLETLYALTSSTKIIKEAMAKGALIYLLDMFCNSTHPQVRAQTAELFAKMTADKLIGPKVRITLMKFLPSVFMDAMRDNPEAAVHIFEGTHENPELIWNDNSRDKVSTTVREMMLEHFKNQQDNPEANWKLPEDFAVVFGEAEGELAVGGVFLRIFIAQPAWVLRKPREFLIALLEKLTELLEKNNPHGETLETLTMATVCLFSAQPQLADQVPPLGHLPKVIQAMNHRNNAIPKSAIRVIHALSENELCVRAMASLETIGPLMNGMKKRADTVGLACEAINRMFQKEQSELVAQALKADLVPYLLKLLEGIGLENLDSPAATKAQIVKALKAMTRSLQYGEQVNEILCRSSVWSAFKDQKHDLFISESQTAGYLTGPGVAGYLTAGTSTSVMSNLPPPVDHEAGDLGYQT

SEQ ID NO: 50 - protein, 250 aa, Artificial Sequence
  Synthetic Construct
MEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATHMCMVMRGVQKMNSKTVTSTMLGVFREDPKTREEFLTLIRS

SEQ ID NO: 51 - protein, 503 aa, Artificial Sequence
  Synthetic Construct
MEYMAESTDRSPGHILCCECGVPISPNPANICVACLRSKVDISQGIPKQVSISFCKQCQRYFQPPGTWIQCALESRELLALCLKKIKAPLSKVRLVDAGFVWTEPHSKRLKVKLTIQKEVMNGAILQQVFVVDYVVQSQMCGDCHRVEAKDFWKAVIQVRQKTLHKKTFYYLEQLILKYGMHQNTLRIKEIHDGLDFYYSSKQHAQKMVEFLQCTVPCRYKASQRLISQDIHSNTYNYKSTFSVEIVPICKDNVVCLSPKLAQSLGNMNQICVCIRVTSAIHLIDPNTLQVADIDGSTFWSHPFNSLCHPKQLEEFIVMECSIVQDIKRAAGAGMISKKHTLGEVWVQKTSEMNTDKQYFCRTHLGHLLNPGDLVLGFDLANCNLNDEHVNKMNSDRVPDVVLIKKSYDRTKRQRRRNWKLKELARERENMDTDDERQYQDFLEDLEEDEAIRKNVNIYRDSAIPVESDTDDEGAPRISLAEMLEDLHISQDATGEEGASMLT

SEQ ID NO: 52 - protein, 1055 aa, Artificial Sequence
  Synthetic Construct
MTVEQNVLQQSAAQKHQQTFLNQLREITGINDTQILQQALKDSNGNLELAVAFLTAKNAKTPQQEETTYYQTALPGNDRYISVGSQADTNVIDLTGDDKDDLQRAIALSLAESNRAFRETGITDEEQAISRVLEASIAENKACLKRTPTEVWRDSRNPYDRKRQDKAPVGLKNVGNTCWFSAVIQSLFNLLEFRRLVLNYKPPSNAQDLPRNQKEHRNLPFMRELRYLFALLVGTKRKYVDPSRAVEILKDAFKSNDSQQQDVSEFTHKLLDWLEDAFQMKAEEETDEEKPKNPMVELFYGRFLAVGVLEGKKFENTEMFGQYPLQVNGFKDLHECLEAAMIEGEIESLHSENSGKSGQEHWFTELPPVLTFELSRFEFNQALGRPEKIHNKLEFPQVLYLDRYMHRNREITRIKREEIKRLKDYLTVLQQRLERYLSYGSGPKRFPLVDVLQYALEFASSKPVCTSPVDDIDASSPPSGSIPSQTLPSTTEQQGALSSELPSTSPSSVAAISSRSVIHKPFTQSRIPPDLPMHPAPRHITEEELSVLESCLHRWRTEIENDTRDLQESISRIHRTIELMYSDKSMIQVPYRLHAVLVHEGQANAGHYWAYIFDHRESRWMKYNDIAVTKSSWEELVRDSFGGYRNASAYCLMYINDKAQFLIQEEFNKETGQPLVGIETLPPDLRDFVEEDNQRFEKELEEWDAQLAQKALQEKLLASQKLRESETSVTTAQAAGDPEYLEQPSRSDFSKHLKEETIQIITKASHEHEDKSPETVLQSAIKLEYARLVKLAQEDTPPETDYRLHHVVVYFIQNQAPKKIIEKTLLEQFGDRNLSFDERCHNIMKVAQAKLEMIKPEEVNLEEYEEWHQDYRKFRETTMYLIIGLENFQRESYIDSLLFLICAYQNNKELLSKGLYRGHDEELISHYRRECLLKLNEQAAELFESGEDREVNNGLIIMNEFIVPFLPLLLVDEMEEKDILAVEDMRNRWCSYLGQEMEPHLQEKLTDFLPKLLDCSMEIKSFHEPPKLPSYSTHELCERFARIMLSLSRTPADGR

SEQ ID NO: 53 - protein, 203 aa, Artificial Sequence
  Synthetic Construct
MGSRDHLFKVLVVGDAAVGKTSLVQRYSQDSFSKHYKSTVGVDFALKVLQWSDYEIVRLQLWDIAGQERFTSMTRLYYRDASACVIMFDVTNATTFSNSQRWKQDLDSKLTLPNGEPVPCLLLANKCDLSPWAVSRDQIDRFSKENGFTGWTETSVKENKNINEAMRVLIEKMMRNSTEDIMSLSTQGDYINLQTKSSSWSCC

SEQ ID NO: 54 - protein, 1722 aa, Artificial Sequence
  Synthetic Construct
MSDPRQSQEEKHKLGRASSKFKDPPRIMQSDDYFARKFKAINGNMGPTTSLNASNSNETGGGGPANGTPAVPKMGVRARVSEWPPKKDCSKELTCKALWESRSQTSYESITSVLQNGQSDQSEGQQDEQLDLDFVEAKYTIGDIFVHSPQRGLHPIRQRSNSDVTISDIDAEDVLDQNAVNPNTGAALHREYGSTSSIDRQGLSGENFFAMLRGYRVENYDHKAMVPFGFPEFFRCDPAISPSLHAAAQISRGEFVRISGLDYVDSALLMGRDRDKPFKRRLKSESVETSLFRKLRTVKSEHETFKFTSELEESRLERGIRPWNCQRCFAHYDVQSILFNINEAMATRANVGKRKNITTGASAASQTQMPTGQTGNCESPLGSKEDLNSKENLDADEGDGKSNDLVLSCPYFRNETGGEGDRRIALSRANSSSFSSGESCSFESSLSSHCTNAGVSVLEVPRENQPIHREKVKRYIIEHIDLGAYYYRKFFYGKEHQNYFGIDENLGPVAVSIRREKVEDAKEKEGSQFNYRVAFRTSELTTLRGAILEDAIPSTARHGTARGLPLKEVLEYVIPELSIQCLRQASNSPKVSEQLLKLDEQGLSFQHKIGILYCKAGQSTEEEMYNNETAGPAFEEFLDLLGQRVRLKGFSKYRAQLDNKTDSTGTHSLYTTYKDYELMFHVSTLLPYMPNNRQQLLRKRHIGNDIVTIVFQEPGALPFTPKSIRSHFQHVFVIVKVHNPCTENVCYSVGVSRSKDVPPFGPPIPKGVTFPKSAVFRDFLLAKVINAENAAHKSEKFRAMATRTRQEYLKDLAENFVTTATVDTSVKFSFITLGAKKKEKVKPRKDAHLFSIGAIMWHVIARDFGQSADIECLLGISNEFIMLIEKDSKNVVFNCSCRDVIGWTSGLVSIKVFYERGECVLLSSVDNCAEDIREIVQRLVIVTRGCETVEMTLRRNGLGQLGFHVNFEGIVADVEPFGFAWKAGLRQGSRLVEICKVAVATLTHEQMIDLLRTSVTVKVVIIQPHDDGSPRRGCSELCRIPMVEYKLDSEGTPCEYKTPFRRNTTWHRVPTPALQPLSRASPIPGTPDRLPCQQLLQQAQAAIPRSTSFDRKLPDGTRSSPSNQSSSSDPGPGGSGPWRPQVGYDGCQSPLLLEHQGSGPLECDGAREREDTMEASRHPETKWHGPPSKVLGSYKERALQKDGSCKDSPNKLSHIGDKSCSSHSSSNTLSSNTSSNSDDKHFGSGDLMDPELLGLTYIKGASTDSGIDTAPCMPATILGPVHLAGSRSLIHSRAEQWADAADVSGPDDEPAKLYSVHGYASTISAGSAAEGSMGDLSEISSHSSGSHHSGSPSAHCSKSSGSLDSSKVYIVSHSSGQQVPGSMSKPYHRQGAVNKYVIGWKKSEGSPPPEEPEVTECPGMYSEMDVMSTATQHQTVVGDAVAETQHVLSKEDFLKLMLPDSPLVEEGRRKFSFYGNLSPRRSLYRTLSDESICSNRRGSSFGSSRSSVLDQALPNDILFSTTPPYHSTLPPRAHPAPSMGSLRNEFWFSDGSLSDKSKCADPGLMPLPDTATGLDWTHLVDAARAFEGLDSDEELGLLCHHTSYLDQRVASFCTLTDMQHGQDLEGAQELPLCVDPGSGKEFMDTTGERSPSPLTGKVNQLELILRQLQTDLRKEKQDKAVLQAEVQHLRQDNMRLQEESQTATAQLRKFTEWFFTTIDKKS

SEQ ID NO: 55 - protein, 725 aa, Artificial Sequence
  Synthetic Construct
MAAASAVSVLLVAAERNRWHRLPSLLLPPRTWVWRQRTMKYTTATGRNITKVLIANRGEIACRVMRTAKKLGVQTVAVYSEADRNSMHVDMADEAYSIGPAPSQQSYLSMEKIIQVAKTSAAQAIHPGCGFLSENMEFAELCKQEGIIFIGPPPSAIRDMGIKSTSKSIMAAAGVPVVEGYHGEDQSDQCLKEHARRIGYPVMIKAVRGGGGKGMRIVRSEQEFQEQLESARREAKKSFNDDAMLIEKFVDTPRHVEVQVFGDHHGNAVYLFERDCSVQRRHQKIIEEAPAPGIKSEVRKKLGEAAVRAAKAVNYVGAGTVEFIMDSKHNFCFMEMNTRLQVEHPVTEMITGTDLVEWQLRIAAGEKIPLSQEEITLQGHAFEARIYAEDPSNNFMPVAGPLVHLSTPRADPSTRIETGVRQGDEVSVHYDPMIAKLVVWAADRQAALTKLRYSLRQYNIVGLHTNIDFLLNLSGHPEFEAGNVHTDFIPQHHKQLLLSRKAAAKESLCQAALGLILKEKAMTDTFTLQAHDQFSPFSSSSGRRLNISYTRNMTLKDGKNNVAIAVTYNHDGSYSMQIEDKTFQVLGNLYSEGDCTYLKCSVNGVASKAKLIILENTIYLFSKEGSIEIDIPVPKYLSSVSSQETQGGPLAPMTGTIEKVFVKAGDKVKAGDSLMVMIAMKMEHTIKSPKDGTVKKVFYREGAQANRHTPLVEFEEEESDKRESE

SEQ ID NO: 56 - protein, 1573 aa, Artificial Sequence
  Synthetic Construct
MAFSKGFRIYHKLDPPPFSLIVETRHKEECLMFESGAVAVLSSAEKEAIKGTYSKVLDAYGLLGVLRLNLGDTMLHYLVLVTGCMSVGKIQESEVFRVTSTEFISLRIDSSDEDRISEVRKVLNSGNFYFAWSASGISLDLSLNAHRSMQEQTTDNRFFWNQSLHLHLKHYGVNCDDWLLRLMCGGVEIRTIYAAHKQAKACLISRLSCERAGTRFNVRGTNDDGHVANFVETEQVVYLDDSVSSFIQIRGSVPLFWEQPGLQVGSHRVRMSRGFEANAPAFDRHFRTLKNLYGKQIIVNLLGSKEGEHMLSKAFQSHLKASEHAADIQMVNFDYHQMVKGGKAEKLHSVLKPQVQKFLDYGFFYFNGSEVQRCQSGTVRTNCLDCLDRTNSVQAFLGLEMLAKQLEALGLAEKPQLVTRFQEVFRSMWSVNGDSISKIYAGTGALEGKAKLKDGARSVTRTIQNNFFDSSKQEAIDVLLLGNTLNSDLADKARALLTTGSLRVSEQTLQSASSKVLKSMCENFYKYSKPKKIRVCVGTWNVNGGKQFRSIAFKNQTLTDWLLDAPKLAGIQEFQDKRSKPTDIFAIGFEEMVELNAGNIVSASTTNQKLWAVELQKTISRDNKYVLLASEQLVGVCLFVFIRPQHAPFIRDVAVDTVKTGMGGATGNKGAVAIRMLFHTTSLCFVCSHFAAGQSQVKERNEDFIEIARKLSFPMGRMLFSHDYVFWCGDFNYRIDLPNEEVKELIRQQNWDSLIAGDQLINQKNAGQVFRGFLEGKVTFAPTYKYDLFSDDYDTSEKCRTPAWTDRVLWRRRKWPFDRSAEDLDLLNASFQDESKILYTWTPGTLLHYGRAELKTSDHRPVVALIDIDIFEVEAEERQNIYKEVIAVQGPPDGTVLVSIKSSLPENNFFDDALIDELLQQFASFGEVILIRFVEDKMWVTFLEGSSALNVLSLNGKELLNRTITIALKSPDWIKNLEEEMSLEKISIALPSSTSSTLLGEDAEVAADFDMEGDVDDYSAEVEELLPQHLQPSSSSGLGTSPSSSPRTSPCQSPTISEGPVPSLPIRPSRAPSRTPGPPSAQSSPIDAQPATPLPQKDPAQPLEPKRPPPPRPVAPPTRPAPPQRPPPPSGARSPAPTRKEFGGIGAPPSPGVARREMEAPKSPGTTRKDNIGRSQPSPQAGLAGPGPAGYSTARPTIPPRAGVISAPQSHARASAGRLTPESQSKTSETSKGSTFLPEPLKPQAAFPPQSSLPPPAQRLQEPLVPVAAPMPQSGPQPNLETPPQPPPRSRSSHSLPSEASSQPQVKTNGISDGKRESPLKIDPFEDLSFNLLAVSKAQLSVQTSPVPTPDPKRLIQLPSATQSNVLSSVSCMPTMPPIPARSQSQENMRSSPNPFITGLTRTNPFSDRTAAPGNPFRAKSEESEATSWFSKEEPVTISPFPSLQPLGHNKSRASSSLDGFKDSFDLQGQSTLKISNPKGWVTFEEEEDFGVKGKSKSACSDLLGNQPSSFSGSNLTLNDDWNKGTNVSFCVLPSRRPPPPPVPLLPPGTSPPVDPFTTLASKASPTLDFTER

SEQ ID NO: 57 - protein, 2527 aa, Artificial Sequence
  Synthetic Construct
MASGSCQGCEEDEETLKKLIVRLNNVQEGKQIETLVQILEDLLVFTYSERASKLFQGKNIHVPLLIVLDSYMRVASVQQVGWSLLCKLIEVCPGTMQSLMGPQDVGNDWEVLGVHQLILKMLTVHNASVNLSVIGLKTLDLLLTSGKITLLILDEESDIFMLIFDAMHSFPANDEVQKLGCKALHVLFERVSEEQLTEFVENKDYMILLSALTNFKDEEEIVLHVLHCLHSLAIPCNNVEVLMSGNVRCYNIVVEAMKAFPMSERIQEVSCCLLHRLTLGNFFNILVLNEVHEFVVKAVQQYPENAALQISALSCLALLTETIFLNQDLEEKNENQENDDEGEEDKLFWLEACYKALTWHRKNKHVQEAACWALNNLLMYQNSLHEKIGDEDGHFPAHREVMLSMLMHSSSKEVFQASANALSTLLEQNVNFRKILLSKGIHLNVLELMQKHIHSPEVAESGCKMLNHLFEGSNTSLDIMAAVVPKILTVMKRHETSLPVQLEALRAILHFIVPGMPEESREDTEFHHKLNMVKKQCFKNDIHKLVLAALNRFIGNPGIQKCGLKVISSIVHFPDALEMLSLEGAMDSVLHTLQMYPDDQEIQCLGLSLIGYLITKKNVFIGTGHLLAKILVSSLYRFKDVAEIQTKGFQTILAILKLSASFSKLLVHHSFDLVIFHQMSSNIMEQKDQQFLNLCCKCFAKVAMDDYLKNVMLERACDQNNSIMVECLLLLGADANQAKEGSSLICQVCEKESSPKLVELLLNSGSREQDVRKALTISIGKGDSQIISLLLRRLALDVANNSICLGGFCIGKVEPSWLGPLFPDKTSNLRKQTNIASTLARMVIRYQMKSAVEEGTASGSDGNFSEDVLSKFDEWTFIPDSSMDSVFAQSDDLDSEGSEGSFLVKKKSNSISVGEFYRDAVLQRCSPNLQRHSNSLGPIFDHEDLLKRKRKILSSDDSLRSSKLQSHMRHSDSISSLASEREYITSLDLSANELRDIDALSQKCCISVHLEHLEKLELHQNALTSFPQQLCETLKSLTHLDLHSNKFTSFPSYLLKMSCIANLDVSRNDIGPSVVLDPTVKCPTLKQFNLSYNQLSFVPENLTDVVEKLEQLILEGNKISGICSPLRLKELKILNLSKNHISSLSENFLEACPKVESFSARMNFLAAMPFLPPSMTILKLSQNKFSCIPEAILNLPHLRSLDMSSNDIQYLPGPAHWKSLNLRELLFSHNQISILDLSEKAYLWSRVEKLHLSHNKLKEIPPEIGCLENLTSLDVSYNLELRSFPNEMGKLSKIWDLPLDELHLNFDFKHIGCKAKDIIRFLQQRLKKAVPYNRMKLMIVGNTGSGKTTLLQQLMKTKKSDLGMQSATVGIDVKDWPIQIRDKRKRDLVLNVWDFAGREEFYSTHPHFMTQRALYLAVYDLSKGQAEVDAMKPWLFNIKARASSSPVILVGTHLDVSDEKQRKACMSKITKELLNKRGFPAIRDYHFVNATEESDALAKLRKTIINESLNFKIRDQLVVGQLIPDCYVELEKIILSERKNVPIEFPVIDRKRLLQLVRENQLQLDENELPHAVHFLNESGVLLHFQDPALQLSDLYFVEPKWLCKIMAQILTVKVEGCPKHPKGIISRRDVEKFLSKKRKFPKNYMSQYFKLLEKFQIALPIGEEYLLVPSSLSDHRPVIELPHCENSEIIIRLYEMPYFPMGFWSRLINRLLEISPYMLSGRERALRPNRMYWRQGIYLNWSPEAYCLVGSEVLDNHPESFLKITVPSCRKGCILLGQVVDHIDSLMEEWFPGLLEIDICGEGETLLKKWALYSFNDGEEHQKILLDDLMKKAEEGDLLVNPDQPRLTIPISQIAPDLILADLPRNIMLNNDELEFEQAPEFLLGDGSFGSVYRAAYEGEEVAVKIFNKHTSLRLLRQELVVLCHLHHPSLISLLAAGIRPRMLVMELASKGSLDRLLQQDKASLTRTLQHRIALHVADGLRYLHSAMIIYRDLKPHNVLLFTLYPNAAIIAKIADYGIAQYCCRMGIKTSEGTPGFRAPEVARGNVIYNQQADVYSFGLLLYDILTTGGRIVEGLKFPNEFDELEIQGKLPDPVKEYGCAPWPMVEKLIKQCLKENPQERPTSAQVFDILNSAELVCLTRRILLPKNVIVECMVATHHNSRNASIWLGCGHTDRGQLSFLDLNTEGYTSEEVADSRILCLALVHLPVEKESWIVSGTQSGTLLVINTEDGKKRHTLEKMTDSVTCLYCNSFSKQSKQKNFLLVGTADGKLAIFEDKTVKLKGAAPLKILNIGNVSTPLMCLSESTNSTERNVMWGGCGTKIFSFSNDFTIQKLIETRTSQLFSYAAFSDSNIITVVVDTALYIAKQNSPVVEVWDKKTEKLCGLIDCVHFLREVMVKENKESKHKMSYSGRVKTLCLQKNTALWIGTGGGHILLLDLSTRRLIRVIYNFCNSVRVMMTAQLGSLKNVMLVLGYNRKNTEGTQKQKEIQSCLTVWDINLPHEVQNLEKHIEVRKELAEKMRRTSVE

SEQ ID NO: 58 - protein, 140 aa, Artificial Sequence
  Synthetic Construct
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 59 - protein, 140 aa, Artificial Sequence
  Synthetic Construct
MHRGVGPAFRVVRKMAASGAEPQVLVQYLVLRKDLSQAPFSWPAGALVAQACHAATAALHTHRDHPHTAAYLQELGRMRKVVLEAPDETTLKELAETLQQKNIDHMLWLEQPENIATCIALRPYPKEEVGQYLKKFRLFK

SEQ ID NO: 60 - protein, 581 aa, Artificial Sequence
  Synthetic Construct
MAVRQALGRGLQLGRALLLRFTGKPGRAYGLGRPGPAAGCVRGERPGWAAGPGAEPRRVGLGLPNRLRFFRQSVAGLAARLQRQFVVRAWGCAGPCGRAVFLAFGLGLGLIEEKQAESRRAVSACQEIQAIFTQKSKPGPDPLDTRRLQGFRLEEYLIGQSIGKGCSAAVYEATMPTLPQNLEVTKSTGLLPGRGPGTSAPGEGQERAPGAPAFPLAIKMMWNISAGSSSEAILNTMSQELVPASRVALAGEYGAVTYRKSKRGPKQLAPHPNIIRVLRAFTSSVPLLPGALVDYPDVLPSRLHPEGLGHGRTLFLVMKNYPCTLRQYLCVNTPSPRLAAMMLLQLLEGVDHLVQQGIAHRDLKSDNILVELDPDGCPWLVIADFGCCLADESIGLQLPFSSWYVDRGGNGCLMAPEVSTARPGPRAVIDYSKADAWAVGAIAYEIFGLVNPFYGQGKAHLESRSYQEAQLPALPESVPPDVRQLVRALLQREASKRPSARVAANVLHLSLWGEHILALKNLKLDKMVGWLLQQSAATLLANRLTEKCCVETKMKMLFLANLECETLCQAALLLCSWRAAL

SEQ ID NO: 61 - protein, 289 aa, Artificial Sequence
  Synthetic Construct
MEPAAGIQRRSSQGPTVPPPPRGHAPPAAAPGPAPLSSPVREPPQLEEERQVRISESGQFSDGLEDRGLLESSTRLKPHEAQNYRKKALWVSWFSIIVTLALAVAAFTVSVMRYSASAFGFAFDAILDVLSSAIVLWRYSNAAAVHSAHREYIACVILGVIFLLSSICIVVKAIHDLSTRLLPEVDDFLFSVSILSGILCSILAVLKFMLGKVLTSRALITDGFNSLVGGVMGFSILLSAEVFKHDSAVWYLDGSIGVLIGLTIFAYGVKLLIDMVPRVRQTRHYEMFE

SEQ ID NO: 62 - protein, 1311 aa, Artificial Sequence
  Synthetic Construct
MSLLQSALDFLAGPGSLGGASGRDQSDFVGQTVELGELRLRVRRVLAEGGFAFVYEAQDVGSGREYALKRLLSNEEEKNRAIIQEVCFMKKLSGHPNIVQFCSAASIGKEESDTGQAEFLLLTELCKGQLVEFLKKMESRGPLSCDTVLKIFYQTCRAVQHMHRQKPPIIHRDLKVENLLLSNQGTIKLCDFGSATTISHYPDYSWSAQRRALVEEEITRNTTPMYRTPEIIDLYSNFPIGEKQDIWALGCILYLLCFRQHPFEDGAKLRIVNGKYSIPPHDTQYTVFHSLIRAMLQVNPEERLSIAEVVHQLQEIAAARNVNPKSPITELLEQNGGYGSATLSRGPPPPVGPAGSGYSGGLALAEYDQPYGGFLDILRGGTERLFTNLKDTSSKVIQSVANYAKGDLDISYITSRIAVMSFPAEGVESALKNNIEDVRLFLDSKHPGHYAVYNLSPRTYRPSRFHNRVSECGWAARRAPHLHTLYNICRNMHAWLRQDHKNVCVVHCMDGRAASAVAVCSFLCFCRLFSTAEAAVYMFSMKRCPPGIWPSHKRYIEYMCDMVAEEPITPHSKPILVRAVVMTPVPLFSKQRSGCRPFCEVYVGDERVASTSQEYDKMRDFKIEDGKAVIPLGVTVQGDVLIVIYHARSTLGGRLQAKMASMKMFQIQFHTGFVPRNATTVKFAKYDLDACDIQEKYPDLFQVNLEVEVEPRDRPSREAPPWENSSMRGLNPKILFSSREEQQDILSKFGKPELPRQPGSTAQYDAGAGSPEAEPTDSDSPPSSSADASRFLHTLDWQEEKEAETGAENASSKESESALMEDRDESEVSDEGGSPISSEGQEPRADPEPPGLAAGLVQQDLVFEVETPAVLPEPVPQEDGVDLLGLHSEVGAGPAVPPQACKAPSSNTDLLSCLLGPPEAASQGPPEDLLSEDPLLLASPAPPLSVQSTPRGGPPAAADPFGPLLPSSGNNSQPCSNPDLFGEFLNSDSVTVPPSFPSAHSAPPPSCSADFLHLGDLPGEPSKMTASSSNPDLLGGWAAWTETAASAVAPTPATEGPLFSPGGQPAPCGSQASWTKSQNPDPFADLGDLSSGLQGSPAGFPPGGFIPKTATTPKGSSSWQTSRPPAQGASWPPQAKPPPKACTQPRPNYASNFSVIGAREERGVRAPSFAQKPKVSENDFEDLLSNQGFSSRSDKKGPKTIAEMRKQDLAKDTDPLKLKLLDWIEGKERNIRALLSTLHTVLWDGESRWTPVGMADLVAPEQVKKHYRRAVLAVHPDKAAGQPYEQHAKMIFMELNDAWSEFENQGSRPLF

SEQ ID NO: 63 - protein, 211 aa, Artificial Sequence
  Synthetic Construct
MAPLAEVGGFLGGLEGLGQQVGSHFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 64 - protein, 870 aa, Artificial Sequence
  Synthetic Construct
MFFACYCALRTNVKKYRYQDEDAPHDHSLPRLTHEVRGPELVHVSEKNLSQIENVHGYVLQSHISPLKASPAPIIVNTDTLDTIPYVNGTEIEYEFEEITLERGNSGLGFSIAGGTDNPHIGDDPGIFITKIIPGGAAAEDGRLRVNDCILRVNEVDVSEVSHSKAVEALKEAGSIVRLYVRRRRPILETVVEIKLFKGPKGLGFSIAGGVGNQHIPGDNSIYVTKIIDGGAAQKDGRLQVGDRLLMVNNYSLEEVTHEEAVAILKNTSEVVYLKVGKPTTIYMTDPYGPPDITHSYSPPMENHLLSGNNGTLEYKTSLPPISPGRYSPIPKHMLVDDDYTRPPEPVYSTVNKLCDKPASPRHYSPVECDKSFLLSAPYSHYHLGLLPDSEMTSHSQHSTATRQPSMTLQRAVSLEGEPRKVVLHKGSTGLGFNIVGGEDGEGIFVSFILAGGPADLSGELQRGDQILSVNGIDLRGASHEQAAAALKGAGQTVTIIAQYQPEDYARFEAKIHDLREQMMNHSMSSGSGSLRTNQKRSLYVRAMFDYDKSKDSGLPSQGLSFKYGDILHVINASDDEWWQARRVMLEGDSEEMGVIPSKRRVERKERARLKTVKFNAKPGVIDSKGSFNDKRKKSFIFSRKFPFYKNKEQSEQETSDPERGQEDLILSYEPVTRQEINYTRPVIILGPMKDRINDDLISEFPDKFGSCVPHTTRPKRDYEVDGRDYHFVISREQMEKDIQEHKFIEAGQYNDNLYGTSVQSVRFVAERGKHCILDVSGNAIKRLQVAQLYPIAIFIKPRSLEPLMEMNKRLTEEQAKKTYDRAIKLEQEFGEYFTAIVQGDTLEDIYNQCKLVIEEQSGPFIWIPSKEKL

SEQ ID NO: 65 - protein, 314 aa, Artificial Sequence
  Synthetic Construct
MVAPVWYLVAAALLVGFILFLTRSRGRAASAGQEPLHNEELAGAGRVAQPGPLEPEEPRAGGRPRRRRDLGSRLQAQRRAQRVAWAEADENEEEAVILAQEEEGVEKPAETHLSGKIGAKKLRKLEEKQARKAQREAEEAEREERKRLESQREAEWKKEEERLRLEEEQKEEEERKAREEQAQREHEEYLKLKEAFVVEEEGVGETMTEEQSQSFLTEFINYIKQSKVVLLEDLASQVGLRTQDTINRIQDLLAEGTITGVIDDRGKFIYITPEELAAVANFIRQRGRVSIAELAQASNSLIAWGRESPAQAPA

SEQ ID NO: 66 - protein, 1147 aa, Artificial Sequence
  Synthetic Construct
MDEPPFSEAALEQALGEPCDLDAALLTDIEDMLQLINNQDSDFPGLFDPPYAGSGAGGTDPASPDTSSPGSLSPPPATLSSSLEAFLSGPQAAPSPLSPPQPAPTPLKMYPSMPAFSPGPGIKEESVPLSILQTPTPQPLPGALLPQSFPAPAPPQFSSTPVLGYPSPPGGFSTGSPPGNTQQPLPGLPLASPPGVPPVSLHTQVQSVVPQQLLTVTAAPTAAPVTTTVTSQIQQVPVLLQPHFIKADSLLLTAMKTDGATVKAAGLSPLVSGTTVQTGPLPTLVSGGTILATVPLVVDAEKLPINRLAAGSKAPASAQSRGEKRTAHNAIEKRYRSSINDKIIELKDLVVGTEAKLNKSAVLRKAIDYIRFLQHSNQKLKQENLSLRTAVHKSKSLKDLVSACGSGGNTDVLMEGVKTEVEDTLTPPPSDAGSPFQSSPLSLGSRGSGSGGSGSDSEPDSPVFEDSKAKPEQRPSLHSRGMLDRSRLALCTLVFLCLSCNPLASLLGARGLPSPSDTTSVYHSPGRNVLGTESRDGPGWAQWLLPPVVWLLNGLLVLVSLVLLFVYGEPVTRPHSGPAVYFWRHRKQADLDLARGDFAQAAQQLWLALRALGRPLPTSHLDLACSLLWNLIRHLLQRLWVGRWLAGRAGGLQQDCALRVDASASARDAALVYHKLHQLHTMGKHTGGHLTATNLALSALNLAECAGDAVSVATLAEIYVAAALRVKTSLPRALHFLTRFFLSSARQACLAQSGSVPPAMQWLCHPVGHRFFVDGDWSVLSTPWESLYSLAGNPVDPLAQVTQLFREHLLERALNCVTQPNPSPGSADGDKEFSDALGYLQLLNSCSDAAGAPAYSFSISSSMATTTGVDPVAKWWASLTAVVIHWLRRDEEAAERLCPLVEHLPRVLQESERPLPRAALHSFKAARALLGCAKAESGPASLTICEKASGYLQDSLATTPASSSIDKAVQLFLCDLLLVVRTSLWRQQQPPAPAPAAQGTSSRPQASALELRGFQRDLSSLRRLAQSFRPAMRRVFLHEATARLMAGASPTRTHQLLDRSLRRRAGPGGKGGAVAELEPRPTRREHAEALLLASCYLPPGFLSAPGQRVGMLAEAARTLEKLGDRRLLHDCQQMLMRLGGGTTVTSS

SEQ ID NO: 67 - protein, 412 aa, Artificial Sequence
  Synthetic Construct
MILASVLRSGPGGGLPLRPLLGPALALRARSTSATDTHHVEMARERSKTVTSFYNQSAIDAAAEKPSVRLTPTMMLYAGRSQDGSHLLKSARYLQQELPVRIAHRIKGFRCLPFIIGCNPTILHVHELYIRAFQKLTDFPPIKDQADEAQYCQLVRQLLDDHKDVVTLLAEGLRESRKHIEDEKLVRYFLDKTLTSRLGIRMLATHHLALHEDKPDFVGIICTRLSPKKIIEKWVDFARRLCEHKYGNAPRVRINGHVAARFPFIPMPLDYILPELLKNAMRATMESHLDTPYNVPDVVITIANNDVDLIIRISDRGGGIAHKDLDRVMDYHFTTAEASTQDPRISPLFGHLDMHSGAQSGPMHGFGFGLPTSRAYAEYLGGSLQLQSLQGIGTDVYLRLRHIDGREESFRI

SEQ ID NO: 68 - protein, 465 aa, Artificial Sequence
  Synthetic Construct
MIVFVRFNSSHGFPVEVDSDTSIFQLKEVVAKRQGVPADQLRVIFAGKELRNDWTVQNCDLDQQSIVHIVQRPWRKGQEMNATGGDDPRNAAGGCEREPQSLTRVDLSSSVLPGDSVGLAVILHTDSRKDSPPAGSPAGRSIYNSFYVYCKGPCQRVQPGKLRVQCSTCRQATLTLTQGPSCWDDVLIPNRMSGECQSPHCPGTSAEFFFKCGAHPTSDKETSVALHLIATNSRNITCITCTDVRSPVLVFQCNSRHVICLDCFHLYCVTRLNDRQFVHDPQLGYSLPCVAGCPNSLIKELHHFRILGEEQYNRYQQYGAEECVLQMGGVLCPRPGCGAGLLPEPDQRKVTCEGGNGLGCGFAFCRECKEAYHEGECSAVFEASGTTTQAYRVDERAAEQARWEAASKETIKKTTKPCPRCHVPVEKNGGCMHMKCPQPQCRLEWCWNCGCEWNRVCMGDHWFDV

SEQ ID NO: 69 - protein, 213 aa, Artificial Sequence
  Synthetic Construct
MEAIWLYQFRLIVIGDSTVGKSCLIRRFTEGRFAQVSDPTVGVDFFSRLVEIEPGKRIKLQIWDTAGQERFRSITRAYYRNSVGGLLLFDITNRRSFQNVHEWLEETKVHVQPYQIVFVLVGHKCDLDTQRQVTRHEAEKLAAAYGMKYIETSARDAINVEKAFTDLTRDIYELVKRGEITIQEGWEGVKSGFVPNVVHSSEEVVKSERRCLC

SEQ ID NO: 70 - protein, 913 aa, Artificial Sequence
  Synthetic Construct
MKDSENKGASSPDMEPSYGGGLFDMVKGGAGRLFSNLKDNLKDTLKDTSSRVIQSVTSYTKGDLDFTYVTSRIIVMSFPLDNVDIGFRNQVDDIRSFLDSRHLDHYTVYNLSPKSYRTAKFHSRVSECSWPIRQAPSLHNLFAVCRNMYNWLLQNPKNVCVVHCLDGRAASSILVGAMFIFCNLYSTPGPAIRLLYAKRPGIGLSPSHRRYLGYMCDLLADKPYRPHFKPLTIKSITVSPIPFFNKQRNGCRPYCDVLIGETKIYSTCTDFERMKEYRVQDGKIFIPLNITVQGDVVVSMYHLRSTIGSRLQAKVTNTQIFQLQFHTGFIPLDTTVLKFTKPELDACDVPEKYPQLFQVTLDVELQPHDKVIDLTPPWEHYCTKDVNPSILFSSHQEHQDTLALGGQAPIDIPPDNPRHYGQSGFFASLCWQDQKSEKSFCEEDHAALVNQESEQSDDELLTLSSPHGNANGDKPHGVKKPSKKQQEPAAPPPPEDVDLLGLEGSAMSNSFSPPAAPPTNSELLSDLFGGGGAAGPTQAGQSGVEDVFHPSGPASTQSTPRRSATSTSASPTLRVGEGATFDPFGAPSKPSGQDLLGSFLNTSSASSDPFLQPTRSPSPTVHASSTPAVNIQPDVSGGWDWHAKPGGFGMGSKSAATSPTGSSHGTPTHQSKPQTLDPFADLGTLGSSSFASKPTTPTGLGGGFPPLSSPQKASPQPMGGGWQQGGAYNWQQPQPKPQPSMPHSSPQNRPNYNVSFSAMPGGQNERGKGSSNLEGKQKAADFEDLLSGQGFNAHKDKKGPRTIAEMRKEEMAKEMDPEKLKILEWIEGKERNIRALLSTMHTVLWAGETKWKPVGMADLVTPEQVKKVYRKAVLVVHPDKATGQPYEQYAKMIFMELNDAWSEFENQGQKPLY

SEQ ID NO: 71 - protein, 631 aa, Artificial Sequence
  Synthetic Construct
MPRYGASLRQSCPRSGREQGQDGTAGAPGLLWMGLVLALALALALALALSDSRVLWAPAEAHPLSPQGHPARLHRIVPRLRDVFGWGNLTCPICKGLFTAINLGLKKEPNVARVGSVAIKLCNLLKIAPPAVCQSIVHLFEDDMVEVWRRSVLSPSEACGLLLGSTCGHWDIFSSWNISLPTVPKPPPKPPSPPAPGAPVSRILFLTDLHWDHDYLEGTDPDCADPLCCRRGSGLPPASRPGAGYWGEYSKCDLPLRTLESLLSGLGPAGPFDMVYWTGDIPAHDVWHQTRQDQLRALTTVTALVRKFLGPVPVYPAVGNHESTPVNSFPPPFIEGNHSSRWLYEAMAKAWEPWLPAEALRTLRIGGFYALSPYPGLRLISLNMNFCSRENFWLLINSTDPAGQLQWLVGELQAAEDRGDKVHIIGHIPPGHCLKSWSWNYYRIVARYENTLAAQFFGHTHVDEFEVFYDEETLSRPLAVAFLAPSATTYIGLNPGYRVYQIDGNYSGSSHVVLDHETYILNLTQANIPGAIPHWQLLYRARETYGLPNTLPTAWHNLVYRMRGDMQLFQTFWFLYHKGHPPSEPCGTPCRLATLCAQLSARADSPALCRHLMPDGSLPEAQSLWPRPLFC

SEQ ID NO: 72 - protein, 504 aa, Artificial Sequence
  Synthetic Construct
MSQPRTPEQALDTPGDCPPGRRDEDAGEGIQCSQRMLSFSDALLSIIATVMILPVTHTEISPEQQFDRSVQRLLATRIAVYLMTFLIVTVAWAAHTRLFQVVGKTDDTLALLNLACMMTITFLPYTFSLMVTFPDVPLGIFLFCVCVIAIGVVQALIVGYAFHFPHLLSPQIQRSAHRALYRRHVLGIVLQGPALCFAAAIFSLFFVPLSYLLMVTVILLPYVSKVTGWCRDRLLGHREPSAHPVEVFSFDLHEPLSKERVEAFSDGVYAIVATLLILDICEDNVPDPKDVKERFSGSLVAALSATGPRFLAYFGSFATVGLLWFAHHSLFLHVRKATRAMGLLNTLSLAFVGGLPLAYQQTSAFARQPRDELERVRVSCTIIFLASIFQLAMWTTALLHQAETLQPSVWFGGREHVLMFAKLALYPCASLLAFASTCLLSRFSVGIFHLMQIAVPCAFLLLRLLVGLALATLRVLRGLARPEHPPPAPTGQDDPQSQLLPAPC

SEQ ID NO: 73 - protein, 545 aa, Artificial Sequence
  Synthetic Construct
MAEPSGSPVHVQLPQQAAPVTAAAAAAPAAATAAPAPAAPAAPAPAPAPAAQAVGWPICRDAYELQEVIGSGATAVVQAALCKPRQERVAIKRINLEKCQTSMDELLKEIQAMSQCSHPNVVTYYTSFVVKDELWLVMKLLSGGSMLDIIKYIVNRGEHKNGVLEEAIIATILKEVLEGLDYLHRNGQIHRDLKAGNILLGEDGSVQIADFGVSAFLATGGDVTRNKVRKTFVGTPCWMAPEVMEQVRGYDFKADMWSFGITAIELATGAAPYHKYPPMKVLMLTLQNDPPTLETGVEDKEMMKKYGKSFRKLLSLCLQKDPSKRPTAAELLKCKFFQKAKNREYLIEKLLTRTPDIAQRAKKVRRVPGSSGHLHKTEDGDWEWSDDEMDEKSEEGKAAFSQEKSRRVKEENPEIAVSASTIPEQIQSLSVHDSQGPPNANEDYREASSCAVNLVLRLRNSRKELNDIRFEFTPGRDTADGVSQELFSAGLVDGHDVVIVAANLQKIVDDPKALKTLTFKLASGCDGSEIPDEVKLIGFAQLSVS

SEQ ID NO: 74 - protein, 318 aa, Artificial Sequence
  Synthetic Construct
MAAQGCAASRLLQLLLQLLLLLLLLAAGGARARWRGEGTSAHLRDIFLGRCAEYRALLSPEQRNKNCTAIWEAFKVALDKDPCSVLPSDYDLFINLSRHSIPRDKSLFWENSHLLVNSFADNTRRFMPLSDVLYGRVADFLSWCRQKNDSGLDYQSCPTSEDCENNPVDSFWKRASIQYSKDSSGVIHVMLNGSEPTGAYPIKGFFADYEIPNLQKEKITRIEIWVMHEIGGPNVESCGEGSMKVLEKRLKDMGFQYSCINDYRPVKLLQCVDHSTHPDCALKSAAAATQRKAPSLYTEQRAGLIIPLFLVLASRTQL

SEQ ID NO: 75 - protein, 607 aa, Artificial Sequence
  Synthetic Construct
MILLTFSTGRRLDFVHHSGVFFLQTLLWILCATVCGTEQYFNVEVWLQKYGYLPPTDPRMSVLRSAETMQSALAAMQQFYGINMTGKVDRNTIDWMKKPRCGVPDQTRGSSKFHIRRKRYALTGQKWQHKHITYSIKNVTPKVGDPETRKAIRRAFDVWQNVTPLTFEEVPYSELENGKRDVDITIIFASGFHGDSSPFDGEGGFLAHAYFPGPGIGGDTHFDSDEPWTLGNPNHDGNDLFLVAVHELGHALGLEHSNDPTAIMAPFYQYMETDNFKLPNDDLQGIQKIYGPPDKIPPPTRPLPTVPPHRSIPPADPRKNDRPKPPRPPTGRPSYPGAKPNICDGNFNTLAILRREMFVFKDQWFWRVRNNRVMDGYPMQITYFWRGLPPSIDAVYENSDGNFVFFKGNKYWVFKDTTLQPGYPHDLITLGSGIPPHGIDSAIWWEDVGKTYFFKGDRYWRYSEEMKTMDPGYPKPITVWKGIPESPQGAFVHKENGFTYFYKGKEYWKFNNQILKVEPGYPRSILKDFMGCDGPTDRVKEGHSPPDDVDIVIKLDNTASTVKAIAIVIPCILALCLLVLVYTVFQFKRKGTPRHILYCKRSMQEWV

SEQ ID NO: 76 - protein, 217 aa, Artificial Sequence
  Synthetic Construct
MEVENEASCSPGSASGGSREYKVVMLGAGGVGKSAMTMQFISHQFPDYHDPTIEDAYKTQVRIDNEPAYLDILDTAGQAEFTAMREQYMRGGEGFIICYSVTDRQSFQEAAKFKELIFQVRHTYEIPLVLVGNKIDLEQFRQVSTEEGLSLAQEYNCGFFETSAALRFCIDDAFHGLVREIRKKESMPSLMEKKLKRKDSLWKKLKGSLKKKRENMT

SEQ ID NO: 77 - protein, 393 aa, Artificial Sequence
  Synthetic Construct
MADRRRRLRPGTLAPVREGVNCRSRCFTKHKNGLKFPTSLHSRQLVFPRKGLDDFRKGCPPCTGLVTQVPVEGFLPQIYHRAPQLAPKKRQIKLLKEADVLSKLSPAQQARKAFLEDVEAHLTPHPLALYLNLEEAMPIELLSKVLEVLDPDRKLEDTWAYCQDTRKGMKEPTKLLKKHSTQVYLGPSKKTSVSNAGQWLYEEKPHKMDLLHENGPRPGLHENGISDIDEEFILKQFDIDYETKPSHDALHTMKLNQVPLELKRSVGLSKLQETEFFQKLGYERKLQKPQNPYKPKWVKMRYGAWYLNPKLWKKQRVDEPLVDPEVSHKAQEENFKKELQEQEELLADLHGTVAFKDFILSRGYRTPRFLENMYIGKECKRACNKTPIKRTQA

SEQ ID NO: 78 - protein, 684 aa, Artificial Sequence
  Synthetic Construct
MNPPAAFLAGRQNIGSEVEISTIEKQRKELQLLIGELKDRDKELNDMVAVHQQQLLSWEEDRQKVLTLEERCSKLEGELHKRTEIIRSLTKKVKALESNQMECQTALQKTQLQLQEMAQKATHSSLLSEDLEARNETLSNTLVELSAQVGQLQAREQALTTMIKLKDKDIIEAVNHIADCSGKFKMLEHALRDAKMAETCIVKEKQDYKQKLKALKIEVNKLKEDLNEKTTENNEQREEIIRLKQEKSCLHDELLFTVEREKRKDELLNIAKSKQERTNSELHNLRQIYVKQQSDLQFLNFNVENSQELIQMYDSKMEESKALDSSRDMCLSDLENNHPKVDIKREKNQKSLFKDQKFEAMLVQQNRSDKSSCDECKEKKQQIDTVFGEKSVITLSSIFTKDLVEKHNLPWSLGGKTQIEPENKITLCKIHTKSPKCHGTGVQNEGKQPSETPTLSDEKQWHDVSVYLGLTNCPSSKHPEKLDVECQDQMERSEISCCQKNEACLGESGMCDSKCCHPSNFIIEAPGHMSDVEWMSIFKPSKMQRIVRLKSGCTCSESICGTQHDSPASELIAIQDSHSLGSSKSALREDETESSSNKKNSPTSLLIYKDAPAFNEKASIVLPSQDDFSPTSKLQRLLAESRQMVTDLELSTLLPISHENLTGSATNKSEVPEESAQKNTFVSY

SEQ ID NO: 79 - protein, 261 aa, Artificial Sequence
  Synthetic Construct
MSWKKALRIPGGLRAATVTLMLAMLSTPVAEGRDSPEDFVYQFKAMCYFTNGTERVRYVTRYIYNREEYARFDSDVEVYRAVTPLGPPDAEYWNSQKEVLERTRAELDTVCRHNYQLELRTTLQRRVEPTVTISPSRTEALNHHNLLVCSVTDFYPAQIKVRWFRNDQEETTGVVSTPLIRNGDWTFQILVMLEMTPQHGDVYTCHVEHPSLQNPITVEWRAQSESAQSKMLSGIGGFVLGLIFLGLGLIIHHRSQKGLLH

SEQ ID NO: 80 - protein, 167 aa, Artificial Sequence
  Synthetic Construct
MASAEPLTALSRWYLYAIHGYFCEVMFTAAWEFVVNLNWKFPGVTSVWALFIYGTSILIVERMYLRLRGRCPLLLRCLIYTLWTYLWEFTTGFILRQFNACPWDYSQFDFDFMGLITLEYAVPWFCGALIMEQFIIRNTLRLRFDKDAEPGEPSGALALANGHVKTD

SEQ ID NO: 81 - protein, 758 aa, Artificial Sequence
  Synthetic Construct
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKHPTPGSSDPLIQPSSPAVCPEPPSSPKYVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 82 - protein, 592 aa, Artificial Sequence
  Synthetic Construct
MAAAVAAALARLLAAFLLLAAQVACEYGMVHVVSQAGGPEGKDYCILYNPQWAHLPHDLSKASFLQLRNWTASLLCSAADLPARGFSNQIPLVARGNCTFYEKVRLAQGSGARGLLIVSRERLVPPGGNKTQYDEIGIPVALLSYKDMLDIFTRFGRTVRAALYAPKEPVLDYNMVIIFIMAVGTVAIGGYWAGSRDVKKRYMKHKRDDGPEKQEDEAVDVTPVMTCVFVVMCCSMLVLLYYFYDLLVYVVIGIFCLASATGLYSCLAPCVRRLPFGKCRIPNNSLPYFHKRPQARMLLLALFCVAVSVVWGVFRNEDQWAWVLQDALGIAFCLYMLKTIRLPTFKACTLLLLVLFLYDIFFVFITPFLTKSGSSIMVEVATGPSDSATREKLPMVLKVPRLNSSPLALCDRPFSLLGFGDILVPGLLVAYCHRFDIQVQSSRVYFVACTIAYGVGLLVTFVALALMQRGQPALLYLVPCTLVTSCAVALWRRELGVFWTGSGFAKVLPPSPWAPAPADGPQPPKDSATPLSPQPPSEEPATSPWPAEQSPKSRTSEEMGAGAPMREPGSPAESEGRDQAQPSPVTQPGASA

SEQ ID NO: 83 - protein, 1063 aa, Artificial Sequence
  Synthetic Construct
MSPGASRGPRGSQAPLIAPLCCAAAALGMLLWSPACQAFNLDVEKLTVYSGPKGSYFGYAVDFHIPDARTASVLVGAPKANTSQPDIVEGGAVYYCPWPAEGSAQCRQIPFDTTNNRKIRVNGTKEPIEFKSNQWFGATVKAHKGKVVACAPLYHWRTLKPTPEKDPVGTCYVAIQNFSAYAEFSPCRNSNADPEGQGYCQAGFSLDFYKNGDLIVGGPGSFYWQGQVITASVADIIANYSFKDILRKLAGEKQTEVAPASYDDSYLGYSVAAGEFTGDSQQELVAGIPRGAQNFGYVSIINSTDMTFIQNFTGEQMASYFGYTVVVSDVNSDGLDDVLVGAPLFMEREFESNPREVGQIYLYLQVSSLLFRDPQILTGTETFGRFGSAMAHLGDLNQDGYNDIAIGVPFAGKDQRGKVLIYNGNKDGLNTKPSQVLQGVWASHAVPSGFGFTLRGDSDIDKNDYPDLIVGAFGTGKVAVYRARPVVTVDAQLLLHPMIINLENKTCQVPDSMTSAACFSLRVCASVTGQSIANTIVLMAEVQLDSLKQKGAIKRTLFLDNHQAHRVFPLVIKRQKSHQCQDFIVYLRDETEFRDKLSPINISLNYSLDESTFKEGLEVKPILNYYRENIVSEQAHILVDCGEDNLCVPDLKLSARPDKHQVIIGDENHLMLIINARNEGEGAYEAELFVMIPEEADYVGIERNNKGFRPLSCEYKMENVTRMVVCDLGNPMVSGTNYSLGLRFAVPRLEKTNMSINFDLQIRSSNKDNPDSNFVSLQINITAVAQVEIRGVSHPPQIVLPIHNWEPEEEPHKEEEVGPLVEHIYELHNIGPSTISDTILEVGWPFSARDEFLLYIFHIQTLGPLQCQPNPNINPQDIKPAASPEDTPELSAFLRNSTIPHLVRKRDVHVVEFHRQSPAKILNCTNIECLQISCAVGRLEGGESAVLKVRSRLWAHTFLQRKNDPYALASLVSFEVKKMPYTDQPAKLPEGSIVIKTSVIWATPNVSFSIPLWVIILAILLGLLVLAILTLALWKCGFFDRARPPQEDMTDREQLTNDKTPEA

SEQ ID NO: 84 - protein, 1180 aa, Artificial Sequence
  Synthetic Construct
MSADSSPLVGSTPTGYGTLTIGTSIDPLSSSVSSVRLSGYCGSPWRVIGYHVVVWMMAGIPLLLFRWKPLWGVRLRLRPCNLAHAETLVIEIRDKEDSSWQLFTVQVQTEAIGEGSLEPSPQSQAEDGRSQAAVGAVPEGAWKDTAQLHKSEEAVSVGQKRVLRYYLFQGQRYIWIETQQAFYQVSLLDHGRSCDDVHRSRHGLSLQDQMVRKAIYGPNVISIPVKSYPQLLVDEALNPYYGFQAFSIALWLADHYYWYALCIFLISSISICLSLYKTRKQSQTLRDMVKLSMRVCVCRPGGEEEWVDSSELVPGDCLVLPQEGGLMPCDAALVAGECMVNESSLTGESIPVLKTALPEGLGPYCAETHRRHTLFCGTLILQARAYVGPHVLAVVTRTGFCTAKGGLVSSILHPRPINFKFYKHSMKFVAALSVLALLGTIYSIFILYRNRVPLNEIVIRALDLVTVVVPPALPAAMTVCTLYAQSRLRRQGIFCIHPLRINLGGKLQLVCFDKTGTLTEDGLDVMGVVPLKGQAFLPLVPEPRRLPVGPLLRALATCHALSRLQDTPVGDPMDLKMVESTGWVLEEEPAADSAFGTQVLAVMRPPLWEPQLQAMEEPPVPVSVLHRFPFSSALQRMSVVVAWPGATQPEAYVKGSPELVAGLCNPETVPTDFAQMLQSYTAAGYRVVALASKPLPTVPSLEAAQQLTRDTVEGDLSLLGLLVMRNLLKPQTTPVIQALRRTRIRAVMVTGDNLQTAVTVARGCGMVAPQEHLIIVHATHPERGQPASLEFLPMESPTAVNGVKDPDQAASYTVEPDPRSRHLALSGPTFGIIVKHFPKLLPKVLVQGTVFARMAPEQKTELVCELQKLQYCVGMCGDGANDCGALKAADVGISLSQAEASVVSPFTSSMASIECVPMVIREGRCSLDTSFSVFKYMALYSLTQFISVLILYTINTNLGDLQFLAIDLVITTTVAVLMSRTGPALVLGRVRPPGALLSVPVLSSLLLQMVLVTGVQLGGYFLTLAQPWFVPLNRTVAAPDNLPNYENTVVFSLSSFQYLILAAAVSKGAPFRRPLYTNVPFLVALALLSSVLVGLVLVPGLLQGPLALRNITDTGFKLLLLGLVTLNFVGAFMLESVLDQCLPACLRRLRPKRASKKRFKQLERELAEQPWPPLPAGPLR

SEQ ID NO: 85 - protein, 942 aa, Artificial Sequence
  Synthetic Construct
MAAAAEPGARAWLGGGSPRPGSPACSPVLGSGGRARPGPGPGPGPERAGVRAPGPAAAPGHSFRKVTLTKPTFCHLCSDFIWGLAGFLCDVCNFMSHEKCLKHVRIPCTSVAPSLVRVPVAHCFGPRGLHKRKFCAVCRKVLEAPALHCEVCELHLHPDCVPFACSDCRQCHQDGHQDHDTHHHHWREGNLPSGARCEVCRKTCGSSDVLAGVRCEWCGVQAHSLCSAALAPECGFGRLRSLVLPPACVRLLPGGFSKTQSFRIVEAAEPGEGGDGADGSAAVGPGRETQATPESGKQTLKIFDGDDAVRRSQFRLVTVSRLAGAEEVLEAALRAHHIPEDPGHLELCRLPPSSQACDAWAGGKAGSAVISEEGRSPGSGEATPEAWVIRALPRAQEVLKIYPGWLKVGVAYVSVRVTPKSTARSVVLEVLPLLGRQAESPESFQLVEVAMGCRHVQRTMLMDEQPLLDRLQDIRQMSVRQVSQTRFYVAESRDVAPHVSLFVGGLPPGLSPEEYSSLLHEAGATKATVVSVSHIYSSQGAVVLDVACFAEAERLYMLLKDMAVRGRLLTALVLPDLLHAKLPPDSCPLLVFVNPKSGGLKGRDLLCSFRKLLNPHQVFDLTNGGPLPGLHLFSQVPCFRVLVCGGDGTVGWVLGALEETRYRLACPEPSVAILPLGTGNDLGRVLRWGAGYSGEDPFSVLLSVDEADAVLMDRWTILLDAHEAGSAENDTADAEPPKIVQMSNYCGIGIDAELSLDFHQAREEEPGKFTSRLHNKGVYVRVGLQKISHSRSLHKQIRLQVERQEVELPSIEGLIFINIPSWGSGADLWGSDSDTRFEKPRMDDGLLEVVGVTGVVHMGQVQGGLRSGIRIAQGSYFRVTLLKATPVQVDGEPWVQAPGHMIISAAGPKVHMLRKAKQKPRRAGTTRDARADAAPAPESDPR

SEQ ID NO: 86 - protein, 288 aa, Artificial Sequence
  Synthetic Construct
MKDRTQELRSAKDSDDEEEVVHVDRDHFMDEFFEQVEEIRGCIEKLSEDVEQVKKQHSAILAAPNPDEKTKQELEDLTADIKKTANKVRSKLKAIEQSIEQEEGLNRSSADLRIRKTQHSTLSRKFVEVMTEYNATQSKYRDRCKDRIQRQLEITGRTTTNEELEDMLESGKLAIFTDDIKMDSQMTKQALNEIETRHNEIIKLETSIRELHDMFVDMAMLVESQGEMIDRIEYNVEHSVDYVERAVSDTKKAVKYQSKARRKKIMIIICCVVLGVVLASSIGGTLGL

SEQ ID NO: 87 - protein, 243 aa, Artificial Sequence
  Synthetic Construct
MSRPVRNRKVVDYSQFQESDDADEDYGRDSGPPTKKIRSSPREAKNKRRSGKNSQEDSEDSEDKDVKTKKDDSHSAEDSEDEKEDHKNVRQQRQAASKAASKQREMLMEDVGSEEEQEEEDEAPFQEKDSGSDEDFLMEDDDDSDYGSSKKKNKKMVKKSKPERKEKKMPKPRLKATVTPSPVKGKGKVGRPTASKASKEKTPSPKEEDEEPESPPEKKTSTSPPPEKSGDEGSEDEAPSGED

SEQ ID NO: 88 - protein, 336 aa, Artificial Sequence
  Synthetic Construct
MKIDIHSHILPKEWPDLKKRFGYGGWVQLQHHSKGEAKLLKDGKVFRVVRENCWDPEVRIREMDQKGVTVQALSTVPVMFSYWAKPEDTLNLCQLLNNDLASTVVSYPRRFVGLGTLPMQAPELAVKEMERCVKELGFPGVQIGTHVNEWDLNAQELFPVYAAAERLKCSLFVHPWDMQMDGRMAKYWLPWLVGMPAETTIAICSMIMGGVFEKFPKLKVCFAHGGGAFPFTVGRISHGFSMRPDLCAQDNPMNPKKYLGSFYTDALVHDPLSLKLLTDVIGKDKVILGTDYPFPLGELEPGKLIESMEEFDEETKNKLKAGNALAFLGLERKQFE

SEQ ID NO: 89 - protein, 254 aa, Artificial Sequence
  Synthetic Construct
MAISGVPVLGFFIIAVLMSAQESWAIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTENVVCALGLTVGLVGIIIGTIFIIKGVRKSNAAERRGPL

SEQ ID NO: 90 - protein, 481 aa, Artificial Sequence
  Synthetic Construct
MSTLCPPPSPAVAKTEIALSGKSPLLAATFAYWDNILGPRVRHIWAPKTEQVLLSDGEITFLANHTLNGEILRNAESGAIDVKFFVLSEKGVIIVSLIFDGNWNGDRSTYGLSIILPQTELSFYLPLHRVCVDRLTHIIRKGRIWMHKERQENVQKIILEGTERMEDQGQSIIPMLTGEVIPVMELLSSMKSHSVPEEIDIADTVLNDDDIGDSCHEGFLLNAISSHLQTCGCSVVVGSSAEKVNKIVRTLCLFLTPAERKCSRLCEAESSFKYESGLFVQGLLKDSTGSFVLPFRQVMYAPYPTTHIDVDVNTVKQMPPCHEHIYNQRRYMRSELTAFWRATSEEDMAQDTIIYTDESFTPDLNIFQDVLHRDTLVKAFLDQVFQLKPGLSLRSTFLAQFLLVLHRKALTLIKYIEDDTQKGKKPFKSLRNLKIDLDLTAEGDLNIIMALAEKIKPGLHSFIFGRPFYTSVQERDVLMTF

SEQ ID NO: 91 - protein, 440 aa, Artificial Sequence
  Synthetic Construct
MASLTVKAYLLGKEDAAREIRRFSFCCSPEPEAEAEAAAGPGPCERLLSRVAALFPALRPGGFQAHYRDEDGDLVAFSSDEELTMAMSYVKDDIFRIYIKEKKECRRDHRPPCAQEAPRNMVHPNVICDGCNGPVVGTRYKCSVCPDYDLCSVCEGKGLHRGHTKLAFPSPFGHLSEGFSHSRWLRKVKHGHFGWPGWEMGPPGNWSPRPPRAGEARPGPTAESASGPSEDPSVNFLKNVGESVAAALSPLGIEVDIDVEHGGKRSRLTPVSPESSSTEEKSSSQPSSCCSDPSKPGGNVEGATQSLAEQMRKIALESEGRPEEQMESDNCSGGDDDWTHLSSKEVDPSTGELQSLQMPESEGPSSLDPSQEGPTGLKEAALYPHLPPEADPRLIESLSQMLSMGFSDEGGWLTRLLQTKNYDIGAALDTIQYSKHPPPL

SEQ ID NO: 92 - protein, 414 aa, Artificial Sequence
  Synthetic Construct
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 93 - protein, 729 aa, Artificial Sequence
  Synthetic Construct
MQSTSNHLWLLSDILGQGATANVFRGRHKKTGDLFAIKVFNNISFLRPVDVQMREFEVLKKLNHKNIVKLFAIEEETTTRHKVLIMEFCPCGSLYTVLEEPSNAYGLPESEFLIVLRDVVGGMNHLRENGIVHRDIKPGNIMRVIGEDGQSVYKLTDFGAARELEDDEQFVSLYGTEEYLHPDMYERAVLRKDHQKKYGATVDLWSIGVTFYHAATGSLPFRPFEGPRRNKEVMYKIITGKPSGAISGVQKAENGPIDWSGDMPVSCSLSRGLQVLLTPVLANILEADQEKCWGFDQFFAETSDILHRMVIHVFSLQQMTAHKIYIHSYNTATIFHELVYKQTKIISSNQELIYEGRRLVLEPGRLAQHFPKTTEENPIFVVSREPLNTIGLIYEKISLPKVHPRYDLDGDASMAKAITGVVCYACRIASTLLLYQELMRKGIRWLIELIKDDYNETVHKKTEVVITLDFCIRNIEKTVKVYEKLMKINLEAAELGEISDIHTKLLRLSSSQGTIETSLQDIDSRLSPGGSLADAWAHQEGTHPKDRNVEKLQVLLNCMTEIYYQFKKDKAERRLAYNEEQIHKFDKQKLYYHATKAMTHFTDECVKKYEAFLNKSEEWIRKMLHLRKQLLSLTNQCFDIEEEVSKYQEYTNELQETLPQKMFTASSGIKHTMTPIYPSSNTLVEMTLGMKKLKEEMEGVVKELAENNHILERFGSLTMDGGLRNVDCL

SEQ ID NO: 94 - protein, 806 aa, Artificial Sequence
  Synthetic Construct
MASGADSKGDDLSTAILKQKNRPNRLIVDEAINEDNSVVSLSQPKMDELQLFRGDTVLLKGKKRREAVCIVLSDDTCSDEKIRMNRVVRNNLRVRLGDVISIQPCPDVKYGKRIHVLPIDDTVEGITGNLFEVYLKPYFLEAYRPIRKGDIFLVRGGMRAVEFKVVETDPSPYCIVAPDTVIHCEGEPIKREDEEESLNEVGYDDIGGCRKQLAQIKEMVELPLRHPALFKAIGVKPPRGILLYGPPGTGKTLIARAVANETGAFFFLINGPEIMSKLAGESESNLRKAFEEAEKNAPAIIFIDELDAIAPKREKTHGEVERRIVSQLLTLMDGLKQRAHVIVMAATNRPNSIDPALRRFGRFDREVDIGIPDATGRLEILQIHTKNMKLADDVDLEQVANETHGHVGADLAALCSEAALQAIRKKMDLIDLEDETIDAEVMNSLAVTMDDFRWALSQSNPSALRETVVEVPQVTWEDIGGLEDVKRELQELVQYPVEHPDKFLKFGMTPSKGVLFYGPPGCGKTLLAKAIANECQANFISIKGPELLTMWFGESEANVREIFDKARQAAPCVLFFDELDSIAKARGGNIGDGGGAADRVINQILTEMDGMSTKKNVFIIGATNRPDIIDPAILRPGRLDQLIYIPLPDEKSRVAILKANLRKSPVAKDVDLEFLAKMTNGFSGADLTEICQRACKLAIRESIESEIRRERERQTNPSAMEVEEDDPVPEIRRDHFEEAMRFARRSVSDNDIRKYEMFAQTLQQSRGFGSFRFPSGNQGGAGPSQGSGGGTGGSVYTEDNDDDLYG

SEQ ID NO: 95 - protein, 526 aa, Artificial Sequence
  Synthetic Construct
MASNDYTQQATQSYGAYPTQPGQGYSQQSSQPYGQQSYSGYSQSTDTSGYGQSSYSSYGQSQNTGYGTQSTPQGYGSTGGYGSSQSSQSSYGQQSSYPGYGQQPAPSSTSGSYGSSSQSSSYGQPQSGSYSQQPSYGGQQQSYGQQQSYNPPQGYGQQNQYNSSSGGGGGGGGGGNYGQDQSSMSSGGGSGGGYGNQDQSGGGGSGGYGQQDRGGRGRGGSGGGGGGGGGGYNRSSGGYEPRGRGGGRGGRGGMGGSDRGGFNKFGGPRDQGSRHDSEQDNSDNNTIFVQGLGENVTIESVADYFKQIGIIKTNKKTGQPMINLYTDRETGKLKGEATVSFDDPPSAKAAIDWFDGKEFSGNPIKVSFATRRADFNRGGGNGRGGRGRGGPMGRGGYGGGGSGGGGRGGFPSGGGGGGGQQRAGDWKCPNPTCENMNFSWRNECNQCKAPKPDGPGGGPGGSHMGGNYGDDRRGGRGGYDRGGYRGRGGDRGGFRGGRGGGDRGGFGPGKMDSRGEHRQDRRERPY

SEQ ID NO: 96 - protein, 213 aa, Artificial Sequence
  Synthetic Construct
MASLFKKKTVDDVIKEQNRELRGTQRAIIRDRAALEKQEKQLELEIKKMAKIGNKEACKVLAKQLVHLRKQKTRTFAVSSKVTSMSTQTKVMNSQMKMAGAMSTTAKTMQAVNKKMDPQKTLQTMQNFQKENMKMEMTEEMINDTLDDIFDGSDDEEESQDIVNQVLDEIGIEISGKMAKAPSAARSLPSASTSKATISDEEIERQLKALGVD

SEQ ID NO: 97 - protein, 624 aa, Artificial Sequence
  Synthetic Construct
MAENGESSGPPRPSRGPAAAQGSAAAPAEPKIIKVTVKTPKEKEEFAVPENSSVQQFKEAISKRFKSQTDQLVLIFAGKILKDQDTLIQHGIHDGLTVHLVIKSQNRPQGQSTQPSNAAGTNTTSASTPRSNSTPISTNSNPFGLGSLGGLAGLSSLGLSSTNFSELQSQMQQQLMASPEMMIQIMENPFVQSMLSNPDLMRQLIMANPQMQQLIQRNPEISHLLNNPDIMRQTLEIARNPAMMQEMMRNQDLALSNLESIPGGYNALRRMYTDIQEPMLNAAQEQFGGNPFASVGSSSSSGEGTQPSRTENRDPLPNPWAPPPATQSSATTSTTTSTGSGSGNSSSNATGNTVAAANYVASIFSTPGMQSLLQQITENPQLIQNMLSAPYMRSMMQSLSQNPDLAAQMMLNSPLFTANPQLQEQMRPQLPAFLQQMQNPDTLSAMSNPRAMQALMQIQQGLQTLATEAPGLIPSFTPGVGVGVLGTAIGPVGPVTPIGPIGPIVPFTPIGPIGPIGPTGPAAPPGSTGSGGPTGPTVSSAAPSETTSPTSESGPNQQFIQQMVQALAGANAPQLPNPEVRFQQQLEQLNAMGFLNREANLQALIATGGDINAAIERLLGSQPS

SEQ ID NO: 98 - protein, 142 aa, Artificial Sequence
  Synthetic Construct
MPRGSRSAASRPASRPAAPSAHPPAHPPPSAAAPAPAPSGQPGLMAQMATTAAGVAVGSAVGHVMGSALTGAFSGGSSEPSQPAVQQAPTPAAPQPLQMGPCAYEIRQFLDCSTTQSDLSLCEGFSEALKQCKYYHGLSSLP

SEQ ID NO: 99 - protein, 211 aa, Artificial Sequence
  Synthetic Construct
MQAPHKEHLYKLLVIGDLGVGKTSIIKRYVHQNFSSHYRATIGVDFALKVLHWDPETVVRLQLWDIAGQERFGNMTRVYYREAMGAFIVFDVTRPATFEAVAKWKNDLDSKLSLPNGKPVSVVLLANKCDQGKDVLMNNGLKMDQFCKEHGFVGWFETSAKENINIDEASRCLVKHILANECDLMESIEPDVVKPHLTSTKVASCSGCAKS

SEQ ID NO: 100 - protein, 484 aa, Artificial Sequence [sequence omitted]

SEQ ID NO: 101 - protein, 531 aa, Artificial Sequence
  Synthetic Construct
MAALGCARLRWALRGAGRGLCPHGARAKAAIPAALPSDKATGAPGAGPGVRRRQRSLEEIPRLGQLRFFFQLFVQGYALQLHQLQVLYKAKYGPMWMSYLGPQMHVNLASAPLLEQVMRQEGKYPVRNDMELWKEHRDQHDLTYGPFTTEGHHWYQLRQALNQRLLKPAEAALYTDAFNEVIDDFMTRLDQLRAESASGNQVSDMAQLFYYFALEAICYILFEKRIGCLQRSIPEDTVTFVRSIGLMFQNSLYATFLPKWTRPVLPFWKRYLDGWNAIFSFGKKLIDEKLEDMEAQLQAAGPDGIQVSGYLHFLLASGQLSPREAMGSLPELLMAGVDTTSNTLTWALYHLSKDPEIQEALHEEVVGVVPAGQVPQHKDFAHMPLLKAVLKETLRLYPVVPTNSRIIEKEIEVDGFLFPKNTQFVFCHYVVSRDPTAFSEPESFQPHRWLRNSQPATPRIQHPFGSVPFGYGVRACLGRRIAELEMQLLLARLIQKYKVVLAPETGELKSVARIVLVPNKKVGLQFLQRQC

SEQ ID NO: 102 - protein, 455 aa, Artificial Sequence
  Synthetic Construct
MVDFPGYNLSGAVASFLFILLTMKQSEDFRVIGPAHPILAGVGEDALLTCQLLPKRTTMHVEVRWYRSEPSTPVFVHRDGVEVTEMQMEEYRGWVEWIENGIAKGNVALKIHNIQPSDNGQYWCHFQDGNYCGETSLLLKVAGLGSAPSIHMEGPGESGVQLVCTARGWFPEPQVYWEDIRGEKLLAVSEHRIQDKDGLFYAEATLVVRNASAESVSCLVHNPVLTEEKGSVISLPEKLQTELASLKVNGPSQPILVRVGEDIQLTCYLSPKANAQSMEVRWDRSHRYPAVHVYMDGDHVAGEQMAEYRGRTVLVSDAIDEGRLTLQILSARPSDDGQYRCLFEKDDVYQEASLDLKVVSLGSSPLITVEGQEDGEMQPMCSSDGWFPQPHVPWRDMEGKTIPSSSQALTQGSHGLFHVQTLLRVTNISAVDVTCSISIPFLGEEKIATFSLSGW

SEQ ID NO: 103 - protein, 230 aa, Artificial Sequence
  Synthetic Construct
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHVEHSISRSLLEGEIPFPPTSILLLLACIFLIKILAASALWAAAWHGQKPGTHPPSELDCGHDPGYQLQTLPGLRDT

SEQ ID NO: 104 - protein, 536 aa, Artificial Sequence
  Synthetic Construct
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 105 - protein, 317 aa, Artificial Sequence [sequence omitted]

SEQ ID NO: 106 - protein, 593 aa, Artificial Sequence
  Synthetic Construct
MWTLVSWVALTAGLVAGTRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLL